(19) United States
(12) Patent Application Publication
(10) Pub. No.: US 2016/0012749 A1
(43) Pub. Date: Jan. 14, 2016

(54) Eyewear System for Monitoring and Modifying Nutritional Intake

(71) Applicant: Robert A. Connor, Forest Lake, MN (US)

(72) Inventor: Robert A. Connor, Forest Lake, MN (US)

(21) Appl. No.: 14/330,649

(22) Filed: Jul. 14, 2014

(51) Int. Cl. G09B 19/00; A61N 2/02; A61N 2/00; G09B 5/00; A61M 21/00; A61M 31/00; A61F 5/00; A61B 5/11; A61B 5/00; A61B 5/04

(52) U.S. Cl. CPC G09B 19/0092; A61B 5/7285; A61N 2/02; A61N 2/006; A61B 5/04; A61M 21/00; A61M 31/002; A61F 5/0003; A61B 5/486; A61B 5/4866; A61B 5/11; G09B 5/00; A61M 2021/0016; A61M 2021/005

(57) ABSTRACT

This invention is an eyewear-based system and device for monitoring and modifying a person's nutritional intake. This invention can comprise eyewear with an imaging member which automatically records images of food when the person consumes food. These food images are automatically analyzed to estimate the type and quantity of food. This invention can also comprise a nutritional intake modification component which modifies the person's nutritional intake based on the type and quantity of food. This invention can reduce a person's nutritional intake of unhealthy types and/or quantities of food without reducing their nutritional intake of healthy types and/or quantities of food. It can serve as part of an overall system for better nutrition, weight management, and improved health.

### CROSS-REFERENCE TO RELATED APPLICATIONS

This patent application is: (a) a continuation in part of U.S. patent application Ser. No. 13/523,739 by Robert A. Connor entitled “The Willpower Watch™: A Wearable Food Consumption Monitor” filed on Jun. 14, 2012; and (b) also a continuation in part of U.S. patent application Ser. No. 13/797,955 by Robert A. Connor entitled “Device for Selectively Reducing Absorption of Unhealthy Food” filed on Mar. 12, 2013, which claimed the priority benefit of the priority benefit of U.S. Provisional Patent Application No. 61/729,494 by Robert A. Connor entitled “Device for Selectively Reducing Absorption of Unhealthy Food” filed on Nov. 23, 2012. The entire contents of these related applications are incorporated herein by reference.

### FEDERALLY SPONSORED RESEARCH

Not Applicable

### SEQUENCE LISTING OR PROGRAM

Not Applicable

### BACKGROUND

1. Field of Invention

This invention relates to energy balance, weight loss, and proper nutrition.

### Introduction to Energy Balance and Proper Nutrition

The United States population has some of the highest prevalence rates of obese and overweight people in the world. Further, these rates have increased dramatically during recent decades. In the late 1990's, around one in five Americans was obese. Today, that figure has increased to around one in three. It is estimated that around one in five American children is now obese. The prevalence of Americans who are generally overweight is estimated to be as high as two out of three.

This increase in the prevalence of Americans who are overweight or obese has become one of the most common causes of health problems in the United States. Potential adverse health effects from obesity include: cancer (especially endometrial, breast, prostate, and colon cancers); cardiovascular disease (including heart attack and arterial sclerosis); diabetes (type 2); digestive diseases; gallbladder disease; hypertension; kidney failure; obstructive sleep apnea; orthopedic complications; osteoarthritis; respiratory problems; stroke; metabolic syndrome (including hypertension, abnormal lipid levels, and high blood sugar); impairment of quality of life in general including stigma and discrimination; and even death.

There are estimated to be over a quarter-million obesity-related deaths each year in the United States. The tangible costs to American society of obesity have been estimated at over $100 billion dollars per year. This does not include the intangible costs of human pain and suffering. Despite the considerable effort that has been focused on developing new approaches for preventing and treating obesity, the problem is growing. There remains a serious unmet need for new ways to help people to moderate their consumption of unhealthy food, better manage their energy balance, and lose weight in a healthy and sustainable manner.

Obesity is a complex disorder with multiple interacting causal factors including genetic factors, environmental factors, and behavioral factors. A person's behavioral factors include the person's caloric intake (the types and quantities of food which the person consumes) and caloric expenditure (the calories that the person burns in regular activities and exercise). Energy balance is the net difference between caloric intake and caloric expenditure. Other factors being equal, energy balance surplus (caloric intake greater than caloric expenditure) causes weight gain and energy balance deficit (caloric intake less than caloric expenditure) causes weight loss.

Since many factors contribute to obesity, good approaches to weight management are comprehensive in nature. Proper nutrition and management of caloric intake are key parts of a comprehensive approach to weight management. Consumption of “junk food” that is high in simple sugars and saturated fats has increased dramatically during the past couple decades, particularly in the United States. This has contributed significantly to the obesity epidemic. For many people, relying on willpower and dieting is not sufficient to moderate their consumption of unhealthy “junk food.” The results are dire consequences for their health and well-being.

The invention that is disclosed herein directly addresses this problem by helping a person to monitor and modify their nutritional intake. The invention that is disclosed herein is an innovative technology that can be a key part of a comprehensive system that helps a person to reduce their consumption of unhealthy food, to better manage their energy balance, and to lose weight in a healthy and sustainable manner. In the following sections, we categorize and review the prior art, provide a summary of this invention and its advantages over the prior art, and then provide some detailed examples of how this invention can be embodied to help a person to improve their nutrition and to manage their weight.

2. Categorization and Review of the Prior Art

It can be challenging to classify prior art into discrete categories. This is the certainly the case in the field of energy balance, weight management, and proper nutrition. There are numerous examples of potentially-relevant prior art. However, classification of the prior art into categories, even if imperfect, is an invaluable tool for reviewing the prior art, identifying its limitations, and setting the stage for discussion of the advantages of the invention that is disclosed in subsequent sections. Towards this end, I now identify 50 general categories of prior art and discuss those categories which appear to be most relevant. The categories of prior art that are most relevant are marked as follows with an asterisk “*”. One of the original patent applications of which this present application is a continuation in part (and which is incorporated in its entirety by reference) lists examples in all 50 categories. This present application only discusses those categories which are most relevant. The reader can see examples for all categories in the original application if so desired.

The 50 categories of prior art are as follows: (1) little or no automated measurement of food consumption, (2) consumed manufactured compound or specifically-isolated natural substance, (3) substance sprinkled on food, (4) manually-ingested spray or pulse, (5) substance-emitting lipstick or toothpaste, (6) substance-emitting adhesive patch in the mouth, (7) dissolving film in mouth, (8) tablet or gum in mouth, (9) intraoral drug delivery, (10) motion guided or directed pill, (11) general implanted drug pump, (12) food purchasing monitoring or modification, (13) food scale, (14) portion size control, (15) mouth size or function modification, (16*) chewing and swallowing monitoring, (17*) hand and/or arm motion monitoring and modification (wrist), (18*) hand and/or arm motion monitoring and modification (utensil), (19*) utensil with sensor other than motion sensor, (20) other modification of eating speed, (21*) photo identification of food (bar code or other packaging-based code), (22*) photo identification of food (manual picture taking and identification), (23*) photo identification of food (manual picture taking and automated identification), (24*) photo identification of food (automated picture taking and identification), (25*) gastric band, (26*) gastric band with sensor, (27) gastrointestinal (GI) bypass and tissue plication, (28) pumping food out of the stomach through an intra-abdominal pathway, (29) gastric tube, (30) enzyme flow modification, (31*) gastrointestinal (GI) volume or pressure or flow modification, (32*) gastrointestinal (GI) volume or pressure or flow modification (with drug), (33) gastrointestinal (GI) sleeve or liner, (34) gastrointestinal (GI) sleeve or liner (with drug), (35*) electrical stimulation (general), (36*) electrical stimulation (with glucose sensor), (37*) electrical stimulation (with general sensor), (38*) electrical stimulation (with taste modification), (39*) electrical stimulation (with drug), (40*) electrical stimulation (with drug and sensor), (41) salivation stimulation, (42*) general sensor (glucose), (43*) general sensor (electromagnetic), (44*) general sensor (chemical), (45*) general sensor (microwave), (46*) sensor (intraoral), (47) sensor (general), (48) blood analysis, (49*) general energy balance feedback, and (50*) miscellaneous energy balance related.

### 16. Chewing and Swallowing Monitoring

Prior art in this category includes devices that monitor the chewing and/or swallowing actions that are associated with food consumption. In various examples, such devices can monitor chewing and/or swallowing by a method selected from the group consisting of: monitoring and analyzing sounds from a person's body to differentiate chewing and/or swallowing sounds from other sounds such as speaking; monitoring electromagnetic energy from a person's mouth muscles or internal gastrointestinal organs; and monitoring movement of a person's mouth or internal gastrointestinal organs.

Prior art in this category can be more automatic than art in many of the prior categories with respect to detecting when a person consumes food. Some art in this category can even generally differentiate between consumption of solid food vs. liquid food based on differences in sonic energy or electromagnetic energy. However, art in this category is generally very limited with respect to more-specific identification of what type of food a person is consuming. Also, a person can confuse or circumvent such a device by putting generally-solid food in a blender or by freezing generally-liquid food. Art in this category still relies on specific human actions to record food type apart from the actual action of eating. Also, since there can be different amounts of food per swallow, determination of food quantity based on the number of swallows can be problematic. Accordingly, prior art in this category has a number of limitations for measuring and modifying the types and quantities of food consumed.

Examples of prior art that appear to be best classified in this category include: U.S. Pat. No. 4,355,645 (Oct. 26, 1982 Mitani et al.) “Device for Displaying Masticatory Muscle Activities”, U.S. Pat. No. 5,067,488 (Nov. 26, 1991 Fukada et al.) “Mastication Detector and Measurement Apparatus and Method of Measuring Mastication”, U.S. Pat. No. 5,263,491 (Nov. 23, 1993 Thornton) “Ambulatory Metabolic Monitor”, U.S. Pat. No. 6,135,950 (Oct. 24, 2000 Adams) “E-fit Monitor”, U.S. Pat. No. 7,330,753 (Feb. 12, 2008 Policker et al.) “Analysis of Eating Habits”, U.S. Pat. No. 7,840,269 (Nov. 23, 2010 Policker et al.) “Analysis of Eating Habits”, U.S. Pat. No. 7,840,269 (Nov. 23, 2010 Policker et al.) “Analysis of Eating Habits”, and U.S. Pat. No. 7,914,468 (Mar. 29, 2011 Shalon et al.) “Systems and Methods for Monitoring and Modifying Behavior”; and U.S. patent applications 20040147816 (Jul. 29, 2004 Policker et al.) “Analysis of Eating Habits”, 20050283096 (Dec. 22, 2005 Chau et al.) “Apparatus and Method for Detecting Swallowing Activity”, 20060064037 (Mar. 23, 2006 Shalon et al.) “Systems and Methods for Monitoring and Modifying Behavior”, 20060064037 (Mar. 23, 2006 Shalon et al.) “Systems and Methods for Monitoring and Modifying Behavior”, 20060064037 (Mar. 23, 2006 Shalon et al.) “Systems and Methods for Monitoring and Modifying Behavior”, 20070299320 (Dec. 27, 2007 Policker et al.) “Analysis of Eating Habits”, 20070299320 (Dec. 27, 2007 Policker et al.) “Analysis of Eating Habits”, 20100076345 (Mar. 25, 2010 Soffer et al.) “Method, Device and System for Automatic Detection of Eating and Drinking”, 20110125063 (May 26, 2011 Shalon et al.) “Systems and Methods for Monitoring and Modifying Behavior”, 20110276312 (Nov. 10, 2011 Shalon et al.) “Device for Monitoring and Modifying Eating Behavior”, 20120101874 (Apr. 26, 2012 Ben-Haim et al.) “Charger With Data Transfer Capabilities”, and 20120203081 (Aug. 9, 2012 Leboeuf et al.) “Physiological and Environmental Monitoring Apparatus and Systems”. Another example of prior art that appears to be best classified in this category is WO 2002082968 (Policker) “Analysis of Eating Habits.”

17. Hand and/or Arm Motion Monitoring and Modification (Wrist)

This is the first of two categories of prior art wherein the intent is to detect and estimate food consumption by monitoring and analyzing hand and/or arm motion. This first category includes devices that are worn on a person's wrist or arm to directly monitor hand or arm motion. The second category (that follows this one) includes food utensils that indirectly monitor hand or arm motion when the utensil is held by a person and is used to bring food up to the person's mouth.

We have separated these devices into two categories because, even though they both monitor hand and arm motion, they have some different advantages and disadvantages. Devices worn on a person's wrist or arm have the advantage that they can be worn relatively continuously to monitor food consumption on a relatively continuous basis. Wrist-worn devices do not require that a person carry a specific motion-sensing food utensil everywhere that they go. However, a device that is worn on a person's wrist or arm can be subject to more false alarms (compared to a food utensil) due to non-food-consumption motions such as covering one's mouth when coughing, bringing a cigarette to one's mouth, or other hand-to-face gestures.

Many examples of devices in this category monitor hand and/or arm motion with an accelerometer. To the extent that there is a distinctive pattern of hand and/or arm movement associated with bringing food up to one's mouth, such a device can detect when food consumption is occurring. Such a device can also measure how rapidly or often the person brings their hand up to their mouth. A common use of such information is to encourage a person to eat at a slower pace. The idea that a person will eat less if they eat at a slower pace is based on the lag between food consumption and the feeling of satiety from internal gastric organs. If a person eats slower, then they will tend to not overeat past the point of internal identification of satiety. Detection of food consumption and approximate measurement of food consumption quantity that is based on hand or arm motion can also be useful for purposes other than slowing the pace of eating.

However, there are significant limitations to devices and methods in this category. First, such devices and methods do not provide good information concerning the types of food consumed. In this respect, they generally still rely on manual food identification methods. Second, although progress has been made to differentiate hand and/or arm motions that indicate food consumption from other types of hand and/or arm motions (such as covering one's mouth or brushing one's teeth), there remains imprecision with respect to quantification of food consumed based on analysis of hand-to-mouth movements. Third, it is tough to make such devices and methods tamper-resistant. A person can use non-conventional hand movements to eat, use a non-monitored hand to eat, eat larger bite sizes with each hand movement, remove the device from their wrist, or just ignore feedback from the device when they eat.

Examples of prior art that appear to be best classified in this category include: U.S. Pat. No. 3,885,576 (May 27, 1975 Symmes) “Wrist Band Including a Mercury Switch to Induce an Electric Shock”, U.S. Pat. No. 4,965,553 (Oct. 23, 1990 DelBiondo et al.) “Hand-Near-Mouth Warning Device”, U.S. Pat. No. 5,424,719 (Jun. 13, 1995 Ravid) “Consumption Control”, U.S. Pat. No. 5,563,850 (Oct. 8, 1996 Hanapole) “Food Intake Timer”, U.S. Pat. No. 8,112,281 (Feb. 7, 2012 Yeung et al.) “Accelerometer-Based Control of Wearable Audio Recorders”, and U.S. Pat. No. 8,310,368 (Nov. 13, 2012 Hoover et al.) “Weight Control Device”; and U.S. patent applications 20060197670 (Sep. 7, 2006 Breibart) “Method and Associated Device for Personal Weight Control”, 20080137486 (Jun. 12, 2008 Czarenk et al.) “Diet Watch”, and 20100194573 (Aug. 5, 2010 Hoover et al.) “Weight Control Device”.

18. Hand and/or Arm Motion Monitoring and Modification (Utensil)

Prior art in this category includes hand-held food serving utensils (such as forks or spoons) that indirectly monitor hand and/or arm motion to detect and estimate food consumption. Compared to the wrist-worn motion-detection devices that were discussed in the previous category, motion-detecting utensils can be less subject to false alarms because they are only used when the person consumes food. There are some recent examples of sophisticated food-analyzing utensils with sensors other than motion-sensors. Since they are qualitatively different than utensils with only motion sensors, we have put these more-sophisticated food-analyzing utensils in a separate category that follows in this categorization scheme.

Many examples of utensils in this category monitor motion with an accelerometer. Since the utensil is only used for food consumption, analysis of complex motion and differentiation of food consumption actions vs. other hand gestures is less important with a utensil than it is with a wrist-mounted device. Accordingly, some of the utensils in this category are quite simple. In the extreme, although crude, a single-axis accelerometer can be used. Other simple methods of measuring hand-to-mouth movement by a utensil are based on a simple holder or button on which the utensil is placed between mouthfuls. Another simple method is an internal fluid “horizontal level” or “lava lamp” feature attached to the utensil that is used to regulate the timing of hand-to-mouth motions.

The idea is that a person will eat less if they eat slower because there can be a lag between food consumption and identification of satiety by internal organs. If the person eats slower, then they will tend to not overeat past the point of internal identification of satiety. Detection of food consumption and approximate measurement of food consumption quantity based on hand or arm motion can also be useful for purposes other than slowing the pace of eating.

However, utensils with just a motion sensor do not provide good information concerning the type of food consumed. Also, compliance can be a huge issue for this approach. In order to be successful, a person has to bring the special utensil with them constantly and use it consistently whenever they eat. What happens when they are eating out in a social setting or eating a snack with their hands? For these reasons, special eating utensils with just a motion sensor are limited in their ability to consistently monitor and modify a person's food consumption.

Examples of prior art that appear to be best classified in this category include: U.S. Pat. No. 4,207,673 (Jun. 17, 1980 DiGirolamo et al.) “Cuttlery”, U.S. Pat. No. 4,914,819 (Apr. 10, 1990 Ash) “Eating Utensil for Indicating When Food May be Eaten Therewith and a Method for Using the Utensil”, U.S. Pat. No. 4,975,682 (Dec. 4, 1990 Kerr et al.) “Meal Minder Device”, U.S. Pat. No. 5,299,356 (Apr. 5, 1994 Maxwell) “Diet Eating Utensil”, U.S. Pat. No. 5,421,089 (Jun. 6, 1995 Dubus et al.) “Fork with Timer”, and U.S. Pat. No. 8,299,930 (Oct. 30, 2012 Schmid-Schonbein et al.) “Devices, Systems and Methods to Control Caloric Intake”; and U.S. patent applications 20070098856 (May 3, 2007 LePine) “Mealtime Eating Regulation Device”, 20080276461 (Nov. 13, 2008 Gold) “Eating Utensil Capable of Automatic Bite Counting”, 20090253105 (Oct. 8, 2009 Lepine) “Device for Regulating Eating by Measuring Potential”, 20100109876 (May 6, 2010 Schmid-Schonbein et al.) “Devices, Systems and Methods to Control Caloric Intake”, 20100240962 (Sep. 23, 2010 Contant) “Eating Utensil to Monitor and Regulate Dietary Intake”, and 20120115111 (May 10, 2012 Lepine) “Mealtime Eating Regulation Device”.

19. Utensil with Sensor Other than Motion Sensor

Prior art in this category includes food utensils with sensors other than motion sensors that are used to measure food consumption. Such art in this category is relatively innovative and there are relatively few examples to date. Prior art in this category represents an important step toward automated measurement of food consumption. In various examples, a utensil in this category can measure the volume, mass, density, or general composition of a bite-size portion of food that is transported by the utensil to a person's mouth.

However, a significant limitation of art in this category is that it relies on a person's compliance. The person must use the utensil each time that they eat anything in order for the system to successfully monitor food consumption. If a person eats food without using the utensil (e.g. when dining in a social setting or when eating a snack by hand), then the system is unaware of this food consumption. This can be problematic and the prior art does not offer a solution to this problem.

Examples of prior art that appear to be best classified in this category include: U.S. Pat. No. 8,229,676 (Jul. 24, 2012 Hyde et al.) “Food Content Detector”, U.S. Pat. No. 8,285,488 (Oct. 9, 2012 Hyde et al.) ibid., U.S. Pat. No. 8,290,712 (Oct. 16, 2012 Hyde et al.) ibid., U.S. Pat. No. 8,321,141 (Nov. 27, 2012 Hyde et al.) ibid., and U.S. Pat. No. 8,355,875 (Jan. 15, 2013 Hyde et al.) ibid.; and U.S. patent applications 20100125176 (May 20, 2010 Hyde et al.) ibid., 20100125177 (May 20, 2010 Hyde et al.) ibid., 20100125178 (May 20, 2010 Hyde et al.) ibid., 20100125179 (May 20, 2010 Hyde et al.) ibid., 20100125180 (May 20, 2010 Hyde et al.) ibid., 20100125181 (May 20, 2010 Hyde et al.) ibid., 20100125417 (May 20, 2010 Hyde et al.) ibid., 20100125418 (May 20, 2010 Hyde et al.) ibid., 20100125419 (May 20, 2010 Hyde et al.) ibid., 20100125420 (May 20, 2010 Hyde et al.) ibid., and 20110184247 (Jul. 28, 2011 Contant et al.) “Comprehensive Management of Human Health”.

### 21. Photo Identification of Food (Bar Code or Other Packaging-Based Code)

Prior art in this category includes devices and methods for identifying food consumption based on photo identification of food using bar codes or other packaging-based codes. If consumed food has a bar code (or other packaging-based code) then it is relatively easy for a system to associate specific nutrients and/or total calories with that food.

However, there are several limitations to this approach. First, a person may eat food that is not identified by bar codes or other packaging-based codes. Food served in restaurants or in other people's homes is unlikely to be identified by such codes. Also, even in a grocery store, not all food is identified by such codes. Second, a person may not eat all of the food that is identified by such codes. Other people may eat some of the food in a given package. Also, some of the food in a given package may be thrown out.

Also, depending on the longevity of the food, some food in a given package may be eaten soon after purchase and the rest may be eaten long afterwards. Accordingly, it can be problematic using such codes to make associations between food eaten by a specific person in a specific time period and the person's success in achieving weight management goals during that time period.

Examples of prior art that appear to be best classified in this category include: U.S. Pat. No. 5,819,735 (Oct. 13, 1998 Mansfield et al.) “Device and Method for Monitoring Dietary Intake of Calories And Nutrients” and U.S. Pat. No. 6,283,914 (Sep. 4, 2001 Mansfield et al.) “Device and Method for Monitoring Dietary Intake of Calories and Nutrients”; and U.S. patent applications 20030163354 (Aug. 28, 2003 Shamoun) “Device for Collecting and Analyzing Nutritional Data and Method Therefor”, 20030208110 (Nov. 6, 2003 Mault et al.) “Physiological Monitoring using Wrist-Mounted Device”, 20060189853 (Aug. 24, 2006 Brown) “Method and System for Improving Adherence with a Diet Program or Other Medical Regimen”, 20060229504 (Oct. 12, 2006 Johnson) “Methods and Systems for Lifestyle Management”, 20070059672 (Mar. 15, 2007 Shaw) “Nutrition Tracking Systems and Methods”, and 20090176526 (Jul. 9, 2009 Altman) “Longitudinal Personal Health Management System Using Mobile Data Capture”.

### 22. Photo Identification of Food (Manual Picture Taking and Identification)

Prior art in this category includes image-based devices and methods that require specific voluntary human action associated with each food consumption event (apart from the actual act of eating) in order: to take pictures of food during food consumption; and to identify the types and quantities of food consumed based on those pictures. In this category, neither picture taking nor food identification is automated. In an example, such art can include having a person aim a camera-equipped mobile electronic device toward food each time that the person eats and requiring that the person identify the type and quantity of food consumed based on the resulting pictures.

In an example, food identification by a person can occur in real-time (before, during, or immediately after a meal) using voice recognition or a menu-driven user interface. In another example, food identification by a person can occur later, long after the meal. In an example, food identification can be done by the person whose food consumption is being monitored and measured. In an example, food identification can be done by someone else.

Such image-based food logging systems are an improvement over recording food consumed with a pencil and paper. However, these devices and systems still require manual intervention to aim an imaging device toward a food source and to take at least one picture each time that the person eats something. Accordingly, they depend heavily on the person's compliance. These devices and methods can be time-consuming (having to aim the field of vision toward food), easy to circumvent (a person may simply not take pictures of some food consumed), and embarrassing to use social dining situations. This can lead to low long-term compliance.

Any approach that depends on voluntary human action each time that a person eats anything is difficult to make tamper-resistant. It is very easy for someone to “cheat” by simply not taking pictures of some consumed food items. Also, even if the person does consistently takes pictures of every meal or snack that they eat, then they may be tempted to postpone the manual task of food identification for hours or days after a meal has occurred. This can cause inaccuracy. How many chips were left in that bag in the picture? Is that a “before” or “after” picture of that gallon of ice cream? Delays in food identification can lead to imprecision in identification of the types and quantities of food consumed.

Examples of prior art that appear to be best classified in this category include U.S. patent applications: 20020047867 (Apr. 25, 2002 Mault et al.) “Image Based Diet Logging”, 20020109600 (Aug. 15, 2002 Mault et al.) “Body Supported Activity and Condition Monitor”, 20070030339 (Feb. 8, 2007 Findlay et al.) “Method, System and Software for Monitoring Compliance”, 20090112800 (Apr. 30, 2009 Athsani) “System and Method for Visual Contextual Search”, and 20090219159 (Sep. 3, 2009 Morgenstern) “Method and System for an Electronic Personal Trainer”.

### 23. Photo Identification of Food (Manual Picture Taking and Automatic Identification)

Prior art in this category includes image-based devices and methods that require specific voluntary human actions associated with each food consumption event (apart from the actual act of eating) in order to take pictures of food during consumption. However, these devices and methods automatically identify the types and quantities of food consumed based on these pictures. In various examples, automatic identification of food types and quantities can be based on: color and texture analysis; image segmentation; image pattern recognition; volumetric analysis based on a fiduciary market or other object of known size; and/or three-dimensional modeling based on pictures from multiple perspectives. In an example, food identification can occur before or during a meal. In an example, a mobile phone application can transmit images to a remote location where automatic food identification occurs.

In some examples, food identification is an interactive process that combines automatic and manual methods of food identification. In this category, picture taking is not automated. In an example, such art can include having a person aim a camera-equipped mobile electronic device toward food to take pictures every time that the person eats food.

Such image-based consumption monitoring systems are useful, but still require specific actions by the person to aim an imaging device toward a food source and to take at least one picture of food each time that the person eats something. Accordingly, such art depends on the person's compliance. Such devices and methods can be time-consuming, easy to circumvent, and embarrassing in social dining situations. Any approach that depends on voluntary human action each time that a person eats anything is difficult to make tamper-resistant. It is very easy for someone to eat something without first taking a picture of it.

Examples of prior art that appear to be best classified in this category include: U.S. Pat. No. 6,513,532 (Feb. 4, 2003 Mault et al.) “Diet and Activity Monitoring Device”, U.S. Pat. No. 8,345,930 (Jan. 1, 2013 Tamrakar et al.) “Method for Computing Food Volume in a Method for Analyzing Food”, and U.S. Pat. No. 8,363,913 (Jan. 29, 2013 Boushey et al.) “Dietary Assessment System and Method”; and U.S. patent applications 20010049470 (Dec. 6, 2001 Mault et al.) “Diet and Activity Monitoring Device”, 20020027164 (Mar. 7, 2002 Mault et al.) “Portable Computing Apparatus Particularly Useful in a Weight Management Program”, 20030065257 (Apr. 3, 2003 Mault et al.) “Diet and Activity Monitoring Device”, 20030076983 (Apr. 24, 2003 Cox) “Personal Food Analyzer”, 20080267444 (Oct. 30, 2008 Simons-Nikolova) “Modifying a Person's Eating and Activity Habits”, 20100111383 (May 6, 2010 Boushey et al.) “Dietary Assessment System and Method”, 20100173269 (Jul. 8, 2010 Puri et al.) “Food Recognition Using Visual Analysis and Speech Recognition”, 20100191155 (Jul. 29, 2010 Kim et al.) “Apparatus for Calculating Calories Balance by Classifying User's Activity”, 20100332571 (Dec. 30, 2010 Healey et al.) “Device Augmented Food Identification”, 20110182477 (Jul. 28, 2011 Tamrakar et al.) “Method for Computing Food Volume in a Method for Analyzing Food”, 20110318717 (Dec. 29, 2011 Adamowicz) “Personalized Food Identification and Nutrition Guidance System”, 20120170801 (Jul. 5, 2012 De Oliveira et al.) “System for Food Recognition Method Using Portable Devices Having Digital Cameras”, 20120179665 (Jul. 12, 2012 Baarman et al.) “Health Monitoring System”, 20120313776 (Dec. 13, 2012 Utter) “General Health and Wellness Management Method and Apparatus for a Wellness Application Using Data from a Data-Capable Band”, 20120326873 (Dec. 27, 2012 Utter) “Activity Attainment Method and Apparatus for a Wellness Application Using Data from a Data-Capable Band”, and 20130004923 (Jan. 3, 2013 Utter) “Nutrition Management Method and Apparatus for a Wellness Application Using Data from a Data-Capable Band”.

### 24. Photo Identification of Food (Automatic Picture Taking and Identification)

Prior art in this category includes image-based devices and methods that automatically take and analyze pictures of food in order to identify the types and quantities of food consumed without the need for specific human action associated with each food consumption event (apart from the actual act of eating). In an example, automatic picture taking can occur using a camera that the person wears continually. In an example, a wearable camera can take pictures continually. In various examples, automatic identification of food types and quantities can be based on: color and texture analysis; image segmentation; image pattern recognition; volumetric analysis based on a fiduciary market or other object of known size; and/or three-dimensional modeling based on pictures from multiple perspectives. As an advantage over freestanding mobile imaging devices, wearable imaging devices offer a higher degree of automation.

Although art in this category is an innovative advance in the field, it still has at least three significant limitations that have not been fully addressed by the prior art. First, there is a trade-off between the measurement advantages of a continually-imaging wearable camera and the potential intrusion into a person's privacy. How can one achieve the measurement advantages of the wearable-imaging approach to food consumption monitoring with minimal intrusion into a person's privacy? Second, how does one address the possibility that a person can tamper with, or circumvent, such a device? Prior art in this category does not offer a tamper-resistant device.

Third, there are limitations to how accurately an image-based system can identify the composition of food. For example, many types of food, especially liquids, look similar. For example, if a beverage is not consumed in its original container, how can an image-based system know whether the beverage is high sugar vs. low sugar, or unhealthy vs. healthy? What is that sandwiched between two buns in a burger? Is it beef or turkey or a “veggie burger”? For these reasons, even though image-based prior art in this category is innovative and useful, there remains a need for better methods for automatically measuring the types and quantities of food consumption.

Examples of prior art that appear to be best classified in this category include U.S. Pat. No. 6,508,762 (Jan. 21, 2003 Karnieli) “Method for Monitoring Food Intake” and patent applications 20020022774 (Feb. 21, 2002 Karnieli) “Method for Monitoring Food Intake”, and 20090012433 (Jan. 8, 2009 Fernstrom et al.) “Method, Apparatus and System for Food Intake and Physical Activity Assessment”.

### 25. Gastric Band

With this category, we now move from devices and methods that are primarily used externally to the human body to devices and methods that are primarily implanted within the human body. Prior art in this particular category includes implantable devices that externally constrain the cross-sectional size of a member of a person's gastrointestinal tract (such as their stomach) to constrain the volume or amount of food that a person consumes. In an example, art in this category includes gastric bands that externally encircle and constrain expansion of the upper portion of a person's stomach in order to limit the volume or amount of food that passes into the person's stomach. Many of the devices in this category are adjustable in size, allowing post-operative adjustment of the external circumference of the portion of the gastrointestinal organ which the device encircles. We have separated out such devices which include sensors (and can self-adjust) in a category following this one.

Although devices in this category are innovative and have benefited many people, such devices still have limitations. First, such devices in the prior art are relatively food blind. They blindly reduce intake of all types of food. The prior art does not specify how they could be used to selectively reduce intake of unhealthy food while allowing normal consumption of healthy food. Second, such devices can irritate or harm the tissue of the gastrointestinal organ which they encircle. Third, although such devices can limit the size and flow of food entering a person's stomach, such devices do not limit the overall quantity of food that enters a person's stomach over time. For example, if a person wishes to melt an entire gallon of ice cream and then ingest it, a gastric band will not prevent this. There remains a need for better approaches for selectively modifying a person's food consumption.

Examples of prior art that appear to be best classified in this category include: U.S. Pat. No. 6,547,801 (Apr. 15, 2003 Dargent et al.) “Gastric Constriction Device”, U.S. Pat. No. 6,551,235 (Apr. 22, 2003 Forsell) “Implantable Pump”, U.S. Pat. No. 6,966,875 (Nov. 22, 2005 Longobardi) “Adjustable Gastric Implant”, U.S. Pat. No. 7,775,967 (Aug. 17, 2010 Gertner) “Closed Loop Gastric Restriction Devices and Methods”, U.S. Pat. No. 7,798,954 (Sep. 21, 2010 Birk et al.) “Hydraulic Gastric Band with Collapsible Reservoir”, U.S. Pat. No. 7,909,754 (Mar. 22, 2011 Hassler et al.) “Non-Invasive Measurement of Fluid Pressure in an Adjustable Gastric Band”, U.S. Pat. No. 7,972,346 (Jul. 5, 2011 Bachmann et al.) “Telemetrically Controlled Band for Regulating Functioning of a Body Organ or Duct, and Methods of Making, Implantation And Use”, U.S. Pat. No. 8,034,065 (Oct. 11, 2011 Coe et al.) “Controlling Pressure in Adjustable Restriction Devices”, U.S. Pat. No. 8,043,206 (Oct. 25, 2011 Birk) “Self-Regulating Gastric Band with Pressure Data Processing”, U.S. Pat. No. 8,100,870 (Jan. 24, 2012 Marcotte et al.) “Adjustable Height Gastric Restriction Devices and Methods”, U.S. Pat. No. 8,137,261 (Mar. 20, 2012 Kierath et al.) “Device for the Treatment of Obesity”, U.S. Pat. No. 8,292,800 (Oct. 23, 2012 Stone et al.) “Implantable Pump System”, U.S. Pat. No. 8,317,677 (Nov. 27, 2012 Bertolote et al.) “Mechanical Gastric Band with Cushions”, and U.S. Pat. No. 8,323,180 (Dec. 4, 2012 Birk et al.) “Hydraulic Gastric Band with Collapsible Reservoir”; and U.S. patent applications 20070156013 (Jul. 5, 2007 Birk) “Self-Regulating Gastric Band with Pressure Data Processing”, 20070265645 (Nov. 15, 2007 Birk et al.) “Hydraulic Gastric Band Collapsible Reservoir”, 20070265646 (Nov. 15, 2007 Mccoy et al.) “Dynamically Adjustable Gastric Implants”, and 20080275484 (Nov. 6, 2008 Gertner) “Per Os Placement of Extragastric Devices”.

Examples of prior art that appear to be best classified in this category also include U.S. patent applications: 20090157106 (Jun. 18, 2009 Marcotte et al.) “Adjustable Height Gastric Restriction Devices and Methods”, 20090171375 (Jul. 2, 2009 Coe et al.) “Controlling Pressure in Adjustable Restriction Devices”, 20090204131 (Aug. 13, 2009 Ortiz et al.) “Automatically Adjusting Band System with MEMS Pump”, 20090204132 (Aug. 13, 2009 Ortiz et al.) “Automatically Adjusting Band System”, 20090216255 (Aug. 27, 2009 Coe et al.) “Controlling Pressure in Adjustable Restriction Devices”, 20090270904 (Oct. 29, 2009 Birk et al.) “Remotely Adjustable Gastric Banding System”, 20090312785 (Dec. 17, 2009 Stone et al.) “Implantable Pump System”, 20100228080 (Sep. 9, 2010 Tavori et al.) “Apparatus and Methods for Corrective Guidance of Eating Behavior after Weight Loss Surgery”, 20100234682 (Sep. 16, 2010 Gertner) “Closed Loop Gastric Restriction Devices and Methods”, 20100324358 (Dec. 23, 2010 Birk et al.) “Hydraulic Gastric Band with Collapsible Reservoir”, 20110130626 (Jun. 2, 2011 Hassler et al.) “Non-Invasive Measurement of Fluid Pressure in an Adjustable Gastric Band”, 20110184229 (Jul. 28, 2011 Raven et al.) “Laparoscopic Gastric Band with Active Agents”, 20110201874 (Aug. 18, 2011 Birk et al.) “Remotely Adjustable Gastric Banding System”, 20110207994 (Aug. 25, 2011 Burrell et al.) “Methods and Devices for Treating Morbid Obesity Using Hydrogel”, 20110207995 (Aug. 25, 2011 Snow et al.) “Inductively Powered Remotely Adjustable Gastric Banding System”, 20110208216 (Aug. 25, 2011 Fobi et al.) “Gastric Bypass Band and Surgical Method”, and 20110270025 (Nov. 3, 2011 Fridez et al.) “Remotely Powered Remotely Adjustable Gastric Band System”.

Examples of prior art that appear to be best classified in this category also include U.S. patent applications: 20110270030 (Nov. 3, 2011 Birk et al.) “Hydraulic Gastric Band with Collapsible Reservoir”, 20110275887 (Nov. 10, 2011 Birk) “Self-Regulating Gastric Band with Pressure Data Processing”, 20110306824 (Dec. 15, 2011 Perron et al.) “Remotely Adjustable Gastric Banding System”, 20110313240 (Dec. 22, 2011 Phillips et al.) “Flow Restrictor and Method for Automatically Controlling Pressure for a Gastric Band”, 20120046674 (Feb. 23, 2012 Augarten et al.) “Power Regulated Implant”, 20120059216 (Mar. 8, 2012 Perron) “Remotely Adjustable Gastric Banding System”, 20120067937 (Mar. 22, 2012 Menzel) “Internal Gastric Bander for Obesity”, 20120083650 (Apr. 5, 2012 Raven) “Systems and Methods for Adjusting Gastric Band Pressure”, 20120088962 (Apr. 12, 2012 Franklin et al.) “Self-Adjusting Gastric Band”, 20120095288 (Apr. 19, 2012 Snow et al.) “Self-Adjusting Gastric Band”, 20120130273 (May 24, 2012 Hassler et al.) “Non-Invasive Measurement of Fluid Pressure in an Adjustable Gastric Band”, 20120190919 (Jul. 26, 2012 Phillips et al.) “Assembly and Method for Automatically Controlling Pressure for a Gastric Band”, 20120197069 (Aug. 2, 2012 Lau et al.) “Assembly and Method for Automatically Controlling Pressure for a Gastric Band”, 20120215061 (Aug. 23, 2012 Fridez et al.) “Hydraulic Gastric Band with Reversible Self-Opening Mechanism”, 20120215062 (Aug. 23, 2012 Coe) “Remotely Adjustable Gastric Banding Device”, 20120296157 (Nov. 22, 2012 Tozzi et al.) “Medical Device Comprising an Artificial Contractile Structure”, and 20120302936 (Nov. 29, 2012 Belhe et al.) “External Anchoring Configurations for Modular Gastrointestinal Prostheses”.

26. Gastric Band with Sensor

Prior art in this category is similar to that of the previous category except for the addition of a sensor and the possibility of self-adjusting operation. The vast majority of sensors in this category are pressure sensors. The addition of a pressure sensor to a gastric band enables remote or automatic adjustment of the size of the constraining band in response to pressure from the external circumference of the encircled gastrointestinal organ. This can help to reduce irritation or harm of organ tissue by a constraining band, can enable post-operative refinement of therapy, and can help to reduce undesirable regurgitation. However, the other limitations that were identified with respect to gastric bands in the above category are still generally applicable to gastric bands in this category.

Examples of prior art that appear to be best classified in this category include: U.S. Pat. No. 7,775,966 (Aug. 17, 2010 Dlugos et al.) “Non-Invasive Pressure Measurement in a Fluid Adjustable Restrictive Device”, U.S. Pat. No. 7,879,068 (Feb. 1, 2011 Dlugos et al.) “Feedback Sensing for a Mechanical Restrictive Device”, U.S. Pat. No. 8,251,888 (Aug. 28, 2012 Roslin et al.) “Artificial Gastric Valve”, and U.S. Pat. No. 8,308,630 (Nov. 13, 2012 Birk et al.) “Hydraulic Gastric Band with Collapsible Reservoir”; and U.S. patent applications 20060173238 (Aug. 3, 2006 Starkebaum) “Dynamically Controlled Gastric Occlusion Device”, 20060199997 (Sep. 7, 2006 Hassler et al.) “Monitoring of a Food Intake Restriction Device”, 20060235448 (Oct. 19, 2006 Roslin et al.) “Artificial Gastric Valve”, 20080172072 (Jul. 17, 2008 Pool et al.) “Internal Sensors for Use with Gastric Restriction Devices”, 20090192534 (Jul. 30, 2009 Ortiz et al.) “Sensor Trigger”, 20100152532 (Jun. 17, 2010 Marcotte) “Gastric Band System with Esophageal Sensor”, 20100274274 (Oct. 28, 2010 Roslin et al.) “Artificial Gastric Valve”, 20110034760 (Feb. 10, 2011 Brynelsen et al.) “Feedback Systems and Methods to Enhance Obstructive and Other Obesity Treatments”, 20110245598 (Oct. 6, 2011 Gertner) “Closed Loop Gastric Restriction Devices and Methods”, and 20120108921 (May 3, 2012 Raven et al.) “Gastric Banding System Adjustment Based on a Satiety Agent Concentration Level”.

### 31. Gastrointestinal (GI) Volume or Pressure or Flow Modification

This relatively-broad category of prior art includes various devices that modify the interior volume of a gastrointestinal organ (such as the stomach), interior wall pressure of a gastrointestinal organ (such as the stomach), and/or food flow through a valve in a gastro-intestinal organ (such as the pyloric valve in the stomach). In various examples, art in this category can: occupy some of the interior volume of a gastrointestinal organ (such as an expandable gastric balloon in the stomach); apply pressure to the interior walls of a gastrointestinal organ (such as an expandable stomach stent); or mechanically modify the operation of a gastrointestinal valve (such as the operation of the pyloric valve within the stomach).

In an example, reducing the available space for food to occupy within the stomach can reduce the amount of food consumed and/or cause an earlier sensation of fullness. In an example, applying pressure to the interior walls of the stomach can cause an earlier sensation of fullness and reduce the amount of food consumed. In an example, reducing the outflow of food from the stomach by modifying the operation of the pyloric valve can lead to an earlier sensation of fullness and reduce food consumed.

However, there can be limitations to such devices. For example, the stomach can stretch even further when a balloon is implanted inside it or a stent is expanded within it, thwarting efforts to cause an earlier sensation of fullness or reduce food consumption. Also, even if a temporary balloon or stent is effective while implanted, that effect can be lost (or reversed) when the temporary balloon or stent is removed. In a worst case scenario, such a device can make the person worse off. After removal of a balloon or stent, a stretched stomach can accommodate even more food than normal, causing the person to eat more than ever in the long run.

Examples of prior art that appear to be best classified in this category include U.S. patents: U.S. Pat. No. 4,133,315 (Jan. 9, 1979 Berman et al.) “Method and Apparatus for Reducing Obesity”, U.S. Pat. No. 4,416,267 (Nov. 22, 1983 Garren et al.) “Method and Apparatus for Treating Obesity”, U.S. Pat. No. 4,592,339 (Jun. 3, 1986 Kuzmak et al.) “Gastric Banding Device”, U.S. Pat. No. 4,694,827 (Sep. 22, 1987 Weiner et al.) “Inflatable Gastric Device for Treating Obesity and Method of Using the Same”, U.S. Pat. No. 5,074,868 (Dec. 24, 1991 Kuzmak) “Reversible Stoma-Adjustable Gastric Band”, U.S. Pat. No. 5,226,429 (Jul. 13, 1993 Kuzmak) “Laparoscopic Gastric Band and Method”, U.S. Pat. No. 5,234,454 (Aug. 10, 1993 Bangs) “Percutaneous Intragastric Balloon Catheter and Method for Controlling Body Weight Therewith”, U.S. Pat. No. 5,259,399 (Nov. 9, 1993 Brown) “Device and Method of Causing Weight Loss Using Removable Variable Volume Intragastric Bladder”, U.S. Pat. No. 5,449,368 (Sep. 12, 1995 Kuzmak) “Laparoscopic Adjustable Gastric Banding Device and Method for Implantation and Removal Thereof”, U.S. Pat. No. 5,601,604 (Feb. 11, 1997 Vincent) “Universal Gastric Band”, U.S. Pat. No. 5,868,141 (Feb. 9, 1999 Ellias) “Endoscopic Stomach Insert for Treating Obesity and Method for Use”, U.S. Pat. No. 5,993,473 (Nov. 30, 1999 Chan et al.) “Expandable Body Device for the Gastric Cavity and Method”, U.S. Pat. No. 6,067,991 (May 30, 2000 Forsell) “Mechanical Food Intake Restriction Device”, U.S. Pat. No. 6,454,785 (Sep. 24, 2002 De Hoyos Garza) “Percutaneous Intragastric Balloon Catheter for the Treatment Of Obesity”, U.S. Pat. No. 6,579,301 (Jun. 17, 2003 Bales et al.) “Intragastric Balloon Device Adapted to be Repeatedly Varied in Volume Without External Assistance”, U.S. Pat. No. 6,675,809 (Jan. 13, 2004 Stack et al.) “Satiation Devices and Methods”, U.S. Pat. No. 6,733,512 (May 11, 2004 Mcghan) “Self-Deflating Intragastric Balloon”, U.S. Pat. No. 6,981,980 (Jan. 3, 2006 Sampson et al.) “Self-Inflating Intragastric Volume-Occupying Device”, U.S. Pat. No. 7,033,373 (Apr. 25, 2006 DeLaTorre et al.) “Method and Device for Use in Minimally Invasive Placement of Space-Occupying Intragastric Devices”, U.S. Pat. No. 7,066,945 (Jun. 27, 2006 Hashiba et al.) “Intragastric Device for Treating Obesity”, and U.S. Pat. No. 7,112,186 (Sep. 26, 2006 Shah) “Gastro-Occlusive Device”.

Examples of prior art that appear to be best classified in this category also include U.S. patents: U.S. Pat. No. 7,354,454 (Apr. 8, 2008 Stack et al.) “Satiation Devices and Methods”, U.S. Pat. No. 7,470,251 (Dec. 30, 2008 Shah) “Gastro-Occlusive Device”, U.S. Pat. No. 7,682,306 (Mar. 23, 2010 Shah) “Therapeutic Intervention Systems Employing Implantable Balloon Devices”, U.S. Pat. No. 7,699,863 (Apr. 20, 2010 Marco et al.) “Bioerodible Self-Deployable Intragastric Implants”, U.S. Pat. No. 7,717,843 (May 18, 2010 Balbierz et al.) “Restrictive and/or Obstructive Implant for Inducing Weight Loss”, U.S. Pat. No. 7,758,493 (Jul. 20, 2010 Gingras) “Gastric Constriction Device”, U.S. Pat. No. 7,771,382 (Aug. 10, 2010 Levine et al.) “Resistive Anti-Obesity Devices”, U.S. Pat. No. 7,785,291 (Aug. 31, 2010 Marco et al.) “Bioerodible Self-Deployable Intragastric Implants”, U.S. Pat. No. 7,841,978 (Nov. 30, 2010 Gertner) “Methods and Devices for to Treatment of Obesity”, U.S. Pat. No. 7,963,907 (Jun. 21, 2011 Gertner) “Closed Loop Gastric Restriction Devices and Methods”, U.S. Pat. No. 8,001,974 (Aug. 23, 2011 Makower et al.) “Devices and Methods for Treatment of Obesity”, U.S. Pat. No. 8,016,744 (Sep. 13, 2011 Dlugos et al.) “External Pressure-Based Gastric Band Adjustment System and Method”, U.S. Pat. No. 8,016,745 (Sep. 13, 2011 Hassler et al.) “Monitoring of a Food Intake Restriction Device”, U.S. Pat. No. 8,029,455 (Oct. 4, 2011 Stack et al.) “Satiation Pouches and Methods of Use”, U.S. Pat. No. 8,048,169 (Nov. 1, 2011 Burnett et al.) “Pyloric Valve Obstructing Devices and Methods”, U.S. Pat. No. 8,066,780 (Nov. 29, 2011 Chen et al.) “Methods for Gastric Volume Control”, U.S. Pat. No. 8,083,756 (Dec. 27, 2011 Gannoe et al.) “Methods and Devices for Maintaining a Space Occupying Device in a Relatively Fixed Location Within a Stomach”, U.S. Pat. No. 8,083,757 (Dec. 27, 2011 Gannoe et al.) “Methods and Devices for Maintaining a Space Occupying Device in a Relatively Fixed Location Within a Stomach”, U.S. Pat. No. 8,142,469 (Mar. 27, 2012 Sosnowski et al.) “Gastric Space Filler Device, Delivery System, and Related Methods”, U.S. Pat. No. 8,142,513 (Mar. 27, 2012 Shalon et al.) “Devices and Methods for Altering Eating Behavior”, U.S. Pat. No. 8,187,297 (May 29, 2012 Makower et al.) “Devices and Methods for Treatment of Obesity”, U.S. Pat. No. 8,192,455 (Jun. 5, 2012 Brazzini et al.) “Compressive Device for Percutaneous Treatment of Obesity”, U.S. Pat. No. 8,202,291 (Jun. 19, 2012 Brister et al.) “Intragastric Device”, U.S. Pat. No. 8,226,593 (Jul. 24, 2012 Graham et al.) “Pyloric Valve”, U.S. Pat. No. 8,236,023 (Aug. 7, 2012 Birk et al.) “Apparatus and Method for Volume Adjustment of Intragastric Balloons”, U.S. Pat. No. 8,241,202 (Aug. 14, 2012 Balbierz et al.) “Restrictive and/or Obstructive Implant for Inducing Weight Loss”, U.S. Pat. No. 8,267,888 (Sep. 18, 2012 Marco et al.) “Bioerodible Self-Deployable Intragastric Implants”, U.S. Pat. No. 8,282,666 (Oct. 9, 2012 Birk) “Pressure Sensing Intragastric Balloon”, U.S. Pat. No. 8,292,911 (Oct. 23, 2012 Brister et al.) “Intragastric Device”, U.S. Pat. No. 8,292,911 (Oct. 23, 2012 Brister et al.) “Intragastric Device”, U.S. Pat. No. 8,295,932 (Oct. 23, 2012 Bitton et al.) “Ingestible Capsule for Appetite Regulation”, and U.S. Pat. No. 8,337,566 (Dec. 25, 2012 Stack et al.) “Method and Apparatus for Modifying the Exit Orifice of a Satiation Pouch”.

Examples of prior art that appear to be best classified in this category also include U.S. patent applications: 20010037127 (Nov. 1, 2001 De Hoyos Garza) “Percutaneous Intragastric Balloon Catheter for the Treatment of Obesity”, 20060252983 (Nov. 9, 2006 Lembo et al.) “Dynamically Adjustable Gastric Implants and Methods of Treating Obesity Using Dynamically Adjustable Gastric Implants”, 20060264699 (Nov. 23, 2006 Gertner) “Extragastric Minimally Invasive Methods and Devices to Treat Obesity”, 20070149994 (Jun. 28, 2007 Sosnowski et al.) “Intragastric Space Filler and Methods of Manufacture”, 20070207199 (Sep. 6, 2007 Sogin) “Appetite Suppression Device”, 20070276293 (Nov. 29, 2007 Gertner) “Closed Loop Gastric Restriction Devices and Methods”, 20070293885 (Dec. 20, 2007 Binmoeller) “Methods and Devices to Curb Appetite and/or to Reduce Food Intake”, 20080051824 (Feb. 28, 2008 Gertner) “Methods and Devices for to Treatment of Obesity”, 20080065168 (Mar. 13, 2008 Bitton et al.) “Ingestible Capsule for Appetite Regulation”, 20080147002 (Jun. 19, 2008 Gertner) “Obesity Treatment Systems”, 20080161717 (Jul. 3, 2008 Gertner) “Obesity Treatment Systems”, 20080188766 (Aug. 7, 2008 Gertner) “Obesity Treatment Systems”, 20080208240 (Aug. 28, 2008 Paz) “Implantable Device for Obesity Prevention”, 20080319471 (Dec. 25, 2008 Sosnowski et al.) “Gastric Space Filler Device, Delivery System, and Related Methods”, 20090131968 (May 21, 2009 Birk) “Pressure Sensing Intragastric Balloon”, 20090192535 (Jul. 30, 2009 Kasic) “Swallowable Self-Expanding Gastric Space Occupying Device”, 20090247992 (Oct. 1, 2009 Shalon et al.) “Devices and Methods for Altering Eating Behavior”, 20090259246 (Oct. 15, 2009 Eskaros et al.) “Intragastric Volume-Occupying Device”, 20090275973 (Nov. 5, 2009 Chen et al.) “Devices and Systems for Gastric Volume Control”, 20090306462 (Dec. 10, 2009 Lechner) “System for Controlling a Controllable Stomach Band”, 20100100117 (Apr. 22, 2010 Brister et al.) “Intragastric Device”, 20100114125 (May 6, 2010 Albrecht et al.) “Method of Remotely Adjusting a Satiation and Satiety-Inducing Implanted Device”, 20100114125 (May 6, 2010 Albrecht et al.) “Method of Remotely Adjusting a Satiation and Satiety-Inducing Implanted Device”, 20100130998 (May 27, 2010 Alverdy) “Balloon System and Methods for Treating Obesity”, 20100137897 (Jun. 3, 2010 Brister et al.) “Intragastric Device”, 20100152764 (Jun. 17, 2010 Merkle) “Device for Treating Obesity”, 20100286660 (Nov. 11, 2010 Gross) “Gastroretentive Duodenal Pill”, and 20100298632 (Nov. 25, 2010 Levine et al.) “Resistive Anti-Obesity Devices”.

Examples of prior art that appear to be best classified in this category also include U.S. patent applications: 20100312049 (Dec. 9, 2010 Forsell) “Apparatus for Treating Obesity”, 20100312050 (Dec. 9, 2010 Forsell) “Method and Instrument for Treating Obesity”, 20100312147 (Dec. 9, 2010 Gertner) “Obesity Treatment Systems”, 20100324361 (Dec. 23, 2010 Forsell) “Apparatus for Treating Obesity”, 20100331616 (Dec. 30, 2010 Forsell) “Method and Instrument for Treating Obesity”, 20100331617 (Dec. 30, 2010 Forsell) “Device, System and Method for Treating Obesity”, 20100332000 (Dec. 30, 2010 Forsell) “Device for Treating Obesity”, 20110009895 (Jan. 13, 2011 Gertner) “Methods and Devices to Treat Obesity”, 20110009896 (Jan. 13, 2011 Forsell) “Apparatus for Treating Obesity”, 20110015665 (Jan. 20, 2011 Marco et al.) “Bioerodible Self-Deployable Intragastric Implants”, 20110015666 (Jan. 20, 2011 Marco et al.) “Bioerodible Self-Deployable Intragastric Implants”, 20110022072 (Jan. 27, 2011 Marco et al.) “Bioerodible Self-Deployable Intragastric Implants”, 20110040318 (Feb. 17, 2011 Marco et al.) “Bioerodible Self-Deployable Intragastric Implants”, 20110060308 (Mar. 10, 2011 Stokes et al.) “Methods and Implants for Inducing Satiety in the Treatment of Obesity”, 20110060358 (Mar. 10, 2011 Stokes et al.) “Methods and Implants for Inducing Satiety in the Treatment of Obesity”, 20110092998 (Apr. 21, 2011 Hirszowicz et al.) “Balloon Hydraulic and Gaseous Expansion System”, 20110106129 (May 5, 2011 Gertner) “Methods and Devices to Treat Obesity”, 20110172693 (Jul. 14, 2011 Forsell) “Apparatus and Method for Treating Obesity”, 20110178544 (Jul. 21, 2011 Sosnowski et al.) “Gastric Space Filler Delivery System and Related Methods”, 20110196411 (Aug. 11, 2011 Forsell) “Apparatus for Treating Obesity”, 20110213448 (Sep. 1, 2011 Kim) “Apparatus and Methods for Minimally Invasive Obesity Treatment”, 20110213469 (Sep. 1, 2011 Chin et al.) “Systems and Methods for Bariatric Therapy”, 20110224714 (Sep. 15, 2011 Gertner) “Methods and Devices for the Surgical Creation of Satiety and Biofeedback Pathways”, 20110269711 (Nov. 3, 2011 Adden et al.) “Methods and Compositions for Inducing Satiety”, and 20110295056 (Dec. 1, 2011 Aldridge et al.) “Systems and Methods for Gastric Volume Regulation”.

Examples of prior art that appear to be best classified in this category also include U.S. patent applications: 20110295057 (Dec. 1, 2011 Aldridge et al.) “Systems and Methods for Gastric Volume Regulation”, 20110307075 (Dec. 15, 2011 Sharma) “Intragastric Device for Treating Obesity”, 20110319924 (Dec. 29, 2011 Cole et al.) “Gastric Space Occupier Systems and Methods of Use”, 20120004590 (Jan. 5, 2012 Stack et al.) “Satiation Pouches and Methods of Use”, 20120022322 (Jan. 26, 2012 Pasricha) “Methods and Devices for Treating Obesity”, 20120029550 (Feb. 2, 2012 Forsell) “Obesity Treatment”, 20120041463 (Feb. 16, 2012 Forsell) “Obesity Treatment”, 20120053613 (Mar. 1, 2012 Weitzner et al.) “Gastric Filler Devices for Obesity Therapy”, 20120089168 (Apr. 12, 2012 Baker et al.) “Bariatric Device and Method”, 20120089170 (Apr. 12, 2012 Dominguez) “Intragastric Balloon Geometries”, 20120089172 (Apr. 12, 2012 Babkes et al.) “Re-Shaping Intragastric Implants”, 20120095384 (Apr. 19, 2012 Babkes et al.) “Stomach-Spanning Gastric Implants”, 20120095492 (Apr. 19, 2012 Babkes et al.) “Variable Size Intragastric Implant Devices”, 20120095494 (Apr. 19, 2012 Dominguez et al.) “Intragastric Implants with Collapsible Frames”, 20120095495 (Apr. 19, 2012 Babkes et al.) “Space-Filling Intragastric Implants with Fluid Flow”, 20120095496 (Apr. 19, 2012 Dominguez et al.) “Reactive Intragastric Implant Devices”, 20120095497 (Apr. 19, 2012 Babkes et al.) “Non-Inflatable Gastric Implants and Systems”, 20120095499 (Apr. 19, 2012 Babkes et al.) “Upper Stomach Gastric Implants”, 20120123465 (May 17, 2012 Nihalani) “Method and Apparatus for Treating Obesity and Controlling Weight Gain using Self-Expanding Intragastric Devices”, 20120150316 (Jun. 14, 2012 Carvalho) “Esophageal Flow Controller”, 20120165855 (Jun. 28, 2012 Shalon et al.) “Devices and Methods for Altering Eating Behavior”, 20120165855 (Jun. 28, 2012 Shalon et al.) “Devices and Methods for Altering Eating Behavior”, 20120191123 (Jul. 26, 2012 Brister et al.) “Intragastric Device”, and 20120191124 (Jul. 26, 2012 Brister et al.) “Intragastric Device”.

Examples of prior art that appear to be best classified in this category also include U.S. patent applications: 20120191125 (Jul. 26, 2012 Babkes et al.) “Intragastric Implants with Multiple Fluid Chambers”, 20120191126 (Jul. 26, 2012 Pecor et al.) “Inflation and Deflation Mechanisms for Inflatable Medical Devices”, 20120203061 (Aug. 9, 2012 Birk) “Bariatric Device and Method for Weight Loss”, 20120215249 (Aug. 23, 2012 Brazzini et al.) “Compressive Device for Percutaneous Treatment of Obesity”, 20120221037 (Aug. 30, 2012 Birk et al.) “Bariatric Device and Method for Weight Loss”, 20120232576 (Sep. 13, 2012 Brister et al.) “Intragastric Device”, 20120232577 (Sep. 13, 2012 Birk et al.) “Bariatric Device and Method for Weight Loss”, 20120253378 (Oct. 4, 2012 Makower et al.) “Devices and Methods for Treatment of Obesity”, 20120259427 (Oct. 11, 2012 Graham et al.) “Pyloric Valve”, 20120265030 (Oct. 18, 2012 Li) “Devices Systems Kits and Methods for Treatment of Obesity”, 20120265234 (Oct. 18, 2012 Brister et al.) “Intragastric Device”, 20120283766 (Nov. 8, 2012 Makower et al.) “Devices and Methods for Treatment of Obesity”, 20120289992 (Nov. 15, 2012 Quijano et al.) “Intragastric Balloon System and Therapeutic Processes and Products”, and 20120316387 (Dec. 13, 2012 Volker) “Adjustable Gastric Wrap (AGW)”.

32. Gastrointestinal (GI) Volume or Pressure or Flow Modification (with Drug)

Prior art in this category is similar to that in the previous category, except that it also includes delivery of a pharmaceutical agent. In various examples, this category can include drug-eluting gastric balloons, gastric balloons with an integral drug pump, and drug-eluting gastric stents. Although drug delivery can provide another therapeutic modality for these devices, the addition of drug delivery does not correct most of the potential limitations of devices that were discussed in the previous category. Accordingly, most of these limitations still apply to devices in this present category.

Examples of prior art that appear to be best classified in this category include: U.S. Pat. No. 6,627,206 (Sep. 30, 2003 Lloyd) “Method and Apparatus for Treating Obesity and for Delivering Time-Released Medicaments”, U.S. Pat. No. 7,121,283 (Oct. 17, 2006 Stack et al.) “Satiation Devices and Methods”, U.S. Pat. No. 7,152,607 (Dec. 26, 2006 Stack et al.) “Satiation Devices and Methods”, U.S. Pat. No. 7,833,280 (Nov. 16, 2010 Stack et al.) “Satiation Devices and Methods”, U.S. Pat. No. 7,854,745 (Dec. 21, 2010 Brister et al.) “Intragastric Device”, U.S. Pat. No. 8,070,768 (Dec. 6, 2011 Kim et al.) “Devices and Methods for Treatment of Obesity”, U.S. Pat. No. 8,162,969 (Apr. 24, 2012 Brister et al.) “Intragastric Device”, U.S. Pat. No. 8,177,853 (May 15, 2012 Stack et al.) “Satiation Devices and Methods”, and U.S. Pat. No. 8,226,602 (Jul. 24, 2012 Quijana et al.) “Intragastric Balloon System and Therapeutic Processes and Products”; and U.S. patent applications 20030021822 (Jan. 30, 2003 Lloyd) “Method and Apparatus for Treating Obesity and for Delivering Time-Released Medicaments”, 20040172142 (Sep. 2, 2004 Stack et al.) “Satiation Devices and Methods”, 20070265598 (Nov. 15, 2007 Karasik) “Device and Method for Treating Weight Disorders”, 20080243071 (Oct. 2, 2008 Quijano et al.) “Intragastric Balloon System and Therapeutic Processes and Products”, 20100100116 (Apr. 22, 2010 Brister et al.) “Intragastric Volume-Occupying Device and Method for Fabricating Same”, 20100114150 (May 6, 2010 Magal) “Duodenal Stimulation Devices and Methods for the Treatment of Conditions Relating to Eating Disorders”, 20120016287 (Jan. 19, 2012 Stack et al.) “Satiation Devices and Methods”, 20120022430 (Jan. 26, 2012 Stack et al.) “Satiation Devices and Methods”, 20120245553 (Sep. 27, 2012 Raven et al.) “Intragastric Volume Occupying Device with Active Agents”, and 20120271217 (Oct. 25, 2012 Stack et al.) “Satiation Devices and Methods”.

### 35. Electrical Stimulation (General)

Prior art in this category includes implantable devices that deliver electromagnetic energy to a portion of a person's gastrointestinal tract or to a nerve that innervates a portion of the person's gastrointestinal tract. In an example, electrical stimulation can be applied directly to a person's stomach in order to induce a sense of satiety and/or modify gastric motility. The intent of such gastric stimulation is to reduce a person's food consumption. In another example, electrical energy can be applied to block normal neural transmissions in a nerve that innervates a person's stomach in order to reduce gastric functioning and thereby reduce food consumption. This category of art has considerable potential (no pun intended) to modify food consumption. It is relatively non-invasive with respect to other internal procedures, is adjustable, and is reversible.

In order for devices in this category to be successful in modifying food consumption, the gastrointestinal organ or nerve to which electrical energy is applied must not accommodate (ie: become inured to) the application of electrical energy. If an organ or nerve does accommodate the application of electrical energy, then the organ or nerve stops responding to the applied energy in a therapeutic manner. For this reason, devices in this category generally apply electrical energy in a non-continuous manner.

The ability to differentiate between consumption of healthy vs unhealthy food could enable such devices to selectively deliver electrical energy only when a person eats unhealthy food. This differentiating ability would allow use of higher power levels without the problem of accommodation and make such devices more effective for modifying food consumption. Such ability could also encourage the person to have a healthier diet and extend a device's battery life. However, prior art devices in this category do not appear to offer the ability to differentiate between consumption of healthy vs unhealthy food.

Examples of prior art that appear to be best classified in this category include U.S. patents: U.S. Pat. No. 3,411,507 (Nov. 19, 1968 Wingrove) “Method of Gastrointestinal Stimulation with Electrical Pulses”, U.S. Pat. No. 5,188,104 (Feb. 23, 1993 Wernicke et al.) “Treatment of Eating Disorders by Nerve Stimulation”, U.S. Pat. No. 5,423,872 (Jun. 13, 1995 Cigaina) “Process and Device for Treating Obesity and Syndromes Related to Motor Disorders of the Stomach of a Patient”, U.S. Pat. No. 5,690,691 (Nov. 25, 1997 Chen et al.) “Gastro-Intestinal Pacemaker Having Phased Multi-Point Stimulation”, U.S. Pat. No. 5,716,385 (Feb. 10, 1998 Mittal et al.) “Crural Diaphragm Pacemaker and Method for Treating Esophageal Reflux Disease (Mittal)”, U.S. Pat. No. 5,891,185 (Apr. 6, 1999 Freed et al.) “Method and Apparatus for Treating Oropharyngeal Disorders with Electrical Stimulation”, U.S. Pat. No. 6,091,992 (Jul. 18, 2000 Bourgeois et al.) “Method and Apparatus for Electrical Stimulation of the Gastrointestinal Tract”, U.S. Pat. No. 6,243,607 (Jun. 5, 2001 Mintchev et al.) “Gastro-Intestinal Electrical Pacemaker”, U.S. Pat. No. 6,564,101 (May 13, 2003 Zikria) “Electrical System for Weight Loss and Laparoscopic Implantation Thereof”, U.S. Pat. No. 6,587,719 (Jul. 1, 2003 Barrett et al.) “Treatment of Obesity by Bilateral Vagus Nerve Stimulation”, U.S. Pat. No. 6,609,025 (Aug. 19, 2003 Barrett et al.) “Treatment of Obesity by Bilateral Sub-Diaphragmatic Nerve Stimulation”, U.S. Pat. No. 6,684,104 (Jan. 27, 2004 Gordon et al.) “Gastric Stimulator Apparatus and Method for Installing”, U.S. Pat. No. 6,760,626 (Jul. 6, 2004 Boveja) “Apparatus and Method for Treatment of Neurological and Neuropsychiatric Disorders Using Programmerless Implantable Pulse Generator System”, U.S. Pat. No. 6,879,859 (Apr. 12, 2005 Boveja) “External Pulse Generator for Adjunct (Add-On) Treatment of Obesity Eating Disorders Neurological Neuropsychiatric and Urological Disorders”, U.S. Pat. No. 7,072,720 (Jul. 4, 2006 Puskas) “Devices and Methods for Vagus Nerve Stimulation”, U.S. Pat. No. 7,167,750 (Jan. 23, 2007 Knudson et al.) “Obesity Treatment with Electrically Induced Vagal Down Regulation”, U.S. Pat. No. 7,177,693 (Feb. 13, 2007 Starkebaum) “Gastric Stimulation for Altered Perception to Treat Obesity”, and U.S. Pat. No. 7,236,822 (Jun. 26, 2007 Dobak) “Wireless Electric Modulation of Sympathetic Nervous System”.

Examples of prior art that appear to be best classified in this category also include U.S. patents: U.S. Pat. No. 7,239,912 (Jul. 3, 2007 Dobak) “Electric Modulation of Sympathetic Nervous System”, U.S. Pat. No. 7,299,091 (Nov. 20, 2007 Barrett et al.) “Treatment of Obesity by Bilateral Vagus Nerve Stimulation”, U.S. Pat. No. 7,529,582 (May 5, 2009 Dilorenzo) “Method and Apparatus for Neuromodulation and Physiologic Modulation for the Treatment of Metabolic and Neuropsychiatric Disease”, U.S. Pat. No. 7,551,964 (Jun. 23, 2009 Dobak) “Splanchnic Nerve Stimulation for Treatment of Obesity”, U.S. Pat. No. 7,580,751 (Aug. 25, 2009 Starkebaum) “Intra-Luminal Device for Gastrointestinal Stimulation”, U.S. Pat. No. 7,599,736 (Oct. 6, 2009 Dilorenzo) “Method and Apparatus for Neuromodulation and Physiologic Modulation for the Treatment of Metabolic and Neuropsychiatric Disease”, U.S. Pat. No. 7,657,310 (Feb. 2, 2010 Buras) “Treatment of Reproductive Endocrine Disorders by Vagus Nerve Stimulation”, U.S. Pat. No. 7,664,551 (Feb. 16, 2010 Cigaina) “Treatment of the Autonomic Nervous System”, U.S. Pat. No. 7,689,276 (Mar. 30, 2010 Dobak) “Dynamic Nerve Stimulation for Treatment of Disorders”, U.S. Pat. No. 7,689,277 (Mar. 30, 2010 Dobak) “Neural Stimulation for Treatment of Metabolic Syndrome and Type 2 Diabetes”, U.S. Pat. No. 7,702,386 (Apr. 20, 2010 Dobak et al.) “Nerve Stimulation for Treatment of Obesity Metabolic Syndrome and Type 2 Diabetes”, U.S. Pat. No. 7,729,771 (Jun. 1, 2010 Knudson et al.) “Nerve Stimulation and Blocking for Treatment of Gastrointestinal Disorders”, U.S. Pat. No. 7,756,582 (Jul. 13, 2010 Imran et al.) “Gastric Stimulation Anchor and Method”, U.S. Pat. No. 7,840,278 (Nov. 23, 2010 Puskas) “Devices and Methods for Vagus Nerve Stimulation”, U.S. Pat. No. 7,945,323 (May 17, 2011 Jaax et al.) “Treatment of Obesity and/or Type II Diabetes by Stimulation of the Pituitary Gland”, U.S. Pat. No. 7,979,127 (Jul. 12, 2011 Imran) “Digestive Organ Retention Device”, U.S. Pat. No. 7,986,995 (Jul. 26, 2011 Knudson et al.) “Bulimia Treatment”, U.S. Pat. No. 8,082,039 (Dec. 20, 2011 Kim et al.) “Stimulation Systems”, U.S. Pat. No. 8,145,299 (Mar. 27, 2012 Dobak) “Neural Stimulation for Treatment of Metabolic Syndrome and Type 2 Diabetes”, U.S. Pat. No. 8,150,508 (Apr. 3, 2012 Craig) “Vagus Nerve Stimulation Method”, U.S. Pat. No. 8,280,505 (Oct. 2, 2012 Craig) “Vagus Nerve Stimulation Method”, U.S. Pat. No. 8,301,256 (Oct. 30, 2012 Policker et al.) “GI Lead Implantation”, and U.S. Pat. No. 8,340,772 (Dec. 25, 2012 Vase et al.) “Brown Adipose Tissue Utilization Through Neuromodulation”.

Examples of prior art that appear to be best classified in this category also include U.S. patent applications: 20040167583 (Aug. 26, 2004 Knudson et al.) “Electrode Band Apparatus and Method”, 20070027498 (Feb. 1, 2007 Maschino et al.) “Selective Nerve Stimulation for the Treatment of Eating Disorders”, 20070135846 (Jun. 14, 2007 Knudson et al.) “Vagal Obesity Treatment”, 20070150021 (Jun. 28, 2007 Chen et al.) “Gastrointestinal Electrical Stimulation”, 20070203521 (Aug. 30, 2007 Dobak et al.) “Nerve Stimulation for Treatment of Obesity Metabolic Syndrome and Type 2 Diabetes”, 20080046013 (Feb. 21, 2008 Lozano) “Method for Treating Eating Disorders”, 20080183238 (Jul. 31, 2008 Chen) “Process for Electrostimulation Treatment of Morbid Obesity”, 20080195171 (Aug. 14, 2008 Sharma) “Method and Apparatus for Electrical Stimulation of the Pancreatico-Biliary System”, 20090018606 (Jan. 15, 2009 Sparks et al.) “Methods and Devices for Stimulation of an Organ with the Use of a Transectionally Placed Guide Wire”, 20090259274 (Oct. 15, 2009 Simon et al.) “Methods and Apparatus for Electrical Treatment Using Balloon and Electrode”, 20090259279 (Oct. 15, 2009 Dobak) “Splanchnic Nerve Stimulation for Treatment of Obesity”, 20100087706 (Apr. 8, 2010 Syed et al.) “Lead Access”, 20100094375 (Apr. 15, 2010 Donders et al.) “Neural Electrode Treatment”, 20100168815 (Jul. 1, 2010 Knudson et al.) “Nerve Stimulation and Blocking for Treatment of Gastrointestinal Disorders”, 20100183700 (Jul. 22, 2010 Stojanovic-Susulic et al.) “Implantable Pump for Protein Delivery for Obesity Control by Drug Infusion into the Brain”, 20100234917 (Sep. 16, 2010 Imran) “Digestive Organ Retention Device”, and 20100286745 (Nov. 11, 2010 Imran) “Radially Expandable Gastrointestinal Stimulation Device”.

Examples of prior art that appear to be best classified in this category also include U.S. patent applications: 20110034967 (Feb. 10, 2011 Chen et al.) “Gastrointestinal Electrical Stimulation”, 20110034968 (Feb. 10, 2011 Knudson et al.) “Controlled Vagal Blockage Therapy”, 20110166582 (Jul. 7, 2011 Syed et al.) “Endoscopic Device Delivery System”, 20110230938 (Sep. 22, 2011 Simon et al.) “Device and Methods for Non-Invasive Electrical Stimulation and Their Use for Vagal Nerve Stimulation”, 20110238035 (Sep. 29, 2011 Jaax et al.) “Treatment of Obesity and/or Type II Diabetes by Stimulation of the Pituitary Gland”, 20110270344 (Nov. 3, 2011 Knudson et al.) “Bulimia Treatment”, 20110307023 (Dec. 15, 2011 Tweden et al.) “Neural Modulation Devices and Methods”, 20110319969 (Dec. 29, 2011 Dobak) “Electric Modulation of Sympathetic Nervous System”, 20120041509 (Feb. 16, 2012 Knudson et al.) “Controlled Vagal Blockage Therapy”, 20120053653 (Mar. 1, 2012 Hiernaux et al.) “Gastrointestinal Device”, 20120053660 (Mar. 1, 2012 Dobak) “Splanchnic Nerve Stimulation for Treatment of Obesity”, 20120071947 (Mar. 22, 2012 Gupta et al.) “Method and Apparatus for Event-Triggered Reinforcement of a Favorable Brain State”, 20120143279 (Jun. 7, 2012 Ekchian et al.) “Methods and Kits for Treating Appetite Suppressing Disorders and Disorders with an Increased Metabolic Rate”, 20120209354 (Aug. 16, 2012 Raykhman) “System and Methods for Producing and Delivering Electrical Impulses”, and 20120310295 (Dec. 6, 2012 Libbus et al.) “Systems and Methods for Avoiding Neural Stimulation Habituation”.

36. Electrical Stimulation (with Glucose Sensor)

Devices in this category are similar to devices in the previous category of general electrical stimulation except that they also include a glucose sensor. They deliver electromagnetic energy to person's gastrointestinal tract or to a nerve that innervates their gastrointestinal tract. In an example, a person's blood glucose level can be monitored and gastrointestinal electrical stimulation can be triggered when the person's glucose level indicates that such stimulation is most needed. Selective electrical stimulation can help to target therapeutic benefit.

Examples of prior art that appear to be best classified in this category include U.S. patents: U.S. Pat. No. 6,093,167 (Jul. 25, 2000 Houben et al.) “System for Pancreatic Stimulation and Glucose Measurement”, U.S. Pat. No. 6,185,452 (Feb. 6, 2001 Schulman et al.) “Battery-Powered Patient Implantable Device”, U.S. Pat. No. 6,571,127 (May 27, 2003 Ben-Haim et al.) “Method of Increasing the Motility of a GI Tract”, U.S. Pat. No. 6,600,953 (Jul. 29, 2003 Flesler et al.) “Acute and Chronic Electrical Signal Therapy for Obesity”, U.S. Pat. No. 6,832,114 (Dec. 14, 2004 Whitehurst et al.) “Systems and Methods for Modulation of Pancreatic Endocrine Secretion and Treatment of Diabetes”, U.S. Pat. No. 6,922,590 (Jul. 26, 2005 Whitehurst) “Systems and Methods for Treatment of Diabetes by Electrical Brain Stimulation and/or Drug Infusion”, U.S. Pat. No. 6,993,391 (Jan. 31, 2006 Flesler et al.) “Acute and Chronic Electrical Signal Therapy for Obesity”, U.S. Pat. No. 7,020,531 (Mar. 28, 2006 Colliou et al.) “Gastric Device and Suction Assisted Method for Implanting a Device on a Stomach Wall”, U.S. Pat. No. 7,440,806 (Oct. 21, 2008 Whitehurst et al.) “Systems and Methods for Treatment of Diabetes by Electrical Brain Stimulation and/or Drug Infusion”, U.S. Pat. No. 7,477,944 (Jan. 13, 2009 Whitehurst et al.) “Systems and Methods for Modulation of Pancreatic Endocrine Secretion and Treatment of Diabetes”, U.S. Pat. No. 7,502,649 (Mar. 10, 2009 Ben-Haim et al.) “Gastrointestinal Methods and Apparatus for Use in Treating Disorders”, U.S. Pat. No. 7,512,442 (Mar. 31, 2009 Flesler et al.) “Acute and Chronic Electrical Signal Therapy for Obesity”, U.S. Pat. No. 7,558,629 (Jul. 7, 2009 Keimel et al.) “Energy Balance Therapy for Obesity Management”, U.S. Pat. No. 7,937,145 (May 3, 2011 Dobak) “Dynamic Nerve Stimulation Employing Frequency Modulation”, U.S. Pat. No. 8,019,421 (Sep. 13, 2011 Darvish et al.) “Blood Glucose Level Control”, U.S. Pat. No. 8,095,218 (Jan. 10, 2012 Gross et al.) “GI and Pancreatic Device for Treating Obesity and Diabetes”, U.S. Pat. No. 8,135,470 (Mar. 13, 2012 Keimel et al.) “Energy Balance Therapy for Obesity Management”, U.S. Pat. No. 8,209,037 (Jun. 26, 2012 Laufer et al.) “Methods and Devices for Medical Treatment”, U.S. Pat. No. 8,321,030 (Nov. 27, 2012 Maniak et al.) “Esophageal Activity Modulated Obesity Therapy”, U.S. Pat. No. 8,321,030 (Nov. 27, 2012 Maniak et al.) “Esophageal Activity Modulated Obesity Therapy”, and U.S. Pat. No. 8,346,363 (Jan. 1, 2013 Darvish et al.) “Blood Glucose Level Control”.

Examples of prior art that appear to be best classified in this category also include U.S. patent applications: 20040044376 (Mar. 4, 2004 Flesler et al.) “Acute and Chronic Electrical Signal Therapy for Obesity”, 20050149142 (Jul. 7, 2005 Starkebaum) “Gastric Stimulation Responsive to Sensing Feedback”, 20050222638 (Oct. 6, 2005 Foley et al.) “Sensor Based Gastrointestinal Electrical Stimulation for the Treatment of Obesity or Motility Disorders”, 20060074459 (Apr. 6, 2006 Flesler et al.) “Acute and Chronic Electrical Signal Therapy for Obesity”, 20070016262 (Jan. 18, 2007 Gross et al.) “GI and Pancreatic Device for Treating Obesity and Diabetes”, 20070027493 (Feb. 1, 2007 Ben-Haim et al.) “Gastrointestinal Methods and Apparatus for Use in Treating Disorders and Controlling Blood Sugar”, 20070179556 (Aug. 2, 2007 Ben-Haim et al.) “Gastrointestinal Methods and Apparatus for Use in Treating Disorders”, 20070255334 (Nov. 1, 2007 Keimel et al.) “Energy Balance Therapy for Obesity Management”, 20090018594 (Jan. 15, 2009 Laufer et al.) “Methods and Devices for Medical Treatment”, 20090030474 (Jan. 29, 2009 Brynelsen et al.) “Sensor Driven Gastric Stimulation for Patient Management”, 20090062881 (Mar. 5, 2009 Gross et al.) “GI and Pancreatic Device for Treating Obesity and Diabetes”, 20090088816 (Apr. 2, 2009 Harel et al.) “Gastrointestinal Methods and Apparatus for Use in Treating Disorders and Controlling Blood Sugar”, 20090240194 (Sep. 24, 2009 Keimel et al.) “Energy Balance Therapy for Obesity Management”, 20100268306 (Oct. 21, 2010 Maniak et al.) “Esophageal Activity Modulated Obesity Therapy”, 20110087076 (Apr. 14, 2011 Brynelsen et al.) “Feedback Systems and Methods for Communicating Diagnostic and/or Treatment Signals to Enhance Obesity Treatments”, 20120083855 (Apr. 5, 2012 Gross et al.) “GI and Pancreatic Device for Treating Obesity and Diabetes”, 20120214140 (Aug. 23, 2012 Brynelsen et al.) “Feedback Systems and Methods for Communicating Diagnostic and/or Treatment Signals to Enhance Obesity Treatments”, 20120259389 (Oct. 11, 2012 Starkebaum et al.) “Treatment of Postprandial Hyperglycemia by Gastric Electrical Stimulation”, and 20120323099 (Dec. 20, 2012 Mothilal et al.) “Implantable Medical Device Electrode Assembly”.

37. Electrical Stimulation (with General Sensor)

Devices in this category are similar to devices in the prior category of general electrical stimulation except that they also include one or more sensors other than a glucose sensor. Like devices in prior categories, they deliver electromagnetic energy to person's gastrointestinal tract or to a nerve that innervates their gastrointestinal tract. In an example, the electromagnetic properties of a person's esophagus or stomach can be monitored by an electromagnetic sensor and gastrointestinal electrical stimulation can be triggered when the sensor indicates that a person is consuming food. Selective electrical stimulation can help to target therapeutic benefit.

Examples of prior art that appear to be best classified in this category include U.S. patents: U.S. Pat. No. 5,263,480 (Nov. 23, 1993 Wernicke et al.) “Treatment of Eating Disorders by Nerve Stimulation”, U.S. Pat. No. 5,292,344 (Mar. 8, 1994 Douglas) “Percutaneously Placed Electrical Gastrointestinal Pacemaker Stimulatory System, Sensing System, and PH Monitoring System, with Optional Delivery Port”, U.S. Pat. No. 5,540,730 (Jul. 30, 1996 Terry et al.) “Treatment of Motility Disorders by Nerve Stimulation”, U.S. Pat. No. 5,836,994 (Nov. 17, 1998 Bourgeois) “Method and Apparatus for Electrical Stimulation of the Gastrointestinal Tract”, U.S. Pat. No. 5,861,014 (Jan. 19, 1999 Familoni) “Method and Apparatus for Sensing a Stimulating Gastrointestinal Tract On-Demand”, U.S. Pat. No. 5,995,872 (Nov. 30, 1999 Bourgeois) “Method and Apparatus for Electrical Stimulation of the Gastrointestinal Tract”, U.S. Pat. No. 6,083,249 (Jul. 4, 2000 Familoni) “Apparatus for Sensing and Stimulating Gastrointestinal Tract On-Demand”, U.S. Pat. No. 6,104,955 (Aug. 15, 2000 Bourgeois) “Method and Apparatus for Electrical Stimulation of the Gastrointestinal Tract”, U.S. Pat. No. 6,115,635 (Sep. 5, 2000 Bourgeois) “Method and Apparatus for Electrical Stimulation of the Gastrointestinal Tract”, U.S. Pat. No. 6,216,039 (Apr. 10, 2001 Bourgeois) “Method and Apparatus for Treating Irregular Gastric Rhythms”, U.S. Pat. No. 6,327,503 (Dec. 4, 2001 Familoni) “Method and Apparatus for Sensing and Stimulating Gastrointestinal Tract On-Demand”, U.S. Pat. No. 6,535,764 (Mar. 18, 2003 Imran et al.) “Gastric Treatment and Diagnosis Device and Method (Intrapace: Imran)”, U.S. Pat. No. 6,591,137 (Jul. 8, 2003 Fischell et al.) “Implantable Neuromuscular Stimulator for the Treatment of Gastrointestinal Disorders”, and U.S. Pat. No. 6,735,477 (May 11, 2004 Levine) “Internal Monitoring System with Detection of Food Intake”.

Examples of prior art that appear to be best classified in this category also include U.S. patents: U.S. Pat. No. 6,826,428 (Nov. 30, 2004 Chen et al.) “Gastrointestinal Electrical Stimulation”, U.S. Pat. No. 6,993,391 (Jan. 31, 2006 Flesler et al.) “Acute and Chronic Electrical Signal Therapy for Obesity”, U.S. Pat. No. 7,054,690 (May 30, 2006 Imran) “Gastrointestinal Stimulation Device”, U.S. Pat. No. 7,120,498 (Oct. 10, 2006 Imran et al.) “Method and Device for Securing a Functional Device to a Stomach”, U.S. Pat. No. 7,430,450 (Sep. 30, 2008 Imran) “Device and Method for Treating Obesity”, U.S. Pat. No. 7,437,195 (Oct. 14, 2008 Policker et al.) “Regulation of Eating Habits”, U.S. Pat. No. 7,509,174 (Mar. 24, 2009 Imran et al.) “Gastric Treatment/Diagnosis Device and Attachment Device and Method”, U.S. Pat. No. 7,620,454 (Nov. 17, 2009 Dinsmoor et al.) “Gastro-Electric Stimulation for Reducing the Acidity of Gastric Secretions or Reducing the Amounts Thereof”, U.S. Pat. No. 7,643,887 (Jan. 5, 2010 Imran) “Abdominally Implanted Stimulator and Method”, U.S. Pat. No. 7,702,394 (Apr. 20, 2010 Imran) “Responsive Gastric Stimulator”, U.S. Pat. No. 7,738,961 (Jun. 15, 2010 Sharma) “Method and Apparatus for Treatment of the Gastrointestinal Tract”, U.S. Pat. No. 7,742,818 (Jun. 22, 2010 Dinsmoor et al.) “Gastro-Electric Stimulation for Increasing the Acidity of Gastric Secretions or Increasing the Amounts Thereof”, U.S. Pat. No. 7,881,797 (Feb. 1, 2011 Griffin et al.) “Methods and Devices for Gastrointestinal Stimulation”, U.S. Pat. No. 7,941,221 (May 10, 2011 Foley) “Method and Apparatus for Intentional Impairment of Gastric Motility and/or Efficiency by Triggered Electrical Stimulation of the Gastrointestinal . . . ”, U.S. Pat. No. 8,214,049 (Jul. 3, 2012 Brynelsen et al.) “Gastric Stimulation Systems and Methods Utilizing a Transgastric Probe”, and U.S. Pat. No. 8,239,027 (Aug. 7, 2012 Imran) “Responsive Gastric Stimulator”.

Examples of prior art that appear to be best classified in this category also include U.S. patent applications: 20020072780 (Jun. 13, 2002 Foley) “Method and Apparatus for Intentional Impairment of Gastric Motility and/or Efficiency by Triggered Electrical Stimulation of the Gastrointestinal Tract . . . ”, 20030009202 (Jan. 9, 2003 Levine) “Internal Monitoring System with Detection of Food Intake”, 20040059393 (Mar. 25, 2004 Policker et al.) “Regulation of Eating Habits”, 20040088023 (May 6, 2004 Imran et al.) “Gastric Treatment and Diagnosis Device and Method”, 20040162595 (Aug. 19, 2004 Foley) “Method and Apparatus for Intentional Impairment of Gastric Motility and/or Efficiency by Triggered Electrical Stimulation of the Gastrointestinal Tract . . . ”, 20050065571 (Mar. 24, 2005 Imran) “Responsive Gastric Stimulator”, 20050090873 (Apr. 28, 2005 Imran) “Gastrointestinal Stimulation Device”, 20060079944 (Apr. 13, 2006 Imran) “Device and Method for Treating Obesity”, 20060089699 (Apr. 27, 2006 Imran) “Abdominally Implanted Stimulator and Method”, 20070060812 (Mar. 15, 2007 Harel et al.) “Sensing of Pancreatic Electrical Activity”, 20070162085 (Jul. 12, 2007 Dilorenzo) “Method Apparatus Surgical Technique and Stimulation Parameters for Autonomic Neuromodulation for the Treatment of Obesity”, 20080058887 (Mar. 6, 2008 Griffin et al.) “Methods and Devices for Gastrointestinal Stimulation”, 20080086179 (Apr. 10, 2008 Sharma) “Method and Apparatus for Treatment of the Gastrointestinal Tract”, 20090018605 (Jan. 15, 2009 Imran et al.) “Gastric Treatment/Diagnosis Device and Attachment Device and Method”, 20090018605 (Jan. 15, 2009 Imran et al.) “Gastric Treatment/Diagnosis Device and Attachment Device and Method”, 20090030475 (Jan. 29, 2009 Brynelsen et al.) “Gastric Stimulation Systems and Methods Utilizing a Transgastric Probe”, and 20090149910 (Jun. 11, 2009 Imran et al.) “Gastric Treatment/Diagnosis Device and Attachment Device and Method”.

Examples of prior art that appear to be best classified in this category also include U.S. patent applications: 20090264951 (Oct. 22, 2009 Sharma) “Device and Implantation System for Electrical Stimulation of Biological Systems”, 20100049274 (Feb. 25, 2010 Cholette) “Detection of Feeding Intent for Use in Treatment of Eating Disorders”, 20100049274 (Feb. 25, 2010 Cholette) “Detection of Feeding Intent for Use in Treatment of Eating Disorders”, 20100094374 (Apr. 15, 2010 Imran) “Responsive Gastric Stimulator”, 20100305656 (Dec. 2, 2010 Imran et al.) “Gastric Stimulation Anchor and Method”, 20100324432 (Dec. 23, 2010 Bjorling et al.) “Method and Device to Detect Eating to Control Artificial Gastric Stimulation”, 20110004266 (Jan. 6, 2011 Sharma) “Method and Apparatus for Treatment of the Gastrointestinal Tract”, 20110066207 (Mar. 17, 2011 Imran) “Responsive Gastric Stimulator”, 20110125211 (May 26, 2011 Griffin et al.) “Methods and Devices for Gastrointestinal Stimulation”, 20110251495 (Oct. 13, 2011 Province et al.) “Diagnostic Sensors and/or Treatments for Gastrointestinal Stimulation or Monitoring Devices”, 20110295335 (Dec. 1, 2011 Sharma et al.) “Device and Implantation System for Electrical Stimulation of Biological Systems”, 20110295336 (Dec. 1, 2011 Sharma et al.) “Device and Implantation System for Electrical Stimulation of Biological Systems”, 20110307027 (Dec. 15, 2011 Sharma et al.) “Device and Implantation System for Electrical Stimulation of Biological Systems”, 20110307028 (Dec. 15, 2011 Sharma et al.) “Device and Implantation System for Electrical Stimulation of Biological Systems”, 20120277619 (Nov. 1, 2012 Starkebaum et al.) “Detecting Food Intake Based on Impedance”, and 20120316451 (Dec. 13, 2012 Province et al.) “Event Evaluation Using Heart Rate Variation for Ingestion Monitoring and Therapy”.

38. Electrical Stimulation (with Taste Modification)

Devices in this category are similar to devices in the prior category of general electrical stimulation except that they specifically modify a person's sense of taste. In an example, nerves that innervate a person's taste buds can be stimulated to modify a person's sense of taste and thereby modify their food consumption.

Examples of prior art that appear to be best classified in this category include U.S. patent applications: 20060173508 (Aug. 3, 2006 Stone et al.) “Method and System for Treatment of Eating Disorders by Means of Neuro-Electrical Coded Signals”, 20060206169 (Sep. 14, 2006 Schuler) “Method and System for Modulating Eating Behavior by Means of Neuro-Electrical Coded Signals”, 20060235487 (Oct. 19, 2006 Meyer et al.) “Method and System for Treatment of Eating Disorders by Means of Neuro-Electrical Coded Signals”, 20110276112 (Nov. 10, 2011 Simon et al.) “Devices and Methods for Non-Invasive Capacitive Electrical Stimulation and Their Use for Vagus Nerve Stimulation on the Neck of a Patient”, 20120029591 (Feb. 2, 2012 Simon et al.) “Devices and Methods for Non-Invasive Capacitive Electrical Stimulation and Their Use for Vagus Nerve Stimulation on the Neck of a Patient”, 20120029601 (Feb. 2, 2012 Simon et al.) “Devices and Methods for Non-Invasive Capacitive Electrical Stimulation and Their Use for Vagus Nerve Stimulation on the Neck of a Patient”, 20120277814 (Nov. 1, 2012 Schuler) “Method and System for Modulating Eating Behavior by Means of Neuro-Electrical Coded Signals”, and 20120277837 (Nov. 1, 2012 Schuler) “Method and System for Modulating Eating Behavior by Means of Neuro-Electrical Coded Signals”.

39. Electrical Stimulation (with Drug)

Devices in this category are similar to devices in the prior category of general electrical stimulation except that they also include a drug delivery mechanism. In addition to delivering electromagnetic energy to person's gastrointestinal tract or to a nerve that innervates their gastrointestinal tract, devices in this category can also include an implantable drug pump. In an example, electrical stimulation can be used in conjunction with drug delivery to create combined therapeutic effects.

Examples of prior art that appear to be best classified in this category include: U.S. Pat. No. 5,782,798 (Jul. 21, 1998 Rise) “Techniques for Treating Eating Disorders by Brain Stimulation and Drug Infusion”, U.S. Pat. No. 7,493,171 (Feb. 17, 2009 Whitehurst et al.) “Treatment of Pathologic Craving and Aversion Syndromes and Eating Disorders by Electrical Brain Stimulation and/or Drug Infusion”, U.S. Pat. No. 7,835,796 (Nov. 16, 2010 Maschino et al.) “Weight Loss Method and Device”, U.S. Pat. No. 8,010,204 (Aug. 30, 2011 Knudson et al.) “Nerve Blocking for Treatment of Gastrointestinal Disorders”, U.S. Pat. No. 8,185,206 (May 22, 2012 Starkebaum et al.) “Electrical Stimulation Therapy to Promote Gastric Distention for Obesity Management”, and U.S. Pat. No. 8,295,926 (Oct. 23, 2012 Dobak) “Dynamic Nerve Stimulation in Combination with Other Eating Disorder Treatment Modalities”; and U.S. patent applications 20080021512 (Jan. 24, 2008 Knudson et al.) “Nerve Stimulation and Blocking for Treatment of Gastrointestinal Disorders”, 20080262411 (Oct. 23, 2008 Dobak) “Dynamic Nerve Stimulation in Combination with Other Eating Disorder Treatment Modalities”, 20110282411 (Nov. 17, 2011 Knudson et al.) “Nerve Stimulation and Blocking for Treatment of Gastrointestinal Disorders”, 20110282411 (Nov. 17, 2011 Knudson et al.) “Nerve Stimulation and Blocking for Treatment of Gastrointestinal Disorders”, and 20120277661 (Nov. 1, 2012 Bernard et al.) “Method and Apparatus for Delivery of Therapeutic Agents”.

40. Electrical Stimulation (with Drug and Sensor)

Devices in this category are similar to devices in a prior category of general electrical stimulation except that they also include a drug delivery mechanism and at least one sensor. In an example, electrical stimulation can be used in conjunction with drug delivery to create combined therapeutic effects. Further, the sensor can be used to create a self-adjusting, closed-loop stimulation and/or drug delivery system for modification of food consumption.

Examples of prior art that appear to be best classified in this category include: U.S. Pat. No. 6,950,707 (Sep. 27, 2005 Whitehurst) “Systems and Methods for Treatment of Obesity and Eating Disorders by Electrical Brain Stimulation and/or Drug Infusion”, U.S. Pat. No. 7,076,305 (Jul. 11, 2006 Imran et al.) “Gastric Device and Instrument System and Method”, U.S. Pat. No. 7,483,746 (Jan. 27, 2009 Lee et al.) “Stimulation of the Stomach in Response to Sensed Parameters to Treat Obesity”, U.S. Pat. No. 7,590,452 (Sep. 15, 2009 Imran et al.) “Endoscopic System for Attaching a Device to a Stomach”, and U.S. Pat. No. 8,095,219 (Jan. 10, 2012 Lee et al.) “Stimulation of the Stomach in Response to Sensed Parameters to Treat Obesity”; and U.S. patent applications 20030167024 (Sep. 4, 2003 Imran et al.) “Gastric Device and Instrument System and Method”, 20040243195 (Dec. 2, 2004 Imran et al.) “Endoscopic System for Attaching a Device to a Stomach”, 20060129201 (Jun. 15, 2006 Lee et al.) “Stimulation of the Stomach in Response to Sensed Parameters to Treat Obesity”, and 20090299434 (Dec. 3, 2009 Imran et al.) “Endoscopic System for Attaching a Device to a Stomach”.

### 42. General Sensor (Glucose)

This category of prior art includes sensors and monitors which detect and analyze glucose levels (such as blood glucose levels). These sensors and monitors can be used for a variety of applications other than modification of food consumption or food absorption. For example, they can be used to determine when a diabetic person needs insulin. Nonetheless, overall, they are sufficiently relevant to be included in this review.

Examples of prior art that appear to be best classified in this category include: U.S. Pat. No. 5,497,772 (Mar. 12, 1996 Schulman et al.) “Glucose Monitoring System”, U.S. Pat. No. 7,727,147 (Jun. 1, 2010 Osorio et al.) “Method and System for Implantable Glucose Monitoring and Control of a Glycemic State of a Subject”, U.S. Pat. No. 7,974,672 (Jul. 5, 2011 Shults et al.) “Device and Method for Determining Analyte Levels”, U.S. Pat. No. 7,988,630 (Aug. 2, 2011 Osorio et al.) “Method and System for Implantable Glucose Monitoring and Control of a Glycemic State of a Subject”, U.S. Pat. No. 8,158,082 (Apr. 17, 2012 Imran) “Micro-Fluidic Device”, U.S. Pat. No. 8,236,242 (Aug. 7, 2012 Drucker et al.) “Blood Glucose Tracking Apparatus and Methods”, U.S. Pat. No. 8,275,438 (Sep. 25, 2012 Simpson et al.) “Analyte Sensor”, U.S. Pat. No. 8,287,453 (Oct. 16, 2012 Li et al.) “Analyte Sensor”, and U.S. Pat. No. 8,298,142 (Oct. 30, 2012 Simpson et al.) “Analyte Sensor”; and U.S. patent applications 20050096637 (May 5, 2005 Heruth) “Sensing Food Intake”, 20120078071 (Mar. 29, 2012 Bohm et al.) “Advanced Continuous Analyte Monitoring System”, 20120149996 (Jun. 14, 2012 Stivoric et al.) “Method and Apparatus for Providing Derived Glucose Information Utilizing Physiological and/or Contextual Parameters”, and 20120201725 (Aug. 9, 2012 Imran) “Micro-Fluidic Device”.

### 43. General Sensor (Electromagnetic)

This category of prior art includes sensors and monitors which detect selected patterns of electromagnetic energy that are emitted from a member of a person's body. Such sensors and monitors can be used for a variety of applications other than modification of food consumption or food absorption. Nonetheless, overall, they are sufficiently relevant to be included in this review.

Examples of prior art that appear to be best classified in this category include: U.S. Pat. No. 5,795,304 (Aug. 18, 1998 Sun et al.) “System and Method for Analyzing Electrogastrophic Signal”, U.S. Pat. No. 6,285,897 (Sep. 4, 2001 Kilcoyne et al.) “Remote Physiological Monitoring System”, U.S. Pat. No. 8,192,350 (Jun. 5, 2012 Ortiz et al.) “Methods and Devices for Measuring Impedance in a Gastric Restriction System”, U.S. Pat. No. 8,265,758 (Sep. 11, 2012 Policker et al.) “Wireless Leads for Gastrointestinal Tract Applications”, and U.S. Pat. No. 8,328,420 (Dec. 11, 2012 Abreu) “Apparatus and Method for Measuring Biologic Parameters”; and U.S. patent applications 20080262557 (Oct. 23, 2008 Brown) “Obesity Management System”, 20090281449 (Nov. 12, 2009 Thrower et al.) “Optimization of Thresholds for Eating Detection”, 20100305468 (Dec. 2, 2010 Policker et al.) “Analysis and Regulation of Food Intake”, and 20120316459 (Dec. 13, 2012 Abreu) “Apparatus and Method for Measuring Biologic Parameters”.

### 44. General Sensor (Chemical)

This category of prior art includes sensors which can detect specific types of chemicals. Such sensors can be used for a variety of applications other than modification of food consumption or food absorption. Some are not even directed toward biomedical applications. Nonetheless, overall, they are sufficiently relevant to be included in this review.

Examples of prior art that appear to be best classified in this category include: U.S. Pat. No. 6,218,358 (Apr. 17, 2001 Firestein et al.) “Functional Expression of, and Assay for, Functional Cellular Receptors In Vivo”, U.S. Pat. No. 6,387,329 (May 14, 2002 Lewis et al.) “Use of an Array of Polymeric Sensors of Varying Thickness for Detecting Analytes in Fluids”, U.S. Pat. No. 6,610,367 (Aug. 26, 2003 Lewis et al.) “Use of an Array of Polymeric Sensors of Varying Thickness for Detecting Analytes in Fluids”, U.S. Pat. No. 7,122,152 (Oct. 17, 2006 Lewis et al.) “Spatiotemporal and Geometric Optimization of Sensor Arrays for Detecting Analytes Fluids”, U.S. Pat. No. 7,241,880 (Jul. 10, 2007 Adler et al.) “T1R Taste Receptors and Genes Encoding Same”, U.S. Pat. No. 7,595,023 (Sep. 29, 2009 Lewis et al.) “Spatiotemporal and Geometric Optimization of Sensor Arrays for Detecting Analytes in Fluids”, U.S. Pat. No. 7,651,868 (Jan. 26, 2010 Mcdevitt et al.) “Method and System for the Analysis of Saliva using a Sensor Array”, U.S. Pat. No. 8,067,185 (Nov. 29, 2011 Zoller et al.) “Methods of Quantifying Taste of Compounds for Food or Beverages”, U.S. Pat. No. 8,314,224 (Nov. 20, 2012 Adler et al.) “T1R Taste Receptors and Genes Encoding Same”, and U.S. Pat. No. 8,334,367 (Dec. 18, 2012 Adler) “T2R Taste Receptors and Genes Encoding Same”; and U.S. patent applications 20090261987 (Oct. 22, 2009 Sun) “Sensor Instrument System Including Method for Detecting Analytes in Fluids”, and 20120015432 (Jan. 19, 2012 Adler) “Isolated Bitter Taste Receptor Polypeptides”.

### 45. General Sensor (Microwave)

This category of prior art includes sensors which can detect selected patterns of microwave energy. Such sensors can be used for a variety of applications other than modification of food consumption or food absorption. Nonetheless, overall, they are sufficiently relevant to be included in this review. Examples of prior art that appear to be best classified in this category include U.S. patent applications 20120053426 (Mar. 1, 2012 Webster et al.) “System and Method for Measuring Calorie Content of a Food Sample” and 20130027060 (Jan. 31, 2013 Tralshawala et al.) “Systems and Methods for Non-Destructively Measuring Calorie Contents of Food Items”.

### 46. Sensor (Intraoral)

This category of prior art includes sensors and monitors which are specifically attached or implanted within a person's oral cavity. Examples of prior art that appear to be best classified in this category include: U.S. Pat. No. 8,233,954 (Jul. 31, 2012 Kling et al.) “Mucosal Sensor for the Assessment of Tissue and Blood Constituents and Technique for Using the Same”; and U.S. patent applications 20050263160 (Dec. 1, 2005 Utley et al.) “Intraoral Aversion Devices and Methods”, 20060020298 (Jan. 26, 2006 Camilleri et al.) “Systems and Methods for Curbing Appetite”, 20070106138 (May 10, 2007 Beiski et al.) “Intraoral Apparatus for Non-Invasive Blood and Saliva Monitoring & Sensing”, and 20100209897 (Aug. 19, 2010 Utley et al.) “Intraoral Behavior Monitoring and Aversion Devices and Methods”.

### 49. General Energy Balance Feedback

This category of prior art includes a wide variety of relatively-general systems, devices, and methods that are intended to provide a person with support and feedback concerning their energy balance and weight management. In various examples, systems, devices, and methods in this category can involve: general feedback and behavior modification concerning diet and exercise patterns; broadly-defined use of general types of sensors for energy balance and weight management; interactive communication between people and healthcare providers, or between people and social support networks; internet websites that provide online support for energy balance and weight management; and general meal planning systems and methods. Much of the prior art in this category can be very useful, but is very general compared to the specificity of this present invention. Nonetheless, this general category is included in this review in order to be thorough.

Examples of prior art that appear to be best classified in this category include: U.S. Pat. No. 4,951,197 (Aug. 21, 1990 Mellinger) “Weight Loss Management System”, U.S. Pat. No. 5,720,771 (Feb. 24, 1998 Snell) “Method and Apparatus for Monitoring Physiological Data from an Implantable Medical Device”, U.S. Pat. No. 6,154,676 (Nov. 28, 2000 Levine) “Internal Monitoring and Behavior Control System (Robert Levine)”, U.S. Pat. No. 6,334,073 (Dec. 25, 2001 Levine) “Internal Monitoring and Behavior Control System”, U.S. Pat. No. 6,735,479 (May 11, 2004 Fabian et al.) “Lifestyle Management System”, U.S. Pat. No. 7,247,023 (Jul. 24, 2007 Peplinski et al.) “System and method for monitoring weight and nutrition (Daniel Peplinski)”, and U.S. Pat. No. 7,882,150 (Feb. 1, 2011 Badyal) “Health Advisor”; and U.S. patent applications 20050113649 (May 26, 2005 Bergantino) “Method and Apparatus for Managing a User's Health”, 20060015016 (Jan. 19, 2006 Thornton) “Caloric Balance Weight Control System and Methods of Making and Using Same”, 20060122468 (Jun. 8, 2006 Tavor) “Nutritional Counseling Method and Server”, 20070021979 (Jan. 25, 2007 Cosentino et al.) “Multiuser Wellness Parameter Monitoring System”, 20080221644 (Sep. 11, 2008 Vallapureddy et al.) “Remote Monitoring and Control of Implantable Devices”, and 20120065706 (Mar. 15, 2012 Vallapureddy et al.) “Remote Monitoring and Control of Implantable Devices”.

### 50. Miscellaneous Energy Balance Related Devices and Methods

Lastly, this category of prior art includes a variety of devices and methods that may be generally relevant to the measurement and modification of food consumption, but which resist neat categorization. Examples of prior art in this miscellaneous category include: altering food perception through the use of special tableware; devices that a person activates to emit a bad smell to reduce their appetite; devices that a person uses to shock their tongue when they have a craving; devices to increase airflow through the nose; methods for identifying olfactory cells; time-restricted food containers to control access to food; and using tongue stimulation as a sensory substitute for vision.

Examples of prior art that appear to be best classified in this category include U.S. patents: U.S. Pat. No. 4,582,492 (Apr. 15, 1986 Etter et al.) “Method for Behavior Modification Using Olfactory Stimuli”, U.S. Pat. No. 5,792,210 (Aug. 11, 1998 Wamubu et al.) “Electrical Tongue Stimulator and Method for Addiction Treatment”, U.S. Pat. No. 6,145,503 (Nov. 14, 2000 Smith) “Olfactory Activator”, U.S. Pat. No. 6,159,145 (Dec. 12, 2000 Satoh) “Appetite Adjusting Tool”, U.S. Pat. No. 7,409,647 (Aug. 5, 2008 Elber et al.) “Control of Interactions Within Virtual Environments”, and U.S. Pat. No. 8,060,220 (Nov. 15, 2011 Liebergesell et al.) “Promotion of Oral Hygiene and Treatment of Gingivitis Other Periodontal Problems and Oral Mal Odor”.

Examples of prior art that appear to be best classified in this category also include U.S. patent applications: 20020049482 (Apr. 25, 2002 Fabian et al.) “Lifestyle Management System”, 20040186528 (Sep. 23, 2004 Ries et al.) “Subcutaneous Implantable Medical Devices with Anti-Microbial Agents for Chronic Release”, 20050146419 (Jul. 7, 2005 Porter) “Programmable Restricted Access Food Storage Container and Behavior Modification Assistant”, 20050240253 (Oct. 27, 2005 Tyler et al.) “Systems and Methods for Altering Vestibular Biology”, 20080141282 (Jun. 12, 2008 Elber et al.) “Control of Interactions Within Virtual Environments”, 20080270947 (Oct. 30, 2008 Elber et al.) “Control of Interactions Within Virtual Environments”, 20090197963 (Aug. 6, 2009 Llewellyn) “Method and Compositions for Suppressing Appetite or Treating Obesity”, 20090312817 (Dec. 17, 2009 Hogle et al.) “Systems and Methods for Altering Brain and Body Functions and for Treating Conditions and Diseases of the Same”, 20100055245 (Mar. 4, 2010 Havekotte et al.) “Modifying Flavor Experience Via Aroma Delivery”, 20100291515 (Nov. 18, 2010 Pinnisi et al.) “Regulating Food and Beverage Intake”, 20110314849 (Dec. 29, 2011 Park et al.) “Storage Container with Sensor Device and Refrigerator Having the Same”, 20120009551 (Jan. 12, 2012 Pinnisi) “Cues to Positively Influence Eating Habits”, 20120036875 (Feb. 16, 2012 Yun et al.) “Storage Container with Sensor Device and Refrigerator Having the Same”, and 20120299723 (Nov. 29, 2012 Hafezi et al.) “Communication System Incorporated in a Container”.

### SUMMARY OF THIS INVENTION

This invention can be embodied in an eyewear-based system, device, and method for monitoring a person's nutritional intake comprising eyeglasses, wherein these eyeglasses further comprise at least one camera, wherein this camera automatically takes pictures or records images of food when a person is near food, purchasing food, ordering food, preparing food, and/or consuming food, and wherein these food pictures or images are automatically analyzed to estimate the type and quantity of food. The term food as used herein refers to beverages as well as solid food.

This invention can also be embodied in an eyewear-based system, device, and method for monitoring and modifying a person's nutritional intake comprising eyewear, wherein this eyewear further comprises at least one imaging member, wherein this imaging member automatically takes pictures or records images of food when a person is near food, purchasing food, ordering food, preparing food, and/or consuming food, and wherein these food pictures or images are automatically analyzed to estimate the type and quantity of food; a data processing unit; and a nutritional intake modification component, wherein this component modifies the person's nutritional intake based on the type and quantity of food.

This invention can also be embodied in an eyewear-based system, device, and method for monitoring and modifying a person's nutritional intake comprising: a support member which is configured to be worn on a person's head; at least one optical member which is configured to be held in proximity to an eye by the support member; at least one imaging member, wherein the imaging member is part of or attached to the support member or optical member, wherein this imaging member automatically takes pictures or records images of food when a person is near food, purchasing food, ordering food, preparing food, and/or consuming food, and wherein these food pictures or images are automatically analyzed to estimate the type and quantity of food; a data processing unit; and a nutritional intake modification component, wherein this component modifies the person's nutritional intake based on the type and quantity of food.

### INTRODUCTION TO THE FIGURES

FIGS. 1 through 60 show examples of how this invention may be embodied, but they do not limit the full generalizability of the claims.

FIGS. 1 and 2 show two sequential views of an example of this invention comprising two opposite-facing cameras that are worn on band around a person's wrist.

FIGS. 3 and 4 show pictures of the person's mouth and of a food source from the perspectives of these two cameras.

FIGS. 5 and 6 show an example of this invention with only one camera worn on a band around the person's wrist.

FIGS. 7 and 8 show an example of this invention wherein a camera's field of vision automatically shifts as food moves toward the person's mouth.

FIGS. 9 through 14 show an example of how this invention functions in a six-picture sequence of food consumption.

FIGS. 15 and 16 show a two-picture sequence of how the field of vision from a single wrist-worn camera shifts as the person brings food up to their mouth.

FIGS. 17 and 18 show a two-picture sequence of how the fields of vision from two wrist-worn cameras shift as the person brings food up to their mouth.

FIGS. 19 through 21 show an example of how this invention can be tamper resistant by monitoring the line of sight to the person's mouth and responding if this line of sight is obstructed.

FIG. 22 shows an example of how this invention can be tamper-resistant using a first imaging member to monitor the person's mouth and a second imaging member to scan for food sources.

FIGS. 23 through 30 show two four-picture sequences taken by a wrist-worn prototype of this invention wherein these picture sequences encompass the person's mouth and a food source.

FIGS. 31 through 34 show an example of how this invention can be embodied in a device for selectively and automatically reducing absorption of nutrients from unhealthy food in the context of a longitudinal cross-sectional view of a person's torso.

FIGS. 31 and 32 show an example of how this invention can allow normal absorption of healthy food.

FIGS. 33 and 34 show an example of how this invention can selectively and automatically reduce absorption of nutrients from unhealthy food by coating the walls of a portion of the gastrointestinal tract.

FIGS. 35 and 36 show an example of how this invention can selectively and automatically reduce absorption of nutrients from unhealthy food by coating unhealthy food as it passes through the gastrointestinal tract.

FIGS. 37 and 38 show an example of how this invention can include a mouth-based sensor that triggers the release of a substance into a person's stomach in response to consumption of unhealthy food.

FIGS. 39 and 40 show an example of how this invention can include a mouth-based sensor that triggers electrical stimulation of a person's stomach in response to consumption of unhealthy food.

FIG. 41 shows an eyewear-based system for monitoring and modifying a person's nutritional intake comprising eyewear with an imaging member, a data processing unit, and an implanted electromagnetic energy emitter.

FIG. 42 shows an eyewear-based system for monitoring and modifying a person's nutritional intake comprising eyewear with an imaging member, a motion sensor, a data processing unit, and an implanted electromagnetic energy emitter.

FIG. 43 shows an eyewear-based system for monitoring and modifying a person's nutritional intake comprising eyewear with an imaging member, an electromagnetic energy sensor, a data processing unit, and an implanted electromagnetic energy emitter.

FIG. 44 shows an eyewear-based system for monitoring and modifying a person's nutritional intake comprising eyewear with an imaging member, an intra-oral sensor, a data processing unit, and an implanted electromagnetic energy emitter.

FIG. 45 shows an eyewear-based system for monitoring and modifying a person's nutritional intake comprising eyewear with an imaging member, a wrist-worn sensor, a data processing unit, and an implanted substance-releasing device.

FIG. 46 shows an eyewear-based system for monitoring and modifying a person's nutritional intake comprising eyewear with an imaging member, a wrist-worn sensor, a data processing unit, and an implanted electromagnetic energy emitter.

FIG. 47 shows an eyewear-based system for monitoring and modifying a person's nutritional intake comprising eyewear with an imaging member, a wrist-worn sensor, a data processing unit, and an implanted taste-or-smell-affecting electromagnetic energy emitter.

FIG. 48 shows an eyewear-based system for monitoring and modifying a person's nutritional intake comprising eyewear with an imaging member, a wrist-worn sensor, a data processing unit, and an implanted taste-or-smell-affecting substance-releasing device.

FIG. 49 shows an eyewear-based system for monitoring and modifying a person's nutritional intake comprising eyewear with an imaging member, a wrist-worn sensor, a data processing unit, and an implanted gastrointestinal constriction device.

FIG. 50 shows an eyewear-based system for monitoring and modifying a person's nutritional intake comprising eyewear with an imaging member, a wrist-worn sensor, a data processing unit, and virtually-displayed information.

FIG. 51 shows an eyewear-based system for monitoring and modifying a person's nutritional intake comprising eyewear with an imaging member, a wrist-worn sensor, a data processing unit, and a computer-to-human communication interface.

FIG. 52 shows an eyewear-based system for monitoring and modifying a person's nutritional intake comprising eyewear with an imaging member and at least one electromagnetic brain activity sensor, a wrist-worn sensor, a data processing unit, and an implanted substance-releasing device.

FIG. 53 shows an eyewear-based system for monitoring and modifying a person's nutritional intake comprising eyewear with an imaging member and at least one electromagnetic brain activity sensor, a wrist-worn sensor, a data processing unit, and an implanted electromagnetic energy emitter.

FIG. 54 shows an eyewear-based system for monitoring and modifying a person's nutritional intake comprising eyewear with an imaging member and at least one electromagnetic brain activity sensor, a wrist-worn sensor, a data processing unit, and an implanted taste-or-smell-affecting electromagnetic energy emitter.

FIG. 55 shows an eyewear-based system for monitoring and modifying a person's nutritional intake comprising eyewear with an imaging member and at least one electromagnetic brain activity sensor, a wrist-worn sensor, a data processing unit, and an implanted taste-or-smell-affecting substance-releasing device.

FIG. 56 shows an eyewear-based system for monitoring and modifying a person's nutritional intake comprising eyewear with an imaging member and at least one electromagnetic brain activity sensor, a wrist-worn sensor, a data processing unit, and an implanted gastrointestinal constriction device.

FIG. 57 shows an eyewear-based system for monitoring and modifying a person's nutritional intake comprising eyewear with an imaging member and at least one electromagnetic brain activity sensor, a wrist-worn sensor, a data processing unit, and virtually-displayed information.

FIG. 58 shows an eyewear-based system for monitoring and modifying a person's nutritional intake comprising eyewear with an imaging member and at least one electromagnetic brain activity sensor, a wrist-worn sensor, a data processing unit, and a computer-to-human communication interface.

FIGS. 59 and 60 show examples of eyewear for monitoring a person's electromagnetic brain activity comprising at least one optical member, a support member with at least one upward protrusion, and at least one electromagnetic brain activity sensor.

### DETAILED DESCRIPTION OF THE FIGURES

The examples shown in these figures are not exhaustive and do not limit the full generalizability of the claims. Before going into a detailed description of the figures, it is important to first define three terms that are used repeatedly in the description.

The first term, “food,” is broadly defined to include liquid nourishment, such as beverages, in addition to solid food. The second term, “reachable food source,” is defined as a source of food that a person can access and from which they can bring a piece (or portion) of food to their mouth by moving their arm and hand. Arm and hand movement can include movement of the person's shoulder, elbow, wrist, and finger joints. In various examples, a reachable food source can be selected from the group consisting of: food on a plate, food in a bowl, food in a glass, food in a cup, food in a bottle, food in a can, food in a package, food in a container, food in a wrapper, food in a bag, food in a box, food on a table, food on a counter, food on a shelf, and food in a refrigerator.

The third term, “food consumption pathway,” is defined as a path in space that is traveled by (a piece of) food from a reachable food source to a person's mouth as the person eats. The distal endpoint of a food consumption pathway is the reachable food source and the proximal endpoint of a food consumption pathway is the person's mouth. In various examples, food may be moved along the food consumption pathway by contact with a member selected from the group consisting of: a utensil; a beverage container; the person's fingers; and the person's hand.

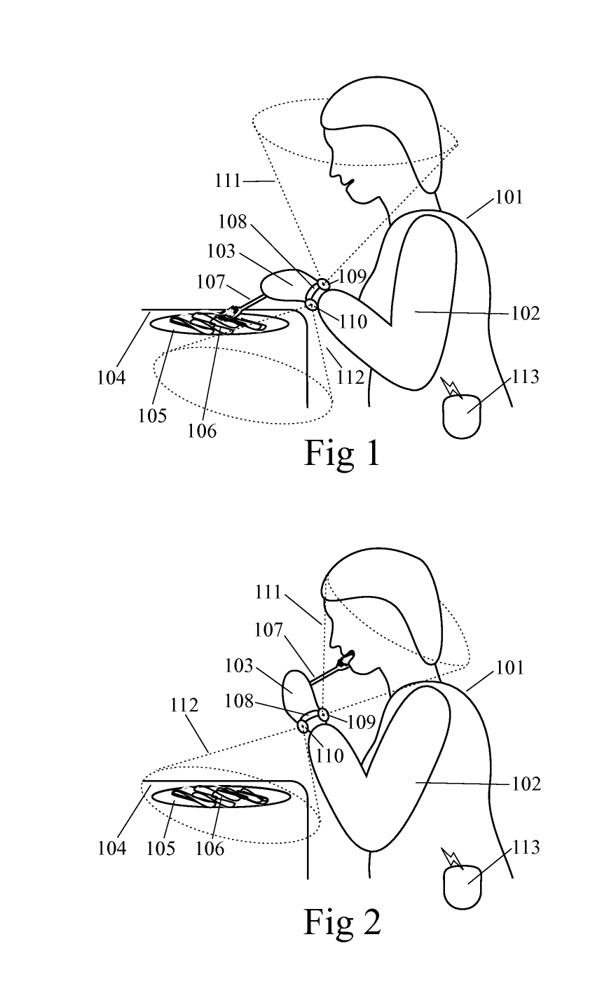

We now begin the description of FIGS. 1 and 2 with an introductory overview. A detailed description will follow. FIGS. 1 and 2 show one example of how this invention may be embodied in a device and method for automatically monitoring and estimating human caloric intake. In this example, the device and method comprise an automatic-imaging member that is worn on a person's wrist. This imaging member has two cameras attached to a wrist band on opposite (narrow) sides of the person's wrist.

These two cameras take pictures of a reachable food source and the person's mouth. These pictures are used to estimate, in an automatic and tamper-resistant manner, the types and quantities of food consumed by the person. Information on food consumed, in turn, is used to estimate the person's caloric intake. As the person eats, these two cameras of the automatic-imaging member take pictures of a reachable food source and the person's mouth. These pictures are analyzed, using pattern recognition or other image-analyzing methods, to estimate the types and quantities of food that the person consumes. In this example, these pictures are motion pictures (e.g. videos). In another example, these pictures may be still-frame pictures.

We now discuss FIGS. 1 and 2, including their components, in detail. FIG. 1 shows person 101 seated at table 104 wherein this person is using their arm 102 and hand 103 to access food 106 on plate 105 located on table 104. In this example in FIGS. 1 and 2, food 106 on plate 105 comprises a reachable food source. In this example, person 101 is shown picking up a piece of food 106 from the reachable food source using utensil 107. In various examples, a food source may be selected from the group consisting of: food on a plate, food in a bowl, food in a glass, food in a cup, food in a bottle, food in a can, food in a package, food in a container, food in a wrapper, food in a bag, food in a box, food on a table, food on a counter, food on a shelf, and food in a refrigerator.

In this example, the person is wearing an automatic-imaging member comprised of a wrist band 108 to which are attached two cameras, 109 and 110, on the opposite (narrow) sides of the person's wrist. Camera 109 takes pictures within field of vision 111. Camera 110 takes pictures within field of vision 112. Each field of vision, 111 and 112, is represented in these figures by a dotted-line conical shape. The narrow tip of the dotted-line cone is at the camera's aperture and the circular base of the cone represents the camera's field of vision at a finite focal distance from the camera's aperture.

In this example, camera 109 is positioned on the person's wrist at a location from which it takes pictures along an imaging vector that is directed generally upward from the automatic-imaging member toward the person's mouth as the person eats. In this example, camera 110 is positioned on the person's wrist at a location from which it takes pictures along an imaging vector that is directed generally downward from the automatic-imaging member toward a reachable food source as the person eats. These imaging vectors are represented in FIG. 1 by the fields of vision, 111 and 112, indicated by cone-shaped dotted-line configurations. The narrow end of the cone represents the aperture of the camera and the circular end of the cone represents a focal distance of the field of vision as seen by the camera. Although theoretically the field of vision could extend outward in an infinite manner from the aperture, we show a finite length cone to represent a finite focal length for a camera's field of vision.

Field of vision 111 from camera 109 is represented in FIG. 1 by a generally upward-facing cone-shaped configuration of dotted lines that generally encompasses the person's mouth and face as the person eats. Field of vision 112 from camera 110 is represented in FIG. 1 by a generally downward-facing cone-shaped configuration of dotted lines that generally encompasses the reachable food source as the person eats.

This device and method of taking pictures of both a reachable food source and the person's mouth, while a person eats, can do a much better job of estimating the types and quantities of food actually consumed than one of the devices or methods in the prior art that only takes pictures of either a reachable food source or the person's mouth. There is prior art that uses imaging to identify food that requires a person to manually aim a camera toward a food source and then manually take a picture of the food source. Such prior art does not take also pictures of the person's mouth. There are multiple disadvantages with this prior art. We will discuss later the disadvantages of requiring manual intervention to aim a camera and push a button to take a picture. For now, we discuss the disadvantages of prior art that only takes pictures of a reachable food source or only takes pictures of the person's mouth, but not both.

First, let us consider a “source-only” imaging device, such as those in the prior art, that only takes pictures of a food source within a reachable distance of the person and does not also take pictures of the person's mouth. Using a “source-only” device, it is very difficult to know whether the person actually consumes the food that is seen in the pictures. A “source-only” imaging device can be helpful in identifying what types of foods the person has reachable access to, and might possibly eat, but such a device is limited as means for measuring how much of these foods the person actually consumes. For example, consider a person walking through a grocery store. As the person walks through the store, a wide variety of food sources in various packages and containers come into a wearable camera's field of vision. However, the vast majority of these food sources are ones that the person never consumes. The person only actually consumes those foods that the person buys and consumes later. An automatic wearable imaging system that only takes pictures of reachable food sources would be very limited for determining how many of these reachable food sources are actually consumed by the person.

One could try to address this problem by making the picture-taking process a manual process rather than an automatic process. One could have an imaging system that requires human intervention to actively aim a camera (e.g. a mobile imaging device) at a food source and also require human intervention (to click a button) to indicate that the person is actually going to consume that food. However, relying on such a manual process for caloric intake monitoring makes this process totally dependent on the person's compliance. Even if a person wants to comply, it can be tough for a person to manually aim a camera and take pictures each time that the person snacks on something. If the person does not want to comply, the situation is even worse. It is easy for a person to thwart a monitoring process that relies on manual intervention. All that a person needs to do to thwart the process is to not take pictures of something that they eat.

A manual imaging system is only marginally better than old-fashioned “calorie counting” by writing down what a person eats on a piece of paper or entering it into a computer. If a person buys a half-gallon of ice cream and consumes it without manually taking a picture of the ice-cream, either intentionally or by mistaken omission, then the device that relies on a manual process is clueless with respect to those calories consumed. A “source-only” imaging device makes it difficult, if not impossible, to track food actually consumed without manual intervention. Further, requiring manual intervention to record consumption makes it difficult, if not impossible, to fully automate calorie monitoring and estimation.

As another example of the limitations of a “source-only” imaging device, consider the situation of a person sitting at a table with many other diners wherein the table is set with food in family-style communal serving dishes. These family-style dishes are passed around to serve food to everyone around the table. It would be challenging for a “source-only” imaging device to automatically differentiate between these communal serving dishes and a person's individual plate. What happens when the person's plate is removed or replaced? What happens when the person does not eat all of the food on their plate? These examples highlight the limitations of a device and method that only takes pictures of a reachable food source, without also taking pictures of the person's mouth.

This present invention overcomes these limitations by automatically taking pictures of both a reachable food source and the person's mouth. With images of both a reachable food source and the person's mouth, as the person eats, this present device and method can determine not only what food the person has access to, but how much of that food the person actually eats.

We have considered the limitations of devices and methods in the prior art that only take pictures of a reachable food source. We now also consider the limitations of “mouth-only” imaging devices and methods, wherein these devices only take pictures of the person's mouth while they eat. It is very difficult for a “mouth-only” imaging device to use pattern recognition, or some other image-based food identification method, on a piece of food approaching the person's mouth to identify the food, without also having pictures of the total food source.

For example, pattern recognition software can identify the type of food at a reachable food source by: analyzing the food's shape, color, texture, and volume; or by analyzing the food's packaging. However, it is much more difficult for a device to identify a piece (or portion) of food that is obscured within in the scoop of a spoon, hidden within a cup, cut and then pierced by the tines of a fork, or clutched in partially-closed hand as it is brought up to the person's mouth.

For example, pattern recognition software could identify a bowl of peanuts on a table, but would have a tough time identifying a couple peanuts held in the palm of a person's partially-closed hand as they move from the bowl to the person's mouth. It is difficult to get a line of sight from a wearable imaging member to something inside the person's hand as it travels along the food consumption pathway. For these reasons, a “mouth-only” imaging device may be useful for estimating the quantity of food consumed (possibly based on the number of food consumption pathway motions, chewing motions, swallowing motions, or a combination thereof) but is limited for identifying the types of foods consumed, without having food source images as well.

We have discussed the limitations of “source-only” and “mouth-only” prior art that images only a reachable food source or only a person's mouth. This present invention is an improvement over this prior art because it comprises a device and method that automatically estimates the types and quantities of food actually consumed based on pictures of both a reachable food source and the person's mouth. Having both such images provides better information than either separately. Pictures of a reachable food source may be particularly useful for identifying the types of food available to the person for potential consumption. Pictures of the person's mouth (including food traveling the food consumption pathway and food-mouth interaction such as chewing and swallowing) may be particularly useful for identifying the quantity of food consumed by the person. Combining both images in an integrated analysis provides more accurate estimation of the types and quantities of food actually consumed by the person. This information, in turn, provides better estimation of caloric intake by the person.

The fact that this present invention is wearable further enhances its superiority over prior art that is non-wearable. It is possible to have a non-wearable imaging device that can be manually positioned (on a table or other surface) to be aimed toward an eating person, such that its field of vision includes both a food source and the person's mouth. In theory, every time the person eats a meal or takes a snack, the person could: take out an imaging device (such as a smart phone); place the device on a nearby surface (such as a table, bar, or chair); manually point the device toward them so that both the food source and their mouth are in the field of vision; and manually push a button to initiate picture taking before they start eating. However, this manual process with a non-wearable device is highly dependent on the person's compliance with this labor-intensive and possibly-embarrassing process.

Even if a person has good intentions with respect to compliance, it is expecting a lot for a person to carry around a device and to set it up at just the right direction each time that the person reaches for a meal or snack. How many people, particularly people struggling with their weight and self-image, would want to conspicuously bring out a mobile device, place it on a table, and manually aim it toward themselves when they eat, especially when they are out to eat with friends or on a date? Even if this person has good intentions with respect to compliance with a non-wearable food-imaging device, it is very unlikely that compliance would be high. The situation would get even worse if the person is tempted to obstruct the operation of the device to cheat on their “diet.” With a non-wearable device, tampering with the operation of the device is easy as pie (literally). All the person has to do is to fail to properly place and activate the imaging device when they snack.

It is difficult to design a non-wearable imaging device that takes pictures, in an automatic and tamper-resistant manner, of both a food source and the person's mouth whenever the person eats. Is it easier to design a wearable imaging device that takes pictures, in an automatic and tamper-resistant manner, of a food source and the person's mouth whenever the person eats. Since the device and method disclosed herein is wearable, it is an improvement over non-wearable prior art, even if that prior art could be used to manually take pictures of a food source and a person's mouth.

The fact that the device and method disclosed herein is wearable makes it less dependent on human intervention, easier to automate, and easier to make tamper-resistant. With the present invention, there is no requirement that a person must carry around a mobile device, place it on an external surface, and aim it toward a food source and their mouth every time that they eat in order to track total caloric intake. This present device, being wearable and automatic, goes with the person where ever they go and automatically takes pictures whenever they eat, without the need for human intervention.

In an example, this device may have an unobtrusive, or even attractive, design like a piece of jewelry. In various examples, this device may look similar to an attractive wrist watch, bracelet, finger ring, necklace, or ear ring. As we will discuss further, the wearable and automatic imaging nature of this invention allows the incorporation of tamper-resistant features into this present device to increase the accuracy and compliance of caloric intake monitoring and estimation.

For measuring total caloric intake, ideally it is desirable to have a wearable device and method that automatically monitors and estimates caloric intake in a comprehensive and involuntary manner. The automatic and involuntary nature of a device and method will enhance accuracy and compliance. This present invention makes significant progress toward this goal, especially as compared to the limitations of relevant prior art. There are devices and methods in the prior art that assist in manual calorie counting, but they are heavily reliant on the person's compliance. The prior art does not appear to disclose a wearable, automatic, tamper-resistant, image-based device or method that takes pictures of a food source and a person's mouth in order to estimate the person's caloric intake.

The fact that this device and method incorporates pictures of both a food source and the person's mouth, while a person eats, makes it much more accurate than prior art that takes pictures of only a food source or only the person's mouth. The wearable nature of this invention makes it less reliant on manual activation, and much more automatic in its imaging operation, than non-wearable devices. This present device does not depend on properly placing, aiming, and activating an imaging member every time a person eats. This device and method operates in an automatic manner and is tamper resistant. All of these features combine to make this invention a more accurate and dependable device and method of monitoring and measuring human caloric intake than devices and methods in the prior art. This present invention can serve well as the caloric-intake measuring component of an overall system of human energy balance and weight management.

In the example of this invention that is shown in FIG. 1, the pictures of the person's mouth and the pictures of the reachable food source that are taken by cameras 109 and 110 (part of a wrist-worn automatic-imaging member) are transmitted wirelessly to image-analyzing member 113 that is worn elsewhere on the person. In this example, image-analyzing member 113 automatically analyzes these images to estimate the types and quantities of food consumed by the person. There are many methods of image analysis and pattern recognition in the prior art and the precise method of image analysis is not central to this invention. Accordingly, the precise method of image analysis is not specified herein.

In an example, this invention includes an image-analyzing member that uses one or more methods selected from the group consisting of: pattern recognition or identification; human motion recognition or identification; face recognition or identification; gesture recognition or identification; food recognition or identification; word recognition or identification; logo recognition or identification; bar code recognition or identification; and 3D modeling.

In an example, this invention includes an image-analyzing member that analyzes one or more factors selected from the group consisting of: number of reachable food sources; types of reachable food sources; changes in the volume of food at a reachable food source; number of times that the person brings food to their mouth; sizes of portions of food that the person brings to their mouth; number of chewing movements; frequency or speed of chewing movements; and number of swallowing movements.

In an example, this invention includes an image-analyzing member that provides an initial estimate of the types and quantities of food consumed by the person and this initial estimate is then refined by human interaction and/or evaluation.

In an example, this invention includes wireless communication from a first wearable member (that takes pictures of a reachable food source and a person's mouth) to a second wearable member (that analyzes these pictures to estimate the types and quantities of food consumed by the person). In another example, this invention may include wireless communication from a wearable member (that takes pictures of a reachable food source and a person's mouth) to a non-wearable member (that analyzes these pictures to estimate the types and quantities of food consumed by the person). In another example, this invention may include a single wearable member that takes and analyzes pictures, of a reachable food source and a person's mouth, to estimate the types and quantities of food consumed by the person.

In the example of this invention that is shown in FIG. 1, an automatic-imaging member is worn around the person's wrist. Accordingly, the automatic-imaging member moves as food travels along the food consumption pathway. This means that the imaging vectors and the fields of vision, 111 and 112, from the two cameras, 109 and 110, that are located on this automatic-imaging member, shift as the person eats.

In this example, the fields of vision from these two cameras on the automatic-imaging member automatically and collectively encompass the person's mouth and a reachable food source, from at least some locations, as the automatic-imaging member moves when food travels along the food consumption pathway. In this example, this movement allows the automatic-imaging member to take pictures of both the person's mouth and the reachable food source, as the person eats, without the need for human intervention to manually aim cameras toward either the person's mouth or a reachable food source, when the person eats.

The reachable food source and the person's mouth do not need to be within the fields of vision, 111 and 112, at all times in order for the device and method to accurately estimate food consumed. As long as the reachable food source and the person's mouth are encompassed by the field of vision from at least one of the two cameras at least once during each movement cycle along the food consumption pathway, the device and method should be able to reasonably interpolate missing intervals and to estimate the types and quantities of food consumed.

FIG. 2 shows the same example of the device and method for automatically monitoring and estimating caloric intake that was shown in FIG. 1, but at a later point as food moves along the food consumption pathway. In FIG. 2, a piece of food has traveled from the reachable food source to the person's mouth via utensil 107. In FIG. 2, person 101 has bent their arm 102 and rotated their hand 103 to bring this piece of food, on utensil 107, up to their mouth. In FIG. 2, field of vision 112 from camera 110, located on the distal side of the person's wrist, now more fully encompasses the reachable food source. Also, field of vision 111 from camera 109, located on the proximal side of the person's wrist, now captures the interaction between the piece of food and the person's mouth.

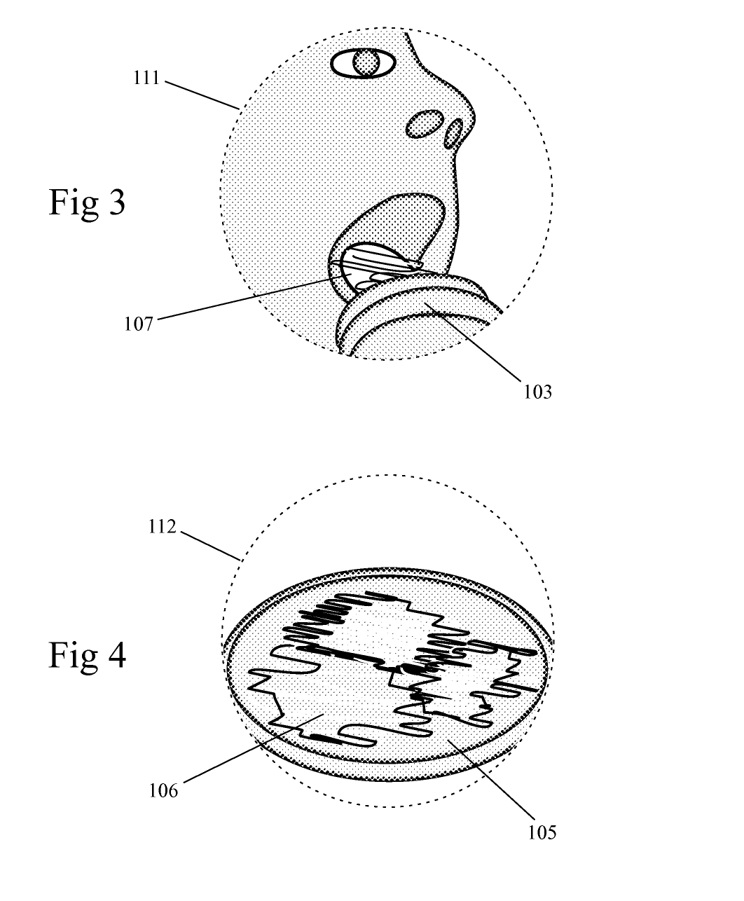

FIGS. 3 and 4 provide additional insight into how this device and method for monitoring and estimating caloric intake works. FIGS. 3 and 4 show still-frame views of the person's mouth and the reachable food source as captured by the fields of vision, 111 and 112, from the two cameras, 109 and 110, worn on the person's wrist, as the person eats. In FIGS. 3 and 4, the boundaries of fields of vision 111 and 112 are represented by dotted-line circles. These dotted-line circles correspond to the circular ends of the dotted-line conical fields of vision that are shown in FIG. 2.

For example, FIG. 2 shows a side view of camera 109 with conical field of vision 111 extending outwards from the camera aperture and upwards toward the person's mouth. FIG. 3 shows this same field of vision 111 from the perspective of the camera aperture. In FIG. 3, the person's mouth is encompassed by the circular end of the conical field of vision 111 that was shown in FIG. 2. In this manner, FIG. 3 shows a close-up view of utensil 107, held by hand 103, as it inserts a piece of food into the person's mouth.

As another example, FIG. 2 shows a side view of camera 110 with conical field of vision 112 extending outwards from the camera aperture and downwards toward the reachable food source. In this example, the reachable food source is food 106 on plate 105. FIG. 4 shows this same field of vision 112 from the perspective of the camera aperture. In FIG. 4, the reachable food source is encompassed by the circular end of the conical field of vision 112 that was shown in FIG. 2. In this manner, FIG. 4 shows a close-up view of food 106 on plate 105.

The example of this invention for monitoring and estimating human caloric intake that is shown in FIGS. 1-4 comprises a wearable imaging device. In various examples, this invention can be a device and method for measuring caloric intake that comprises one or more automatic-imaging members that are worn on a person at one or more locations from which these members automatically take (still or motion) pictures of the person's mouth as the person eats and automatically take (still or motion) pictures of a reachable food source as the person eats. In this example, these images are automatically analyzed to estimate the types and quantities of food actually consumed by the person.

In an example, there may be one automatic-imaging member that takes pictures of both the person's mouth and a reachable food source. In an example, there may be two or more automatic-imaging members, worn on one or more locations on a person, that collectively and automatically take pictures of the person's mouth when the person eats and pictures of a reachable food source when the person eats. In an example, this picture taking can occur in an automatic and tamper-resistant manner as the person eats.

In various examples, one or more imaging devices worn on a person's body take pictures of food at multiple points as it moves along the food consumption pathway. In various examples, this invention comprises a wearable, mobile, calorie-input-measuring device that automatically records and analyzes food images in order to detect and measure human caloric input. In various examples, this invention comprises a wearable, mobile, energy-input-measuring device that automatically analyzes food images to measure human energy input.

In an example, this device and method comprise one or more imaging members that take pictures of: food at a food source; a person's mouth; and interaction between food and the person's mouth. The interaction between the person's mouth and food can include biting, chewing, and swallowing. In an example, utensils or beverage-holding members may be used as intermediaries between the person's hand and food. In an example, this invention comprises an imaging device that automatically takes pictures of the interaction between food and the person's mouth as the person eats. In an example, this invention comprises a wearable device that takes pictures of a reachable food source that is located in front of the person.

In an example, this invention comprises a method of estimating a person's caloric intake that includes the step of having the person wear one or more imaging devices, wherein these imaging devices collectively and automatically take pictures of a reachable food source and the person's mouth. In an example, this invention comprises a method of measuring a person's caloric intake that includes having the person wear one or more automatic-imaging members, at one or more locations on the person, from which locations these members are able to collectively and automatically take pictures of the person's mouth as the person eats and take pictures of a reachable food source as the person eats.

In the example of this invention that is shown in FIGS. 1 and 2, two cameras, 109 and 110, are worn on the narrow sides of the person's wrist, between the posterior and anterior surfaces of the wrist, such that the moving field of vision from the first of these cameras automatically encompasses the person's mouth (as the person moves their arm when they eat) and the moving field of vision from the second of these cameras automatically encompasses the reachable food source (as the person moves their arm when they eat). This embodiment of the invention is comparable to a wrist-watch that has been rotated 90 degrees around the person's wrist, with a first camera located where the watch face would be and a second camera located on the opposite side of the wrist.

In another example, this device and method can comprise an automatic-imaging member with a single wide-angle camera that is worn on the narrow side of a person's wrist or upper arm, in a manner similar to wearing a watch or bracelet that is rotated approximately 90 degrees. This automatic-imaging member can automatically take pictures of the person's mouth, a reachable food source, or both as the person moves their arm and hand as the person eats. In another example, this device and method can comprise an automatic-imaging member with a single wide-angle camera that is worn on the anterior surface of a person's wrist or upper arm, in a manner similar to wearing a watch or bracelet that is rotated approximately 180 degrees. This automatic-imaging member automatically takes pictures of the person's mouth, a reachable food source, or both as the person moves their arm and hand as the person eats. In another example, this device and method can comprise an automatic-imaging member that is worn on a person's finger in a manner similar to wearing a finger ring, such that the automatic-imaging member automatically takes pictures of the person's mouth, a reachable food source, or both as the person moves their arm and hand as the person eats.

In various examples, this invention comprises a caloric-input measuring member that automatically estimates a person's caloric intake based on analysis of pictures taken by one or more cameras worn on the person's wrist, hand, finger, or arm. In various examples, this invention includes one or more automatic-imaging members worn on a body member selected from the group consisting of: wrist, hand, finger, upper arm, and lower arm. In various examples, this invention includes one or more automatic-imaging members that are worn in a manner similar to a wearable member selected from the group consisting of: wrist watch; bracelet; arm band; and finger ring.

In various examples of this device and method, the fields of vision from one or more automatic-imaging members worn on the person's wrist, hand, finger, or arm are shifted by movement of the person's arm bringing food to their mouth along the food consumption pathway. In an example, this movement causes the fields of vision from these one or more automatic-imaging members to collectively and automatically encompass the person's mouth and a reachable food source.

In various examples, this invention includes one or more automatic-imaging members that are worn on a body member selected from the group consisting of: neck; head; and torso. In various examples, this invention includes one or more automatic-imaging members that are worn in a manner similar to a wearable member selected from the group consisting of: necklace; pendant, dog tags; brooch; cuff link; ear ring; eyeglasses; wearable mouth microphone; and hearing aid.

In an example, this device and method comprise at least two cameras or other imaging members. A first camera may be worn on a location on the human body from which it takes pictures along an imaging vector which points toward the person's mouth while the person eats. A second camera may be worn on a location on the human body from which it takes pictures along an imaging vector which points toward a reachable food source. In an example, this invention may include: (a) an automatic-imaging member that is worn on the person's wrist, hand, arm, or finger such that the field of vision from this member automatically encompasses the person's mouth as the person eats; and (b) an automatic-imaging member that is worn on the person's neck, head, or torso such that the field of vision from this member automatically encompasses a reachable food source as the person eats.

In other words, this device and method can comprise at least two automatic-imaging members that are worn on a person's body. One of these automatic-imaging members may be worn on a body member selected from the group consisting of the person's wrist, hand, lower arm, and finger, wherein the field of vision from this automatic-imaging member automatically encompasses the person's mouth as the person eats. A second of these automatic-imaging members may be worn on a body member selected from the group consisting of the person's neck, head, torso, and upper arm, wherein the field of vision from the second automatic-imaging member automatically encompasses a reachable food source as the person eats.

In various examples, one or more automatic-imaging members may be integrated into one or more wearable members that appear similar to a wrist watch, wrist band, bracelet, arm band, necklace, pendant, brooch, collar, eyeglasses, ear ring, headband, or ear-mounted bluetooth device. In an example, this device may comprise two imaging members, or two cameras mounted on a single member, which are generally perpendicular to the longitudinal bones of the upper arm. In an example, one of these imaging members may have an imaging vector that points toward a food source at different times while food travels along the food consumption pathway. In an example, another one of these imaging members may have an imaging vector that points toward the person's mouth at different times while food travels along the food consumption pathway. In an example, these different imaging vectors may occur simultaneously as food travels along the food consumption pathway. In another example, these different imaging vectors may occur sequentially as food travels along the food consumption pathway. This device and method may provide images from multiple imaging vectors, such that these images from multiple perspectives are automatically and collectively analyzed to identify the types and quantities of food consumed by the person.

In an example of this invention, multiple imaging members may be worn on the same body member. In another example, multiple imaging members may be worn on different body members. In an example, an imaging member may be worn on each of a person's wrists or each of a person's hands. In an example, one or more imaging members may be worn on a body member and a supplemental imaging member may be located in a non-wearable device that is in proximity to the person. In an example, wearable and non-wearable imaging members may be in wireless communication with each other. In an example, wearable and non-wearable imaging members may be in wireless communication with an image-analyzing member.

In an example, a wearable imaging member may be worn on the person's body, a non-wearable imaging member may be positioned in proximity to the person's body, and a tamper-resisting mechanism may ensure that both the wearable and non-wearable imaging members are properly positioned to take pictures as the person eats. In various examples, this device and method may include one or more imaging members that are worn on the person's neck, head, or torso and one or more imaging devices that are positioned on a table, counter, or other surface in front of the person in order to simultaneously, or sequentially, take pictures of a reachable food source and the person's mouth as the person eats.

In an example, this invention comprises an imaging device with multiple imaging components that take images along different imaging vectors so that the device takes pictures of a reachable food source and a person's mouth simultaneously. In an example, this invention comprises an imaging device with a wide-angle lens that takes pictures within a wide field of vision so that the device takes pictures of a reachable food source and a person's mouth simultaneously.

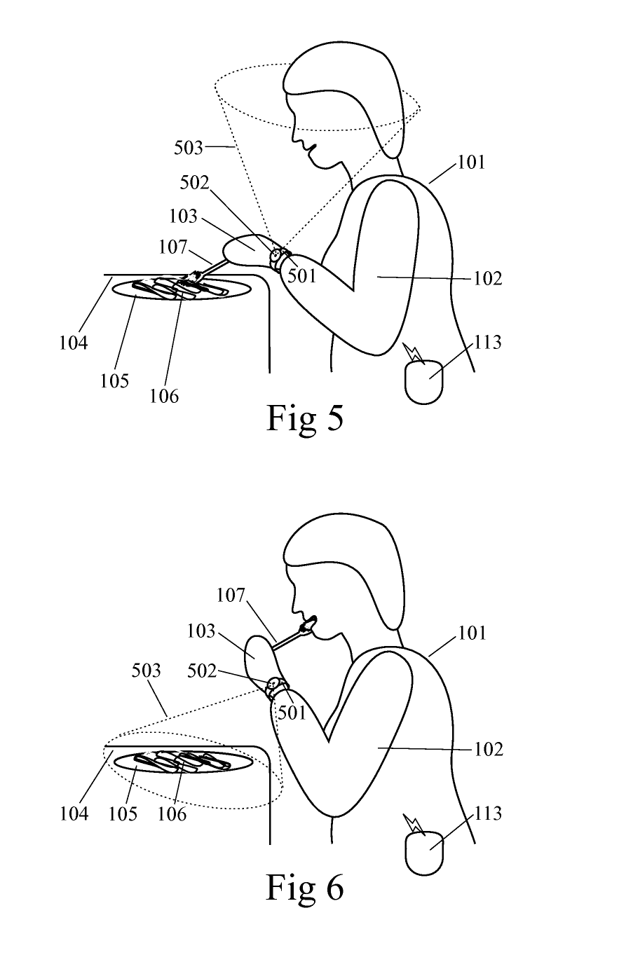

FIGS. 5 through 8 show additional examples of how this device and method for monitoring and estimating human caloric intake can be embodied. These examples are similar to the examples shown previously in that they comprise one or more automatic-imaging members that are worn on a person's wrist. These examples similar to the example shown in FIGS. 1 and 2, except that now in FIGS. 5 through 8 there is only one camera 502 located a wrist band 501.

This automatic-imaging member has features that enable the one camera, 502, to take pictures of both the person's mouth and a reachable food source with only a single field of vision 503. In an example, this single wrist-mounted camera has a wide-angle lens that allows it to take pictures of the person's mouth when a piece of food is at a first location along the food consumption pathway (as shown in FIG. 5) and allows it to take pictures of a reachable food source when a piece food is at a second location along the food consumption pathway (as shown in FIG. 6).

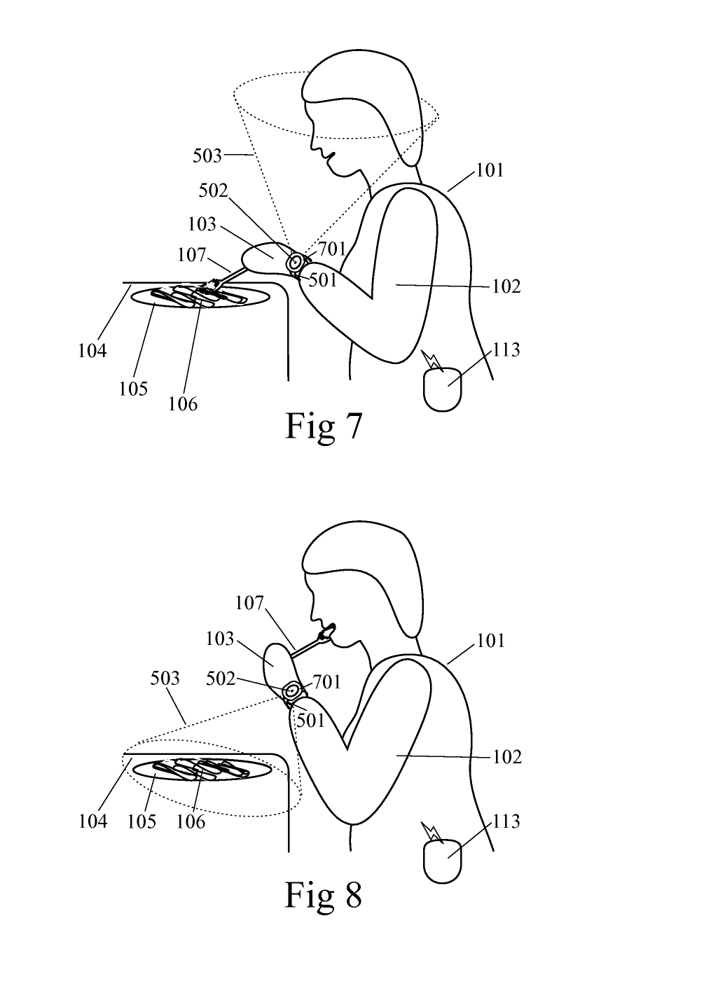

In an example, such as that shown in FIGS. 7 and 8, a single wrist-mounted camera is linked to a mechanism that shifts the camera's imaging vector (and field of vision) automatically as food moves along the food consumption pathway. This shifting imaging vector allows a single camera to encompass a reachable food source and the person's mouth, sequentially, from different locations along the food consumption pathway.

In the example of this invention that is shown in FIGS. 7 and 8, an accelerometer 701 is worn on the person's wrist and linked to the imaging vector of camera 502. Accelerometer 701 detects arm and hand motion as food moves along the food consumption pathway. Information concerning this arm and hand movement is used to automatically shift the imaging vector of camera 502 such that the field of vision, 503, of camera 502 sequentially captures images of the reachable food source and the person's mouth from different positions along the food consumption pathway. In an example, when accelerometer 701 indicates that the person's arm is in the downward phase of the food consumption pathway (in proximity to the reachable food source) then the imaging vector of camera 502 is directed upwards to get a good picture of the person's mouth interacting with food. Then, when accelerometer 701 indicates that the person's arm is in the upward phase of the food consumption pathway (in proximity to the person's mouth), the imaging vector of camera 502 is directed downwards to get a good picture of the reachable food source.

A key advantage of this present invention for monitoring and measuring a person's caloric intake is that it works in an automatic and (virtually) involuntary manner. It does not require human intervention each time that a person eats to aim a camera and push a button in order to take the pictures necessary to estimate the types and quantities of food consumed. This is a tremendous advantage over prior art that requires human intervention to aim a camera (at a food source, for example) and push a button to manually take pictures. The less human intervention that is required to make the device work, the more accurate the device and method will be in measuring total caloric intake. Also, the less human intervention that is required, the easier it is to make the device and method tamper-resistant.

Ideally, one would like an automatic, involuntary, and tamper-resistant device and method for monitoring and measuring caloric intake—a device and method which not only operates independently from human intervention at the time of eating, but which can also detect and respond to possible tampering or obstruction of the imaging function. At a minimum, one would like a device and method that does not rely on the person to manually aim a camera and manually initiate pictures each time the person eats. A manual device puts too much of a burden on the person to stay in compliance. At best, one would like a device and method that detects and responds if the person tampers with the imaging function of the device and method. This is critical for obtaining an accurate overall estimate of a person's caloric intake. The device and method disclosed herein is a significant step toward an automatic, involuntary, and tamper-resistant device, system, and method of caloric intake monitoring and measuring.

In an example, this device and method comprise one or more automatic-imaging members that automatically and collectively take pictures of a person's mouth and pictures of a reachable food source as the person eats, without the need for human intervention to initiate picture taking when the person starts to eat. In an example, this invention comprises one or more automatic-imaging members that collectively and automatically take pictures of the person's mouth and pictures of a reachable food source, when the person eats, without the need for human intervention, when the person eats, to activate picture taking by pushing a button on a camera.

In an example, one way to design a device and method to take pictures when a person eats without the need for human intervention is to simply have the device take pictures continuously. If the device is never turned off and takes pictures all the time, then it necessarily takes pictures when a person eats. In an example, such a device and method can: continually track the location of, and take pictures of, the person's mouth; continually track the location of, and take pictures of, the person's hands; and continually scan for, and take pictures of, any reachable food sources nearby.

However, having a wearable device that takes pictures all the time can raise privacy concerns. Having a device that continually takes pictures of a person's mouth and continually scans space surrounding the person for potential food sources may be undesirable in terms of privacy, excessive energy use, or both. People may be so motivated to monitor caloric intake and to lose weight that the benefits of a device that takes pictures all the time may outweigh privacy concerns. Accordingly, this invention may be embodied in a device and method that takes pictures all the time. However, for those for whom such privacy concerns are significant, we now consider some alternative approaches for automating picture taking when a person eats.

In an example, an alternative approach to having imaging members take pictures automatically when a person eats, without the need for human intervention, is to have the imaging members start taking pictures only when sensors indicate that the person is probably eating. This can reduce privacy concerns as compared to a device and method that takes pictures all the time. In an example, an imaging device and method can automatically begin taking images when wearable sensors indicate that the person is probably consuming food.

In an example of this alternative approach, this device and method may take pictures of the person's mouth and scan for a reachable food source only when a wearable sensor, such as the accelerometer 701 in FIGS. 7 and 8, indicates that the person is (probably) eating. In various examples, one or more sensors that detect when the person is (probably) eating can be selected from the group consisting of: accelerometer, inclinometer, motion sensor, sound sensor, smell sensor, blood pressure sensor, heart rate sensor, EEG sensor, ECG sensor, EMG sensor, electrochemical sensor, gastric activity sensor, GPS sensor, location sensor, image sensor, optical sensor, piezoelectric sensor, respiration sensor, strain gauge, electrogoniometer, chewing sensor, swallow sensor, temperature sensor, and pressure sensor.

In various examples, indications that a person is probably eating may be selected from the group consisting of: acceleration, inclination, twisting, or rolling of the person's hand, wrist, or arm; acceleration or inclination of the person's lower arm or upper arm; bending of the person's shoulder, elbow, wrist, or finger joints; movement of the person's jaw, such as bending of the jaw joint; smells suggesting food that are detected by an artificial olfactory sensor; detection of chewing, swallowing, or other eating sounds by one or more microphones; electromagnetic waves from the person's stomach, heart, brain, or other organs; GPS or other location-based indications that a person is in an eating establishment (such as a restaurant) or food source location (such as a kitchen).

In previous paragraphs, we discussed how this present invention is superior to prior art because this present invention does not require manual activation of picture taking each time that a person eats. This present invention takes pictures automatically when a person eats. We now discuss how this present invention is also superior to prior art because this present invention does not require manual aiming of a camera (or other imaging device) toward the person's mouth or a reachable food source each time that a person eats. This present invention automatically captures the person's mouth and a reachable food source within imaging fields of vision when a person eats.

In an example, this device and method comprise one or more automatic-imaging members that automatically and collectively take pictures of a person's mouth and pictures of a reachable food source as the person eats, without the need for human intervention to actively aim or focus a camera toward a person's mouth or a reachable food source. In an example, this device and method takes pictures of a person's mouth and a food source automatically by eliminating the need for human intervention to aim an imaging member, such as a camera, towards the person's mouth and the food source. This device and method includes imaging members whose locations, and/or the movement of those locations while the person eats, enables the fields of vision of the imaging members to automatically encompass the person's mouth and a food source.

In an example, the fields of vision from one or more automatic-imaging members in this invention collectively and automatically encompass the person's mouth and a reachable food source, when the person eats, without the need for human intervention (when the person eats) to manually aim an imaging member toward the person's mouth or toward the reachable food source. In an example, the automatic-imaging members have wide-angle lenses that encompass a reachable food source and the person's mouth without any need for aiming or moving the imaging members. Alternatively, an automatic-imaging member may sequentially and iteratively focus on the food source, then on the person's mouth, then back on the food source, and so forth.

In an example, this device can automatically adjust the imaging vectors or focal lengths of one or more imaging components so that these imaging components stay focused on a food source and/or the person's mouth. Even if the line of sight from an automatic-imaging member to a food source, or to the person's mouth, becomes temporarily obscured, the device can track the last-known location of the food source, or the person's mouth, and search near that location in space to re-identify the food source, or mouth, to re-establish imaging contact. In an example, the device may track movement of the food source, or the person's mouth, relative to the imaging device. In an example, the device may extrapolate expected movement of the food source, or the person's mouth, and search in the expected projected of the food source, or the person's mouth, in order to re-establish imaging contact. In various examples, this device and method may use face recognition and/or gesture recognition methods to track the location of the person's face and/or hand relative to a wearable imaging device.

In an example, this device and method comprise at least one camera (or other imaging member) that takes pictures along an imaging vector which points toward the person's mouth and/or face, during certain body configurations, while the person eats. In an example, this device and member uses face recognition methods to adjust the direction and/or focal length of its field of vision in order to stay focused on the person's mouth and/or face. Face recognition methods and/or gesture recognition methods may also be used to detect and measure hand-to-mouth proximity and interaction. In an example, one or more imaging devices automatically stay focused on the person's mouth, even if the device moves, by the use of face recognition methods. In an example, the fields of vision from one or more automatic-imaging members collectively encompass the person's mouth and a reachable food source, when the person eats, without the need for human intervention, when the person eats, because the imaging members remain automatically directed toward the person's mouth, toward the reachable food source, or both.

In various examples, movement of one or more automatic-imaging members allows their fields of vision to automatically and collectively capture images of the person's mouth and a reachable food source without the need for human intervention when the person eats. In an example, this device and method includes an automatic-imaging member that is worn on the person's wrist, hand, finger, or arm, such that this automatic-imaging member automatically takes pictures of the person's mouth, a reachable food source, or both as the person moves their arm and hand when they eat. This movement causes the fields of vision from one or more automatic-imaging members to collectively and automatically encompass the person's mouth and a reachable food source as the person eats. Accordingly, there is no need for human intervention, when the person starts eating, to manually aim a camera (or other imaging member) toward the person's mouth or toward a reachable food source. Picture taking of the person's mouth and the food source is automatic and virtually involuntary. This makes it relatively easy to incorporate tamper-resisting features into this invention.

In an example, one or more imaging members are worn on a body member that moves as food travels along the food consumption pathway. In this manner, these one or more imaging members have lines of sight to the person's mouth and to the food source during at least some points along the food consumption pathway. In various examples, this movement is caused by bending of the person's shoulder, elbow, and wrist joints. In an example, an imaging member is worn on the wrist, arm, or hand of a dominant arm, wherein the person uses this arm to move food along the food consumption pathway. In another example, an imaging member may be worn on the wrist, arm, or hand of a non-dominant arm, wherein this other arm is generally stationery and not used to move food along the food consumption pathway. In another example, automatic-imaging members may be worn on both arms.

In an example, this invention comprises two or more automatic-imaging members wherein a first imaging member is pointed toward the person's mouth most of the time, as the person moves their arm to move food along the food consumption pathway, and wherein a second imaging member is pointed toward a reachable food source most of the time, as the person moves their arm to move food along the food consumption pathway. In an example, this invention comprises one or more imaging members wherein: a first imaging member points toward the person's mouth at least once as the person brings a piece (or portion) of food to their mouth from a reachable food source; and a second imaging member points toward the reachable food source at least once as the person brings a piece (or portion) of food to their mouth from the reachable food source.

In an example, this device and method comprise an imaging device with a single imaging member that takes pictures along shifting imaging vectors, as food travels along the food consumption pathway, so that it take pictures of a food source and the person's mouth sequentially. In an example, this device and method takes pictures of a food source and a person's mouth from different positions as food moves along the food consumption pathway. In an example, this device and method comprise an imaging device that scans for, locates, and takes pictures of the distal and proximal endpoints of the food consumption pathway.

In an example of this invention, the fields of vision from one or more automatic-imaging members are shifted by movement of the person's arm and hand while the person eats. This shifting causes the fields of vision from the one or more automatic-imaging members to collectively and automatically encompass the person's mouth and a reachable food source while the person is eating. This encompassing imaging occurs without the need for human intervention when the person eats. This eliminates the need for a person to manually aim a camera (or other imaging member) toward their mouth or toward a reachable food source.

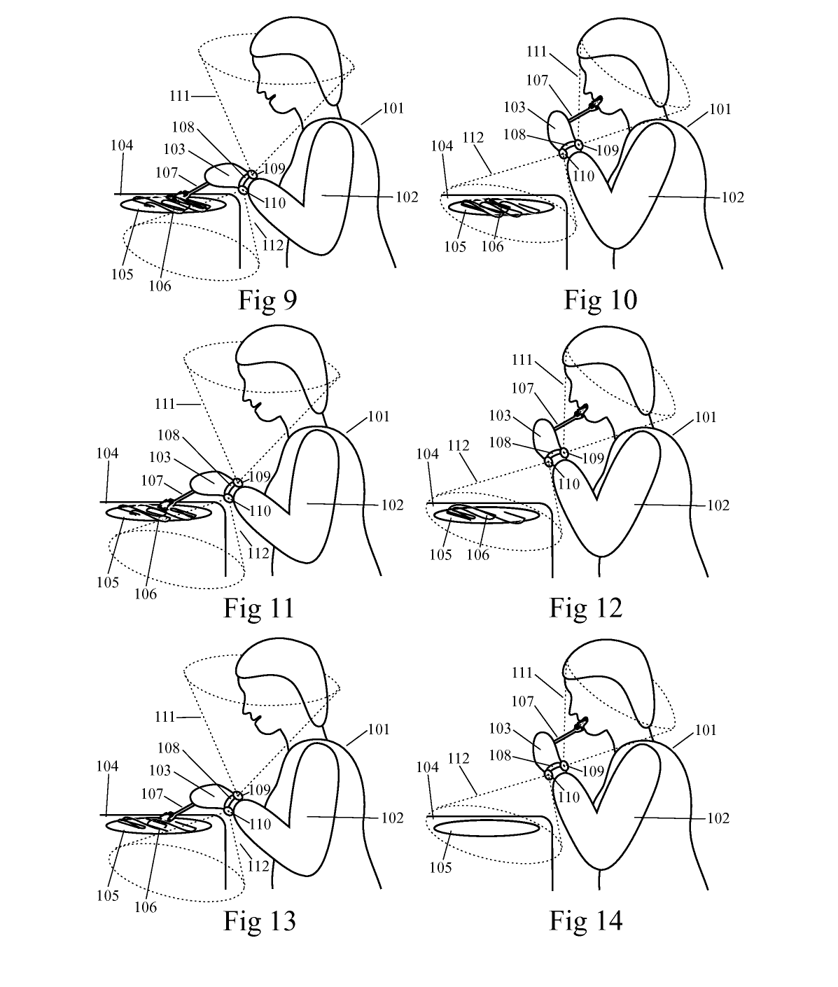

FIGS. 9-14 again show the example of this invention that was introduced in FIGS. 1-2. However, this example is now shown as functioning in a six-picture sequence of food consumption, involving multiple cycles of pieces (or portions) of food moving along the food consumption pathway until the food source is entirely consumed. In FIGS. 9-14, this device and method are shown taking pictures of a reachable food source and the person's mouth, from multiple perspectives, as the person eats until all of the food on a plate is consumed.

FIG. 9 starts this sequence by showing a person 101 engaging food 106 on plate 105 with utensil 107. The person moves utensil 107 by moving their arm 102 and hand 103. Wrist-mounted camera 109, on wrist band 108, has a field of vision 111 that encompasses the person's mouth. Wrist-mounted camera 110, also on wrist band 108, has a field of vision 112 that partially encompasses a reachable food source which, in this example, is food 106 on plate 105 on table 104.

FIG. 10 continues this sequence by showing the person having bent their arm 102 and wrist 103 in order to move a piece of food up to their mouth via utensil 107. In FIG. 10, camera 109 has a field of vision 111 that encompasses the person's mouth (including the interaction of the person's mouth and the piece of food) and camera 110 has a field of vision 112 that now fully encompasses the food source.

FIGS. 11-14 continue this sequence with additional cycles of the food consumption pathway, wherein the person brings pieces of food from the plate 105 to the person's mouth. In this example, by the end of this sequence shown in FIG. 14 the person has eaten all of the food 106 from plate 105.

In the sequence of food consumption pathway cycles that is shown in FIGS. 9-14, pictures of the reachable food source (food 106 on plate 105) taken by camera 110 are particularly useful in identifying the types of food to which the person has reachable access. In this simple example, featuring a single person with a single plate, changes in the volume of food on the plate could also be used to estimate the quantities of food which this person consumes. However, with more complex situations featuring multiple people and multiple food sources, images of the food source only would be limited for estimating the quantity of food that is actually consumed by a given person.

In this example, the pictures of the person's mouth taken by camera 109 are particularly useful for estimating the quantities of food actually consumed by the person. Static or moving pictures of the person inserting pieces of food into their mouth, refined by counting the number or speed of chewing motions and the number of cycles of the food consumption pathway, can be used to estimate the quantity of food consumed. However, images of the mouth only would be limited for identifying the types of food consumed.

Integrated analysis of pictures of both the food source and the person's mouth can provide a relatively accurate estimate of the types and quantities of food actually consumed by this person, even in situations with multiple food sources and multiple diners. Integrated analysis can compare estimates of food quantity consumed based on changes in observed food volume at the food source to estimates of food quantity consumed based on mouth-food interaction and food consumption pathway cycles.

Although it is preferable that the field of vision 111 for camera 109 encompasses the person's mouth all the time and that the field of vision 111 for camera 110 encompasses the reachable food source all the time, integrated analysis can occur even if this is not possible. As long as the field of vision 112 for camera 110 encompasses the food source at least once during a food consumption pathway cycle and the field of vision 111 from camera 109 encompasses the person's mouth at least once during a food consumption pathway cycle, this device and method can extrapolate mouth-food interaction and also changes in food volume at the reachable food source.

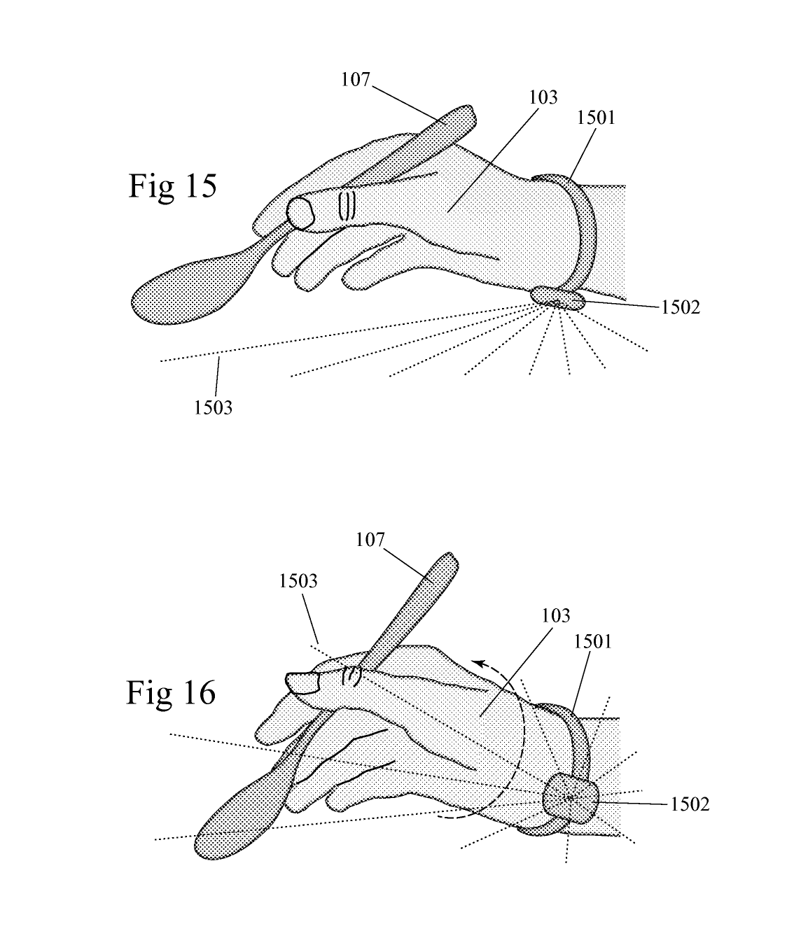

FIGS. 15 and 16 show, in greater detail, how the field of vision from a wrist-worn imaging member can advantageously shift as a person moves and rolls their wrist to bring food up to their mouth along the food consumption pathway. These figures show a person's hand 103 holding utensil 107 from the perspective of a person looking at their hand, as their hand brings the utensil up to their mouth. This rolling and shifting motion can enable a single imaging member, such as a single camera 1502 mounted on wrist band 1501, to take pictures of a reachable food source and the person's mouth, from different points along the food consumption pathway.

FIGS. 15 and 16 show movement of a single camera 1502 mounted on the anterior (inside) surface of wrist band 1501 as the person moves and rolls their wrist to bring utensil 107 up from a food source to their mouth. The manner in which this camera is worn is like a wrist watch, with a camera instead of a watch face, which has been rotated 180 degrees around the person's wrist. In FIG. 15, field of vision 1503 from camera 1502 points generally downward in a manner that would be likely to encompass a reachable food source which the person would engage with utensil 107. In FIG. 16, this field of vision 1503 has been rotated upwards towards the person's mouth by the rotation of the person's wrist as the person brings utensil 107 up to their mouth. These two figures illustrate an example wherein a single wrist-worn imaging member can take pictures of both a reachable food source and the person's mouth, due to the rolling motion of a person's wrist as food is moved along the food consumption pathway.

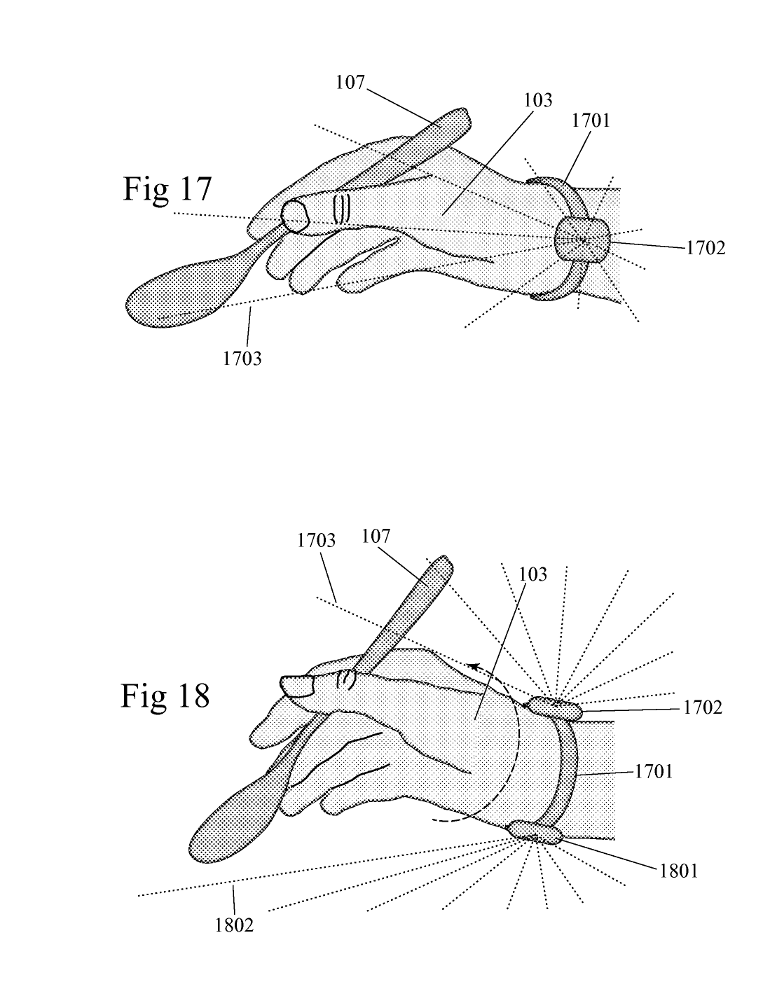

FIGS. 17 and 18 are similar to FIGS. 15 and 16, except that FIGS. 17 and 18 show a wrist-worn automatic-imaging member with two cameras, 1702 and 1801, instead of just one. This is similar to the example introduced in FIGS. 1 and 2. These figures show the person's hand 103 holding utensil 107 from the perspective of a person looking at their hand, as their hand brings the utensil up to their mouth. FIGS. 17 and 18 show how the rolling motion of the wrist, as food is moved along the food consumption pathway, enables a wrist-worn imaging member with two cameras, 1702 and 1801, to collectively and automatically take pictures of a reachable food source and a person's mouth.

The two cameras in FIGS. 17 and 18 are attached to the narrow sides of the person's wrist via wrist band 1701. Camera 1801 is not shown in FIG. 17 because it is on the far-side of the person's wrist which is not visible in FIG. 17. After the person's rolls their wrist to bring the utensil up toward their mouth, as shown in FIG. 18, camera 1801 comes into view. This rolling and shifting motion of the person's wrist, occurring between FIGS. 17 and 18, enables the two cameras, 1702 and 1801, to automatically and collectively take pictures of a reachable food source and the person's mouth, from different points along the food consumption pathway. In FIG. 17, field of vision 1703 from camera 1702 is directed toward the person's mouth. In FIG. 18, after the person has moved their arm and rotated their wrist, field of vision 1802 from camera 1801 is directed toward (the likely location of) a reachable food source. In an example, camera 1801 may scan the vicinity in order to detect and identify a reachable food source.

Having two cameras mounted on opposite sides of a person's wrist increases the probability of encompassing both the person's mouth and a reachable food source as the person rolls their wrist and bends their arm to move food along the food consumption pathway. In other examples, more than two cameras may be attached on a band around the person's wrist to further increase the probability of encompassing both the person's mouth and the reachable food source.

In an example, the location of one or more cameras may be moved automatically, independently of movement of the body member to which the cameras are attached, in order to increase the probability of encompassing both the person's mouth and a reachable food source. In an example, the lenses of one or more cameras may be automatically and independently moved in order to increase the probability of encompassing both the person's mouth and a reachable food source. In various examples, a lens may be automatically shifted or rotated to change the direction or focal length of the camera's field of vision. In an example, the lenses of one or more cameras may be automatically moved to track the person's mouth and hand. In an example, the lenses of one or more cameras may be automatically moved to scan for reachable food sources.

In an example, this device and method comprise a device that is worn on a person so as to take images of food, or pieces of food, at multiple locations as food travels along a food consumption pathway. In an example, this device and method comprise a device that takes a series of pictures of a portion of food as it moves along a food consumption pathway between a reachable food source and the person's mouth. In an example, this device and method comprise a wearable imaging member that takes pictures upwards toward a person's face as the person's arm bends when the person eats. In an example, this invention comprises an imaging member that captures images of the person's mouth when the person's elbow is bent at an angle between 40-140 degrees as the person brings food to their mouth. In various examples, this device and method automatically takes pictures of food at a plurality of positions as food moves along the food consumption pathway. In an example, this device and method estimates the type and quantity of food consumed based, at least partially, on pattern analysis of images of the proximal and distal endpoints of the food consumption pathway.

In an example, this invention comprises a human-energy input measuring device and method that includes a wearable imaging member that identifies the types and quantities of food consumed based on images of food from a plurality of points along a food consumption pathway. In an example, this device and method takes pictures of a person's mouth and a reachable food source from multiple angles, from an imaging member worn on a body member that moves as food travels along the food consumption pathway.

In an example, this invention comprises one or more of imaging devices which are worn on a location on the human body that provides at least one line of sight from the device to the person's mouth and at least one line of sight to a reachable food source, as food travels along the food consumption pathway. In various examples, these one or more imaging devices simultaneously or sequentially record images along at least two different vectors, one which points toward the mouth during at least some portion of the food consumption pathway and one which points toward the food source during at least some portion of the food consumption pathway. In various examples, this device and method comprise multiple imaging members that are worn on a person's wrist, hand, arm, or finger—with some imaging elements pointed toward the person's mouth from certain locations along the food consumption pathway and some imaging elements pointed toward a reachable food source from certain locations along the food consumption pathway.

Thus far in our description of the figures, we have discussed a variety of ways in which the automatic image-taking members and methods of this invention may be embodied. We now turn our attention to discuss, in greater detail, the automatic imaging-analyzing members and methods which are also an important part of this invention. This invention comprises a device and method that includes at least one image-analyzing member. This image-analyzing member automatically analyzes pictures of a person's mouth and pictures of a reachable food source in order to estimate the types and quantities of food consumed by this person. This is superior to prior art that only analyzes pictures of a reachable food source because the person might not actually consume all of the food at this food source.

In various examples, one or more methods to analyze pictures, in order to estimate the types and quantities of food consumed, can be selected from the group consisting of: pattern recognition; food recognition; word recognition; logo recognition; bar code recognition; face recognition; gesture recognition; and human motion recognition. In various examples, a picture of the person's mouth and/or a reachable food source may be analyzed with one or more methods selected from the group consisting of: pattern recognition or identification; human motion recognition or identification; face recognition or identification; gesture recognition or identification; food recognition or identification; word recognition or identification; logo recognition or identification; bar code recognition or identification; and 3D modeling. In an example, images of a person's mouth and a reachable food source may be taken from at least two different perspectives in order to enable the creation of three-dimensional models of food.

In various examples, this invention comprises one or more image-analyzing members that analyze one or more factors selected from the group consisting of: number and type of reachable food sources; changes in the volume of food observed at a reachable food source; number and size of chewing movements; number and size of swallowing movements; number of times that pieces (or portions) of food travel along the food consumption pathway; and size of pieces (or portions) of food traveling along the food consumption pathway. In various examples, one or more of these factors may be used to analyze images to estimate the types and quantities of food consumed by a person.

In an example, this invention is entirely automatic for both food imaging and food identification. In an example, this invention comprises a device and method that automatically and comprehensively analyzes images of food sources and a person's mouth in order to provide final estimates of the types and quantities of food consumed. In an example, the food identification and quantification process performed by this device and method does not require any manual entry of information, any manual initiation of picture taking, or any manual aiming of an imaging device when a person eats. In an example, this device and method automatically analyzes images to estimate the types and quantities of food consumed without the need for real-time or subsequent human evaluation.

In an example, this device identifies the types and quantities of food consumed based on: pattern recognition of food at a reachable food source; changes in food at that source; analysis of images of food traveling along a food consumption pathway from a food source to the person's mouth; and/or the number of cycles of food moving along the food consumption pathway. In various examples, food may be identified by pattern recognition of food itself, by recognition of words on food packaging or containers, by recognition of food brand images and logos, or by recognition of product identification codes (such as “bar codes”). In an example, analysis of images by this device and method occurs in real time, as the person is consuming food. In an example, analysis of images by this device and method occurs after the person has consumed food.

In another example, this invention is partially automatic and partially refined by human evaluation or interaction. In an example, this device and method comprise a device and method that automatically analyzes images of food sources and a person's mouth in order to provide initial estimates of the types and quantities of food consumed. These initial estimates are then refined by human evaluation and/or interaction. In an example, estimation of the types and quantities of food consumed is refined or enhanced by human interaction and/or evaluation.

For example, the device may prompt the person with clarifying questions concerning the types and quantities of food that person has consumed. These questions may be asked in real time, as a person eats, at a subsequent time, or periodically. In an example, this device and method may prompt the person with queries to refine initial automatically-generated estimates of the types and quantities of food consumed. Automatic estimates may be refined by interaction between the device and the person. However, such refinement should have limits and safeguards to guard against possible tampering. For example, the device and method should not allow a person to modify automatically-generated initial estimates of food consumed to a degree that would cause the device and method to under-estimate caloric intake.

In an example, analysis of food images and estimation of food consumed by this device and method may be entirely automatic or may be a mixture of automated estimates plus human refinement. Even a partially-automated device and method for calorie monitoring and estimation is superior to prior art that relies completely on manual calorie counting or manual entry of food items consumed. In an example, the estimates of the types and quantities of food consumed that are produced by this invention are used to estimate human caloric intake. In an example, images of a person's mouth, a reachable food source, and the interaction between the person's mouth and food are automatically, or semi-automatically, analyzed to estimate the types of quantities of food that the person eats. These estimates are, in turn, used to estimate the person's caloric intake.

In an example, the caloric intake estimation provided by this device and method becomes the energy-input measuring component of an overall system for energy balance and weight management. In an example, the device and method can estimate the energy-input component of energy balance. In an example, this invention comprises an automatic and tamper-resistant device and method for estimating human caloric intake.

In an example, the device and method for estimating human caloric intake that is disclosed herein may be used in conjunction with a device and method for estimating human caloric output and/or human energy expenditure. In an example, this present invention can be used in combination with a wearable and mobile energy-output-measuring component that automatically records and analyses images in order to detect activity and energy expenditure. In an example, this present invention may be used in combination with a wearable and mobile device that estimates human energy output based on patterns of acceleration and movement of body members. In an example, this invention may be used in combination with an energy-output-measuring component that estimates energy output by measuring changes in the position and configuration of a person's body.

In an example, this invention may be incorporated into an overall device, system, and method for human energy balance and weight management. In an example, the estimates of the types and quantities of food consumed that are provided by this present invention are used to estimate human caloric intake. These estimates of human caloric intake are then, in turn, used in combination with estimates of human caloric expenditure as part of an overall system for human energy balance and weight management. In an example, estimates of the types and quantities of food consumed are used to estimate human caloric intake and wherein these estimates of human caloric intake are used in combination with estimates of human caloric expenditure as part of an overall system for human energy balance and human weight management.

This invention can include an optional analytic component that analyzes and compares human caloric input vs. human caloric output for a particular person as part of an overall device, system, and method for overall energy balance and weight management. This overall device, system, and method may be used to help a person to lose weight or to maintain a desirable weight. In an example, this device and method can be used as part of a system with a human-energy input measuring component and a human-energy output measuring component. In an example, this invention is part of an overall system for energy balance and weight management.

Thus far in our description of the figures, we have repeatedly described this invention as being tamper resistant, but have not shown details of how tamper-resistant features could be embodied. We now show and discuss, in some detail, some of the specific ways in which this device and method for monitoring and measuring caloric intake can be made tamper resistant. This invention advantageously can be made tamper-resistant because the imaging members are wearable and can operate in an automatic manner.

In an example, this invention includes one or more automatic-imaging members that collectively and automatically take pictures of the person's mouth and pictures of a reachable food source, when the person eats, without the need for human intervention, when the person eats, to activate picture taking. In an example, these one or more automatic-imaging members take pictures continually. In an example, these one or more automatic-imaging members are automatically activated to take pictures when a person eats based on a sensor selected from the group consisting of: accelerometer, inclinometer, motion sensor, sound sensor, smell sensor, blood pressure sensor, heart rate sensor, EEG sensor, ECG sensor, EMG sensor, electrochemical sensor, gastric activity sensor, GPS sensor, location sensor, image sensor, optical sensor, piezoelectric sensor, respiration sensor, strain gauge, electrogoniometer, chewing sensor, swallow sensor, temperature sensor, and pressure sensor.

In an example, the fields of vision from these one or more automatic-imaging members collectively and automatically encompass the person's mouth and a reachable food source, when the person eats, without the need for human intervention, when the person eats, to manually aim an imaging member toward the person's mouth or toward the reachable food source. In an example, the fields of vision from one or more automatic-imaging members are moved as the person moves their arm when the person eats; and wherein this movement causes the fields of vision from one or more automatic-imaging members to collectively and automatically encompass the person's mouth and a reachable food source, when the person eats, without the need for human intervention, when the person eats, to manually aim an imaging member toward the person's mouth or toward the reachable food source.

In an example, these one or more automatic-imaging members are worn on one or more body members selected from the group consisting of the person's wrist, hand, arm, and finger; wherein the fields of vision from one or more automatic-imaging members are moved as the person moves their arm when the person eats; and wherein this movement causes the fields of vision from one or more automatic-imaging members to collectively and automatically encompass the person's mouth and a reachable food source, when the person eats, without the need for human intervention, when the person eats, to manually aim an imaging member toward the person's mouth or toward the reachable food source.

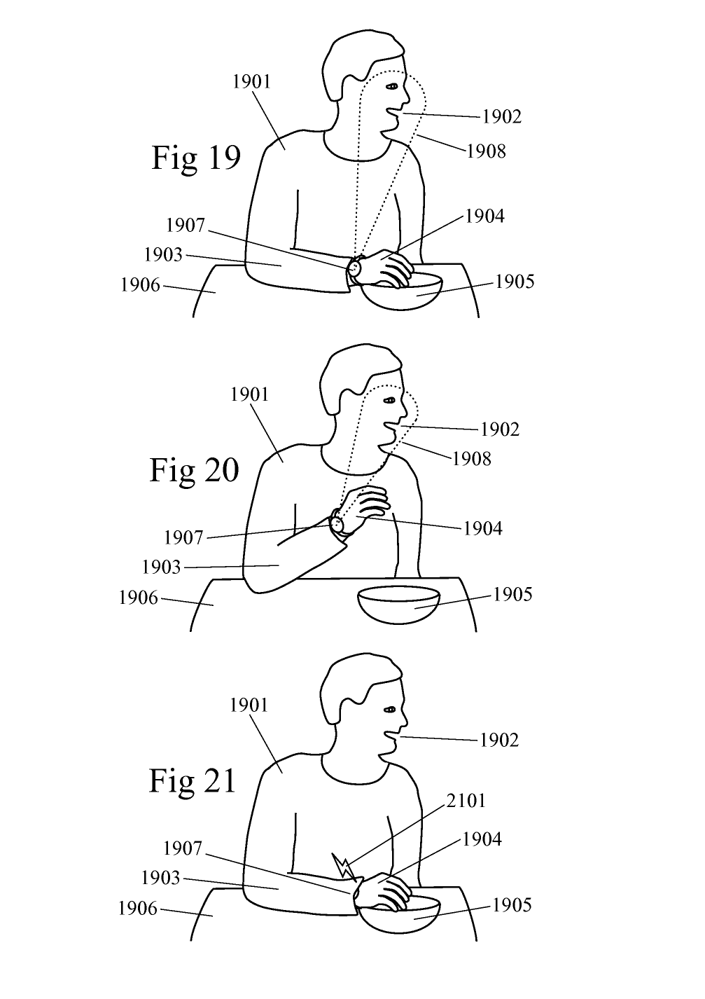

FIGS. 19-21 show one example of how this invention can be made tamper resistant. FIGS. 19-21 show a person, 1901, who can access a reachable food source 1905 (food in a bowl, in this example), on table 1906, by moving their arm 1903 and hand 1904. In this example, the person 1901 is wearing a wrist-based automatic-imaging member 1907 with field of vision 1908. In FIG. 19, this wrist-based automatic-imaging member 1907 is functioning properly because the field of vision 1908 from of this automatic-imaging member 1907 has an unobstructed line of sight to the person's mouth 1902. This imaging member can monitor the person's mouth 1902 to detect if the person is eating and then analyze pictures to estimate the quantity of food consumed.

In FIG. 19, automatic-imaging member 1907 recognizes that the line of sight to the person's mouth is unobstructed because it recognizes the person's mouth using face recognition methods. In other examples, automatic-imaging member 1907 may recognize that the line of sight to the person's mouth is unobstructed by using other pattern recognition or imaging-analyzing means. As long as a line of sight from the automatic-imaging member to the person's mouth is maintained (unobstructed), the device and method can detect if the person starts eating and, in conjunction with images of the reachable food source, it can estimate caloric intake based on quantities and types of food consumed.

In FIG. 20, person 1901 has bent their arm 1903 and moved their hand 1904 in order to bring a piece of food from the reachable food source 1905 up to their mouth 1902. In this example, the piece of food is clutched (hidden) in the person's hand as it travels along the food consumption pathway. In this example, the automatic-imaging member 1907 used face recognition methods to track the relative location of the person's mouth 1902 and has shifted its field of vision 1908 in order to maintain the line of sight to the person's mouth. As long as this line of sight is maintained, this mouth-imaging component of this device and method for estimating caloric intake can function properly.

In FIG. 21, however, the functioning of this imaging member 1907 has been impaired. This impairment may be intentional tampering by the person or it may be accidental. In either event, the device and method detects and responds to the impairment in order to correct the impairment. In FIG. 21, the sleeve of the person's shirt has slipped down over the automatic-imaging device, obstructing the line of sight from the imaging device 1907 to the person's mouth 1902. Thus covered, the obstructed automatic-imaging member cannot function properly. In this example, the automatic-imaging member recognizes that its line of sight to the person's mouth has been lost. In an example, it may recognize this by using face recognition methods. When the person's face is no longer found at an expected location (or nearby), then the device and method recognizes that its functioning is impaired.

Without a line of sight to the person's mouth in FIG. 21, the wrist-worn automatic-imaging device 1907 no longer works properly to monitor and estimate caloric intake. In response, automatic-imaging device 1907 gives a response 2101 that is represented in FIG. 21 by a lightning bolt symbol. In an example, this response 2101 may be an electronic buzzing sound or a ring tone. In another example, response 2101 may include vibration of the person's wrist. In another example, response 2101 may be transmission or a message to a remote location or monitor. In various examples, this invention detects and responds to loss of imaging functionality in a manner that helps to restore proper imaging functionality. In this example, response 2101 prompts the person to move their shirt sleeve upwards to uncover the wrist-worn imaging member 1904 so that this imaging member can work properly once again.

In an example, the line of sight from an automatic-imaging member to the person's mouth may be obstructed by an accidental event, such as the accidental downward sliding of the person's shirt sleeve. In another example, the line of sight from the automatic-imaging member to the person's mouth may be intentionally obstructed by the person. Technically, only the second type of causation should be called “tampering” with the operation of the device and method. However, one can design tamper-resisting features for operation of the device and method that detect and correct operational impairment whether this impairment is accidental or intentional. The device can be designed to detect if the automatic-imaging function is obstructed, or otherwise impaired, and to respond accordingly to restore functionality.

One example of a tamper-resistant design is for the device to constantly monitor the location of the person's mouth and to respond if a line of sight to the person's mouth is ever obstructed. Another example of a tamper-resistant design is for the device to constantly scan and monitor space around the person, especially space in the vicinity of the person's hand, to detect possible reachable food sources. In a variation on these examples, a device may only monitor the location of the person's mouth, or scan for possible reachable food sources, when one or more sensors indicate that the person is probably eating. These one or more sensors may be selected from the group consisting of: accelerometer, inclinometer, motion sensor, pedometer, sound sensor, smell sensor, blood pressure sensor, heart rate sensor, EEG sensor, ECG sensor, EMG sensor, electrochemical sensor, gastric activity sensor, GPS sensor, location sensor, image sensor, optical sensor, piezoelectric sensor, respiration sensor, strain gauge, electrogoniometer, chewing sensor, swallow sensor, temperature sensor, and pressure sensor.

In an example, this invention can be embodied in a tamper-resistant device that automatically monitors caloric intake comprising: one or more automatic-imaging members that are worn on one or more locations on a person from which these members: collectively and automatically take pictures of the person's mouth when the person eats and pictures of a reachable food source when the person eats; wherein a reachable food source is a food source that the person can reach by moving their arm; and wherein food can include liquid nourishment as well as solid food; a tamper-resisting mechanism which detects and responds if the operation of the one or more automatic-imaging members is impaired; and an image-analyzing member which automatically analyzes pictures of the person's mouth and pictures of the reachable food source in order to estimate the types and quantities of food that are consumed by the person.

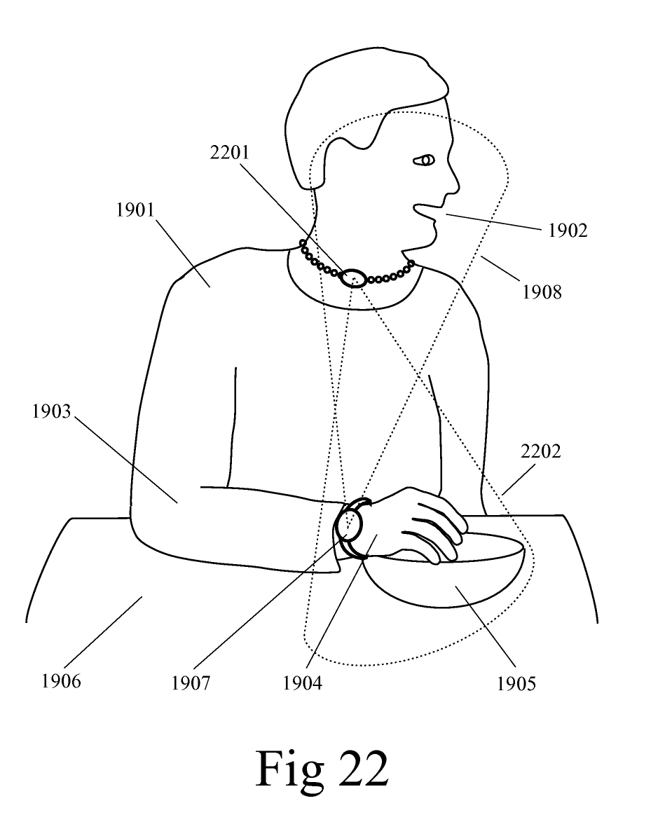

FIG. 22 shows another example of how this invention may be embodied a tamper-resisting device and method to automatically monitor and measure caloric intake. In FIG. 22, this device and method comprise two wearable automatic-imaging members. The first automatic-imaging member, 1907, is worn on a person's wrist like a wrist watch. This first member takes pictures of the person's mouth and detects if the line of sight from this first imaging member to the person's mouth is obstructed or otherwise impaired. The second automatic-imaging member, 2201, is worn on a person's neck like a necklace. This second member takes pictures of the person's hand and a reachable food source and detects if the line of sight from the second imaging member to the person's hand and a reachable food source is obstructed or otherwise impaired. In this example, this device and method is tamper-resistant because it detects and responds if either of these lines of sight are obstructed or otherwise impaired.

Discussing FIG. 22 in further detail, this figure shows person 1901 accessing reachable food source (e.g. a bowl of food) 1905 on table 1906 by moving their arm 1903 and hand 1904. Person 1901 wears a first automatic-imaging member 1907 around their wrist. From its wrist-worn location, this first imaging member 1907 has a field of vision 1908 that encompasses the person's mouth 1902. In an example, this automatic-imaging member 1907 uses face recognition to shift its field of vision 1907, as the person moves their wrist or head, so as to maintain a line of sight from the wrist to the person's mouth. In an example, the field of vision 1907 may be shifted by automatic rotation or shifting of the lens on automatic-imaging member 1907.

In an example, first automatic-imaging member 1907 constantly maintains a line of sight to the person's mouth by constantly shifting the direction and/or focal length of its field of vision 1908. In another example, this first automatic-imaging member 1907 scans and acquires a line of sight to the person's mouth only when a sensor indicates that the person is eating. In an example, this scanning function may comprise changing the direction and/or focal length of the member's field of vision 1908. If the line of sight from this member to the person's mouth is obstructed, or otherwise impaired, then this device and method detects and responds to this impairment as part of its tamper-resisting function. In an example, its response to tampering helps to restore proper imaging function for automatic monitoring and estimation of caloric intake.

In this example, this person 1901 also wears a second automatic-imaging member 2201 around their neck. In this example, automatic-imaging member 2201 is worn like a central pendant on the front of a necklace. From this location, this second imaging member has a forward-and-downward facing field of vision, 2202, that encompasses the person's hand 1904 and a reachable food source 1905. In an example, this second automatic-imaging member 2201 uses gesture recognition, or other pattern recognition methods, to shift its focus so as to always maintain a line of sight to the person's hand and/or to scan for potential reachable food sources.

In an example, this second automatic-imaging member 2201 constantly maintains a line of sight to one or both of the person's hands. In another example, this second automatic-imaging member 2201 scans for (and identifies and maintains a line of sight to) the person's hand only when a sensor indicates that the person is eating. In another example, this second automatic-imaging member 2201 scans for, acquires, and maintains a line of sight to a reachable food source only when a sensor indicates that the person is probably eating. In various examples, the sensors used to activate one or more of these automatic-imaging members may be selected from the group consisting of: accelerometer, inclinometer, motion sensor, pedometer, sound sensor, smell sensor, blood pressure sensor, heart rate sensor, EEG sensor, ECG sensor, EMG sensor, electrochemical sensor, gastric activity sensor, GPS sensor, location sensor, image sensor, optical sensor, piezoelectric sensor, respiration sensor, strain gauge, electrogoniometer, chewing sensor, swallow sensor, temperature sensor, and pressure sensor.

In an example, this device and method comprise one or more imaging members that scan nearby space in order to identify a person's mouth, hand, and/or reachable food source in response to sensors indicating that the person is probably eating. In an example, one of these imaging members: (a) scans space surrounding the imaging member in order to identify the person's hand and acquire a line of sight to the person's hand when a sensor indicates that the person is eating; and then (b) scans space surrounding the person's hand in order to identify and acquire a line of sight to any reachable food source near the person's hand. In an example, the device and method may concentrate scanning efforts on the person's hand at the distal endpoint of a food consumption pathway to detect and identify a reachable food source. If the line of sight from this imaging member to the person's hand and/or a reachable food source is subsequently obstructed or otherwise impaired, then this device and method detects and responds as part of its tamper-resisting features. In an example, this response is designed to restore imaging functionality to enable proper automatic monitoring and estimation of caloric intake.

More generally, in various examples, this invention includes one or more tamper-resisting mechanisms which detect and respond if the operation of one or more automatic-imaging members are obstructed or otherwise impaired. In an example, this invention includes a tamper-resisting mechanism which detects and responds if a person hinders the operation of one or more automatic-imaging members. For example, the device and method disclosed herein can have a tamper-resistant feature that is triggered if the device is removed from the body member as indicated by a sensor selected from the group consisting of: accelerometer, inclinometer, motion sensor, pedometer, sound sensor, smell sensor, blood pressure sensor, heart rate sensor, EEG sensor, ECG sensor, EMG sensor, electrochemical sensor, gastric activity sensor, GPS sensor, location sensor, image sensor, optical sensor, piezoelectric sensor, respiration sensor, strain gauge, electrogoniometer, chewing sensor, swallow sensor, temperature sensor, and pressure sensor.

In an example, this invention comprises a device and method with features that resist tampering with the automatic and involuntary estimation of the types and quantities of food consumed by a person. In an example, this device and method includes an alarm that is triggered if a wearable imaging device is covered up. In various examples, this invention comprises one or more imaging devices which detect and respond if their direct line of sight with the person's mouth or a reachable food source is impaired. In an example, this invention includes a tamper-resisting member that monitors a person's mouth using face recognition methods and responds if the line of sight from an automatic-imaging member to the person's mouth is impaired when a person eats. In another example, this invention includes a tamper-resisting member that detects and responds if the person's actual weight gain or loss is inconsistent with predicted weight gain or loss. Weight gain or loss may be predicted by the net balance of estimated caloric intake and estimated caloric expenditure.

The tamper-resisting features of this invention help to make the operation of this invention relatively automatic, tamper-resistant, and virtually involuntary. This ensures comprehensive and accurate monitoring and measuring of caloric intake.

In an example, this invention can include at least two automatic-imaging members worn on a person's body, wherein the field of vision from a first automatic-imaging member automatically encompasses the person's mouth as the person eats, and wherein the field of vision from a second automatic-imaging member automatically encompasses a reachable food source as the person eats.

In an example, this invention can include at least two automatic-imaging members worn on a person's body: wherein a first automatic-imaging member is worn on a body member selected from the group consisting of the person's wrist, hand, lower arm, and finger; wherein the field of vision from the first automatic-imaging member automatically encompasses the person's mouth as the person eats; wherein a second automatic-imaging member is worn on a body member selected from the group consisting of the person's neck, head, torso, and upper arm; and wherein the field of vision from the second automatic-imaging member automatically encompasses a reachable food source as the person eats.

In an example, this invention can include a tamper-resisting member that comprises a sensor that detects and responds if an automatic-imaging member is removed from the person's body, wherein this sensor is selected from the group consisting of: accelerometer, inclinometer, motion sensor, pedometer, sound sensor, smell sensor, blood pressure sensor, heart rate sensor, EEG sensor, ECG sensor, EMG sensor, electrochemical sensor, gastric activity sensor, GPS sensor, location sensor, image sensor, optical sensor, piezoelectric sensor, respiration sensor, strain gauge, electrogoniometer, chewing sensor, swallow sensor, temperature sensor, and pressure sensor.

In an example, this invention can include a tamper-resisting member that comprises a sensor that detects and responds if the line of sight from one or more automatic-imaging members to the person's mouth or to a food source is impaired when a person is probably eating based on a sensor, wherein this sensor is selected from the group consisting of: accelerometer, inclinometer, motion sensor, pedometer, sound sensor, smell sensor, blood pressure sensor, heart rate sensor, EEG sensor, ECG sensor, EMG sensor, electrochemical sensor, gastric activity sensor, GPS sensor, location sensor, image sensor, optical sensor, piezoelectric sensor, respiration sensor, strain gauge, electrogoniometer, chewing sensor, swallow sensor, temperature sensor, and pressure sensor.

In an example, this invention can include a tamper-resisting member that monitors a person's mouth using face recognition methods and responds if the line of sight from an automatic-imaging member to the person's mouth is impaired when a person is probably eating based on a sensor, wherein this sensor is selected from the group consisting of: accelerometer, inclinometer, motion sensor, pedometer, sound sensor, smell sensor, blood pressure sensor, heart rate sensor, EEG sensor, ECG sensor, EMG sensor, electrochemical sensor, gastric activity sensor, GPS sensor, location sensor, image sensor, optical sensor, piezoelectric sensor, respiration sensor, strain gauge, electrogoniometer, chewing sensor, swallow sensor, temperature sensor, and pressure sensor.

In an example, this invention can include a tamper-resisting member that detects and responds if the person's actual weight gain or loss is inconsistent with the predicted weight gain or loss predicted by the combination of the estimated caloric intake and the estimated caloric expenditure.

In an example, this invention can be embodied in a tamper-resistant device that automatically monitors caloric intake comprising: one or more automatic-imaging members that are worn on one or more locations on a person from which these members: collectively and automatically take pictures of the person's mouth when the person eats and take pictures of a reachable food source when the person eats; wherein a reachable food source is a food source that the person can reach by moving their arm; wherein food can include liquid nourishment as well as solid food; wherein one or more automatic-imaging members collectively and automatically take pictures of the person's mouth and pictures of a reachable food source, when the person eats, without the need for human intervention, when the person eats, to activate picture taking; and wherein the fields of vision from one or more automatic-imaging members collectively and automatically encompass the person's mouth and a reachable food source, when the person eats, without the need for human intervention, when the person eats, to manually aim an imaging member toward the person's mouth or toward the reachable food source; a tamper-resisting mechanism which detects and responds if the operation of the one or more automatic-imaging members is impaired; wherein a tamper-resisting member comprises a sensor that detects and responds if the line of sight from one or more automatic-imaging members to the person's mouth or to a food source is impaired when a person is probably eating based on a sensor, wherein this sensor is selected from the group consisting of: accelerometer, inclinometer, motion sensor, pedometer, sound sensor, smell sensor, blood pressure sensor, heart rate sensor, EEG sensor, ECG sensor, EMG sensor, electrochemical sensor, gastric activity sensor, GPS sensor, location sensor, image sensor, optical sensor, piezoelectric sensor, respiration sensor, strain gauge, electrogoniometer, chewing sensor, swallow sensor, temperature sensor, and pressure sensor; and an image-analyzing member which automatically analyzes pictures of the person's mouth and pictures of the reachable food source in order to estimate not just what food is at the reachable food source, but the types and quantities of food that are actually consumed by the person; and wherein the image-analyzing member uses one or more methods selected from the group consisting of: pattern recognition or identification; human motion recognition or identification; face recognition or identification; gesture recognition or identification; food recognition or identification; word recognition or identification; logo recognition or identification; bar code recognition or identification; and 3D modeling.

In an example, this invention can be embodied in a tamper-resistant method for automatically monitoring caloric intake comprising: having a person wear one or more automatic-imaging members at one or more locations on the person from which these members collectively and automatically take pictures of the person's mouth when the person eats and pictures of a reachable food source when the person eats; wherein a reachable food source is a food source that the person can reach by moving their arm; and wherein food can include liquid nourishment as well as solid food; detecting and responding if the operation of the one or more automatic-imaging members is impaired; and automatically analyzing pictures of the person's mouth and pictures of the reachable food source in order to estimate the types and quantities of food that are consumed by the person.

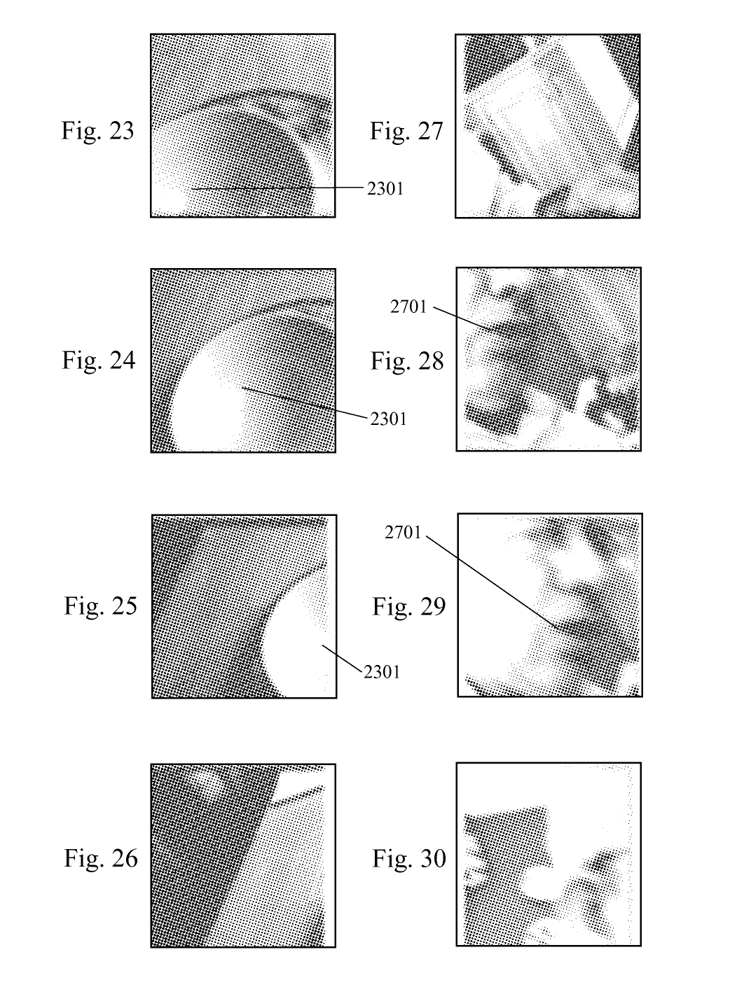

FIGS. 23-30 show two four-frame series of pictures taken by a rough prototype of this invention that was worn on a person's wrist. These four-frame picture series capture movement of the field of vision from two cameras, as the person's arm and hand moved to transport food along the food consumption pathway. These pictures have been transformed from gradient full-color images into black-and-white dot images in order to conform to the figure requirements for a U.S. patent. In practice, these pictures would likely be analyzed as full-gradient full-color images for optimal image analysis and pattern recognition.

FIGS. 23-26 show a four-frame series of pictures taken by the moving field of vision from a first camera that was worn on the anterior surface of the person's wrist, like a wrist watch. This first camera generally pointed away from the person's face and toward a reachable food source as the person moved their arm and hand to transport food along the food consumption pathway. This first camera had an imaging vector that was generally perpendicular to the longitudinal bones of the person's upper arm.

FIG. 23 shows the picture taken by this first camera at the distal endpoint of the food consumption pathway. This first picture shows a portion of a bowl, 2301, which represents a reachable food source. FIGS. 24-26 show subsequent pictures in this series taken by the first camera as the person moved their arm and hand so as to move food up to their mouth along the food consumption pathway. FIGS. 24 and 25 provide additional pictures of portions of the bowl 2301. In FIG. 26, the bowl is no longer in the field of vision of the camera at the proximal endpoint of the food consumption pathway. It is important to note that this camera worn on the person's wrist automatically encompasses the reachable food source in its field of vision as the arm and hand move food along the food consumption pathway, without any need for manual aiming or activation of the camera.

In the figures shown here, bowl 2301 represents a reachable food source, but no actual food is shown in it. In practice, bowl 2301 would have food in it. This device and method would analyze the series of pictures of food in the bowl (in FIGS. 23-25) in order to identify the type, and estimate the volume, of food in the bowl—in conjunction with images of the person's mouth and interaction between the person's mouth and food. In this example, the reachable food source is food in a bowl. In other examples, the reachable food source may be selected from the group consisting of: food on a plate, food in a bowl, food in a glass, food in a cup, food in a bottle, food in a can, food in a package, food in a container, food in a wrapper, food in a bag, food in a box, food on a table, food on a counter, food on a shelf, and food in a refrigerator.

FIGS. 27-30 show a four-frame series of pictures taken by the moving field of vision from a second camera that was also worn on the anterior surface of the person's wrist, like a wrist watch. However, this second camera generally pointed toward the person's face and away from a reachable food source as the person moved their arm and hand to transport food along the food consumption pathway. Like the first camera, this second camera had an imaging vector that was generally perpendicular to the longitudinal bones of the person's upper arm. However, this second camera had an imaging vector that was rotated 180 degrees around the person's wrist as compared to the imaging vector of the first camera.

FIG. 27 shows the picture taken by this first camera at the distal endpoint of the food consumption pathway. This first picture does not include the person's mouth. However, as the person moves their arm and hand upwards during the food consumption pathway, this second camera did capture images of the person's mouth, 2701, as shown in FIGS. 28 and 29. In FIG. 30, the person's mouth is no longer in the field of vision of the camera at the proximal endpoint of the food consumption pathway. This second camera, worn on the person's wrist, automatically encompasses the person's mouth in its field of vision as the arm and hand moves food along the food consumption pathway, without any need for manual aiming or activation of the camera.

The pictures shown in FIGS. 23-30 are only one example of the types of pictures that can be taken by an embodiment of this invention. This embodiment is only a rough prototype comprising a wrist-worn imaging member with two opposite-facing cameras that are perpendicular to the bones of the person's upper arm. As described previously in this description of the figures, there are many variations and refinements that could improve the ability of one or more automatic-imaging members to automatically and collectively encompass a reachable food source and a person's mouth while they eat.

However, even these simple pictures from a rough prototype provide encouraging preliminary evidence that this invention can work. This is early evidence that this invention can comprise one or more wearable automatic-imaging devices that automatically and collectively take pictures of a reachable food source and the person's mouth, when the person eats, without the need for manual aiming or picture activation, when the person eats. These pictures can then be analyzed to estimate the types and quantities of food consumed which, in turn, are used to estimate the person's caloric intake. The relatively automatic, tamper-resistant, and involuntary characteristics of this device and method make it superior to the prior art for monitoring and measuring caloric intake.

As discussed in the specification thus far, this invention can comprise eyeglasses which further comprise one or more automatic food imaging members. As discussed thus far, pictures taken by an imaging member can be automatically analyzed in order to estimate the types and quantities of food which are consumed by a person. Food can refer to beverages as well as solid food. As discussed thus far, an automatic imaging member can automatically take pictures of food consumption because it takes pictures continually. An automatic imaging member can take pictures when it is activated (triggered) by food consumption based on data collected by one or more sensors selected from the group consisting of: accelerometer, inclinometer, motion sensor, sound sensor, smell sensor, blood pressure sensor, heart rate sensor, EEG sensor, ECG sensor, EMG sensor, electrochemical sensor, gastric activity sensor, GPS sensor, location sensor, image sensor, optical sensor, piezoelectric sensor, respiration sensor, strain gauge, electrogoniometer, chewing sensor, swallow sensor, temperature sensor, and pressure sensor. In an example, when data from one or more sensors indicates that a person is probably consuming food, then this can activate (trigger) an imaging member to start taking pictures and/or recording images.

As discussed in the specification thus far, this invention can further comprise methods of pattern recognition which automatically analyze food images in order to estimate food types and quantities. Pattern recognition analysis can comprise analysis of food shape, color, texture, and volume. As discussed, pattern recognition analysis can also identify food type and quantity by analyzing images of food packaging. This invention can take pictures from different angles (different image vectors) and these multiple pictures from different angles can be analyzed together using 3D modeling and/or volumetric analysis in order to better identify the types and quantities of food consumed by a person. This invention can further comprise one or more image analysis methods selected from the group consisting of: pattern recognition; human motion recognition; face recognition; gesture recognition; food recognition; word recognition; logo recognition; bar code recognition; and 3D modeling.

As discussed, this invention can be embodied in an eyewear-based system, device, and method for monitoring a person's nutritional intake comprising eyeglasses, wherein these eyeglasses further comprise at least one camera, wherein this camera automatically takes pictures or records images of food when a person is consuming food, and wherein these food pictures or images are automatically analyzed to estimate the type and quantity of food. The term food as used herein refers to beverages as well as solid food.

In an example, this invention can be used to monitor and modify a person's nutritional intake as part of an overall system for human weight management. In an example, this invention can provide feedback to help a person to manage their weight. In an example, this invention can provide negative stimuli in association with unhealthy types and/or quantities of food. In an example, this invention can provide positive stimuli in association with healthy types and/or quantities of food. In an example, negative stimuli can cause a person to consume less unhealthy food and positive stimuli can cause a person to consume more healthy food. In an example, this invention can modify the absorption of nutrients from food that a person has consumed. In an example, this invention can selectively cause a person to absorb fewer nutrients from unhealthy food. In an example, this invention can cause a person to selectively absorb more nutrients from healthy food. In an example, this invention can modify a person's nutritional intake in order to help the person to manage their weight by modifying the person's food consumption and/or modifying the person's absorption of nutrients from consumed food.

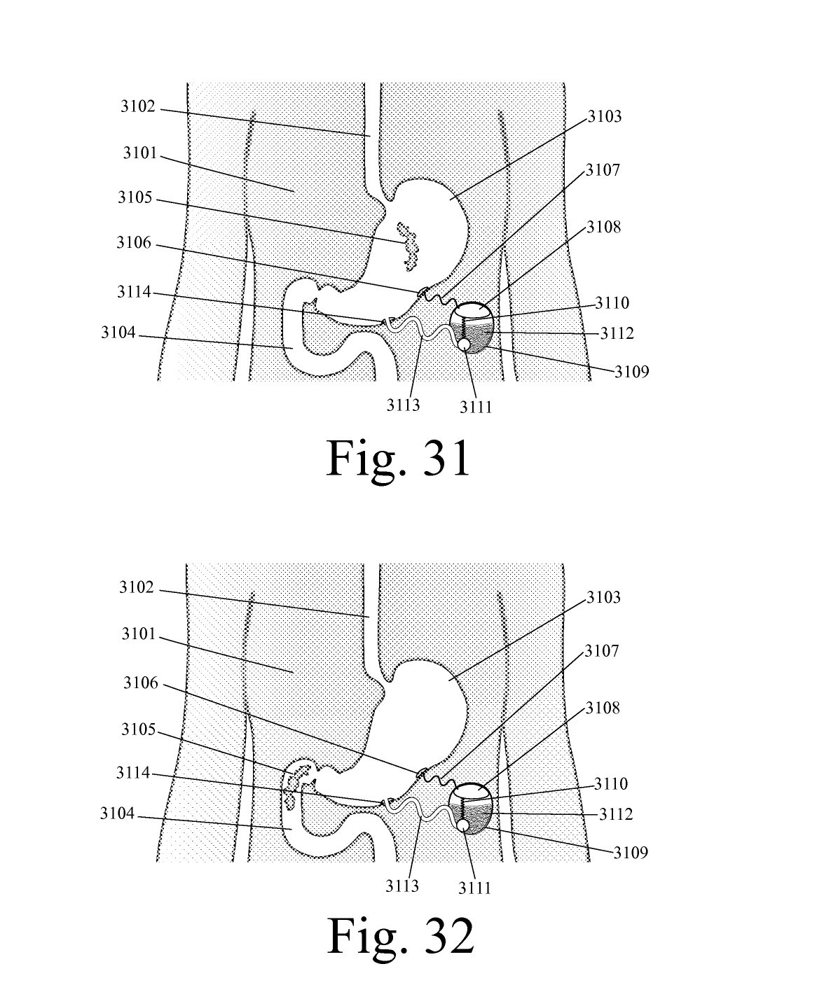

FIGS. 31 through 40 now show some examples of how this invention can be embodied in a device and method for selectively and automatically reducing absorption of nutrients from unhealthy food in a person's gastrointestinal tract. This can help a person to lose weight without the deficiencies of essential nutrients that can occur with food-blind procedures and devices in the prior art that indiscriminately reduce absorption of healthy food as well as unhealthy food. However, these figures are just some examples of how this invention can be embodied. They do not limit the full generalizability of the invention claims.

FIG. 31 shows an example of how this invention can be embodied in a device for selectively and automatically reducing absorption of nutrients from unhealthy food in a person's gastrointestinal tract. FIG. 31 shows a longitudinal cross-sectional view of person's torso 3101. This view includes a longitudinal cross-sectional view of a portion of the person's gastrointestinal tract comprising the esophagus 3102, stomach 3103, and duodenum 3104. This figure also shows a bolus of food 3105 in stomach 3103 that the person has consumed. In FIG. 31, the bolus of food 3105 is healthy food.

FIG. 31 also shows one embodiment of an implanted device for selective malabsorption of unhealthy food. Subsequent figures will provide sequential views showing how this device works to selectively and automatically reduce absorption of nutrients from unhealthy food, while allowing normal absorption of nutrients from healthy food. Selective malabsorption of unhealthy food, while allowing normal absorption of healthy food, can help a person to lose weight without suffering the deficiencies of essential nutrients that can be occur with food-blind bariatric procedures and malabsorption devices in the prior art.

As shown in the example, in FIG. 31, a food-identifying sensor 3106 can be attached to the interior wall of stomach 3103. Food-identifying sensor 3106 can selectively and automatically detect when the person is consuming unhealthy food. In an example, food-identifying sensor 3106 can perform intragastric chemical analysis to differentiate between consumption of unhealthy food versus healthy food. In an example, unhealthy food can be identified based on a high concentration of one or more of the following nutrients: sugars, simple sugars, simple carbohydrates, fats, saturated fats, cholesterol, and sodium.

In various examples, food-identifying sensor 3106 can be selected from the group consisting of: chemical sensor, biochemical sensor, amino acid sensor, biological sensor, chemoreceptor, cholesterol sensor, chromatography sensor, EGG sensor, enzyme-based sensor, fat sensor, particle size sensor, peristalsis sensor, glucose sensor, impedance sensor, membrane-based sensor, Micro Electrical Mechanical System (MEMS) sensor, microfluidic sensor, micronutrient sensor, molecular sensor, motion sensor, nutrient sensor, osmolality sensor, pH level sensor, protein-based sensor, reagent-based sensor, and temperature sensor.

In the embodiment of this invention that is shown in FIG. 31, food-identifying sensor 3106 is connected by wire 3107 to a release-control mechanism 3108 that is contained in an implanted reservoir 3109. Release-control mechanism 3108 is then connected by wire 3110 to pump 3111 which is also contained in reservoir 3109. Pump 3111 is in fluid communication with an absorption-reducing substance 3112 that is contained in reservoir 3109 until this substance is released into the stomach 3103 through lumen 3113 and one-way valve 3114. Absorption-reducing substance 3112 is released into the interior of the person's stomach 3103 to reduce food absorption when food-identifying sensor 3106 detects consumption of unhealthy food.

In an example, absorption-reducing substance 3112 can comprise one or more ingredients that are Generally Recognized As Safe (GRAS) under Sections 201(s) and 409 of the Federal Food, Drug, and Cosmetic Act. In various examples, absorption-reducing substance 3112 can comprise one or more ingredients selected from the group consisting of: psyllium, cellulose, avocado oil, castor oil, chitin, chitosan, beta-glucan, coconut oil, corn oil, flaxseed oil, olive oil, palm oil, safflower oil, soy oil, sunflower oil, gelatin, pectin, agar, guar gum, gum acacia, lignin, xantham gum, other insoluble fiber, other soluble fiber, other gum, and other vegetable oil.

In this embodiment, the sequence of action for this implanted device is as follows. First, a bolus of food 3105 enters the stomach 3103. Then, food-identifying sensor 3106 detects whether food 3105 is unhealthy using intragastric chemical analysis. If food 3105 is unhealthy, then sensor 3106 sends a signal through wire 3107 to release-control mechanism 3108. This signal triggers activation of pump 3111 which releases absorption-reducing substance 3112 through lumen 3113 and one-way valve 3114 into the stomach 3103. After the absorption-reducing substance 3112 is released into the stomach, the absorption-reducing substance 3112 reduces absorption of nutrients from the bolus of unhealthy food 3105 by coating the interior walls of the duodenum 3104, by coating the bolus of food 3105, or by a combination of both coating actions.

In an example, the absorption-reducing substance 3112 can be used to selectively reduce absorption of nutrients from unhealthy food by temporarily coating a portion of the interior walls of the intestine when consumption of unhealthy food is detected. In an example, an absorption-reducing substance 3112 can be used to selectively reduce absorption of nutrients from unhealthy food by coating the food, food particles, nutrients, and/or chyme in the gastrointestinal tract when consumption of unhealthy food is detected.

In an example, a release-control mechanism 3108 can start releasing an absorption-reducing substance 3112 into the person's stomach 3103 in response to detection of consumption of unhealthy food by food-identifying sensor 3106. In an example, a release-control mechanism 3108 can stop releasing absorption-reducing substance 3112 into the person's stomach 3103 in response to detection of consumption of healthy food by the food-identifying sensor 3106.

In an example, a release-control mechanism 3108 can communicate wirelessly with a source external to the person's body. In an example, a release-control mechanism 3108 can be programmed, or otherwise adjusted, to change the types of selected foods or nutrients to which it responds by releasing an absorption-reducing substance 3112 into the person's gastrointestinal tract.

In various examples, a release-control mechanism 3108 can be programmed to adjust one or more of the following aspects of its response to food-identifying sensor 3106: the type of food which triggers decreased food absorption; the quantity of food which triggers decreased food absorption; the time of day, day of the week, or other timing parameter concerning food consumption which triggers decreased food absorption; the effect of the person's past food consumption on decreased food absorption; the effect of the person's caloric expenditure on decreased food absorption; and the effect of a personalized diet plan created for the person by a health care professional.

FIGS. 31 and 32 show how this embodiment of this invention can respond (or, more precisely, not respond) to a bolus of healthy food 3105. These figures show that the device does not interfere with the normal absorption of healthy food 3105. This is an advantage over malabsorption procedures and devices that blindly reduce absorption of all food, including healthy food. FIG. 31 shows a bolus of healthy food 3105 that has entered the person's stomach 3103. Food-identifying sensor 3106 recognizes that bolus of food 3105 is healthy, based on intragastric chemical analysis, and does not trigger any reduction in absorption of its nutrients. Accordingly, FIG. 32 shows bolus of food 3105 (or a resulting bolus of chyme that contains particles of food 3105) passing normally through the person's duodenum 3104 for full nutrient absorption. This avoids the deficiencies of essential nutrients that can be caused by food-blind malabsorption procedures and devices in the prior art.

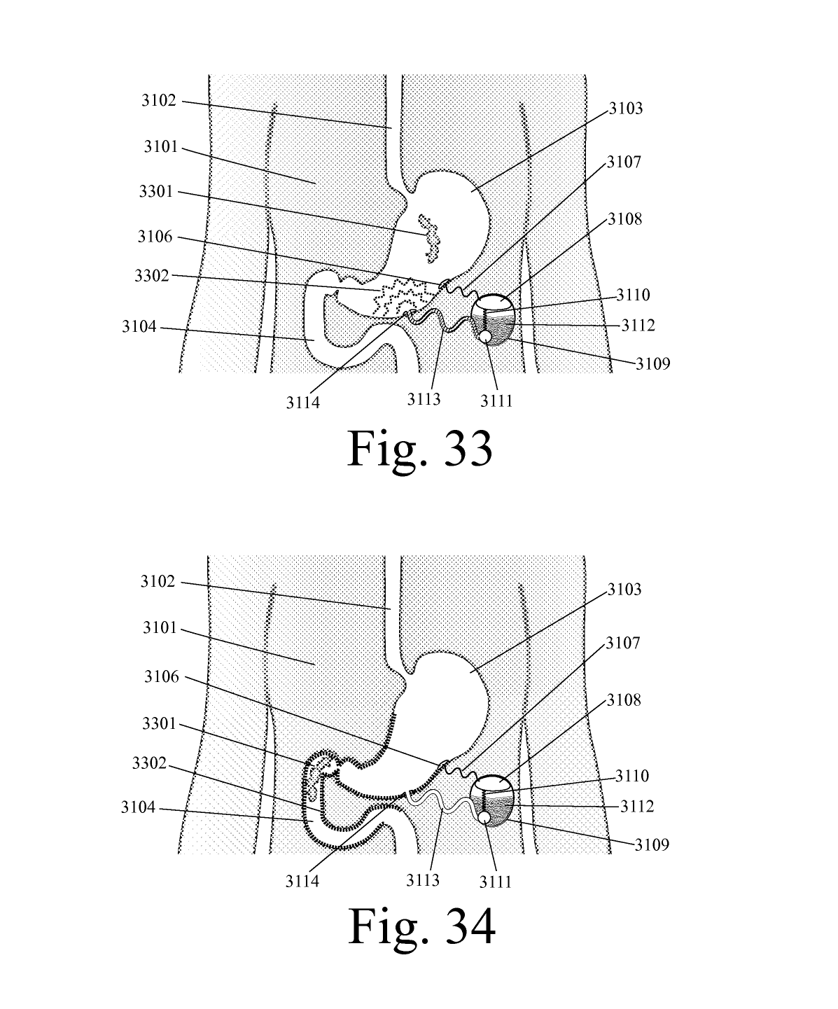

FIGS. 33 and 34 show how this embodiment can selectively and automatically respond to a bolus of unhealthy food 3301. In FIG. 33, a bolus of unhealthy food 3301 has entered the person's stomach 3103. The bolus of unhealthy food 3301 is identified as unhealthy by food-identifying sensor 3106. In an example, this identification can be done using intragastric chemical analysis. Next, sensor 3106 sends a signal indication, via wire 3107, that the person has consumed unhealthy food 3301 to release-control mechanism 3108. Then, release-control mechanism 3108 activates pump 3111 to release a quantity of the absorption-reducing substance 3112, through lumen 3113 and one-way valve 3114, into the interior of stomach 3103. The release of the absorption-reducing substance 3112 into stomach 3103 is represented by concentric wavy dotted lines 3302 that radiate outwards from one-way valve 3114 into the interior of the person's stomach 3103.

FIG. 34 shows an example of what can happen when the absorption-reducing substance 3112 is released into the person's stomach 3103. In this example, the absorption-reducing substance 3112 temporarily coats the lower portion of person's stomach 3103 and, more importantly for malabsorption of nutrients, the absorption-reducing substance 3112 also coats the interior walls of the person's duodenum 3104. This temporary coating action is represented in FIG. 34 by thick dashed lines 3302 on the interior surface of the person's lower stomach 3103 and on the interior walls of the person's duodenum 3104. In this example, coating 3302 on the walls of the duodenum reduces absorption of nutrients from the bolus of unhealthy food 3301 (or a resulting bolus of chyme that contains particles of food 3301) as this bolus passes through the duodenum.

In an example, this temporary reduction in nutrient absorption occurs because of an increase in the speed or motility with which a bolus of food 3301 passes through the duodenum 3104. In an example, this temporary reduction in nutrient absorption can occur because of a temporary decrease in the nutrient permeability of the mucus that covers the interior walls of the duodenum 3104. In an example, this temporary reduction in nutrient absorption can occur because the absorption-reducing substance temporarily binds to the nutrient-absorbing organelles along the interior walls of the duodenum 3104. The temporary nature of this duodenal coating is important because it allows the duodenum 3104 to return to normal absorption status for later consumption and absorption of healthy food. This is a significant improvement over food-blind procedures and devices in the prior art that cause permanent and indiscriminant malabsorption of all types of food.

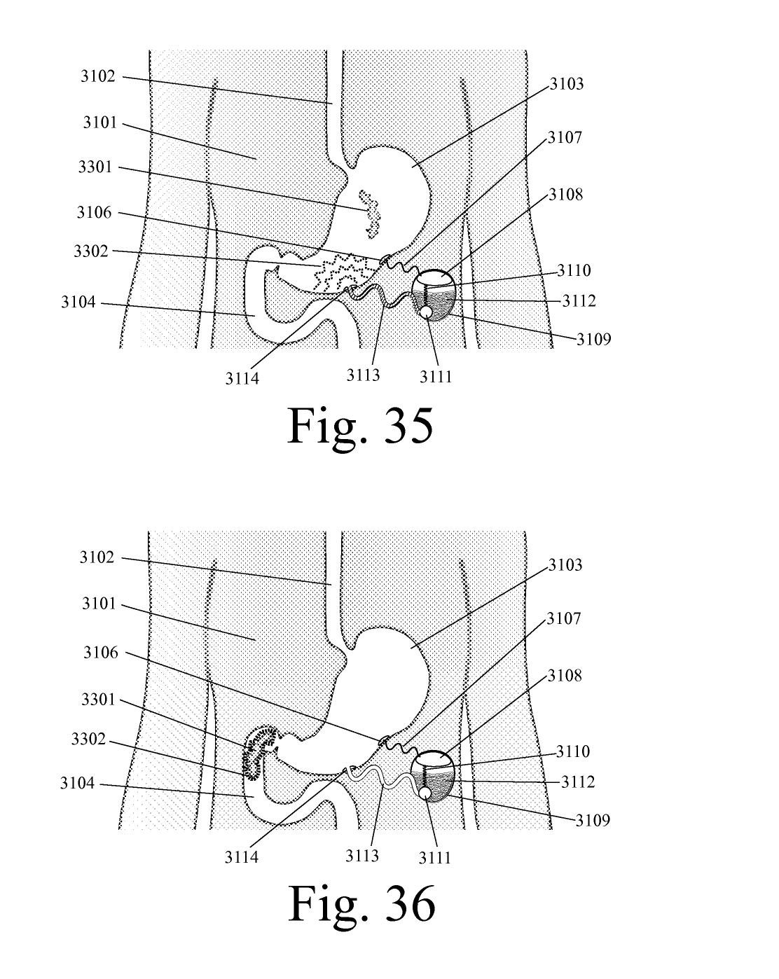

FIGS. 35 and 36 show another example of how this embodiment can selectively and automatically reduce absorption of a bolus of unhealthy food 3301. As was the case in FIG. 33, FIG. 35 shows that a bolus of unhealthy food 3301 has entered stomach 3103. Also, as shown in FIG. 33, FIG. 35 shows that the food-identifying sensor 3106 identifies that bolus of food 3301 is unhealthy. In an example, this identification is done using intragastric chemical analysis. Identification of bolus of food 3301 as being unhealthy triggers release-control mechanism 3108. This, in turn, activates pump 3111 which releases absorption-reducing substance 3112 into the person's stomach 3103. The release of absorption-reducing substance 3112 into stomach 3103 is again represented by wavy dotted lines 3302 which radiate outwards from one-way valve 3114 into the stomach interior 3103.

FIG. 36 is similar to FIG. 34, except that now the absorption-reducing substance 3112 coats the surface of bolus of food 3301 instead of the interior walls of duodenum 3104. This coating action is represented in FIG. 36 by thick dashed lines 3302 around the perimeter of bolus of food 3301 (or the resulting bolus of chyme that contains particles of food 3301) as it passes through the duodenum. In an example, reduced absorption of nutrients from bolus of food 3301 can occur because of an increase in the speed at which this bolus of food 3301 passes through duodenum 3104. In an example, reduced absorption of nutrients from this bolus of food 3301 can occur because the coating around the bolus prevents nutrients in the bolus from coming into contact with the nutrient-absorbing organelles along the interior walls of duodenum 3104.

In this example, with the bolus having been coated instead of the walls of the duodenum, the duodenum is able to normally and fully absorb nutrients from any subsequent bolus of healthy food that comes down the gastrointestinal tract. This is a significant improvement over food-blind procedures and devices in the prior art that cause permanent and indiscriminant malabsorption of all types of food.

FIGS. 31 through 36 show some examples of how this invention can be embodied in a device for selectively and automatically reducing the absorption of selected types of food in a person's gastrointestinal tract. This device comprises: (a) a food-identifying sensor 3106 that selectively detects when the person is consuming and/or digesting selected types of food; (b) an absorption-reducing substance 3112 that is released into the interior of the person's gastrointestinal tract to temporarily reduce absorption of nutrients from food by the gastrointestinal tract; (c) an implanted reservoir 3109 that contains a quantity of the absorption-reducing substance, wherein this reservoir is configured to be implanted within the person's body and wherein there is an opening or lumen through which the absorption-reducing substance is released from the reservoir into the interior of a portion of the person's gastrointestinal tract; and (d) a release-control mechanism 3108 that controls the release of the absorption-reducing substance from the reservoir into the person's gastrointestinal tract, wherein this release-control mechanism can selectively and automatically increase the release of the absorption-reducing substance when the food-identifying sensor detects that the person is consuming and/or digesting selected types of food. I will now discuss each of these four components in greater detail.

I will first discuss the food-identifying sensor in greater detail. In an example, a food-identifying sensor can selectively detect consumption and/or digestion of selected types of food. In an example, food identification can occur as food is entering, or being consumed within, a person's mouth. In an example, food identification can occur as food is passing through, and being digested within, a person's stomach or another portion of a person's gastrointestinal tract. In an example, a food-identifying sensor can selectively detect consumption and/or digestion of unhealthy food. In an example, a food-identifying sensor can selectively discriminate between consumption and/or digestion of unhealthy types or quantities of food versus consumption and/or digestion of healthy types or quantities of food.

In an example, a food-identifying sensor can selectively detect consumption or digestion of unhealthy foods as identified by their having a high concentration or large amount of selected nutrients. In an example, there can be a predefined list of types of food which are classified as unhealthy. In an example, there can be predefined quantities of selected types of food which are classified as unhealthy. In an example, there can be a predefined list of types of food which are classified as healthy. In an example, there can be predefined quantities of selected types of food which are classified as healthy. In an example, lists of the types and quantities of food which are classified as unhealthy or healthy can be compiled and adjusted by experts and professionals who provide the person with nutritional and dietary counseling.

In an example, a food-identifying sensor can selectively detect consumption or digestion of unhealthy food based on their having a high concentration or large amount of nutrients selected from the group consisting of: sugars, simple sugars, simple carbohydrates, fats, saturated fats, fat cholesterol, and sodium. In an example, such a sensor can selectively detect consumption or digestion of foods with a high concentration or quantity of cholesterol. In various examples, a food-identifying sensor can selectively detect consumption and/or digestion of one or more selected types of foods selected from the group consisting of: fried food, high-cholesterol food, high-fat food, high-sugar food, and high-sodium food.

In an example, a food-identifying sensor can selectively detect when a person is consuming or digesting unhealthy types of food and can selectively detect when a person is consuming or digesting healthy types of food. In an example, a food-identifying sensor can selectively differentiate between consumption of unhealthy versus healthy food. In an example, unhealthy food can be identified as having a relatively large amount of sugars, simple carbohydrates, fats, saturated fats, cholesterol, and/or sodium. In an example, unhealthy food can be identified as having a relatively large number of grams of carbohydrates or simple carbohydrates, grams of fats or saturated fats, and/or milligrams of sodium per serving.

In an example, healthy food can be identified in a negative manner, as any food that is not identified as being unhealthy. In an alternative example, healthy food can be identified in a positive manner, as any food with a large concentration or amount of one or more nutrients selected from the group consisting of: food with a lot of soluble fiber, food with a lot of insoluble fiber, food with a lot of essential vitamins, and food with a high concentration of essential nutrients that the person's diet generally lacks.

In various examples, an unhealthy type of food can be identified as being in the group consisting of: fried or deep-fried food, French fries, high-cholesterol food, high-fat food or high-saturated-fat food, food with a high amount of high-fructose corn syrup, high-sodium food, food with a high amount of simple or refined sugar or high-sugar food, food with a high amount of hydrogenated oil, and non-diet soda pop. In an example, a food-identifying sensor can selectively detect when a person is consuming or digesting food that has: at least a selected number of grams of fats per serving, at least a selected number of grams of saturated fats per serving, at least a selected number of milligrams of fat cholesterol per serving, at least a selected number of grams of carbohydrates per serving, and/or at least a selected number of milligrams of sodium per serving. In an example, quantities of food exceeding one or more of these amounts can be automatically classified as unhealthy.

In a variation on this example, serving size for the purposes of food identification can be based on suggested serving sizes and/or population norms. For example, a food-identifying sensor can selectively detect when a person is consuming or digesting food that has: at least a selected number of grams of fats per suggested serving, at least a selected number of grams of saturated fats per suggested serving, at least a selected number of milligrams of fat cholesterol per suggested serving, at least a selected number of grams of carbohydrates per suggested serving, and/or at least a selected number of milligrams of sodium per suggested serving.

In an example, a food-identifying sensor can selectively detect consumption or digestion of food that comprises over: a selected number of grams of fat per suggested serving, a selected number of grams of saturated fat per suggested serving, a selected number of milligrams of fat cholesterol per suggested serving, a selected number of grams of carbohydrate per suggested serving, and/or a selected number of milligrams of sodium per suggested serving. In an example, quantities of food exceeding one or more of these amounts can be automatically classified as unhealthy.

In another example, serving size for the purposes of food identification can be based on a person's past eating habits and/or the actual quantity of food that a person is consuming, in real time, during an eating episode. In an example, an eating episode can be defined as a period of time with continuous eating. In an example, an eating episode can be defined as a period of time with less than a selected amount of time between mouthfuls and/or swallows.

In an example, a food-identifying sensor can selectively detect when a person is consuming or digesting food that has: at least a selected number of grams of fats per actual serving, at least a selected number of grams of saturated fats per actual serving, at least a selected number of milligrams of fat cholesterol per actual serving, at least a selected number of grams of carbohydrates per actual serving, and/or at least a selected number of milligrams of sodium per actual serving. In an example, quantities of food exceeding one or more of these amounts can be automatically classified as unhealthy.

In an example, a food-identifying sensor can selectively detect when a person is consuming or digesting food that has: at least a selected number of grams of fats per eating episode, at least a selected number of grams of saturated fats per eating episode, at least a selected number of milligrams of fat cholesterol per eating episode, at least a selected number of grams of carbohydrates per eating episode, and/or at least a selected number of milligrams of sodium per eating episode. In an example, quantities of food exceeding one or more of these selected amounts can be automatically classified as unhealthy.

In an example, a food-identifying sensor can enable selective detection of cumulative consumption of food during a period of time that totals: at least a selected number of grams of fats, at least a selected number of grams of saturated fats, at least a selected number of milligrams of fat cholesterol, at least a selected number of grams of carbohydrates, and/or at least a selected number of milligrams of sodium. In an example, a food-identifying sensor can enable selective detect of cumulative consumption of food during a period of time that totals: at least a predetermined amount of fat, at least a predetermined amount of saturated fat, at least a predetermined amount of fat cholesterol, at least a predetermined amount of carbohydrates, and/or at least a predetermined amount of sodium. In an example, quantities of food exceeding one or more of these amounts can be automatically classified as unhealthy.

In another variation on these examples, the amount of selected nutrients in a specific type of food can be evaluated as a percentage of the recommended daily intake for such a nutrient. For example, a food-identifying sensor can selectively detect consumption or digestion of food that comprises at least: a selected percentage of the recommended daily intake of fat per suggested serving, a selected percentage of the recommended daily intake of saturated fat per suggested serving, a selected percentage of the recommended daily intake of fat cholesterol per suggested serving, a selected percentage of the recommended daily intake of carbohydrate per suggested serving, and/or a selected percentage of the recommended daily intake of sodium per suggested serving. In an example, quantities of food exceeding one or more of these recommended amounts can be automatically classified as unhealthy.

In an example, food identification can occur as food is being consumed, and beginning to be digested, within a person's mouth. In an example, a food-identifying sensor can detect a selected type of food by analyzing the composition of the person's saliva as that food is being digested in a person's mouth. In an example, a food-identifying sensor can be a chemical sensor that uses chemical analysis to identify particular types of food and/or nutrients. In an example, a food-identifying sensor can analyze the composition of the person's saliva in order to automatically and selectively detect when a person is digesting a food that is high in (simple) sugar or (saturated) fat, while that food is being digested within the person's mouth.

In various examples, a food-identifying sensor which is in fluid communication with a person's oral or nasal cavity can identify food as being unhealthy based on one or more methods selected from the group consisting of: chemical analysis of food as it begins to be digested within a person's mouth; olfactory analysis of food as it beings to be digested within a person's mouth; image analysis of images of food as it approaches the person's mouth; sonic analysis of chewing or swallowing as food is consumed; and analysis of signals from nerves that innervate a person's taste buds and/or olfactory receptors.

There are a number of different types of sensors that can be used to identify a selected type of food and/or a selected quantity of that food. In an example, a food-identifying sensor can be a chemical sensor. In various examples, a chemical sensor can detect the amount or concentration of sugars, simple carbohydrates, fats, saturated fats, cholesterol fat, and/or sodium in food while it is being consumed or digested by a person.

In various examples, a food-identifying sensor can be selected from the group consisting of: chemical sensor, biochemical sensor, accelerometer, amino acid sensor, biological sensor, camera, chemoreceptor, cholesterol sensor, chromatography sensor, electrogastrogram sensor, electrolyte sensor, electromagnetic sensor, EMG sensor, enzymatic sensor, fat sensor, flow sensor, particle size sensor, peristalsis sensor, genetic sensor, glucose sensor, imaging sensor, impedance sensor, interferometer, medichip, membrane-based sensor, Micro Electrical Mechanical System (MEMS) sensor, microfluidic sensor, micronutrient sensor, molecular sensor, motion sensor, muscle activity sensor, nanoparticle sensor, neural impulse sensor, optical sensor, osmolality sensor, pattern recognition sensor, pH level sensor, pressure sensor, protein-based sensor, reagent-based sensor, sound sensor, strain gauge, and temperature sensor.

In various examples, a food-identifying sensor can be located in any location from which it is in fluid and/or gaseous communication with food that the person is consuming or digesting. In an example, a food-identifying sensor can be implanted within a person's body. An implanted sensor is generally less dependent on voluntary action by the person than an external sensor. For example, an implanted sensor can operate in an automatic manner, regardless of the person's behavior. In contrast, an external sensor, such as a picture-taking mobile electronic device or a wearable electronic imaging device can be forgotten, obscured, or just plain unused. An implanted food-identifying sensor is less prone to compliance or circumvention problems than an external sensor. In various examples, an implanted food-identifying sensor can be attached to, or implanted within, the person's body by one or more means selected from the group consisting of: suture, staple, adhesive, glue, clamp, clip, pin, snap, elastic member, tissue pouch, fibrotic tissue, screw, and tissue anchor.

In an example, an implanted food-identifying sensor can be configured to be attached to, or implanted within, a person's stomach. In an example, a food-identifying sensor can detect digestion of selected types of food within a person's stomach. In another example, a food-identifying sensor can be configured to be attached to, or implanted within, a portion of a person's intestine. In an example, a food-identifying sensor can detect digestion of selected types of food within a person's intestine. In various examples, an implanted food-identifying sensor can be configured to be attached to, or implanted within, a person's stomach, duodenum, jejunum, ileum, caecum, colon, or esophagus. In various examples, a food-identifying sensor can be configured to be implanted within a person's abdominal cavity with a means of fluid, neural, or other communication with the person's stomach, duodenum, jejunum, ileum, caecum, colon, or esophagus.

In another example, a food-identifying sensor can be located closer to the initial point of food consumption, such as in a person's mouth or nose. In an example, an implanted food-identifying sensor can be configured to be attached to, implanted within, or otherwise in fluid communication with a person's mouth. In an example, an implanted food-identifying sensor can be configured to be attached to, implanted within, or otherwise in fluid communication with a person's nose.

One advantage of having a food-identifying sensor that is in fluid communication with a person's oral or nasal cavity is that it can identify consumption of a particular bolus of food sooner than a sensor that is in fluid communication with the person's stomach. This can allow time for modification of the person's stomach or intestinal walls before the bolus of food arrives. In an example, a food-identifying sensor in a person's mouth or nose can be in wireless communication with an absorption-reducing member in the person's stomach or intestine.

In an example, a mouth or nose based food-identifying sensor can provide “earlier detection” that a bolus of unhealthy food will be coming down the esophagus into the stomach and intestine. In an example, such advance notice (from a mouth-based sensor) can enable coating the walls of the duodenum with an absorption-reducing coating before a certain bolus of food arrives there. As another example, such advance notice (from a mouth-based sensor) can enable releasing a food-coating substance in the stomach before a certain bolus of food moves down the esophagus to enter the stomach. These actions can more efficiently reduce absorption of a particular bolus of food as it moves through a person's gastrointestinal tract.

In an example, a food-identifying sensor can be configured to be attached to, or implanted within, a person's oral cavity, a person's nasal cavity, or tissue surrounding one of these cavities. In various examples, such a sensor can be configured to be attached to, or implanted within, the person's hard palate, palatal vault and/or upper mouth roof, teeth, tongue, or soft palate. In an example, such a food-identifying sensor can detect consumption or digestion of unhealthy food within the person's mouth.

In an example, a food-identifying sensor can be configured to be implanted in a subcutaneous site or an intraperitoneal site. In an example, a food-identifying sensor can be configured to be attached to a nerve. In an example, a food-identifying sensor can be in communication with a nerve that is connected to the stomach. In an example, a food-identifying sensor can be configured to be implanted in adipose tissue or muscular tissue.

There are advantages to having a food-identifying sensor be implanted in a person's body. For example, having a sensor be implanted can make a sensor more automatic in nature and less susceptible to non-compliance, manipulation, or circumvention. However, there can also be advantages to having a food-identifying sensor be external to the person's body. As one advantage of an external sensor, an external sensor can be less invasive and/or costly than an implanted sensor. As a second potential advantage, an external sensor can detect food consumption earlier than a sensor in a person's mouth or nose. For example, an external food-identifying sensor can identify food as person reaches for it, as the person brings it up to their mouth, or as the person inserts it into their mouth. As a third potential advantage of an external sensor, some forms of food identification (especially image analysis) are easier when performed on food before it is inserted into a person's mouth.

In an example, an external food-identifying sensor can be in wireless communication with an internal absorption-reducing implant. This allows the internal absorption-reducing implant to be selectively activated when the person consumes unhealthy food, but still allow normal absorption of nutrients from healthy food. In an example, a food-identifying sensor can be worn externally on the person's body and be in wireless communication with an implanted member that selectively modifies food absorption.

In an example, a food-identifying sensor can be incorporated into a mobile electronic device, such as a cell phone, mobile phone, or tablet that is carried by the person. In an example, an external sensor can be in wireless communication with an implanted member that selectively modifies consumption of a given bolus of food in order to reduce absorption of unhealthy food and allow normal absorption of healthy food. In an example, an external sensor, or a mobile device of which this sensor is an application or component, can communicate with the internet and/or other mobile devices.

In an example, a food-identifying sensor can be part of a piece of electronically-functional jewelry that is worn by a person. In an example, a food-identifying sensor can be worn on a body member selected from the group consisting of: wrist, hand, finger, arm, torso, neck, head, and ear. In an example, an external food-identifying sensor can be incorporated into a piece of electronically-functional jewelry selected from the group consisting of electronically-functional: necklace, pendant, finger ring, bracelet, nose ring, and earring. In an example, an external food-identifying sensor can be incorporated into an electronically-functional wrist watch, pair of eyeglasses, or hearing aid. In an example, an external sensor, or piece of electronically-functional jewelry of which this sensor is a part, can communicate with the internet and/or other people via other electronic communication means.

I will now discuss the absorption-reducing substance in greater detail. In an example, an absorption-reducing substance can have the property that it reduces absorption of nutrients from food in a person's gastrointestinal tract when this substance is released directly into the person's gastrointestinal tract. In an example, an absorption-reducing substance can reduce absorption of nutrients by temporarily coating the walls of a portion of the person's intestines. In an example, such a substance can reduce absorption of nutrients by selectively coating a particular bolus of food, food particles, or chyme as it moves through the person's gastrointestinal tract. In an example, this substance can coat the walls of a person's intestine and coat a selected bolus of food.

In an example, an absorption-reducing substance can have a local and temporary absorption-reducing effect that allows selective reduction of the absorption of a particular bolus of food. In an example, this selective absorption-reducing effect can be used to selectively reduce absorption of nutrients from unhealthy types and/or quantities of food, while allowing normal absorption of nutrients from healthy types and/or quantities of food. This is an improvement over systemic drugs that have an indiscriminant effect on appetite or food absorption that blindly affect absorption of nutrients from healthy as well as unhealthy food. This is also an improvement over surgical procedures and malabsorption devices in the prior art that blindly reduce absorption of nutrients from healthy food as well as unhealthy food.

In an example, an absorption-reducing substance can be released directly into a person's gastrointestinal tract from an implanted reservoir in order to reduce absorption of nutrients from a selected bolus of unhealthy food. In an example, the food consumed may be of an unhealthy type and/or quantity. It is advantageous for absorption reduction to be temporary so that the substance can be used to selectively reduce food absorption only when the person consumes a bolus of unhealthy food, but still allow normal absorption of nutrients from healthy food. This can help to avoid a deficit of healthy nutrients that can sometimes occur with permanent absorption-reducing methods such as permanent bariatric surgery.

In an example, an absorption-reducing substance can work by creating a coating between a bolus of food and the walls of the gastrointestinal tract. In an example, this coating can reduce fluid communication between food and the walls. In an example, this coating can increase the speed at which food travels through a portion of the gastrointestinal tract. In an example, this coating can coat food (or food particles or chyme) so that nutrients in the food do not come into contact with the walls of the intestine. In another example, this coating can be on the walls of the intestine itself, so that the nutrient-absorbing organelles on the intestinal wall are temporarily blocked from absorbing nutrients from food. In an example, both the food and the walls can be coated.

In various examples, an absorption-reducing substance can be released into the gastrointestinal tract to coat food, food particles, nutrients, or chyme in the gastrointestinal tract. In various examples, an absorption-reducing substance can coat food, food particles, nutrients, or chyme in the gastrointestinal tract in order to increase or decrease the speed at which the coated material moves through the gastrointestinal tract. In various examples, an absorption-reducing substance can coat food, food particles, nutrients, or chyme in the gastrointestinal tract to decrease fluid communication between food in the gastrointestinal tract and the walls of the gastrointestinal tract.

In an example, an absorption-reducing substance can coat a portion of the interior walls of the duodenum or another portion of the intestine. In an example, an absorption-reducing substance can coat, cover, or block the nutrient-absorbing organelles that are located on the walls of a portion of the intestine. In an example, this coating, covering, or blocking action can be temporary. This coating, covering, or blocking action can be timed in advance of the arrival of a bolus of unhealthy food in the intestine so that malabsorption of food is selectively targeted at unhealthy food. Ideally, the adsorption-reducing coating, covering, or blocking action is such that it can wear off by the time that a bolus of healthy food enters the gastrointestinal tract. However, even if there is a lag between when a bolus of unhealthy food passes through the gastrointestinal tract and when the absorption-reducing effect wears off, this device and method can still be superior for absorption of nutrients from healthy food as compared to devices and methods in the prior art that uniformly and indiscriminately reduce absorption of all food.

In an example, an absorption-reducing substance can coat a portion of the interior walls of the gastrointestinal tract in order to increase or decrease the speed at which food moves through the gastrointestinal tract. In an example, an absorption-reducing substance can coat a portion of the interior walls of the gastrointestinal tract in order to decrease fluid communication between food in the gastrointestinal tract and the walls of the gastrointestinal tract. In an example, an absorption-reducing substance can temporarily coat a portion of the interior walls of the duodenum, of another portion of the intestine, or of another portion of the gastrointestinal tract.

In an example, an absorption-reducing substance can temporarily coat or block nutrient-absorbing organelles on a portion of the interior walls of the gastrointestinal tract. In an example, an absorption-reducing substance can temporarily coat a portion of the interior walls of the gastrointestinal tract to increase the speed at which food moves through the gastrointestinal tract. In an example, an absorption-reducing substance can temporarily coat a portion of the interior walls of the gastrointestinal tract to decrease fluid communication between food in the gastrointestinal tract and the walls of the gastrointestinal tract.

In an example, an absorption-reducing substance that is released into the gastrointestinal tract can mechanically, chemically, or biologically bind to, or adhere to, material or tissue in the gastrointestinal tract in order to reduce absorption of food. For example, an absorption-reducing substance can bind to, or adhere to, food, food particles, nutrients, or chyme in the gastrointestinal tract. In an example, an absorption-reducing substance can isolate food, food particles, nutrients, or chyme in the gastrointestinal tract to increase or decrease the speed at which this material moves through the gastrointestinal tract. In an example, an absorption-reducing substance can bind to, or adhere to, food, food particles, nutrients, or chyme in the gastrointestinal tract in order to decrease fluid communication between food nutrients in the gastrointestinal tract and the walls of the gastrointestinal tract.

In an example, an absorption-reducing substance can mechanically, chemically, or biologically bind to, or adhere to, a portion of the interior walls of the duodenum or another portion of the intestine. In an example, an absorption-reducing substance can temporarily bind or adhere to a portion of the interior walls of the gastrointestinal tract. In an example, an absorption-reducing substance can bind to, or adhere to, nutrient-absorbing organelles on a portion of the interior walls of the gastrointestinal tract. Such binding or adhering action can reduce the ability of these organelles to absorb nutrients from a selected bolus of unhealthy food passing through the gastrointestinal tract. When such binding or adhering action is temporary, the body can still absorb required nutrients from a bolus of healthy food consumed some time after the bolus of unhealthy food has passed.

In an example, an absorption-reducing substance can bind to, or adhere to, a portion of the interior walls of the gastrointestinal tract in order to increase or decrease the speed at which food moves through the gastrointestinal tract. In an example, an absorption-reducing substance can have a laxative effect on a bolus of unhealthy food. This laxative effect can reduce unhealthy food absorption by reducing the duration of contact between the unhealthy food and the walls of the duodenum.

In an example, an absorption-reducing substance can temporarily bind to, or adhere to, a portion of the interior walls of the gastrointestinal tract in order to decrease fluid communication between food in the gastrointestinal tract and the walls of the gastrointestinal tract. When this temporary coating is timed in advance of a bolus of unhealthy food, then it can selectively reduce absorption of nutrients from unhealthy food. In an example, an absorption-reducing substance can temporarily block or otherwise disable nutrient-absorbing organelles on a portion of the interior walls of the person's duodenum or another portion of the person's intestine.

In an example, an absorption-reducing substance can work by affecting the mucus that covers the walls of the person's duodenum. In an example, the absorption-reducing substance can temporarily increase the thickness of the mucus on a portion of the interior walls of the person's duodenum. In an example, the absorption-reducing substance can temporarily increase the viscosity of the mucus on a portion of the interior walls of the person's duodenum. This increased thickness or viscosity can temporarily decrease fluid communication between nutrients in a selected bolus of food (or chyme) and the walls of the duodenum. In another example, the absorption-reducing substance can temporarily decreases the nutrient permeability of the mucus on a portion of the interior walls of the person's duodenum or another portion of the intestine. This decreased permeability can decrease the absorption of nutrients by the body from a bolus of unhealthy food moving through the person's gastrointestinal tract.

In various examples, an absorption-reducing substance can reduce absorption of food in the gastrointestinal tract by one or more means selected from the group consisting of: forming a temporary coating on the walls of the duodenum or another portion of the intestine; forming a coating on food or chyme in the gastrointestinal tract; forming a temporary coating on the walls of the intestine to reduce fluid communication between food or chyme in the gastrointestinal tract and the gastrointestinal tract walls; forming a coating on food or chyme in the gastrointestinal tract to reduce fluid communication between food or chyme in the gastrointestinal tract and the gastrointestinal tract walls.

In various examples, an absorption-reducing substance can reduce absorption of food in the gastrointestinal tract by one or more means selected from the group consisting of: forming a temporary coating on the walls of the intestine to increase the speed of food or chyme moving through the gastrointestinal tract; forming a coating on food or chyme moving through the gastrointestinal tract in order to increase the speed of food or chyme moving through the gastrointestinal tract; temporarily binding to the nutrient-absorbing organelles on the interior walls of a portion of the intestine; binding to food or chyme moving through the gastrointestinal tract; temporarily increasing the viscosity of the mucus that coats the interior walls of the duodenum or another portion of the intestine; temporarily decreasing the nutrient permeability of the mucus that coats the interior walls of the duodenum or another portion of the intestine; and temporarily covering or blocking the nutrient-absorbing organelles of the duodenum or another portion of the intestine.

In an example, a quantity of an absorption-reducing substance can be stored in an implanted reservoir. In an example, this substance may be stored in a liquid or gel form. In an example, this substance may be released into the person's gastrointestinal tract by an active pumping or spraying action. In an example, an absorption-reducing substance can be a liquid that coats material or tissue surfaces in the interior of a person's gastrointestinal tract when it is released into the interior of that tract. In an example, a quantity of an absorption-reducing substance can be stored in an implanted reservoir in a powder or solid form and then released into the person's gastrointestinal tract. In various examples, an absorption-reducing substance can be stored in reservoir and/or released into the gastrointestinal tract in a form selected from the group consisting of: liquid, emulsion, erodible formulation, gel, granules, microspheres, capsule, powder, semi-solid, solid, spray, and suspension.

In an example, an absorption-reducing substance can create a lubricious coating that temporarily separates food or food particles in the gastrointestinal tract from fluid communication with the walls of the gastrointestinal tract. In an example, an absorption-reducing substance can create a temporary nutrient barrier that temporarily isolates nutrients in food passing through the gastrointestinal tract from the nutrient-absorbing organelles along the walls of the gastrointestinal tract. In an example, an absorption-reducing substance can reduce absorption of food for a limited period of time after being released into the gastrointestinal tract.

In an example, an absorption-reducing substance can comprise one or more ingredients that are Generally Recognized As Safe (GRAS) under Sections 201(s) and 409 of the Federal Food, Drug, and Cosmetic Act. In an example, an absorption-reducing substance can comprise a composition with insoluble fiber. In an example, an absorption-reducing substance can comprise a composition with soluble fiber. In an example, an absorption-reducing substance can beneficially coat the walls of a portion of the intestine in order to reduce the body's absorption of fats. In various specific examples, an absorption-reducing substance can comprise one or more ingredients that are selected from the group consisting of: psyllium, cellulose, avocado oil, castor oil, chitin, chitosan, beta-glucan, coconut oil, corn oil, flaxseed oil, olive oil, palm oil, safflower oil, soy oil, sunflower oil, gelatin, pectin, agar, guar gum, gum acacia, lignin, xantham gum, other insoluble fiber, other soluble fiber, other gum, and other vegetable oil.

In other specific examples, an absorption-reducing substance can comprise one or more ingredients that are selected from the group consisting of: acai oil, agar, almond oil, amaranth oil, apple seed oil, apricot oil, argan oil, avocado oil, babassu oil, beech nut oil, beta-glucan, bitter gourd oil, black pepper oil, black seed oil, blackcurrant seed oil, borage seed oil, bottle gourd oil, buffalo gourd oil, camellia oil, canola oil, carob oil, cashew oil, castor oil, cellulose, chitin, chitosan, cinnamon oil, citrus oil, clove oil, cocklebur oil, coconut oil, cod liver oil, cohune oil, colza oil, coriander seed oil, corn oil, cottonseed oil, date seed oil, dika oil, egg yolk oil, eucalyptus oil, false flax oil, fennel oil, fish oil, flaxseed oil, garlic oil, gelatin, ginger oil, grape seed oil, grapefruit seed oil, guar gum, gum acacia, hazelnut oil, hemp oil, kapok seed oil, kenaf seed oil, lactulose, lallemantia oil, lemon oil, lignin, lime oil, linseed oil, macadamia oil, mafura oil, marula oil, menthol oil, mineral oil, and mint oil.

In other specific examples, an absorption-reducing substance can comprise one or more ingredients that are selected from the group consisting of: mongongo nut oil, mustard oil, nutmeg oil, okra seed oil, olive oil, olive oil, orange oil, palm oil, papaya seed oil, peanut oil, pecan oil, pectins, pepper oil, peppermint oil, pequi oil, perilla seed oil, persimmon seed oil, pili nut oil, pine nut oil, pistachio oil, polycarbophil, polyethylene glycol, pomegranate seed oil, poppyseed oil, prune kernel oil, psyllium, pumpkin seed oil, quinoa oil, radish oil, ramtil oil, rapeseed oil, royle oil, safflower oil, salicornia oil, sapote oil, seje oil, sesame oil, soybean oil, spearmint oil, sunflower oil, taramira oil, thistle oil, tigernut oil, tomato seed oil, vegetable oil, walnut oil, watermelon seed oil, wheat germ oil, xantham gum, other fish oil, other gum, other insoluble fiber, other soluble fiber, and other vegetable oil.

I will now discuss the implanted reservoir in greater detail. In an example, a quantity of an absorption-reducing substance can be stored in an implanted reservoir before it is released into a person's gastrointestinal tract. In an example, this reservoir can be configured to be implanted within a person's body as part of an integrated device, system, and method for selectively reducing absorption of nutrients from unhealthy food.

In an example, there can be an opening, lumen, or shunt between the interior of an implanted reservoir and the interior of the person's gastrointestinal tract. In an example, an absorption-reducing substance can be released into the gastrointestinal tract through this opening, lumen, or shunt. In an example, this opening, lumen, or shunt enables controllable fluid communication between the interior of the implanted reservoir and the interior of the person's gastrointestinal tract.

In an example, there is a controllable flow of the substance from the interior of the reservoir to the interior of the gastrointestinal tract. In an example, there can be an opening, lumen, or shunt through which an absorption-reducing substance can flow, or be otherwise released, from an implanted reservoir into the interior of a portion of the gastrointestinal tract. In an example, an implanted reservoir, or an opening or lumen connecting it to the interior of the gastrointestinal tract, can have a one-way valve or filter that blocks movement of material from the gastrointestinal tract into the reservoir. This can help to prevent backflow of material from the gastrointestinal tract into the interior of the reservoir. This can prevent contamination of the absorption-reducing substance within the reservoir.

In an example, an implanted reservoir can be configured to be implanted within, or attached to, a body member selected from the group consisting of: stomach, duodenum, jejunum, ileum, caecum, colon, and esophagus. In an example, an implanted reservoir can be attached to the exterior surface of the stomach and have a tube from its interior to the interior of the stomach through which an absorption-reducing substance can be pumped into the stomach. In an example, an implanted reservoir can be configured to be implanted within the abdominal cavity and have a tube or other lumen that connects it to the interior of the gastrointestinal tract. In an example, an implanted reservoir can be configured to be implanted in a subcutaneous site or intraperitoneal site. In an example, an implanted reservoir can be configured to be implanted within, or attached to, adipose tissue or muscular tissue.

In various examples, a reservoir can be implanted within a person's body by one or more means selected from the group consisting of: suture or staple; adhesive or glue; clamp, clip, pin, or snap; elastic member; tissue pouch; fibrotic or scar tissue; screw; and tissue anchor. In an example, a reservoir can be rigid. In an example, a reservoir can be flexible. In various examples, an implanted reservoir, including a possible opening or lumen from the interior of the reservoir to the interior of the person's gastrointestinal tract, can be made from one or more materials selected from the group consisting of: cellulosic polymer, cobalt-chromium alloy, fluoropolymer, glass, latex, liquid-crystal polymer, nitinol, nylon, perflouroethylene, platinum, polycarbonate, polyester, polyether-ether-ketone, polyethylene, polyolefin, polypropylene, polystyrene, polytetrafluoroethylene, polyurethane, pyrolytic carbon material, silicone, stainless steel, tantalum, thermoplastic elastomer, titanium, and urethane.

In an example, an implanted reservoir can have multiple compartments. In an example, these multiple compartments can contain different types of absorption-reducing substances that are released in response to consumption of different types or quantities of food. In an example, these multiple compartments can contain different types of absorption-reducing substances that are released at different times or in different sequences. In an example, an implanted reservoir can have multiple compartments that contain different quantities of the same absorption-reducing substance that are released in response to consumption of different quantities or types of food. In an example, an implanted reservoir can have multiple compartments that contain separate amounts of one or more absorption-reducing substances that are released in discrete doses in response to separate eating events or episodes. In an example, an implanted reservoir can contain different types of absorption-reducing substances in different compartments which can be released and combined in different combinations to create specific and/or unique synergistic effects.

In an example, a reservoir can have an expanding balloon or bladder member to contain a variable quantity of an absorption-reducing substance. In an example, a reservoir can have a level indicator that that detects and communicates how much absorption-reducing substance is contained in the reservoir. In an example, the substance level can be communicated to an external source in a wireless manner. In an example, an implanted reservoir can be refilled or replaced. In an example, an implanted reservoir can be refilled with an absorption-reducing substance by one or more means selected from the group consisting of: an intra-gastric docking mechanism, such as a docking mechanism between a tube inserted orally and the reservoir; a needle or syringe that is temporarily inserted through the skin into the interior of the reservoir; a transdermal access port or tube; and a cartridge containing the substance that fits into the reservoir.

I will now discuss the release-control mechanism in greater detail. In an example, this invention includes a release-control mechanism that controls the manner in which an absorption-reducing substance is released from an implanted reservoir into a person's gastrointestinal tract in response to consumption of unhealthy food. In an example, a release-control mechanism can release an absorption-reducing substance into a person's stomach or intestine when a person consumes and/or digests an unhealthy type of food and/or nutrients. A release-control mechanism can be a key part of an overall system that helps a person to get proper nutrition while they manage their weight.

In an example, a release-control mechanism can activate the flowing, pumping, and/or spraying of an absorption-reducing substance from an implanted reservoir into a person's gastrointestinal tract to selectively reduce absorption of food nutrients. In an example, a release-control mechanism can selectively, temporarily, and automatically release an absorption-reducing substance into a person's gastrointestinal tract in response to consumption or digestion of selected types of food and/or nutrients as detected by a food-identifying sensor.

In an example, a release-control mechanism can selectively and automatically start or increase the flow of an absorption-reducing substance into a person's gastrointestinal tract when a food-identifying sensor identifies that a person is consuming or digesting unhealthy food. In an example, this release-control mechanism can also selectively and automatically stop or decrease the flow of the absorption-reducing substance into the person's gastrointestinal tract when the food-identifying sensor identifies that the person is consuming or digesting healthy food. In this manner, a release-control mechanism can selectively reduce absorption of nutrients from unhealthy food, but not reduce absorption of nutrients from healthy food. This can prevent the adverse potential for malnutrition that sometimes occurs with food-blind malabsorption devices and procedures in the prior art.

In an example, a release control mechanism can release a substance that creates a temporary coating on the interior walls of a portion of a person's gastrointestinal tract when the person eats unhealthy types and/or quantities of food. This can selectively reduce absorption of nutrients from unhealthy types and/or quantities of food. In an example, a release control mechanism can release a substance that creates a coating around a bolus of unhealthy food that is passing through a person's gastrointestinal tract. This can selectively reduce absorption of nutrients from unhealthy types and/or quantities of food.

In an example, a release-control mechanism can actuate a valve, pump, or variable-opening filter to release a flow or spray of an absorption-reducing substance into a person's gastrointestinal tract. In various examples, a release-control mechanism can include one or more valves selected from the group consisting of: biological valve, chemical valve, electromechanical valve, helical valve, piezoelectric valve, MEMS valve, hydraulic valve and micro-valve. In an example, a release-control mechanism can include one or more Micro Electrical Mechanical Systems (MEMS). In various examples, a release-control mechanism can include one or more components selected from the group consisting of: electronic mechanism, MEMS mechanism, microfluidic mechanism, biochemical mechanism, and biological mechanism.

In an example, a release-control mechanism can include a pump that pumps or sprays an absorption-reducing substance directly into a person's gastrointestinal tract. In various examples, a release-control mechanism can include one or more pumps selected from the group consisting of: 360-degree peristaltic pump, axial pump, biochemical pump, biological pump, centrifugal pump, convective pump, diffusion pump, dispensing pump, effervescent pump, elastomeric pump, electrodiffusion pump, electrolytic pump, electromechanical pump, electroosmotic pump, fixed-occlusion peristaltic pump, gravity feed pump, helical pump, hose-type peristaltic pump, hydrolytic pump, infusion pump, mechanical screw-type pump, MEMS pump, micro pump, multiple-roller peristaltic pump, osmotic pump, peristaltic pump, piezoelectric pump, pulsatile pump, rotary pump, spring-loaded roller pump, tube-type peristaltic pump, and vapor pressure pump.

In various examples, a release-control mechanism can be powered by an external power source, by internal power source, or by a combination of external and internal power sources. In an example, a release-control mechanism can transduce kinetic, thermal, or biochemical energy from within the person's body. In an example, a release-control mechanism may be powered by transducing the kinetic energy of stomach movement. In an example, the flow of an absorption-reducing substance from an implanted reservoir to a person's gastrointestinal tract can be caused by a pump that is controlled by a release-control mechanism. In an example, the flow of an absorption-reducing substance from an implanted reservoir to a person's gastrointestinal tract can be caused by the natural movement of a person's body and controlled by a release-control mechanism.

In various examples, a release-control mechanism can be powered from one or more energy sources selected from the group consisting of: a battery, an energy-storing chip, energy harvested or transduced from a bioelectrical cell, energy harvested or transduced from an electromagnetic field, energy harvested or transduced from an implanted biological source, energy harvested or transduced from blood flow or other internal fluid flow, energy harvested or transduced from body kinetic energy, energy harvested or transduced from glucose metabolism, energy harvested or transduced from muscle activity, energy harvested or transduced from organ motion, and energy harvested or transduced from thermal energy.

In various examples, a release-control mechanism can be can be made from one or more materials selected from the group consisting of: cobalt-chromium alloy, fluoropolymer, latex, liquid-crystal polymer, nylon, perflouroethylene, platinum, polycarbonate, polyester, polyethylene, polyolefin, polypropylene, polystyrene, polytetrafluoroethylene, polyurethane, polyvinyl chloride, pyrolytic carbon material, silicon, silicone, silicone rubber, stainless steel, tantalum, titanium, and urethane.

In an example, a release-control mechanism can start releasing an absorption-reducing substance into the gastrointestinal tract when a food-identifying sensor detects that the person has begun consuming unhealthy food and can stop releasing the absorption-reducing substance when the sensor detects that the person has begun consuming healthy food. In an example, the amount of substance that is released can be selectively and automatically increased when the sensor detects that the person is consuming or digesting unhealthy food and the amount of substance that is released can be selectively and automatically decreased when the sensor detects that the person is consuming or digesting healthy food.

In an example, unhealthy types of food can be identified by their having a high concentration of nutrients selected from the group consisting of: sugars, simple sugars, simple carbohydrates, fats, saturated fats, fat cholesterol, and sodium. In an example, unhealthy types and/or quantities of food can be identified by their having a high cumulative amount of one or more nutrients in the group consisting of: sugars, simple sugars, simple carbohydrates, fats, saturated fats, fat cholesterol, and sodium.

In an example, a release-control mechanism can include electronic components. In an example, a release-control mechanism can have one or more microchips or CPUs. In an example, a release-control mechanism can include a memory that tracks the cumulative amounts of nutrients that a person consumes during an episode of eating or during a selected period of time. For example, a release-control mechanism may count how many units of sugar, fat, or sodium are consumed by a person during the course of a day.

In an example, a release-control mechanism can allow up to a certain amount of one or more selected types of food or nutrients to be consumed by a person before it triggers the release of an absorption-reducing substance into the person's gastrointestinal tract. In an example, a release-control mechanism can be programmed to allow moderate consumption of some types of foods, but not excess consumption. In an example, a release-control mechanism can be programmed to allow unmodified absorption of selected foods for a limited time period or up to a certain amount. In an example, a release-control mechanism can be programmed to allow moderate consumption of some foods without malabsorption, but can cause malabsorption if there is excessive consumption of those foods.

In an example, a release-control mechanism can include electronics that can be wirelessly programmed in order to change the types and/or quantities of selected foods or nutrients for which nutrient absorption is automatically reduced. In an example, there can be a list in the device's memory of selected foods or nutrients which will trigger the release of an absorption-reducing substance into the person's gastrointestinal tract. In an example, a release-control mechanism can be programmed to change this list. In an example, the types of foods can be changed by programming. In an example, the quantities of foods can be changed by programming. In an example, the types and/or quantities of foods on the list can be automatically changed by a device with automatic learning capability.

In various examples, the operation of a release-control mechanism can be manually or automatically adjusted based on one or more factors selected from the group consisting of: the person's short-term eating patterns; the person's long-term eating patterns; the person's short-term exercise patterns and caloric expenditure; the person's long-term exercise patterns and caloric expenditure; the person's success in meeting weight reduction goals; holidays or other special events; professional guidance and diet planning; social support networks; financial constraints and incentives; and degree of sensor precision and measurement uncertainty.

In various examples, a release-control mechanism can be designed or programmed to selectively modify the absorption of selected types of food based on: the time of the day (to reduce snacking between meals or binge eating at night); the person's cumulative caloric expenditure (to reward exercise and achieve energy balance); special social events and holidays (to allow temporary relaxation of dietary restrictions); physical location measured by GPS (to discourage eating in locations that are associated with unhealthy consumption); and/or social networking connections and support groups (to provide peer support for willpower enhancement).

In various examples, one or more aspects of the operation of a release-control mechanism can be manually or automatically adjusted, wherein these aspects are selected from the group consisting of: the type of food consumed which triggers decreased food absorption; the quantity of food consumed during a given period of time which triggers decreased food absorption; the time of day, day of the week, or other timing parameter concerning food consumption which triggers decreased food absorption; the effect of past food consumption behavior on decreased food absorption; the effect of caloric expenditure behavior on decreased food absorption; and a personalized dietary plan treatment created for the person by a health care professional.

In an example, a release-control mechanism can include a wireless data transmitter and receiver. In an example, a release-control mechanism can communicate wirelessly with a food-identifying sensor that is implanted in a different part of a person's body. In an example, a release-control mechanism can communicate wirelessly with a source that is external to the person's body. In an example, a release-control mechanism can be programmed, or otherwise adjusted, by an external remote control unit.

In an example, a release-control mechanism can wirelessly communicate with a food-identifying sensor that is carried by, or worn by, a person. In various examples, a release-control mechanism can be in wireless communication with a food-identifying sensor that a person wears on their wrist, hand, finger, arm, torso, neck, head, and/or ear. In various examples, a release-control mechanism can be in wireless communication with a food-identifying sensor that is incorporated into a piece of electronically-functional jewelry such as a necklace, pendant, finger ring, bracelet, nose ring, or earring. In various examples, a release-control mechanism can be in wireless communication with a food-identifying sensor that is incorporated into a person's wrist watch, eyeglasses, hearing aid, or bluetooth device.

In an example, a release-control mechanism can communicate wirelessly with one or more external computers that are linked by a network, such as the internet. In an example, a release-control mechanism can be wirelessly programmed, or otherwise adjusted, by the person in whom the device is implanted. In an example, a release-control mechanism can be wirelessly programmed, or otherwise adjusted, by a care giver or other health care professional. In various examples, a release-control mechanism can have wireless communication with one or more of the following members: a food-identifying sensor that is implanted within, or attached to, in a different area of the person's body; a remote computer, network, or remote control unit that is external to the person's body; and an external mobile, cellular, or tabular electronic communication device. In an example, a release-control mechanism can be a key part of an overall system to ensure that a person gets proper nutrition while this person is losing weight.

As shown in FIGS. 31 through 36, this invention can be embodied in a device for selectively and automatically reducing the absorption of selected types of food in a person's gastrointestinal tract. This device can comprise: (a) a food-identifying sensor that selectively detects when the person is consuming and/or digesting selected types of food; (b) an absorption-reducing substance that is released into the interior of the person's gastrointestinal tract to temporarily reduce absorption of nutrients from food by the gastrointestinal tract; (c) an implanted reservoir that contains a quantity of the absorption-reducing substance, wherein this reservoir is configured to be implanted within the person's body and wherein there is an opening or lumen through which the absorption-reducing substance is released from the reservoir into the interior of a portion of the person's gastrointestinal tract; and (d) a release-control mechanism that controls the release of the absorption-reducing substance from the reservoir into the person's gastrointestinal tract, wherein this release-control mechanism can selectively and automatically increase the release of the absorption-reducing substance when the food-identifying sensor detects that the person is consuming and/or digesting selected types of food.

In an example, the food-identifying sensor of this embodiment can selectively discriminate between consumption and/or digestion of unhealthy food and consumption and/or digestion of healthy food. In an example, unhealthy food can be identified as having a high concentration of one or more nutrients selected from the group consisting of: sugars, simple sugars, simple carbohydrates, fats, saturated fats, cholesterol, and sodium. In an example, unhealthy food can be identified as having a large amount of one or more nutrients selected from the group consisting of: sugars, simple sugars, simple carbohydrates, fats, saturated fats, cholesterol, and sodium. In an example, unhealthy food can be identified as food with an amount of one or more nutrients selected from the group consisting of sugars, simple sugars, simple carbohydrates, fats, saturated fats, cholesterol, and sodium that is more than the recommended amount of such nutrient for the person during a given period of time.

In an example, the food-identifying sensor of this embodiment can be selected from the group consisting of: chemical sensor, biochemical sensor, accelerometer, amino acid sensor, biological sensor, camera, chemoreceptor, cholesterol sensor, chromatography sensor, EGG sensor, electrolyte sensor, electromagnetic sensor, electronic nose, EMG sensor, enzyme-based sensor, fat sensor, flow sensor, particle size sensor, peristalsis sensor, genetic sensor, glucose sensor, imaging sensor, impedance sensor, infrared sensor, interferometer, medichip, membrane-based sensor, Micro Electrical Mechanical System (MEMS) sensor, microfluidic sensor, micronutrient sensor, molecular sensor, motion sensor, muscle activity sensor, nanoparticle sensor, neural impulse sensor, nutrient sensor, optical sensor, osmolality sensor, pH level sensor, pressure sensor, protein-based sensor, reagent-based sensor, smell sensor, sound sensor, strain gauge, taste sensor, and temperature sensor.

In an example, the absorption-reducing substance of this embodiment can coat food, food particles, nutrients, and/or chyme in the gastrointestinal tract. In an example, this absorption-reducing substance can temporarily coat a portion of the interior walls of the intestine. In an example, this absorption-reducing substance can bind to food, food particles, nutrients, and/or chyme in the gastrointestinal tract. In an example, this absorption-reducing substance can temporarily bind to a portion of the interior walls of the intestine. In an example, this absorption-reducing substance can temporarily increase the viscosity, increase the thickness, and/or decrease the nutrient permeability of the mucus that covers a portion of the interior walls of the person's intestine. In an example, the absorption-reducing substance of this embodiment can comprise one or more ingredients that are Generally Recognized As Safe (GRAS) under Sections 201(s) and 409 of the Federal Food, Drug, and Cosmetic Act.

In an example, the release-control mechanism of this embodiment can: start or increase the release of the absorption-reducing substance into the person's gastrointestinal tract in response to detection of consumption or digestion of unhealthy types of food by the food-identifying sensor; and/or stop or decrease the release of the absorption-reducing substance into the person's gastrointestinal tract in response to detection of consumption or digestion of healthy types of food by the food-identifying sensor. In an example, unhealthy food can be identified as having a relatively large amount or concentration of one or more nutrients selected from the group consisting of: sugars, simple sugars, simple carbohydrates, fats, saturated fats, cholesterol, and sodium.

In an example, the release-control mechanism of this embodiment can communicate wirelessly with a source external to the person's body. In an example, this release-control mechanism can be programmed, or otherwise adjusted, to change the types of selected foods or nutrients to which it responds by releasing an absorption-reducing substance into the person's gastrointestinal tract. In an example, this release-control mechanism can be programmed to adjust one or more of the following aspects of its response to the food-identifying sensor: the type of food which triggers decreased food absorption; the quantity of food which triggers decreased food absorption; the time of day, day of the week, or other timing parameter concerning food consumption which triggers decreased food absorption; the effect of the person's past food consumption on decreased food absorption; the effect of the person's caloric expenditure on decreased food absorption; and the effect of a personalized diet plan created for the person by a health care professional.

In an example, this invention can be embodied in a device for selectively and automatically reducing the absorption of unhealthy food by a person's gastrointestinal tract. This device can comprise: (a) a food-identifying sensor that selectively detects when the person is consuming and/or digesting unhealthy food, wherein unhealthy food is identified as food that has a relatively large amount or concentration of one or more nutrients selected from the group consisting of: sugars, simple sugars, simple carbohydrates, fats, saturated fats, cholesterol, and sodium; (b) an absorption-reducing substance that is released into the person's gastrointestinal tract to reduce absorption of nutrients from food in the gastrointestinal tract by one or more means selected from the group consisting of: coating food, food particles, nutrients, and/or chyme in the gastrointestinal tract; temporarily coating a portion of the interior walls of the gastrointestinal tract; binding to food, food particles, nutrients, and/or chyme in the gastrointestinal tract; temporarily binding to a portion of the interior walls of the gastrointestinal tract; temporarily blocking nutrient-absorbing organelles on a portion of the interior walls of the person's duodenum; temporarily increasing the viscosity of the mucus on a portion of the interior walls of the person's intestine; and temporarily decreasing the nutrient permeability of the mucus on a portion of the interior walls of the person's intestine; (c) an implanted reservoir that contains a quantity of the absorption-reducing substance, wherein this reservoir is configured to be implanted within the person's body, and wherein there is an opening or lumen through which the absorption-reducing substance is released from the reservoir into a portion of the person's gastrointestinal tract; and (d) a release-control mechanism that controls the release of the absorption-reducing substance from the reservoir into the person's gastrointestinal tract, wherein the amount of absorption-reducing substance released can be selectively and automatically increased when the food-identifying sensor detects that the person is consuming or digesting unhealthy food and wherein the amount of substance released can be selectively and automatically decreased when the sensor detects that the person is consuming or digesting healthy food.

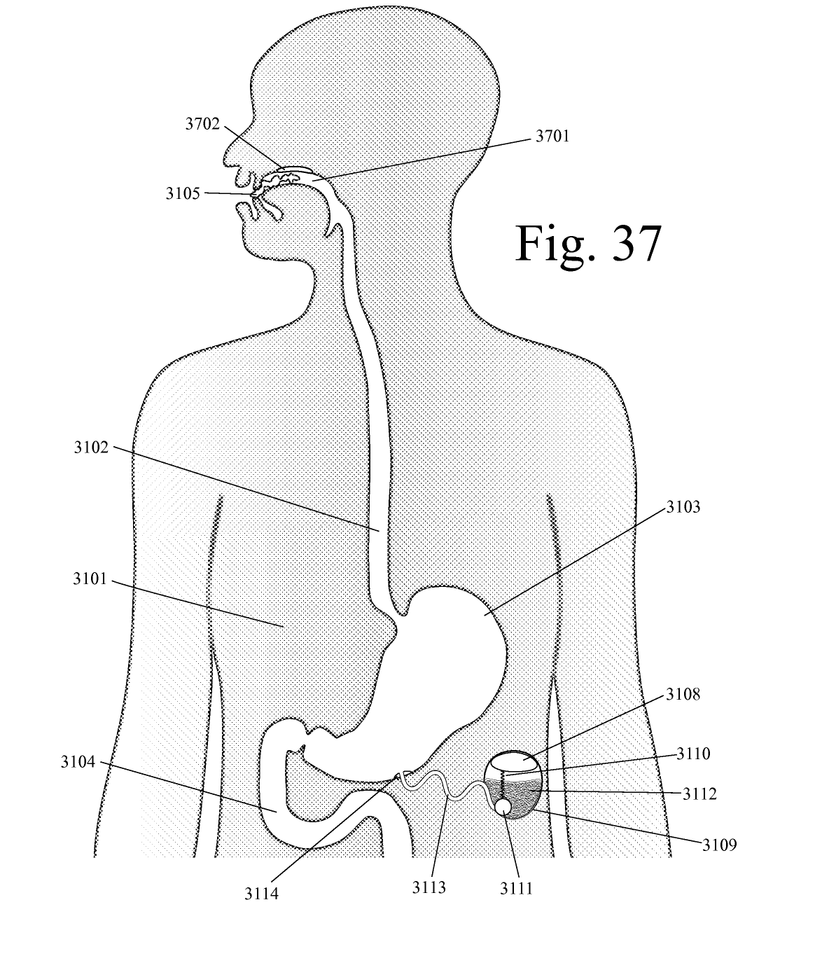

FIGS. 37 through 40 show additional examples of how this invention can be embodied in a device and method for selectively and automatically reducing absorption of nutrients from unhealthy food in a person's gastrointestinal tract. In these examples, the food-identifying sensor is a mouth-based or nose-based sensor that is in fluid communication with the person's mouth or nose.

There are advantages to using a mouth-based or nose-based food-identifying sensor in such a device or method for selective malabsorption of unhealthy food. A mouth-based or nose-based food-identifying sensor can detect consumption of unhealthy food earlier than an intragastric sensor. This provides “earlier detection” that a bolus of unhealthy food will be entering the stomach and intestine, before the food even enters the stomach. This “earlier detection” provides more lead time for the device and method to more-thoroughly modify the gastrointestinal tract in order to more-completely reduce absorption of nutrients from the bolus of unhealthy food.

FIGS. 37 through 40 show examples of how this invention can be embodied in a device for selectively and automatically reducing absorption of unhealthy food in a person's gastrointestinal tract using a mouth-based food-identifying sensor. In an example, this device can comprise: (a) a food-identifying sensor that selectively detects when a person is consuming or digesting selected types of food, wherein this food-identifying sensor is configured to be implanted or attached within the person's oral cavity, the person's nasal cavity, or tissue surrounding one of these cavities; and (b) an absorption-reducing member that is implanted within the person's body, wherein this absorption-reducing member can selectively and automatically reduce the absorption of food within the person's gastrointestinal tract when the sensor detects that the person is consuming or digesting selected types of food.

FIGS. 37 through 40 also show examples of how this invention can be embodied in a method for selectively and automatically reducing absorption of unhealthy food in a person's gastrointestinal tract using a mouth-based food-identifying sensor. In an example, such a method can comprise: (a) selectively and automatically detecting when a person is consuming or digesting selected types of food by means of a sensor that is configured to be implanted or attached within the person's oral cavity, the person's nasal cavity, or tissue surrounding one of these cavities; and (b) selectively and automatically reducing the absorption of food within the person's gastrointestinal tract by means of an implanted absorption-reducing member, wherein this member selectively and automatically reduces food absorption when the sensor detects that the person is consuming or digesting selected types of food.

FIG. 37 shows a longitudinal cross-sectional view of a person's torso 3101 and head, wherein the person's head is turned sideways to provide a lateral cross-sectional view of the person's head. FIG. 37 includes a longitudinal cross-sectional view of the entire upper portion of the person's gastrointestinal tract, including the person's oral cavity 3701, esophagus 3102, stomach 3103, and duodenum 3104. This figure also shows a bolus of food 3105 in oral cavity 3701, wherein this person is starting to consume and digest this bolus of food 3105. In FIG. 37, bolus of food 3105 is healthy food.

FIG. 37 also shows an example of an implanted device that enables selective malabsorption of unhealthy food using a mouth-based sensor. Selective malabsorption of unhealthy food, while also allowing normal absorption of healthy food, can help a person to lose weight without suffering deficiencies of essential nutrients that can occur with food-blind bariatric procedures and malabsorption devices in the prior art.

In the example shown in FIG. 37, food-identifying sensor 3702 is attached to, or implanted within, the palatal vault of the person's oral cavity 3701. In other examples, a food-identifying sensor may be implanted in other locations that are in fluid and/or gaseous communication with the person's oral cavity and/or nasal cavity. Food-identifying sensor 3702 can selectively and automatically detect when the person is beginning to consume and digest unhealthy food. In an example, food-identifying sensor 3702 can identify unhealthy food by performing chemical analysis of saliva in the person's mouth. In an example, unhealthy food can be identified as having a high concentration of one or more of the following nutrients: sugars, simple sugars, simple carbohydrates, fats, saturated fats, cholesterol, and sodium.

In various examples, food-identifying sensor 3702 can be selected from the group of sensors consisting of: chemical sensor, biochemical sensor, amino acid sensor, biological sensor, chemoreceptor, cholesterol sensor, chromatography sensor, EGG sensor, enzyme-based sensor, fat sensor, particle size sensor, peristalsis sensor, glucose sensor, impedance sensor, membrane-based sensor, Micro Electrical Mechanical System (MEMS) sensor, microfluidic sensor, micronutrient sensor, molecular sensor, motion sensor, nutrient sensor, osmolality sensor, pH level sensor, protein-based sensor, reagent-based sensor, and temperature sensor.

In the embodiment of the invention that is shown in FIG. 37, food-identifying sensor 3702 can communicate by wireless transmission with release-control mechanism 3108. Release-control mechanism 3108 is contained in implanted reservoir 3109 that is implanted within the person's abdominal cavity. Release-control mechanism 3108 is connected by wire 3110 to pump 3111 which is also contained in reservoir 3109. Pump 3111 is in fluid communication with absorption-reducing substance 3112 that is contained in reservoir 3109 until this substance is released into the stomach 3103 through lumen 3113 and one-way valve 3114. In an example, absorption-reducing substance 3112 can be selectively and automatically released into the interior of the person's stomach 3103 to reduce food absorption when food-identifying sensor 3702 detects consumption of unhealthy food in the person's oral cavity 3701.

FIG. 37 shows how this embodiment of the invention does not actively respond to the consumption and digestion of bolus of healthy food 3105. In this figure, the device does not interfere with the normal absorption of healthy food 3105. This is an advantage over malabsorption procedures and devices that blindly reduce absorption of all food, including healthy food. This avoids the deficiencies of essential nutrients that can be caused by food-blind malabsorption procedures and devices in the prior art.

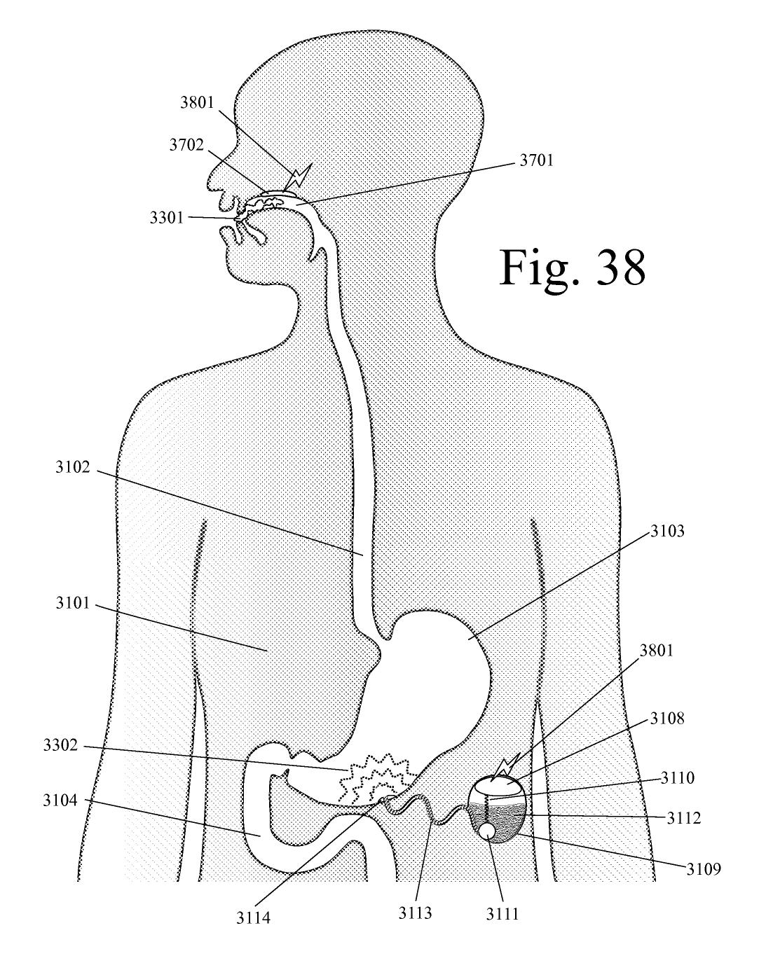

FIG. 38, in contrast, shows how this embodiment can selectively and automatically respond to a bolus of food 3301 that is unhealthy. In an example, bolus of food 3301 can have a high concentration of one or more of the following nutrients: sugars, simple sugars, simple carbohydrates, fats, saturated fats, cholesterol, and sodium. The following is the sequence of actions involved as the device selectively and automatically reduces absorption of nutrients from unhealthy food 3301.

First, in FIG. 38, the person has inserted a bolus of unhealthy food 3301 into their mouth and this bolus of food 3301 is starting to be digested by chewing action and saliva. Next, the bolus of unhealthy food 3301 is identified as unhealthy by food-identifying sensor 3702. In an example, this identification can be done by analyzing the chemical composition of saliva in the mouth as the food begins to be digested. Then, food-identifying sensor 3702 sends a wireless signal 3801 to release-control mechanism 3108. This wireless signal informs release-control mechanism 3801 that the person has consumed a bolus of unhealthy food 3301.

In FIG. 38, the wireless signal that is transmitted from food-identifying sensor 3702 to release-control mechanism 3108 is represented by two “lightning bolt” symbols labeled 3801. The “lightning bolt” symbol (labeled 3801) near the sensor represents the origination point of the wireless signal and the “lightning bolt” symbol (also labeled 3801) near the release-control mechanism represents the destination point of the wireless signal. The same label (801) is used for the wireless signal in both locations because it is the same signal, just interacting with the device at different locations.

In FIG. 38, the receipt of wireless signal 3801 by release-control mechanism 3108 triggers the activation of pump 3111. Pump 3111 then releases a quantity of absorption-reducing substance 3112 (through lumen 3113 and one-way valve 3114) into the interior of stomach 3103. The release of absorption-reducing substance 3112 into stomach 3103 is represented by concentric wavy dotted lines 3302 that radiate outwards from one-way valve 3114 into the person's stomach 3103.

As was shown in previous figures, an absorption-reducing substance 3112 can selectively and automatically reduce absorption of nutrients from unhealthy food by coating the walls of the duodenum 3104 when unhealthy food is detected. As was shown in previous figures, an absorption-reducing substance 3112 can selectively and automatically reduce absorption of nutrients from unhealthy food by coating the bolus of unhealthy food 3301 (or chyme containing food particles from this bolus of unhealthy food) as it passes through the stomach 3103. In an example, absorption-reducing substance 3112 can coat both the duodenal walls and the bolus of food.

In various examples, an absorption-reducing substance 3112 can reduce absorption of nutrients from a bolus of unhealthy food 3301 by one of more actions selected from the group consisting of: temporarily coating the interior walls of duodenum 3104; coating a bolus of unhealthy food 3301 (or chyme containing food particles from this bolus); changing the speed at which a bolus of unhealthy food 3301 travels through the gastrointestinal tract; temporarily binding to the interior walls of duodenum 3104; binding to a bolus of unhealthy food 3301; increasing the thickness of the mucus covering the interior walls of the duodenum; increasing the viscosity of the mucus covering the interior walls of the duodenum; and decreasing the nutrient permeability of the mucus covering the interior walls of the duodenum.

In an example, release-control mechanism 3108 can start releasing an absorption-reducing substance 3112 into the person's stomach 3103 in response to detection of consumption of unhealthy food 3301 by food-identifying sensor 3702. In an example, release-control mechanism 3108 can stop releasing absorption-reducing substance 3112 into the person's stomach 3103 in response to detection of consumption of healthy food 3105 by the food-identifying sensor 3702.

In an example, release-control mechanism 3108 can communicate wirelessly with a source external to the person's body. In an example, release-control mechanism 3108 can be programmed, or otherwise adjusted, to change the types of selected foods or nutrients to which it responds by releasing an absorption-reducing substance 3112 into the person's gastrointestinal tract. In various examples, release-control mechanism 3108 can be programmed to adjust one or more of the following aspects of its response to food-identifying sensor 3702: the types of food and/or nutrients which trigger decreased food absorption; the quantities of food and/or nutrients which trigger decreased food absorption; the time of day, day of the week, or other timing parameters concerning food consumption which trigger decreased food absorption; the effects of the person's past food consumption on decreased food absorption; the effects of the person's caloric expenditure on decreased food absorption; and the effects of a personalized diet plan created for the person by a health care professional.

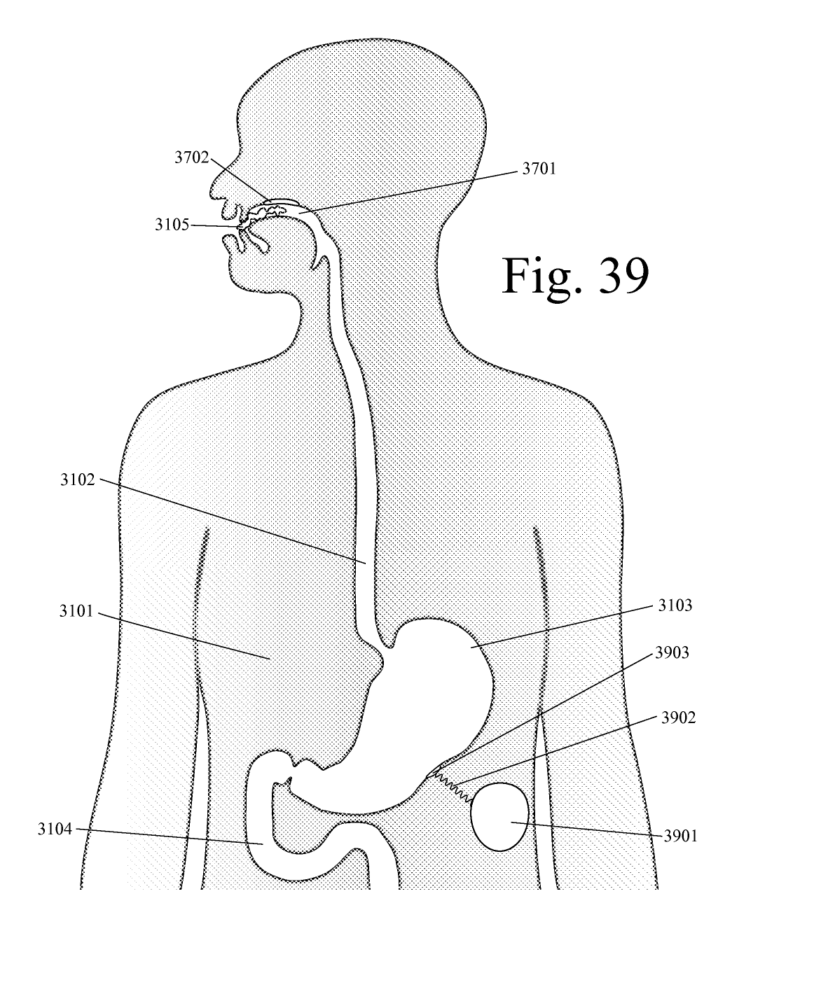

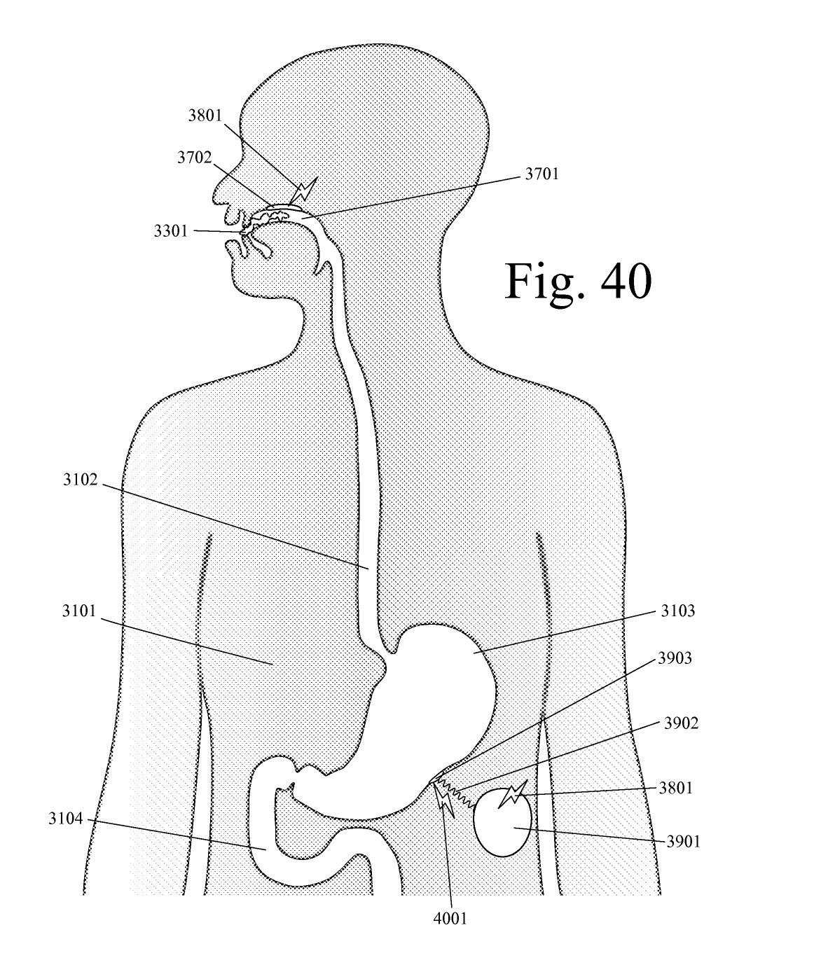

FIGS. 39 and 40 show another example of how this invention can be embodied in a device and method that uses a mouth-based food-identification sensor to selectively and automatically reduce absorption of unhealthy food. Similar to FIG. 37, FIG. 39 shows a longitudinal cross-sectional view of a person's torso 3101 and head. This view includes a longitudinal cross-sectional view of the entire upper portion of the person's gastrointestinal tract, including the person's oral cavity 3701, esophagus 3102, stomach 3103, and duodenum 3104. This figure also shows a bolus of healthy food 3105 in oral cavity 3701. The person is starting to consume and digest this bolus of healthy food 3105.

FIG. 39 also shows another example of an implanted device that enables selective malabsorption of unhealthy food using a mouth-based sensor. In this example, the absorption-reducing member comprises an implanted electrical component 3901. In this example, implanted electrical component 3901 is an implanted electrical impulse generator that delivers an electrical impulse to the walls of the person's stomach 3103 via wire 3902 and electrode 3903. In various examples, implanted electrical component 3901 can deliver electricity to other portions of the person's gastrointestinal tract or to nerves in communication with the person's gastrointestinal tract.

There are many examples of implanted electrical components in the prior art that deliver electricity to portions of the body. The exact type of implanted electrical component that is used is not central to this invention. However, selectively and automatically activating such a device in response to consumption of unhealthy food, as detected early in consumption by a mouth-based food-identifying sensor, is novel. Selective, automatic, and early activation of an implanted electrical component has significant advantages over devices and methods for electrical stimulation of the gastrointestinal tract in the prior art that are blind concerning whether the person is consuming unhealthy or healthy food.

As one advantage, when an electrical stimulation device is only activated when the person is eating unhealthy food, then the person's muscles and/or nerves will be less likely to habituate to the electrical stimulation and cause stimulation to lose its effectiveness. As a second advantage, when an electrical stimulation device is only activated when the person is eating unhealthy food, then the person is less likely to suffer from deficiencies of essential nutrients because there is no interference with the digestion and absorption of healthy food. As a third advantage, when an electrical stimulation device is only activated when the person is eating unhealthy food, the device uses less battery power than a food-blind device.

FIG. 39 shows how this embodiment of the invention does not actively respond to consumption and digestion of bolus of healthy food 3105. In this figure, the device does not interfere with the normal absorption of healthy food 3105. For the three reasons discussed above, this is an advantage over implanted electrical stimulators in the prior art that blindly reduce absorption of all food, including healthy food. Having early detection of unhealthy food consumption by a mouth-based sensor allows the device to prepare the stomach and intestine for malabsorption before the food even reaches the stomach. This is an advantage over intragastric sensors.

FIG. 40, in contrast, shows how this embodiment can selectively and automatically respond to a bolus of food 3301 that is unhealthy. In an example, bolus of unhealthy food 3301 can have a high concentration of one or more of the following nutrients: sugars, simple sugars, simple carbohydrates, fats, saturated fats, cholesterol, and sodium. The following is the sequence of actions involved as the device in FIG. 40 selectively and automatically reduces absorption of nutrients from unhealthy food 3301.

First, in FIG. 40, the person has inserted a bolus of unhealthy food 3301 into their mouth and this bolus of food 3301 is starting to be digested by chewing action and saliva. Next, the bolus of unhealthy food 3301 is identified as unhealthy by food-identifying sensor 3702. In an example, this identification can be done by analyzing the chemical composition of saliva in the mouth as the food begins to be digested. Then, food-identifying sensor 3702 sends a wireless signal 3801 to implanted electrical component 3901. In this example, the absorption-control member of this invention comprises implanted electrical component 3901. The wireless signal informs implanted electrical component 3901 that the person has consumed a bolus of unhealthy food 3301.

In FIG. 40, the receipt of wireless signal 3801 by implanted electrical component 3901 triggers an electrical impulse 4001 through wire 3902 and electrode 3903 to the wall of the person's stomach 3103. In this example, this electrical impulse changes the motility of gastric peristalsis to reduce absorption of the bolus of unhealthy food 3301 by the person's gastrointestinal tract. In an example, this electrical impulse can increase the speed at which bolus of unhealthy food 3301 moves through the person's stomach, duodenum, or other portions of the person's gastrointestinal tract. In an example, this electrical impulse can decrease secretion of enzymes by the person's stomach or adjacent secretory organ's along the person's gastrointestinal tract.

In various examples, application of electricity to one or more portions of the person's gastrointestinal tract, or to the nerves that innervate this tract, can selectively and automatically reduce absorption of nutrients from bolus of unhealthy food 3301, as identified by mouth-based food-identification sensor 3702. In an example, an implanted absorption-reducing member, such as implanted electrical component 3901, can start stimulating an organ along the gastrointestinal tract in response to detection of consumption of unhealthy food 3301 by food-identifying sensor 3702. In an example, an implanted absorption-reducing member, such as implanted electrical component 3901, can stop stimulating an organ along the gastrointestinal tract in response to detection of consumption of healthy food 3105 by food-identifying sensor 3702.

In an example, implanted electrical component 3901 can communicate wirelessly with a source external to the person's body. In an example, an absorption-reducing member, such as implanted electrical component 3901, can be programmed, or otherwise adjusted, to change the types of selected foods or nutrients to which it responds by releasing an absorption-reducing substance 3112 into the person's gastrointestinal tract.

In various examples, an absorption-reducing member, such as implanted electrical component 3901, can be programmed to adjust one or more of the following aspects of its response to food-identifying sensor 3702: the types of food and/or nutrients which trigger decreased food absorption; the quantities of food and/or nutrients which trigger decreased food absorption; the time of day, day of the week, or other timing parameters concerning food consumption which trigger decreased food absorption; the effects of the person's past food consumption on decreased food absorption; the effects of the person's caloric expenditure on decreased food absorption; and the effects of a personalized diet plan created for the person by a health care professional.

As shown in FIGS. 37 through 40, this invention can be embodied in a device for selectively and automatically reducing the absorption of selected types of food in a person's gastrointestinal tract comprising: (a) a mouth-based or nose-based food-identifying sensor that selectively detects when a person is consuming or digesting selected types of food, wherein this food-identifying sensor is configured to be implanted or attached within the person's oral cavity, the person's nasal cavity, or tissue surrounding one of these cavities; and (b) an absorption-reducing member that is implanted within the person's body, wherein this absorption-reducing member can selectively and automatically reduce the absorption of food within the person's gastrointestinal tract when the sensor detects that the person is consuming or digesting selected types of food.

Also, as shown in FIGS. 37 through 40, this invention can be embodied in a method for selectively and automatically reducing the absorption selected types of food in the gastrointestinal tract comprising: (a) selectively and automatically detecting when a person is consuming or digesting selected types of food by means of a sensor that is configured to be implanted or attached within the person's oral cavity, the person's nasal cavity, or tissue surrounding one of these cavities; and (b) selectively and automatically reducing the absorption of food within the person's gastrointestinal tract by means of an implanted absorption-reducing member, wherein this member selectively and automatically reduces food absorption when the sensor detects that the person is consuming or digesting selected types of food.

In the following sections of this disclosure, I discuss various examples of these two device sub-components (mouth-based or nose-based food-identifying sensor and absorption-reducing member) and these two method steps (detecting when a person is consuming unhealthy food and reducing the absorption of this unhealthy food) in greater detail.

First, I will discuss the mouth-based or nose-based food-identifying sensor in greater detail. In an example, a food-identifying sensor can be configured to be attached to, or implanted within, a person's oral cavity, nasal cavity, or tissue surrounding one of these cavities. In an example, an implanted food-identifying sensor can be in fluid or gaseous communication with a person's oral cavity or nasal cavity. In an example, a food-identifying sensor can be configured to be attached to, or implanted within, a person's mouth or nose. In an example, an implanted food-identifying sensor can be in fluid or gaseous communication with a person's mouth or nose. In an example, a food-identifying sensor in a person's mouth or nose can be in wireless communication with an absorption-reducing member that is implanted elsewhere in the person's body. In an example, having a food-identifying sensor in a person's mouth or nose can provide “earlier detection” for activation of an absorption-reducing member elsewhere in the person's body.

A food-identifying sensor in a person's mouth or nose can detect consumption and/or digestion of unhealthy food as it is starting to be digested within a person's mouth. There are advantages of having an implanted food-identifying sensor be configured so as to be in fluid or gaseous communication with a person's oral or nasal cavities. Such a food-identifying sensor can provide “earlier detection” that a particular bolus of unhealthy food will be entering the stomach, before food enters the stomach. As compared to an intragastric sensor, a mouth-based or nose-based sensor provides more time for modification of the stomach or intestine to reduce absorption of nutrients from the bolus of food before the food reaches the stomach.

In an example, “earlier detection” of unhealthy food consumption from a mouth-based or nose-based sensor to an absorption-reducing member that is implanted elsewhere in the person's body can enable the walls of the duodenum to be thoroughly coated with an absorption-reducing coating before the bolus of unhealthy food arrives there. In another example, such “earlier detection” from a mouth-based or nose-based sensor can enable a food-coating substance to be thoroughly dispersed throughout the interior of the stomach before the bolus of unhealthy food even enters the stomach. These actions can more efficiently reduce absorption of a bolus of unhealthy food as it moves through the person's gastrointestinal tract. A mouth-based or nose-based food-identifying sensor can provide “earlier detection” to a release-control mechanism that releases an absorption-reducing substance into a person's stomach or intestine before a selected bolus of unhealthy food enters the stomach. By the time the bolus of food enters the stomach, the absorption-reducing substance can already be well dispersed throughout the stomach and/or intestine.

In an example, “earlier detection” from a mouth-based or nose-based food-identifying sensor can be sent to an absorption-reducing member that reduces absorption by applying electricity to a gastrointestinal organ or to nerves that are in communication with such an organ. For example, when a mouth-based or nose-based sensor detects that a person is starting to consume unhealthy food, such a sensor can send signals to an electrical stimulation device that is implanted elsewhere in the person's body. This electrical stimulation device can selectively apply electricity to the person's stomach, to nerves innervating the stomach, or to other organs or tissues in communication with the person's gastrointestinal tract in order to selectively reduce absorption of nutrients from a particular bolus of unhealthy food.

In an example, electrical stimulation can selectively modify the peristalsis of a gastrointestinal organ in order to selectively decrease absorption of nutrients from a bolus of unhealthy food. In another example, electrical stimulation can selectively decrease secretion of enzymes into the gastrointestinal tract to decrease absorption of nutrients from a selected bolus of unhealthy food. The selective malabsorption that is enabled by a mouth-based or nose-based food-identifying sensor can be superior to the indiscriminant malabsorption provided by devices, methods, and procedures in the prior art that are blind to whether a particular bolus of food passing through the gastrointestinal tract is unhealthy or healthy.

In an example, a mouth-based or nose-based food-identifying sensor can provide “earlier detection” to an absorption-reducing member that reduces food absorption by restricting the size of a portion of the person's gastrointestinal tract. For example, when a mouth-based or nose-based sensor detects that the person is starting to consume unhealthy food, a sensor can send signals to a gastric constriction device that: constrains the external size of the entire stomach; constrains the size of the entrance to the stomach; or changes the length of the gastrointestinal tract that is traveled by a selected bolus of food.

In an example, there can be an adjustable valve in a person's gastrointestinal tract that can direct different boluses of food through a shorter route with less absorption of nutrients versus a longer route with more absorption of nutrients. In an example, the shorter route can be a gastric bypass which can be selectively and remotely activated by the results of a food-identifying sensor. In an example, when a food-identifying sensor detects that the person is eating a bolus of unhealthy food, the sensor sends a wireless signal to an absorption-reducing member (a valve control mechanism in this example) that routes this bolus of unhealthy food through the shorter (bypass) route. When the person stops eating unhealthy food and starts eating healthy food, the sensor changes the valve so that healthy food goes through the longer route.

In an example, a food-identifying sensor can be implanted within, or attached to, a person's oral cavity. In an example, a food-identifying sensor can be configured to be attached to, or implanted within, a person's hard palate, palatal vault and/or upper mouth roof, teeth, tongue, or soft palate. In various examples, an food-identifying sensor can be attached to, or implanted by one or more means selected from the group consisting of: suture, staple, adhesive, glue, clamp, clip, pin, snap, elastic member, tissue pouch, fibrotic tissue, screw, and tissue anchor.

In an example, a sensor can be configured to be attached to, or implanted within, or attached underneath a person's tongue. In an example, a food-identifying sensor can be inserted into a person's tongue. In an example, a sensor can be attached or implanted sublingually. In an example, a sensor can be configured to be attached to, or inserted into, the soft palate tissues at the rear of a person's oral cavity. In an example, a sensor can be configured to be attached to, or implanted within, a person's teeth. In various examples, a sensor can be attached to the lingual, palatal, buccal, and/or labial surfaces of a person's teeth. In an example, a food-identifying sensor can be incorporated into a dental and/or orthodontic appliance. In an example, a food-identifying sensor can be incorporated into a dental bridge, cap, or crown.

In an example, a food-identifying sensor within a person's mouth can analyze saliva to selectively detect consumption of unhealthy food at the point of initial consumption. In various examples, a food-identifying sensor that is in fluid communication with a person's mouth can analyze saliva within the mouth in order to automatically and selectively detect when a person is digesting food that is high in sugar or fat. In an example, a food-identifying sensor in a person's mouth can be a chemical sensor. In various examples, a chemical sensor can detect the amount or concentration of sugars, simple carbohydrates, fats, saturated fats, cholesterol fat, and/or sodium in food.

In various examples, a food-identifying sensor that is in fluid or gaseous communication with a person's mouth or nose can identify food as being unhealthy using one or more methods selected from the group consisting of: chemical analysis of food as it begins to be digested within a person's mouth; olfactory analysis of food as it beings to be digested within a person's mouth; image analysis of images of food as it approaches the person's mouth; sonic analysis of chewing or swallowing as food is consumed; and analysis of signals from nerves that innervate the person's taste buds and/or olfactory receptors.

In various examples, a food-identifying sensor within a person's mouth or nose can be selected from the group of sensors consisting of: chemical sensor, biochemical sensor, accelerometer, amino acid sensor, biological sensor, camera, chemoreceptor, cholesterol sensor, chromatography sensor, electrogastrogram sensor, electrolyte sensor, electromagnetic sensor, EMG sensor, enzymatic sensor, fat sensor, flow sensor, particle size sensor, peristalsis sensor, genetic sensor, glucose sensor, imaging sensor, impedance sensor, interferometer, medichip, membrane-based sensor, Micro Electrical Mechanical System (MEMS) sensor, microfluidic sensor, micronutrient sensor, molecular sensor, motion sensor, muscle activity sensor, nanoparticle sensor, neural impulse sensor, optical sensor, osmolality sensor, pattern recognition sensor, pH level sensor, pressure sensor, protein-based sensor, reagent-based sensor, sound sensor, strain gauge, and temperature sensor.

I will now discuss the absorption-reducing member in greater detail. In an example, this invention can include an implanted absorption-reducing member that is in communication with a food-identifying sensor, wherein this sensor is implanted within a person's oral or nasal cavity and can detect when the person is eating unhealthy food. An absorption-reducing member can be in wireless communication with a food-identifying sensor that, in turn, is in fluid or gaseous communication with a person's oral and/or nasal cavities. In combination with a food-identifying sensor within the person's mouth or nose, an absorption-reducing member can selectively, temporarily, and automatically reduce the absorption of nutrients from unhealthy food while allowing normal absorption of nutrients from healthy food.

In one example, an absorption-reducing member can incorporate functions of the following sub-components that have been discussed previously: an absorption-reducing substance; an implanted reservoir; and a release-control mechanism. However, as shown in FIGS. 39 and 40, an absorption-reducing member is not limited to these three sub-components. An absorption-reducing member can selectively and automatically reduce absorption of nutrients from unhealthy food using other sub-components and means that do not require the release of an absorption-reducing substance into a person's gastrointestinal tract. We will now specify alternative sub-components and means for embodiment of an absorption-reducing member in greater detail.

In an example, an absorption-reducing member can be activated when a food-identifying sensor detects that a person is consuming a selected type of food. In an example, this selected type of food can be unhealthy food. In an example, unhealthy food can be identified as having a high concentration or amount of sugars, simple carbohydrates, fats, saturated fats, cholesterol fat, and/or sodium. In an example, a food-identifying sensor within a person's mouth can analyze saliva to detect one or more of these nutrients and thus identify unhealthy food. In an example, a food-identifying sensor in a person's mouth can be a chemical sensor.

In an example, an absorption-reducing member can be triggered when a food-identifying sensor detects that a person is consuming unhealthy food. In an example, an absorption-reducing member can selectively, temporarily, and automatically reduce the absorption of nutrients from a bolus of unhealthy food and then subsequently allow normal absorption of nutrients from a healthy bolus of food. The selective malabsorption that is enabled by the combination of a mouth-based food-identifying sensor and an absorption-reducing member creates a system for selection malabsorption that is superior to the indiscriminant malabsorption caused by devices and methods in the prior art that cannot differentiate unhealthy food versus healthy food.

In an example, an absorption-reducing member can selectively, temporarily, and automatically reduce absorption of nutrients from unhealthy food by releasing an absorption-reducing substance into a person's gastrointestinal tract when this person consumes unhealthy food. Consumption is detected by a mouth-based or nose-based food-identifying sensor. In an example, an absorption-reducing member can selectively, temporarily, and automatically reduce absorption of nutrients from food in the gastrointestinal tract by temporarily coating the walls of a person's duodenum, or another portion of a person's intestine, when the person consumes unhealthy food. In an example, an absorption-reducing member can reduce food absorption by coating a bolus of food as this bolus travels through the person's stomach or another portion of the person's gastrointestinal tract.

In various examples, an absorption-reducing member can release a substance that: temporarily coats the interior walls of the person's gastrointestinal tract as a bolus of unhealthy food passes through the tract; coats a bolus of unhealthy food as this food passes through the tract; or both. In various examples, an absorption-reducing member can release a substance that: temporarily binds to the interior walls of the person's gastrointestinal tract as a bolus of unhealthy food passes through the tract; binds to unhealthy food as the food passes through the tract; or both.

In an example, an absorption-reducing member can selectively reduce absorption of nutrients from unhealthy food by releasing a systemic pharmaceutical agent when a mouth-based or nose-based food-identifying sensor detects that a person is consuming unhealthy food. In an example, this systemic pharmaceutical agent can be released from an implanted reservoir. In an example, this systemic pharmaceutical agent can effect a rapid and temporary reduction in the ability of the intestine to absorb nutrients from food.

In an example, an absorption-reducing member can selectively, temporarily, and automatically reduce absorption of nutrients from unhealthy food by applying electricity to a gastrointestinal organ (or to nerves innervating that organ) when the person consumes unhealthy food. Consumption can be detected by a mouth-based or nose-based food-identifying sensor. In an example, an absorption-reducing member can apply electricity to the external surface of a person's stomach (or to nerves connected to the stomach) in order to temporarily reduce absorption of nutrients from food. In an example, an absorption-reducing member can apply electricity through an electrode.

In an example, an absorption-reducing member can selectively, temporarily, and automatically reduce absorption of nutrients from unhealthy food by modifying gastric motion when a person consumes unhealthy food. This can temporarily increase the speed at which food travels through the gastrointestinal tract. In an example, an absorption-reducing member can change the rate of gastric motility or gastric peristalsis. This can selectively decrease absorption of nutrients from a bolus of unhealthy food.

In an example, an absorption-reducing member can selectively, temporarily, and automatically reduce absorption of nutrients from unhealthy food by applying electricity to an enzyme-secreting organ (or to nerves connected to that organ) when a person consumes unhealthy food. In an example, this can temporarily reduce secretion of digestive enzymes into the gastrointestinal tract and thereby reduce absorption of nutrients from a bolus of unhealthy food.

In an example, an absorption-reducing member can comprise an electrical stimulation device. In an example, this member can be a neural stimulation or muscle stimulation device. In an example, an absorption-reducing member can selectively apply electrical pulses to a person's stomach, to nerves innervating their stomach, or to other organs or tissues in communication with the person's gastrointestinal tract. In combination with a food-identifying sensor in a person's mouth or nose, selective electrical stimulation in response to consumption of unhealthy food can selectively reduce absorption of nutrients from unhealthy food while allowing normal absorption of nutrients from healthy food,

In an example, an absorption-reducing member can selectively, temporarily, and automatically reduce absorption of nutrients from unhealthy food by constricting the size of a portion of the person's gastrointestinal tract when the person consumes unhealthy food. Such consumption can be detected by a mouth-based or nose-based food-identifying sensor. An absorption-reducing member can selectively, temporarily, and automatically reduce food absorption by restricting the size of a portion of the person's gastrointestinal tract.

In an example, an absorption-reducing member can constrict the size of the opening through which food travels into the stomach only when the person eats unhealthy food. In an example, this constriction can be done by decreasing the size of a gastric band or by inflating the interior of a gastric band around the upper portion of a person's stomach. When a mouth-based or nose-based sensor detects that a person is starting to consume unhealthy food, then this sensor sends signals to a gastric constriction device that constrains the size of the entrance to the stomach. In an example, an absorption-reducing member can constrict the overall size of the stomach with an adjustable-volume device that is external to the stomach wall and presses the stomach wall inward when its volume is increased. In an example, such constraints can change the speed at which a bolus of food travels through the gastrointestinal tract and can change the amount of nutrients absorbed from this bolus of food.

In an example, an absorption-reducing member can selectively, temporarily, and automatically reduce absorption of nutrients from unhealthy food by selectively: directing unhealthy food through a short (bypass) pathway in the gastrointestinal tract; and directing healthy food through a long (normal) pathway in the gastrointestinal tract. Such selective direction is made possible by communication between a mouth-based or nose-based food identification sensor and an absorption-reducing member.

For example, most gastric bypasses in the prior art are permanent and blindly reduce absorption of nutrients from healthy food as well as unhealthy food. As a result, sometimes people with gastric bypass operations suffer from deficiencies of key nutrients and have to take supplements for the rest of their lives. It would be advantageous if a device and method for weight loss could selectively decrease absorption of nutrients from unhealthy food but allow normal absorption of nutrients from healthy food. This can allow weight reduction without deficiencies of key nutrients.

The device and method disclosed herein can solve this problem and meet this need. In an example, an absorption-reducing member can selectively reduce food absorption of unhealthy food by selectively directing unhealthy food down a shorter (bypass) path with lower absorption and directing healthy food down a longer (normal) path with higher absorption. In an example, an absorption-reducing member can include an adjustable valve mechanism that is in communication with a food-identifying sensor in the person's mouth or nose.

When a food-identifying sensor detects that a person is eating unhealthy food, then an adjustable valve can be moved to a position that directs food through a shorter (bypass) digestive path. When the sensor detects that a person is eating healthy food, then the valve can be moved to a position that directs food through a longer (normal) digestive path. This avoids the deficiencies of key nutrients and vitamins that sometimes follow bariatric procedures in the prior art. In an example, a gastric bypass can be created, but an adjustable valve is used so that only unhealthy food is routed through this bypass. An absorption-reducing member selectively directs the flow of unhealthy food through the shorter (bypass) route and directs healthy food through the longer (normal) route.

In an example, an absorption-reducing member can include an adjustable food valve or chyme valve that directs unhealthy food or chyme through a bypass that avoids the duodenum and directs healthy food or chyme through a normal path that includes the duodenum. Adjusting and differentiating the digestion pathways of unhealthy versus healthy food is made possible by interaction between a mouth-based or nose-based food identification sensor and an absorption-reducing member.

In an example, when a food-identifying sensor detects that a person is eating unhealthy food, then the sensor can send a wireless signal to an absorption-reducing member that includes a valve control mechanism. This valve can route a bolus of unhealthy food through a shorter (bypass) route. When the person stops eating unhealthy food and starts eating healthy food, then a sensor detects this and changes the valve so that healthy food goes through the longer (normal) route. In various examples, an absorption-reducing member can include one or more valves selected from the group consisting of: biochemical valve, biological valve, electromagnetic valve, electromechanical valve, electronic valve, helical valve, hydraulic valve, MEMS valve, micro valve, microfluidic valve, and piezoelectric valve.

In an example, an absorption-reducing member can be implanted within a person's abdominal cavity. In various examples, an absorption-reducing member can be configured to be implanted in a subcutaneous site, in an intraperitoneal site, within adipose tissue, and/or within muscular tissue. In various examples, an absorption-reducing member can be configured to be attached to, or in fluid communication with, a body member that is selected from the group consisting of: stomach, duodenum, jejunum, ileum, caecum, colon, and esophagus. In various examples, an absorption-reducing member can be configured to be attached to a nerve that innervates a body member selected from the group consisting of: stomach, duodenum, jejunum, ileum, caecum, colon, and esophagus. In various examples, an absorption-reducing member can be attached or implanted by one or more means selected from the group consisting of: suture, staple, adhesive, glue, clamp, clip, pin, snap, elastic member, tissue pouch, fibrotic tissue, screw, and tissue anchor.

In various examples, an absorption-reducing mechanism can be can be made from one or more materials selected from the group consisting of: cobalt-chromium alloy, fluoropolymer, latex, liquid-crystal polymer, nylon, perflouroethylene, platinum, polycarbonate, polyester, polyethylene, polyolefin, polypropylene, polystyrene, polytetrafluoroethylene, polyurethane, polyvinyl chloride, pyrolytic carbon material, silicon, silicone, silicone rubber, stainless steel, tantalum, titanium, and urethane.

As shown in FIGS. 37 through 40, this invention can be embodied in a device for selectively and automatically reducing the absorption of selected types of food in a person's gastrointestinal tract. This device can comprise: (a) a food-identifying sensor that selectively detects when a person is consuming or digesting selected types of food, wherein this food-identifying sensor is configured to be implanted or attached within the person's oral cavity, the person's nasal cavity, or tissue surrounding one of these cavities; and (b) an absorption-reducing member that is implanted within the person's body, wherein this absorption-reducing member can selectively and automatically reduce the absorption of food within the person's gastrointestinal tract when the sensor detects that the person is consuming or digesting selected types of food.

As shown in FIGS. 37 through 40, this invention can be embodied in a method for selectively and automatically reducing the absorption selected types of food in the gastrointestinal tract. This method can comprise: (a) selectively and automatically detecting when a person is consuming or digesting selected types of food by means of a sensor that is configured to be implanted or attached within the person's oral cavity, the person's nasal cavity, or tissue surrounding one of these cavities; and (b) selectively and automatically reducing the absorption of food within the person's gastrointestinal tract by means of an implanted absorption-reducing member, wherein this member selectively and automatically reduces food absorption when the sensor detects that the person is consuming or digesting selected types of food.

In various examples, this invention can be embodied in a device and method to selectively, temporarily, and automatically interfere with the absorption of nutrients from unhealthy food in a person's gastrointestinal tract while allowing normal absorption of nutrients from healthy food in the person's gastrointestinal tract. In an example, this invention can function like an artificial secretory organ that selectively reduces absorption of unhealthy food within a person's gastrointestinal tract without depriving the person of important nutrients from healthy food. In an example, such a device can selectively differentiate between consumption of unhealthy food and healthy food.

In an example, such a device can selectively reduce absorption of unhealthy food and allow normal absorption of healthy food. In an example, this discriminatory ability can be adjusted or programmed to change the types and/or quantities of food which are classified as unhealthy versus healthy. Such a device and method with food discrimination capability can be superior to bariatric surgery and malabsorption devices in the prior art that are blind to whether a selected bolus of food traveling through the gastrointestinal tract is healthy or unhealthy. This device and method can avoid the deficiencies concerning essential nutrients that can occur with food-blind malabsorption devices and methods in the prior art.

In an example, this invention can be embodied in an eyewear-based system, device, and method for monitoring a person's nutritional intake comprising eyeglasses, wherein these eyeglasses further comprise at least one camera, wherein this camera automatically takes pictures or records images of food when a person is consuming food, and wherein these food pictures or images are automatically analyzed to estimate the type and quantity of food. This invention can be embodied in an eyewear-based system, device, and method for monitoring a person's nutritional intake comprising eyeglasses, wherein these eyeglasses further comprise at least one camera, wherein this camera automatically takes pictures or records images of food when a person is near food, purchasing food, ordering food, preparing food, and/or consuming food, and wherein these food pictures or images are automatically analyzed to estimate the type and quantity of food.

This invention can also be embodied in an eyewear-based system, device, and method for monitoring and modifying a person's nutritional intake comprising eyeglasses, wherein these eyeglasses further comprise at least one camera, wherein this camera automatically takes pictures or records images of food when a person is near food, purchasing food, ordering food, preparing food, and/or consuming food, and wherein these food pictures or images are automatically analyzed to estimate the type and quantity of food; a data processing unit; and a nutritional intake modification component, wherein this component modifies the person's nutritional intake based on the type and quantity of food.

In an example, an imaging member can be a camera. In an example, a nutritional intake modification component can modify a person's nutritional intake by modifying the type and quantity of food which the person consumes. In an example, a nutritional intake modification component can modify a person's nutritional intake by modifying the absorption of food which the person consumes. This invention can also be embodied in an eyewear-based system, device, and method for monitoring and modifying a person's nutritional intake comprising eyewear, wherein this eyewear further comprises at least one imaging member, wherein this imaging member automatically takes pictures or records images of food when a person is near food, purchasing food, ordering food, preparing food, and/or consuming food, and wherein these food pictures or images are automatically analyzed to estimate the type and quantity of food; a data processing unit; and a nutritional intake modification component, wherein this component modifies the person's nutritional intake based on the type and quantity of food.

This invention can also be embodied in an eyewear-based system, device, and method for monitoring and modifying a person's nutritional intake comprising: a support member which is configured to be worn on a person's head; at least one optical member which is configured to be held in proximity to an eye by the support member; at least one imaging member, wherein the imaging member is part of or attached to the support member or optical member, wherein this imaging member automatically takes pictures or records images of food when a person is near food, purchasing food, ordering food, preparing food, and/or consuming food, and wherein these food pictures or images are automatically analyzed to estimate the type and quantity of food; a data processing unit; and a nutritional intake modification component, wherein this component modifies the person's nutritional intake based on the type and quantity of food.

This invention can be embodied in an eyewear-based system and device for monitoring a person's nutritional intake comprising: eyeglasses, wherein these eyeglasses further comprise at least one camera, wherein this camera automatically takes pictures or records images of food when a person is consuming food and wherein these food pictures or images are automatically analyzed to estimate the type and quantity of food. This invention can also be embodied in an eyewear-based system and device for monitoring and modifying a person's nutritional intake comprising: eyewear, wherein this eyewear further comprises at least one imaging member, wherein this imaging member automatically takes pictures or records images of food when a person is consuming food, and wherein these food pictures or images are automatically analyzed to estimate the type and quantity of food; a data processing unit; and a nutritional intake modification component, wherein this component modifies the person's nutritional intake based on the type and quantity of food.

This invention can also be embodied in an eyewear-based system and device for monitoring and modifying a person's nutritional intake comprising: a support member which is configured to be worn on a person's head; at least one optical member which is configured to be held in proximity to an eye by the support member; at least one imaging member, wherein the imaging member is part of or attached to the support member or optical member, wherein this imaging member automatically takes pictures or records images of food when a person is consuming food, and wherein these food pictures or images are automatically analyzed to estimate the type and quantity of food; a data processing unit; and a nutritional intake modification component, wherein this component modifies the person's nutritional intake based on the type and quantity of food.

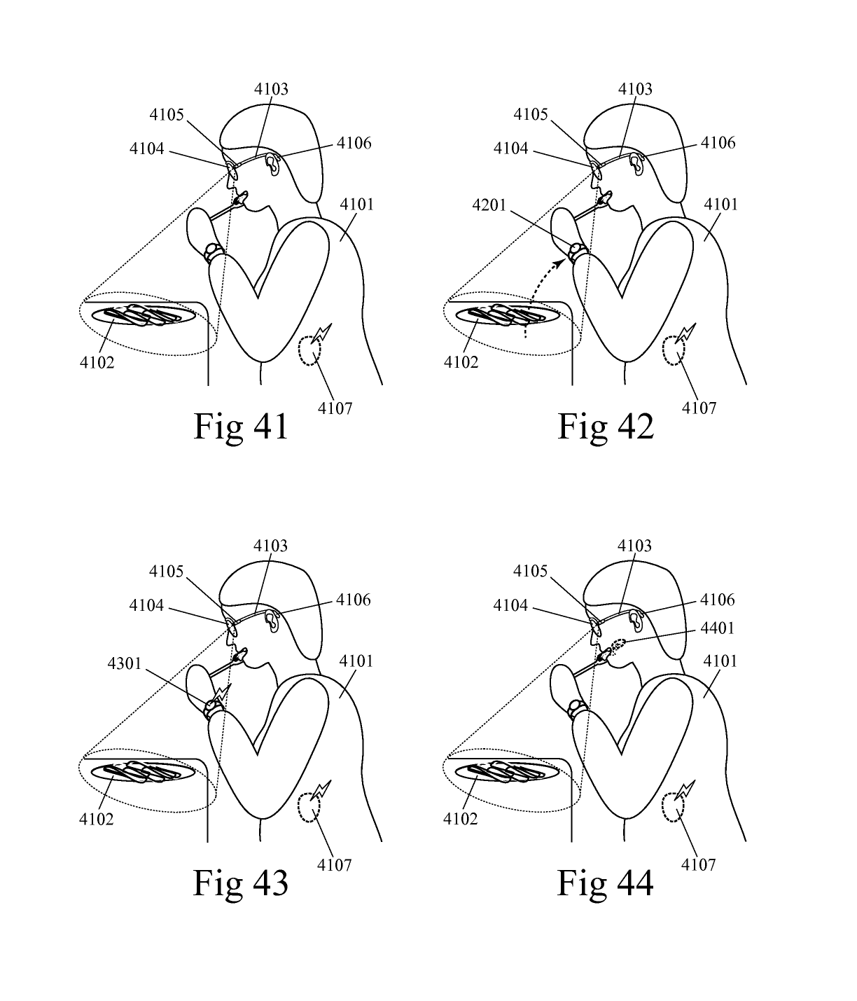

With respect to FIGS. 41 through 60, a support member can comprise one or more of the variations which we now discuss. In an example, a support member and at least one optical member can together comprise a set of eyeglasses, sunglasses, or other eyewear. In an example, a support member can be configured to span the upper portion of a person's face in a lateral (side to side) manner. In an example, the combination of a support member and at least one optical member can be configured to span the upper portion of a person's face in a lateral manner. In an example, a support member can rest on a person's ears to hold the support member in place. In an example, a support member can partially curve around each of a person's ears to better hold the support member in place. In an example, a support member (in combination with at least one optical member) can partially span the circumference of a person's head in a lateral manner. In an example, a support member (in combination with at least one optical member) can span from one ear to the other ear. In an example, a support member (in combination with at least one optical member) can span the entire circumference of a person's head in a lateral manner.

In an example, a support member (in combination with at least one optical member) can span some or all of the circumference of a person's head in a substantially-horizontal manner. In an example, a support member (in combination with at least one optical member) can span some or all of the circumference of a person's head in a plane which intersects the horizontal plane (when the person is standing up) at an angle which is less than 50 degrees. In an example, this angle can be less than 25 degrees. In an example, a support member (in combination with at least one optical member) can span some or all of the circumference of a person's head in lateral manner at substantially the same level as the person's ears. In an example, a support member (in combination with at least one optical member) can span some or all of the circumference of a person's head in lateral manner at substantially the same level as the person's eyes.

In an example, a support member can hold an optical member in proximity to a person's eye. In an example, a support member can hold an optical member within three inches of a person's eye. In an example, a support member can hold two optical members in proximity to a person's eyes, each optical member within three inches of an eye, respectively. In an example, a support member can be substantially symmetric with respect to a central vertical plane which bisects the right and left sides of a person's head. In an example, a support member can be asymmetric with respect to this plane. In an example, a support member can hold an optical member in place by spanning the entire perimeter of an optical member like a frame. In an example, a support member can hold an optical member in place by only being connected to an upper portion of the perimeter of an optical member or to a lower portion of the perimeter of an optical member. In an example, a support member can hold an optical member in place by being connected to one or both sides of the optical member. In an example, a support member can hold an optical member in place by being connected to the front or back of an optical member.

In an example, a support member can comprise a single continuous arcuate piece which wraps around some or all of the circumference of a person's head. In an example, a support member can comprise multiple connected pieces which collectively span some or all of the circumference of a person's head. In an example, a support member can further comprise two side pieces (“ear pieces”) which are connected to a front piece. In an example, this connection can be a hinge mechanism. In an example, a support member can comprise eyeglass frames. In an example, a support member can comprise two side pieces which each span from an ear to the front of the person's face plus a front piece which spans across the person's face (from side piece to side piece). In an example, a side piece can be substantially straight. In an example, a side piece can have a relatively constant cross-sectional size. In an example, a side piece can have a cross-sectional size which increases (flares) from the rear portion of the side piece to the front portion of the side piece. In an example, a side piece can partially curve around a person's ear. In an example, a support member and one or more optical members can comprise a visor. In an example, a support member and one or more optical members can comprise goggles.

In an example, a support member can be configured to laterally span a person's face at substantially the same level as the person's eyebrows. In an example, a support member can be configured to laterally span a person's face at substantially the same level as the person's eyes. In an example, a support member can be configured to laterally span a person's face at substantially the same level as the person's forehead. In an example, a support member can be configured to laterally span the sides of a person's head at substantially the same level as the person's eyebrows. In an example, a support member can be configured to laterally span the sides of a person's head at substantially the same level as the person's eyes. In an example, a support member can be configured to laterally span the sides of a person's head at substantially the same level as the person's forehead. In an example, a support member can be configured to laterally span the sides of a person's head at substantially the same level as the person's eyes and to laterally span a person's face at substantially the same level as the person's eyebrows.

In an example, a support member can be arcuate. In an example, a support member can span a portion of a person's head in a sinusoidal manner. In an example, a support member can further comprise at least one upward protrusion from a frontal portion of the support member which is configured to span a portion of a person's forehead. In an example, an upward protrusion from the front of a support member can have an arcuate shape. In an example, an upward protrusion from the front of a support member can have a sinusoidal section shape. In an example, an upward protrusion from the front of a support member can have a conic section shape. In an example, a support member can further comprise at least one upward protrusion from a side portion of the support member which is configured to span a portion of a person's forehead, temple, and/or a side of the person's head. In an example, an upward protrusion from the side of a support member can have an arcuate shape. In an example, an upward protrusion from the side of a support member can have a sinusoidal section shape. In an example, an upward protrusion from the side of a support member can have a conic section shape.

In an example, a support member can further comprise a single central upward protrusion from a frontal portion of the support member which is configured to span a portion of the middle of a person's forehead. In an example, this upward portion can be substantially straight. In an example, this upward protrusion can have an arcuate shape, conic section shape, and/or sinusoidal section shape. In an example, a support member can further comprise two upward protrusions from a frontal portion of the support member which are configured to span portions of the right side and the left side, respectively, of a person's forehead. In an example, these upward portions can be substantially straight. In an example, these upward protrusions can have arcuate shapes, conic section shapes, and/or sinusoidal section shapes. In an example, a support member can further comprises at least one upward protrusion which is configured to span a portion of a person's forehead, temple, and/or a side of the person's head and wherein this upward protrusion holds an electromagnetic brain activity sensor.

In an example, a support member can further comprise a single central upward protrusion from a side portion of the support member which is configured to span a portion of the side of a person's forehead, temple, and/or side of the person's head. In an example, this upward portion can be substantially straight. In an example, this upward protrusion can have an arcuate shape, conic section shape, undulating shape, and/or sinusoidal section shape. In an example, a support member can further comprise two upward protrusions, one from each side portion of the support member, which are configured to span portions of the right side and the left side, respectively, of a person's forehead and/or the person's right and left temples. In an example, these upward portions can be substantially straight. In an example, these upward protrusions can have arcuate shapes, conic section shapes, undulating shapes, and/or sinusoidal section shapes.

In an example, an upward protrusion from a support member can further comprise and/or hold at least one physiological sensor. In an example, an upward protrusion can further comprise and/or hold an electromagnetic energy sensor. In an example, an upward protrusion can further comprise and/or hold an electroencephalographic (EEG) sensor. In an example, this EEG sensor can be an electrode. In an example, an upward protrusion from a support member can hold an EEG sensor at a location selected from the group consisting of: FP1, FPz, FP2, F7, F5, F3, F1, Fz, F2, F4, F6, and F8. In a more general example, an upward protrusion from a support member can hold an EEG sensor at a location selected from the group consisting of: FP1, FPz, FP2, AF7, AF5, AF3, AFz, AF4, AF6, AF8, F7, F5, F3, F1, Fz, F2, F4, F6, F8, FT7, FC5, FC3, FC1, FCz, FC2, FC4, FC6, FT8, T3/T7, C3, C4, C1, Cz, C2, C5, C6, T4/T8, TP7, CP5, CP3, CP1, CPz, CP2, CP4, CP6, TP8, T5/P7, P5, P3, P1, Pz, P2, P4, P6, T6/P8, PO7, PO5, PO3, POz, PO4, PO6, PO8, O1, Oz, and O2. In an example, an upward protrusion can further comprise and/or hold an electrooculographic (EOG) sensor. In an example, an upward protrusion from a support member can further comprise and/or hold a light energy sensor. In an example, an upward protrusion can further comprise and/or hold a spectroscopic sensor. In an example, an upward protrusion can further comprise and/or hold an ultrasonic sensor.

In an example, a support member can have a longitudinal axis which spans some or all of the circumference of a person's head in a substantially lateral manner. In an example, a support member can be a single continuous piece and its longitudinal axis can have an arcuate shape. In an example, a support member can comprise multiple connected pieces and its longitudinal axis can be a connected sequence of substantially-straight line segments (e.g. a “spline” shape) or a connected sequence of arcuate segments. In an example, a support member can have a series of lateral cross-sections which are perpendicular to its longitudinal axis. In an example, these lateral cross-sections can have vertical heights (assuming that the person is standing up) and horizontal widths (assuming that the person is standing up). In an example, the vertical heights of the lateral cross-sections of a support member are no greater than four inches. In an example, the horizontal widths of the lateral cross-sections of a support member are no greater than two inches. In an example, vertical heights can be substantially constant as the longitudinal axis spans from an ear to the front of the person's face. In an example, vertical heights can increase (flare) as the longitudinal axis spans from an ear to the front of the person's face.

In an example, a support member can be made of metal, a polymer, a textile, or a combination thereof. In an example, a support member can be substantially rigid. In an example, a support member can be flexible. In an example, a support member can be sufficiently flexible to be placed around (a portion of) a person's head, but also sufficiently resilient to be held against a person's head by tension once it is placed around (a portion of) a person's head. In an example, a support member can be elastic. In an example, a support member can be sufficiently elastic that it can be placed around (a portion of) a person's head, but also sufficiently resilient to be held against a person's head by tension once it is placed around (a portion of) a person's head. In an example, a support member can be held onto a person's head by one or more attachment mechanisms selected from the group consisting of: band, elastic, loop, strap, chain, clip, clasp, snap, buckle, clamp, button, hook, pin, plug, hook-and-eye mechanism, adhesive, tape, electronic and/or electromagnetic connector, electronic plug, magnetic connector, threaded member, fiber, thread, and zipper.

With respect to FIGS. 41 through 60, an optical member can comprise one or more of the variations which we now discuss. In an example, an optical member can transmit, channel, focus, refract, and/or guide light from a person's environment into the person's eye. In an example, an optical member can be a lens. In an example, an optical member can be a convex lens or a concave lens. In an example, an optical member can be made from a polymer, glass, or a crystalline material. In an example, an optical member can be a compound lens. In an example, an optical member can be a lens with an adjustable focal length. In an example, the convexity or concavity of a lens can be adjusted automatically by one or more actuators. In an example, the convexity or concavity of a lens can be adjusted automatically by changing the pressure of a liquid or gel within the lens. In an example, the concavity or convexity of a lens can be adjusted automatically based on data from an eye-tracking and/or gaze-tracking mechanism which tracks a person's eyes.

In an example, this invention can comprise a single optical member. In an example, this invention can comprise two optical members, one for each eye. In an example, an optical member can be held at least partially in front of a person's eye by a support member. In an example, an optical member can be held within two inches of a person's eye by a support member. In an example, a support member and two optical members can together comprise a pair of eyeglasses, sunglasses, goggles, or other eyewear. In an example, this invention can comprise electronically-functional eyeglasses, sunglasses, or goggles; an electronically-functional monocle; a visor or helmet; augmented reality or virtual reality eyewear; or an electronically-functional contact lens.

In an example, an optical member can comprise a virtual image display, computer display, and/or electronic display screen which emits light and/or projects an image into a person's eye. In an example, an optical member can display a virtual object and/or virtual image in a person's field of view. In an example, an optical member can display a virtual object in juxtaposition with a real world (physical) object in a person's field of view. In an example, an optical member can display virtual information concerning a real world (physical) object in a person's field of view. In an example, this invention can comprise the visual component of a virtual reality and/or augmented reality system. In an example, an optical member can transition from a first configuration in which it transmits light from the environment into a person's eye to a second configuration in which it emits light comprising a virtual image into the person's eye.

In an example, an optical member can display virtual text and/or virtual images over or near a person+ view of a real world (physical) object. In an example, an optical member can superimpose virtual text and/or a virtual image over a person's view of a real world (physical) object. In an example, an optical member can display virtual text and/or virtual images over or near food which is in a person's field of view. In an example, this invention can display virtual text over or near food, wherein this virtual text provides nutritional information concerning this food. In an example, this virtual text can indicate potential adverse health effects which may occur if this food is consumed. In an example, adverse health effects can include weight gain, elevated blood glucose, and/or an allergic reaction. In an example, virtual text display over or near food in a person's field of view can reduce the person's consumption of that food.

In an example, this invention can display a virtual image over or near food which is in a person's field of vision. In an example, this virtual image can communicate potential adverse health effects which may occur if this food is consumed. In an example, these adverse health effects can include weight gain, elevated blood glucose, and/or an allergic reaction. In an example, a virtual image can be a image with negative meaning concerning potential negative effects of consuming this food. In an example, a negative image can help to modify a person's food consumption in order to help avoid negative consequences. In an example, a virtual image can be a positive image of the positive effects of avoiding consumption of this food. In an example, a positive image can help to modify a person's food consumption to help achieve positive consequences. In an example, an optical member can display unappealing images over (or near) types or quantities of food which are identified as unhealthy. In an example, an optical member can display appealing images over (or near) types or quantities of food which are identified as healthy. In an example, displaying unappealing images in juxtaposition to unhealthy food and displaying appealing images in juxtaposition to healthy food can help to improve the quality of a person's nutrition as part of an overall system for weight management and health improvement.

In an example, an optical member can be selected from the group consisting of: simple lens, concave lens, convex lens, bifocal lens, trifocal lens, asymmetric lens, optoelectronic lens, liquid lens, variable-focal-length lens, microlens, tinted lens, nanoscale grating, etched waveguide, nanoimprint lithography pathway, resonant grating filter, Split Ring Resonator (SRR), thermoplastic nanoimprint pathway, crystalline structure, photonic metamaterial, photonic crystal, optical fibers, polarizing filter, Digital Light Processor (DLP), Electromagnetically Induced Transparency (EIT) structure, birefringent member, nanotube structure, lens array, light-guiding metamaterial structure, light-guiding tubes, metamaterial light channel, prism, mirror, Digital Micromirror Device (DMD); virtual image display, computer screen, heads up display, array or matrix of light-emitting members, infrared display, laser display, light emitting diodes (LED), array or matrix of light emitting diodes (LEDs), waveguide, array or matrix of fiber optic members, optoelectronic lens, computer display, camera or other imaging device, light-emitting member array or matrix, light display array or matrix, liquid crystal display (LCD), and image projector.

With respect to FIGS. 41 through 60, an imaging member can comprise one or more of the variations which we now discuss. In an example, an imaging member can be a camera. In an example, an imaging member can be selected from the group consisting of: digital camera, video camera, motion picture camera, still picture camera, visible light camera, infrared or near-infrared camera, ultraviolet light camera, spectral analysis camera, digital imaging member, video imaging member, visible light imaging member, infrared or near-infrared imaging member, ultraviolet light imaging member, spectral analysis imaging member, chromatography imaging member, coherent light imaging member, electro-optical imaging member, gesture recognition imaging member, and pattern recognition imaging member.

In an example, an imaging member can be part of a support member. In an example, an imaging member can be removably attached to a support member. In an example, an imaging member can be part of an optical member. In an example, an imaging member can be removably attached to an optical member. In an example, this invention can have a first configuration in which an imaging member is retracted into (or behind) a support member so that it is obscured from external view and this invention can have a second configuration in which the imaging member is projected out from (or moved out from behind) the support member so that the imaging member can take pictures and/or record images. In an example, the invention can transition from the first configuration to the second configuration when triggered manually by the person. In an example, the invention can transition from the first configuration to the second configuration automatically based on data from one or more sensors which indicate that the person is near food, purchasing food, ordering food, preparing food, and/or consuming food.

In an example, an imaging member can focus toward the three-dimensional space which is in front of the person who is wearing it. In an example, an imaging member can have a wide-angle field of view which includes space to the right and left of the person, as well as space in front of the person. In an example, the focal range and scope of an imaging member can be automatically reduced based on the privacy expectations associated with a particular location and/or environmental context. In an example, an imaging member can be in electronic communication with a GPS (or other location-finding) system as part of a method to determine a location-specific expectation of privacy. In an example, in a location or environmental context in which (other) people have a high expectation of privacy, an imaging member can have restricted focal range and/or scope in which objects beyond a selected range or scope are out of focus and unrecognizable. In an example, an imaging member can automatically blur or redact the portions of pictures and/or images which include (other) people. In an example, an imaging member can be automatically deactivated (and/or not automatically triggered by food consumption) in a location and/or environmental context in which people have a high expectation of privacy. In an example, pictures or images can be quickly and completely erased after food identification has occurred in a location and/or environmental context in which people have a high expectation of privacy.

In an example, an imaging member can have a longitudinal axis which is substantially parallel with a side portion (“ear piece”) of a support member. In an example, an imaging member can have a longitudinal axis which is substantially perpendicular to a front portion of a support member. In an example, an imaging member can be in electromagnetic communication with a data processing unit which is part of a support member. In an example, an imaging member can be in electromagnetic communication with a data processing unit in a remote location.

In an example, this invention can comprise a single imaging member. In an example, this invention can comprise two imaging members. In an example, this invention can comprise two (stereoscopic) imaging members, one near each eye. In an example, this invention can comprise two imaging members, one for each eye. In an example, this invention can comprise a single wearable camera. In an example, this invention can comprise two wearable cameras. In an example, this invention can comprise two (stereoscopic) wearable cameras, one near each eye. In an example, this invention can comprise two wearable cameras, one for each eye. In an example, this invention can comprise two imaging members which simultaneously take pictures of food from different angles for three-dimensional modeling and/or volumetric analysis of food quantity. In an example, this invention can comprise a single imaging member which takes pictures of food from different angles over time as a person moves their body. As is the case with two stereoscopic imaging members, pictures from different angles from a single imaging member can be used for three-dimensional modeling and/or volumetric analysis of food quantity.

In an example, an imaging member can have a field of view which spans a portion of the three-dimensional space in front of a person's body. In an example, an imaging member can have a field of view which substantially comprises the natural field of view from a person's eye. In an example, the fields of view from two imaging members can substantially comprise the fields of view from the person's eyes. In an example, this invention can further comprise a eye-tracking and/or gaze-tracking function which controls and moves the field of view of an imaging member so that the field of view of the imaging member substantially follows the changing field of view from the person's eye which is being tracked. In an example, an eye-tracking and/or gaze-tracking function can also track the focal direction and distance of a person's eyes and can adjust the focal direction and distance of one or more imaging members. In an example, the field of view of an imaging member can be moved to track the locations of a person's hands some or all of the time. In an example, this invention can further comprise a hand recognition and/or gesture recognition function which tracks the locations of a person's hands in order to capture interactions between the person's hands and food. In an example, the field of view from an imaging member can be directed forward from a person's head. In an example, an imaging member can have central focal axis which is substantially parallel with the longitudinal axis of a support member along a side piece (“ear piece”).

In an example, an imaging member can take pictures and/or record images of the three-dimensional space in front of a person's body in order to capture images of food which is within the person's reach, images of interactions between the person's hands and food, and interactions between food and the person's mouth. In an example, an imaging member can take pictures and/or record images only when the wearer of the device manually and/or voluntarily triggers it to take pictures and/or recording images. With respect to accuracy of nutritional intake monitoring, this approach depends on the person being sufficiently compliant and diligent to capture images of most (or all) of their food consumption. In an example, a person wearing the device can trigger an imaging member to take pictures and/or record images by using voice-based command, touch-based command, gesture-based, and/or body-generated electromagnetic signal.

In an example, this invention can request the wearer's permission for automatic activation to start taking pictures and/or recording images. In an example, this invention can request the permission of all people who would be within the field of view of an imaging member before it starts taking pictures and/or recording images. In an example, when sensor data indicates that the person wearing the device is near food and/or consuming food, then the device can issue a voice-based request for permission to start taking pictures and/or recording images. In an example, if any person within hearing distance says “No”, then the device recognizes this denial of the request and does not start taking pictures and/or recoding images. In an example, the voice-based request can be very courteous—with an accent and sentence construction like that of C3PO, for example. In an example, an imaging member can request bio-identified wearer permission (e.g. voice identification or other biometric identification) for automatic activation to start taking pictures and/or recording images.

In an example, an imaging member can automatically start taking pictures and/or recording images at periodic intervals or at random times, in the hope that this approach will by chance capture images of most of a person's food consumption. In an alternative example, an imaging member can automatically start taking pictures and/or recording images at selected times and/or places which are associated with food consumption. In an example, an imaging member can take automatically start taking pictures and/or record images during selected times which are regularly associated with food consumption (e.g. meal times). In an example, an imaging member can automatically start taking automatically pictures and/or record images at selected places which are regularly associated with food consumption (e.g. restaurants or kitchens). In an example, an imaging member can stop taking pictures and/or recording images when no food consumption is detected during selected duration of time, when a selected time interval concludes, or when a person leaves location that is associated with food consumption.

In an example, an imaging member can take pictures and/or record images all the time, or at least whenever the person is wearing the support member. This is more likely to capture images most (or all) of a person's food consumption than taking pictures at periodic intervals or random times. However, continuous picture taking and/or image recording can be obtrusive with respect to privacy. It may be that the health benefits of monitoring and modifying a person's nutritional intake can outweigh the erosion of privacy from continuous imaging. However, this invention comprises alternative methods and devices which automatically trigger picture taking and/or image recording when a person is near food or consumes food. This can achieve help a person to find the optical balance between nutritional improvement and privacy preservation. Also, in the interest of the privacy of the person wearing the device and the privacy of others nearby, this invention can have an external signal which indicates when it is taking pictures and/or recording images. In an example, this external signal can be a light, a sound, or a movement.

In an example, an imaging member can continually take pictures and/or record images, but these pictures and/or images can be automatically erased after a selected period of time (and/or never stored in long-term memory) unless analysis of these pictures and/or images indicates that the person is near food and/or consuming food. In an example, pictures and/or images may only be kept for a period of time which is just long enough to determine with a high degree of probability whether a person is consuming food; if they are not consuming food, then the pictures and/or images are automatically erased. In an example, this period of time can be less than five minutes. In an example, in the interest of privacy, this invention may not include hardware and/or connectivity which permits transmission of pictures and/or images to external systems. In an example, an imaging member may continually take pictures and/or record images, but these pictures and/or images are automatically erased (and/or never stored in long-term memory) immediately after a selected period of time which is required to analyze these images to estimate the type and quantity of food consumed. In an example, this period of time can be less than five minutes. In an example, continual taking of pictures and/or recording of images can be deactivated by the wearer.

In an example, this invention can further comprise one or more sensors whose data can trigger activation of the imaging member when a person is near food, purchasing food, ordering food, preparing food, and/or consuming food. In an example, this invention can further comprise one or more sensors whose data improves the accuracy of estimation of food types and/or quantities. In an example, these one or more sensors can be wearable sensors. In an example, these one or more sensors can be implanted sensors.

In an example, an imaging member can be activated (or triggered) to automatically start taking pictures and/or recording images when data from one or more wearable or implantable sensors indicates that a person is near food, purchasing food, ordering food, preparing food, and/or consuming food. In an example, an imaging member can automatically starting taking pictures and/or recording pictures of food during (or prior to) consumption without the need for specific action by the person in association with a specific eating event, apart from the actual act of eating.

In an example, the imaging member can be automatically activated to take pictures when a person eats based on a sensor selected from the group consisting of: accelerometer, inclinometer, and motion sensor. In an example, the imaging member can be automatically activated to take pictures when a person eats based on a sensor selected from the group consisting of: EEG sensor, ECG sensor, and EMG sensor. In an example, the imaging member can be automatically activated to take pictures when a person eats based on a sensor selected from the group consisting of: sound sensor, smell sensor, blood pressure sensor, heart rate sensor, electrochemical sensor, gastric activity sensor, GPS sensor, location sensor, image sensor, optical sensor, piezoelectric sensor, respiration sensor, strain gauge, electrogoniometer, chewing sensor, swallow sensor, temperature sensor, and pressure sensor. In an example, the imaging member can be automatically activated to take pictures when data from one or more wearable or implanted sensors indicates that a person is consuming food or will probably consume food soon.

In an example, at least one sensor can be an electromagnetic energy sensor which measures the conductivity, voltage, impedance, or resistance of electromagnetic energy transmitted through body tissue. In an example, at least one sensor can be selected from the group consisting of: glucometer, glucose sensor, glucose monitor, blood glucose monitor, cellular fluid glucose monitor, spectroscopic sensor, food composition analyzer, oximeter, oximetry sensor, pulse oximeter, tissue oximetry sensor, tissue saturation oximeter, wrist oximeter, oxygen consumption monitor, oxygen level monitor, oxygen saturation monitor, ambient air sensor, gas composition sensor, blood oximeter, ear oximeter, cutaneous oxygen monitor, cerebral oximetry monitor, capnography sensor, carbon dioxide sensor, carbon monoxide sensor, artificial olfactory sensor, smell sensor, moisture sensor, humidity sensor, hydration sensor, skin moisture sensor, chemiresistor sensor, chemoreceptor sensor, electrochemical sensor, amino acid sensor, cholesterol sensor, body fat sensor, osmolality sensor, pH level sensor, sodium sensor, taste sensor, and microbial sensor.

In an example, this invention can further comprise one or more wearable or implantable sensors, wherein data from these one or more sensors is jointly analyzed with pictures or images from the imaging member in order to provide more accurate estimation of food types and/or quantities than is possible with analysis of pictures or images from an imaging member alone. In discussion associated with the following figures, different types of wearable or implantable sensors can be used to: collect data which can automatically trigger an imaging member to start taking pictures and/or recording images when a person is near food, purchasing food, ordering food, preparing food, and/or consuming food; generate data which is jointly analyzed with food images from the imaging member in order to provide more accurate estimation of food types and/or quantities; or both.

In an example, this invention can comprise an imaging member which automatically starts taking pictures and/or recording images when data from one or more wearable sensors indicates that a person is near food, purchasing food, ordering food, preparing food, and/or consuming food. In an example, this invention can comprise an imaging member which is automatically activated to start taking pictures and/or recording images when data from one or more wearable sensors indicates that a person is near food, purchasing food, ordering food, preparing food, and/or consuming food. In an example, this invention can comprise an imaging member which is automatically triggered to start taking pictures and/or recording images when data from one or more wearable sensors indicates that a person is near food, purchasing food, ordering food, preparing food, and/or consuming food. In an example, such automatic taking of pictures and/or recording of images when a person is near food, purchasing food, ordering food, preparing food, and/or consuming food can consistently take pictures of nearby food and/or food consumption for consistent monitoring of food consumption, without the high level of privacy erosion which can be caused by continuous picture taking and/or image recording by a wearable camera.

In an example, data from one or more wearable sensors can automatically start, activate, and/or trigger picture taking and/or image recording by this invention without requiring any action by a person during an eating event apart from the actual act of eating. In an example, this invention can be configured so that the field of view of a wearable imaging member automatically spans the three-dimensional space in which hand-to-food and food-to-mouth interaction is likely to occur so that a person does not have to manually direct an imaging member toward food, manually focus an imaging member on food, or manually click an imaging member during an eating event in order to take pictures and/or record images of food. In an example, an imaging member can be configured to automatically start taking pictures and/or recording images of food when analysis of data from one or more wearable sensors indicates that a person is near food, purchasing food, ordering food, preparing food, and/or consuming food. In an example, an imaging member can be configured to automatically start taking pictures and/or recording images of food when analysis of data from one or more wearable sensors indicates that a person may consume food soon. In an example, an imaging member can start taking pictures and/or recording images when analysis of this data from wearable sensors indicates that a person is near food, purchasing food, ordering food, preparing food, and/or consuming food.

In an example, an imaging member can automatically start taking pictures and/or recording images based on data from one or more sensors which are part of eyewear. In an example, an imaging member can automatically start taking pictures and/or recording images based on data from one or more sensors which are part of a support member discussed in this invention. In an example, an imaging member can automatically start taking pictures and/or recording images based on data from one or more sensors which are part of an optical member discussed in this invention. In an example, an imaging member can automatically start taking pictures and/or recording images based on data from one or more sensors which are removably attachable to eyewear. In an example, an imaging member can automatically start taking pictures and/or recording images based on data from one or more sensors which are removably attached to a support member discussed in this invention. In an example, an imaging member can automatically start taking pictures and/or recording images based on data from one or more sensors which are removably attached to an optical member discussed in this invention.

In an example, an imaging member can automatically start taking pictures and/or recording images based on data from one or more wearable or implanted sensors which are separate from eyewear but are part of a system which includes a chain of electronic communication with the imaging member. In an example, an imaging member can automatically start taking pictures and/or recording images based on data from one or more wearable or implanted sensors which are separate from eyewear but are in wireless communication with a data processing unit which is in electronic communication with the imaging member. In an example, an imaging member can automatically start taking pictures and/or recording images of food based on data from one or more sensors which are in locations which are separate from eyewear, but which are in wireless communication with a support member, an optical member, a data processing unit, an imaging member, or a combination thereof. In an example, electronically-functional eyewear which takes pictures and/or records images of food and a separate wearable device with one or more sensors which activate picture taking can together comprise a system and method for monitoring the types and quantities of food near a person and/or consumed by a person.

In an example, a wearable sensor which triggers activation of an imaging member can be worn on a body location which is selected from the group consisting of: finger, hand, wrist, arm, neck, head, ear, mouth, jaw, nose, torso, and abdomen. In an example, a wearable sensor which triggers activation of an imaging member can be part of a wearable device which is selected from the group consisting of: watch, wrist band, bracelet, bangle, wrist cuff, finger ring, electronically-functional glove, arm band, smart shirt, electronically-functional necklace, electronically-functional collar, electronically-functional button, electronically-function pin, electronically-functional pendant or dog tags, ear ring, hearing aid, ear bud or insert, nose ring, tongue ring, dental insert or attachment, palatal insert or attachment, electronically-functional bandage, and electronically-functional tattoo.

In an example, a smart watch which further comprises a food consumption sensor can trigger activation of an imaging member to take pictures when data from this sensor indicates that a person is near food, purchasing food, ordering food, preparing food, and/or consuming food. In an example, a wrist band or arm band which further comprises a food consumption sensor can trigger activation of an imaging member to take pictures when data from this sensor indicates that a person is near food, purchasing food, ordering food, preparing food, and/or consuming food. In an example, a necklace, pendant, or collar which further comprises a food consumption sensor can trigger activation of an imaging member to take pictures when data from this sensor indicates that a person is near food, purchasing food, ordering food, preparing food, and/or consuming food. In an example, a smart shirt which further comprises a food consumption sensor can trigger activation of an imaging member to take pictures when data from this sensor indicates that a person is near food, purchasing food, ordering food, preparing food, and/or consuming food. In an example, a hearing aid, ear bud, or ear insert which further comprises a food consumption sensor can trigger activation of an imaging member to take pictures when data from this sensor indicates that a person is near food, purchasing food, ordering food, preparing food, and/or consuming food. In an example, a dental insert or appliance which further comprises a food consumption sensor can trigger activation of an imaging member to take pictures when data from this sensor indicates that a person is near food, purchasing food, ordering food, preparing food, and/or consuming food.

In an example, this invention can comprise an imaging member which automatically starts taking pictures and/or recording images when data from one or more implanted sensors indicates that a person is near food and/or is consuming food. In an example, this invention can comprise an imaging member which is automatically activated to start taking pictures and/or recording images when data from one or more implanted sensors indicates that a person is consuming food or anticipating consuming food. In an example, this invention can comprise an imaging member which is automatically triggered to start taking pictures and/or recording images when data from one or more implanted sensors indicates that a person is consuming food or anticipating consuming food. In an example, such automatic taking of pictures and/or recording of images when a person is consuming food or anticipating consuming food can consistently take pictures of food for consistent monitoring of food consumption, without the high level of privacy erosion which can be caused by continuous picture taking and/or image recording by a wearable camera.

In an example, an implanted sensor which triggers activation of an imaging member can be implanted so as to be in electromagnetic, fluid, gaseous, mechanical, and/or optical communication with one or more body organs, members, and/or tissues selected from the group consisting of: arm, hand, and/or finger muscles, nerve which innervates arm, hand, and/or finger muscles, jaw muscles, nerve which innervates jaw muscles, oral cavity, upper palate, tooth, tongue, nerve which innervates the tongue, nose, nasal passages, esophagus, nerve which innervates the esophagus, esophageal-gastric junction, stomach, nerve which innervates the stomach, pyloric sphincter, nerve which innervates the pyloric valve, duodenum, nerve which innervates the duodenum, upper intestine, lower intestine, liver, pancreas, spleen, and brain.

In an example, an imaging member can start taking pictures and/or recording images when analysis of data from an implanted sensor indicates that the person is near food, purchasing food, ordering food, preparing food, and/or consuming food. In an example, an implantable sensor can be configured to be in electromagnetic, fluid, gaseous, optical, sonic, and/or biochemical communication with a body member selected from the group consisting of: oral cavity, tongue, teeth, sinus, nose, ear, jaw, hand, abdomen, chest, esophagus, stomach, intestine, bladder, kidney, pancreas, peripheral nerve, and brain. In an example, an implanted sensor can be in wireless communication with a data processing unit, data transmitter, and/or data receiver which is part of eyewear. In an example, an implanted sensor can be in wireless communication with a data processing unit, data transmitter, and/or data receiver which is, in turn, in electronic communication with eyewear.

In an example, an implant in a person's oral cavity which further comprises a food consumption sensor can trigger activation of an imaging member to take pictures when data from this sensor indicates that a person is near food, purchasing food, ordering food, preparing food, and/or consuming food. In an example, a intra-oral sensor can sense when a person begins to salivate. In an example, an intra-oral sensor can sense when a person puts food in their mouth. In an example, an intra-oral sensor can sense when a person begins to chew and swallow food. In an example, an implant in a person's nasal passages which further comprises a food consumption sensor can trigger activation of an imaging member. In an example, a nasal passage sensor can sense when a person begins to smell food.

In an example, an implant with a sensor which is in electromagnetic communication with a person's CN VII (Facial Nerve), CN IX (Glossopharyngeal Nerve) CN X (Vagus Nerve), and/or CN V (Trigeminal Nerve) can trigger activation of an imaging member to take pictures when data from this sensor indicates that a person is near food, purchasing food, ordering food, preparing food, and/or consuming food. In an example, an implant in a person's brain which further comprises a neural activity sensor can trigger activation of an imaging member to take pictures when data from this sensor indicates that a person is near food, purchasing food, ordering food, preparing food, and/or consuming food.

In an example, an implant in a person's abdominal cavity which further comprises a food consumption sensor can trigger activation of an imaging member to take pictures when data from this sensor indicates that a person is near food, purchasing food, ordering food, preparing food, and/or consuming food. In an example, an implant within, or attached to, a person's stomach which further comprises a food consumption sensor can trigger activation of an imaging member to take pictures when data from this sensor indicates that a person is near food, purchasing food, ordering food, preparing food, and/or consuming food.

In an example, a food consumption sensor can be implanted so as to be in electromagnetic, fluid, gaseous, mechanical, and/or optical communication with one or more body organs and/or body tissues selected from the group consisting of: arm, hand, and/or finger muscles, nerve which innervates arm, hand, and/or finger muscles, jaw muscles, nerve which innervates jaw muscles, oral cavity, upper palate, tooth, tongue, nerve which innervates the tongue, salivary gland, nerve which innervates a salivary gland, sinus cavity, olfactory nerve, esophagus, nerve which innervates the esophagus, stomach, nerve which innervates the stomach, muscles which move the stomach, pyloric sphincter, nerve which innervates the pyloric sphincter, muscles which move the pyloric valve, duodenum, nerve which innervates the duodenum, upper intestine, lower intestine, liver, nerve which innervates the liver, pancreas, nerve which innervates the pancreas, spleen, and brain.

In an example, an implanted food consumption sensor can be in wireless communication with a data processing unit, data transmitter, and/or data receiver which is part of eyewear. In an example, an implanted food consumption sensor can be in wireless communication with a data processing unit, data transmitter, and/or data receiver which is, in turn, in electronic communication with eyewear. In an example, electronically-functional eyewear which takes pictures and/or records images of food and an implantable sensor which analyzes the chemical composition of food can together comprise a system for monitoring the types and quantities of food near a person and/or consumed by a person.

In an example, an imaging member can automatically start taking pictures and/or recording images based on data from one or more sensors in a handheld device which is in wireless communication with electronically-functional eyewear. In an example, a handheld device can be a mobile communication device. In an example, a handheld device can be a food utensil or food probe. In an example, data from sensors in a handheld device can be used to analyze the type and/or quantity of food near a person and/or food consumed by a person. In an example, sensors in a handheld device can be in optical, fluid, gaseous, electromagnetic, and/or chemical communication with food. In an example, a sensor in a handheld device can be a spectroscopic sensor. In an example, a sensor in a handheld device can be an electromagnetic sensor. In an example, a sensor in a handheld device can be a biochemical sensor. In an example, electronically-functional eyewear and a handheld device which analyzes the chemical composition of food can together comprise a system for monitoring the types and quantities of food near a person and/or consumed by a person.

In an example, this invention can further comprise one or more sensors selected from the group consisting of: electromagnetic energy sensor, motion sensor, location sensor, sonic energy sensor, light energy sensor, glucose and/or other chemical sensor, pressure sensor, and thermal energy sensor. In an example, this invention can further comprise a food proximity and/or food consumption sensor with a sensing modality which is selected from the group consisting of: electromagnetic energy, motion or location, sonic energy, light energy, glucose and/or other chemical, pressure, and thermal energy. In an example, this invention can further comprise a food proximity and/or food consumption sensor whose data is analyzed to automatically activate or trigger an imaging member, wherein this sensor has a sensing modality which is selected from the group consisting of: electromagnetic energy, motion or location, sonic energy, light energy, glucose and/or other chemical, pressure, and thermal energy. In an example, data from a wearable or implanted sensor combined with food images from an imaging member can provide more accurate estimation of food types and/or quantities than either the data or images alone. In an example, data from multiple sensors with different sensing modalities can be jointly analyzed to estimate food types and/or quantities more accurately than data from a single-mode sensor.

In an example, this invention can further comprise an electromagnetic energy sensor which is configured to be in electromagnetic communication with body tissue. In an example, an imaging member can automatically start taking pictures and/or recording images when data from an electromagnetic energy sensor indicates that a person is near food, purchasing food, ordering food, preparing food, and/or consuming food. In an example, an electromagnetic energy sensor can measure the conductivity, voltage, impedance, or resistance of electromagnetic energy which is transmitted through body tissue. In an example, an electromagnetic energy sensor can be used in combination with an electromagnetic energy emitter which emits electromagnetic energy into body tissue. In an example, an electromagnetic energy sensor can measure the amount of electromagnetic energy from an electromagnetic energy emitter which is transmitted through body tissue. In another example, an electromagnetic energy sensor can measure patterns of electromagnetic energy which are naturally created by body tissue and/or body organs during preparation for food consumption and/or during food consumption. In an example, an electromagnetic energy sensor can measure patterns of electromagnetic energy which are naturally created by nerves and/or muscles when a person is near food, purchasing food, ordering food, preparing food, and/or consuming food.

In an example, this invention can further comprise one or more electroencephalographic (EEG) sensors which are integrated into eyewear. In an example, an EEG sensor can be a dry electrode. In an example, one or more EEG sensors can be held in electromagnetic communication with a person's head by the support member of this invention. In an example, these one or more EEG sensors can be held in electromagnetic communication with a person's head by electronically-functional eyewear. In an example, an EEG can collect data which reveals patterns of electromagnetic brain activity which are associated with preparation for food consumption and/or food consumption.

In an example, one or more EEG sensors can be placed at locations selected from the group consisting of: FP1, FPz, FP2, AF7, AF5, AF3, AFz, AF4, AF6, AF8, F7, F5, F3, F1, Fz, F2, F4, F6, F8, FT7, FC5, FC3, FC1, FCz, FC2, FC4, FC6, and FT8. In an more general example, one or more EEG sensors can be placed at locations selected from the group consisting of: FP1, FPz, FP2, AF7, AF5, AF3, AFz, AF4, AF6, AF8, F7, F5, F3, F1, Fz, F2, F4, F6, F8, FT7, FC5, FC3, FC1, FCz, FC2, FC4, FC6, FT8, T3/T7, C3, C4, C1, Cz, C2, C5, C6, T4/T8, TP7, CP5, CP3, CP1, CPz, CP2, CP4, CP6, TP8, T5/P7, P5, P3, P1, Pz, P2, P4, P6, T6/P8, PO7, PO5, PO3, POz, PO4, PO6, PO8, O1, Oz, and O2.

In an example, an EEG sensor can collect data on electromagnetic energy patterns and/or electromagnetic fields which are naturally generated by electromagnetic brain activity. In an example, an EEG sensor can be used in combination with an electromagnetic energy emitter. In an example, an electromagnetic energy emitter can be in contact with the surface of a person's head. In an example, an EEG sensor can measure the conductivity, voltage, resistance, and/or impedance of electromagnetic energy emitted from an electromagnetic energy emitter and transmitted through a portion of a person's head.

In an example, this device can comprise a plurality of EEG sensors which collect data concerning electromagnetic brain activity from different selected locations. In an example, an EEG sensor can measure the conductivity, voltage, resistance, or impedance of electromagnetic energy that is transmitted between two locations. In an example, the locations for a plurality of EEG sensors can be selected from the group consisting of: FP1, FPz, FP2, AF7, AF5, AF3, AFz, AF4, AF6, AF8, F7, F5, F3, F1, Fz, F2, F4, F6, F8, FT7, FC5, FC3, FC1, FCz, FC2, FC4, FC6, FT8, T3/T7, C3, C4, C1, Cz, C2, C5, C6, T4/T8, TP7, CP5, CP3, CP1, CPz, CP2, CP4, CP6, TP8, T5/P7, P5, P3, P1, Pz, P2, P4, P6, T6/P8, P07, P05, P03, POz, PO4, P06, P08, 01, Oz, and 02. In an example, a plurality of EEG sensors can be located in a symmetric manner with respect to the central longitudinal right-vs.-left plane of a person's head. In an example, electromagnetic brain activity data from a selected recording location (relative to a reference location) is a “channel.” In an example, electromagnetic brain activity data from multiple recording places is a “montage.”

In an example, data from one or more EEG sensors can be filtered to remove artifacts before the application of a primary statistical method. In an example, a filter can be used to remove electromagnetic signals from eye blinks, eye flutters, or other eye movements before the application of a primary statistical method. In an example, a notch filter can be used as well to remove 60 Hz artifacts caused by AC electrical current. In various examples, one or more filters can be selected from the group consisting of: a high-pass filter, a band-pass filter, a loss-pass filter, an electromyographic activity filter, a 0.5-1 Hz filter, and a 35-70 Hz filter. In an example, data from an EEG sensor can be analyzed using Fourier transformation methods in order to identify repeating energy patterns in clinical frequency bands. In an example, these clinical frequency bands can be selected from the group consisting of: Delta, Theta, Alpha, Beta, and Gamma. In an example, the relative and combinatorial power levels of energy in two or more different clinical frequency bands can be analyzed.

In an example, a primary statistical method can comprise finding the mean or average value of data from one or more brain activity channels during a period of time. In an example, a statistical method can comprise identifying a significant change in the mean or average value of data from one or more brain activity channels. In an example, a statistical method can comprise finding the median value of data from one or more brain activity channels during a period of time. In an example, a statistical method can comprise identifying a significant change in the median value of data from one or more brain activity channels. In an example, a statistical method can comprise identifying significant changes in the relative mean or median data values among multiple brain activity channels. In an example, a statistical method can comprise identifying significant changes in mean data values from a first set of electrode locations relative to mean data values from a second set of electrode locations. In an example, a statistical method can comprise identifying significant changes in mean data recorded from a first region of the brain relative to mean data recorded from a second region of the brain.

In an example, a primary statistical method can comprise finding the minimum or maximum value of data from one or more brain activity channels during a period of time. In an example, a statistical method can comprise identifying a significant change in the minimum or maximum value of data from one or more brain activity channels. In an example, a statistical method can comprise identifying significant changes in the relative minimum or maximum data values among multiple brain activity channels. In an example, a statistical method can comprise identifying significant changes in minimum or maximum data values from a first set of electrode locations relative to minimum or maximum data values from a second set of electrode locations. In an example, a statistical method can comprise identifying significant changes in minimum or maximum data values recorded from a first region of the brain relative to minimum or maximum data values recorded from a second region of the brain.

In an example, a primary statistical method can comprise finding the variance or the standard deviation of data from one or more brain activity channels during a period of time. In an example, a statistical method can comprise identifying a significant change in the variance or the standard deviation of data from one or more brain activity channels. In an example, a statistical method can comprise identifying significant changes in the covariation and/or correlation among data from multiple brain activity channels. In an example, a statistical method can comprise identifying significant changes in the covariation or correlation between data from a first set of electrode locations relative and data from a second set of electrode locations. In an example, a statistical method can comprise identifying significant changes in the covariation or correlation of data values recorded from a first region of the brain and a second region of the brain.

In an example, a primary statistical method can comprise finding the mean amplitude of waveform data from one or more channels during a period of time. In an example, a statistical method can comprise identifying a significant change in the mean amplitude of waveform data from one or more channels. In an example, a statistical method can comprise identifying significant changes in the relative means of wave amplitudes from one or more channels. In an example, a statistical method can comprise identifying significant changes in the amplitude of electromagnetic signals recorded from a first region of the brain relative to the amplitude of electromagnetic signals recorded from a second region of the brain.

In an example, a primary statistical method can comprise finding the power of waveform brain activity data from one or more channels during a period of time. In an example, a statistical method can comprise identifying a significant change in the power of waveform data from one or more channels. In an example, a statistical method can comprise identifying significant changes in the relative power levels of one or more channels. In an example, a statistical method can comprise identifying significant changes in the power of electromagnetic signals recorded from a first region of the brain relative to the power of electromagnetic signals recorded from a second region of the brain.

In an example, a primary statistical method can comprise finding a frequency or frequency band of waveform and/or rhythmic brain activity data from one or more channels which repeats over time. In an example, Fourier transformation methods can be used to find a frequency or frequency band of waveform and/or rhythmic data which repeats over time. In an example, a statistical method can comprise decomposing a complex waveform into a combination of simpler waveforms which each repeat at a different frequency or within a different frequency band. In an example, Fourier transformation methods can be used to decomposing a complex waveform into a combination of simpler waveforms which each repeat at a different frequency or within a different frequency band.

In an example, a primary statistical method can comprise identifying significant changes in the amplitude, power level, phase, frequency, and/or oscillation of waveform data from one or more channels. In an example, a primary statistical method can comprise identifying significant changes in the amplitude, power level, phase, frequency, and/or oscillation of waveform data within a selected frequency band. In an example, a primary statistical method can comprise identifying significant changes in the relative amplitudes, power levels, phases, frequencies, and/or oscillations of waveform data among different frequency bands. In various examples, these significant changes can be identified using Fourier transformation methods.

In an example, brainwaves (or other rhythmic, cyclical, and/or repeating electromagnetic signals associated with brain activity) can be measured and analyzed using one or more clinical frequency bands. In an example, complex repeating waveform patterns can be decomposed and identified as a combination of multiple, simpler repeating wave patterns, wherein each simpler wave pattern repeats within a selected clinical frequency band. In an example, brainwaves can be decomposed and analyzed using Fourier transformation methods. In an example, brainwaves can be measured and analyzed using five common clinical frequency bands: Delta, Theta, Alpha, Beta, and Gamma.

In an example, Delta brainwaves can be measured and analyzed within the frequency band of 1 to 4 Hz. In various examples, Delta brainwaves (or other rhythmic, cyclical, and/or repeating electromagnetic signals associated with brain activity) can be measured and analyzed within a frequency band selected from the group consisting of: 0.5-3.5 Hz, 0.5-4 Hz, 1-3 Hz, 1-4 Hz, and 2-4 Hz. In an example, Theta brainwaves can be measured and analyzed within the frequency band of 4 to 8 Hz. In various examples, Theta brainwaves or other rhythmic, cyclical, and/or repeating electromagnetic signals associated with brain activity can be measured and analyzed within a frequency band selected from the group consisting of: 3.5-7 Hz, 3-7 Hz, 4-7 Hz, 4-7.5 Hz, 4-8 Hz, and 5-7 Hz.

In an example, Alpha brainwaves can be measured and analyzed within the frequency band of 7 to 14 Hz. In various examples, Alpha brainwaves or other rhythmic, cyclical, and/or repeating electromagnetic signals associated with brain activity can be measured and analyzed within a frequency band selected from the group consisting of: 7-13 Hz, 7-14 Hz, 8-12 Hz, 8-13 Hz, 7-11 Hz, 8-10 Hz, and 8-10 Hz. In an example, Beta brainwaves can be measured and analyzed within the frequency band of 12 to 30 Hz. In various examples, Beta brainwaves or other rhythmic, cyclical, and/or repeating electromagnetic signals associated with brain activity can be measured and analyzed within a frequency band selected from the group consisting of: 11-30 Hz, 12-30 Hz, 13-18 Hz, 13-22 Hz, 13-26 Hz, 13-26 Hz, 13-30 Hz, 13-32 Hz, 14-24 Hz, 14-30 Hz, and 14-40 Hz. In an example, Gamma brainwaves can be measured and analyzed within the frequency band of 30 to 100 Hz. In various examples, Gamma brainwaves or other rhythmic, cyclical, and/or repeating electromagnetic signals associated with brain activity can be measured and analyzed within a frequency band selected from the group consisting of: 30-100 Hz, 35-100 Hz, 40-100 Hz, and greater than 30 Hz.

In an example, data concerning electromagnetic brain activity which is collected by one or more EEG sensors can be analyzed using one or more statistical methods selected from the group consisting of: multivariate linear regression or least squares estimation; factor analysis; Fourier transformation; mean; median; multivariate logit; principal components analysis; spline function; auto-regression; centroid analysis; correlation; covariance; decision tree analysis; Kalman filter; linear discriminant analysis; linear transform; logarithmic function; logit analysis; Markov model; multivariate parametric classifiers; non-linear programming; orthogonal transformation; pattern recognition; random forest analysis; spectroscopic analysis; variance; artificial neural network; Bayesian filter or other Bayesian statistical method; chi-squared; eigenvalue decomposition; logit model; machine learning; power spectral density; power spectrum analysis; probit model; time-series analysis; inter-band mean; inter-band ratio; inter-channel mean; inter-channel ratio; inter-montage mean; inter-montage ratio; multi-band covariance analysis; multi-channel covariance analysis; and analysis of wave frequency, wave frequency band, wave amplitude, wave phase, and wave form or morphology. In an example, wave form or morphology can be identified from the group consisting of: simple sinusoidal wave, composite sinusoidal wave, simple saw-tooth wave, composite saw-tooth wave, biphasic wave, tri-phasic wave, and spike.

In an example, this invention can further comprise an electromyographic (EMG) sensor which detects patterns of electromagnetic muscle activity (such as chewing, swallowing, or stomach movement) which are associated with preparation for food consumption and/or food consumption. In an example, this invention can further comprise an electrogastrographic (EGG) sensor which detects patterns of electromagnetic stomach activity which are associated with preparation for food consumption and/or food consumption. In an example, this sensor can be a tissue impedance sensor which detects changes in body tissue impedance which are associated with food consumption. In an example, this sensor can be an electrocardiographic (ECG) sensor which detects electromagnetic heart activity which is associated with food consumption. In an example, this sensor can be a peripheral nervous system sensor which detects peripheral nervous system activity which is associated with food consumption and/or preparation for food consumption.

In an example, an imaging member can automatically start taking pictures and/or recording images when data from one or more wearable or implantable electromagnetic energy sensors indicates that a person is consuming food or will probably be consuming food soon. In an example, a wearable sensor can be selected from the group consisting of: action potential sensor, neural impulse sensor, and/or neurosensor; electrocardiographic (ECG) sensor and/or electromagnetic heart activity sensor; electrochemical sensor; electroconductive fiber, electrogoniometer, piezoelectric sensor, electromagnetic conductivity sensor; electroencephalographic (EEG) sensor and/or electromagnetic brain activity sensor; electrogastrographic (EGG) sensor and/or gastric activity sensor; electromyographic (EMG) sensor and/or electromagnetic muscle activity sensor; electrooculographic (EOG) sensor; electroosmotic sensor, electrophoresis sensor, electroporation sensor; galvanic skin response (GSR) sensor, tissue impedance sensor, tissue resistance sensor, tissue conductivity sensor, skin conductance sensor, skin impedance sensor, variable impedance sensor, voltmeter, variable resistance sensor, electromagnetic impedance sensor, and/or electromagnetic resistance sensor; hemoencephalography (HEG) monitor; magnetic field sensor, magnetometer, and/or Hall-effect sensor; micro electromechanical system (MEMS) sensor; and radio frequency (RF) sensor.

In an example, this invention can further comprise one or more motion sensors and/or location sensors which are used to detect food consumption or probable food consumption in the near future. In an example, an imaging member can automatically start taking pictures and/or recording images when data from a wearable or implantable motion sensor indicates that a person is consuming food or will probably be consuming food soon. In an example, a motion sensor can be selected from the group consisting of: accelerometer, gyroscope, inclinometer, tilt sensor, strain gauge, pressure sensor, and electrogoniometer. In an example, one or more motion and/or location sensors can be selected from the group consisting of: inertial sensor, accelerometer, gyroscope, kinematic sensor, tilt sensor, inclinometer, and/or vibration sensor; air pressure sensor, bend sensor, electrogoniometer, force sensor, goniometer, mechanical chewing sensor, mechanical swallowing sensor, microcantilever sensor, piezoelectric sensor, posture sensor, pressure sensor, strain gauge, manometer, and stretch sensor; airflow sensor, altimeter, barometer, blood flow monitor, blood pressure monitor, compass, flow sensor, gesture recognition sensor, global positioning system (GPS) sensor, micro electromechanical system (MEMS) sensor, microfluidic sensor, nanotube sensor, and peak flow sensor.

In an example, a motion sensor which is used to trigger food imaging can be part of a wearable device selected from the group consisting of: watch, wrist band, bracelet, bangle, wrist cuff, finger ring, electronically-functional glove, arm band, smart shirt, smart pants, shoe, sock, electronically-functional necklace, electronically-functional collar, electronically-functional button, electronically-function pin, electronically-functional pendant or dog tags, ear ring, hearing aid, ear bud or insert, nose ring, tongue ring, dental insert or attachment, palatal insert or attachment, electronically-functional bandage, electronically-functional tattoo, and hat.

In an example, an imaging member can automatically start taking pictures and/or recording images when data from a wrist-worn motion sensor shows a pattern of hand and/or arm motion which is generally associated with food consumption. In an example, this pattern of hand and/or arm motion can comprise: hand movement toward a reachable food source; hand movement up to a person's mouth; lateral motion and/or hand rotation to bring food into the mouth; and hand movement back down to the original level. In an example, electronically-functional eyewear can be in wireless communication with a motion sensor which is worn on a person's wrist, finger, hand, or arm. In an example, this motion sensor can detect hand, finger, wrist, and/or arm movements which indicate that a person is preparing food for consumption and/or bringing food up to their mouth.

In an example, an imaging member can automatically start taking pictures and/or recording images when data from a neck-worn or head-worn motion sensor shows a pattern of jaw, tongue, mouth, and/or neck motions which is generally associated with food consumption. In an example, an imaging member can automatically start taking pictures and/or recording images based on data from a chewing sensor and/or swallow sensor. In an example, analysis of data from a neck-worn or head-worn motion sensor can differentiate between motions which are associated with food consumption versus motions which are associated with talking, coughing, yawning, and swallowing that are not part of food consumption.

In an example, this invention can further comprise a wearable or implantable sonic energy sensor. In an example, this invention can further comprise a wearable or implantable sonic energy sensor, wherein an imaging member is automatically activated or triggered to start taking pictures and/or record images when data from this sonic energy sensor indicates that a person is near food, purchasing food, ordering food, preparing food, and/or consuming food. In an example, a sonic energy sensor can be a microphone. In an example, an imaging member can automatically start taking pictures and/or recording images when data from one or more wearable sonic energy sensors indicates that a person is near food, purchasing food, ordering food, preparing food, and/or consuming food. In an example, one or more wearable sonic energy sensors can be selected from the group consisting of: microphone, speech recognition interface, voice recognition interface, breathing sound monitor, sound-based chewing sensor, sound-based swallowing monitor, ambient sound sensor, ultrasonic emitter and sensor, and digital stethoscope.

In an example, an imaging member can automatically start taking pictures and/or recording images when data from a sonic energy sensor indicates that a person is chewing and/or swallowing. In an example, an imaging member can automatically start taking pictures and/or recording images when data from a sonic energy sensor indicates that a person is near food, purchasing food, ordering food, preparing food, and/or consuming food. In an example, an imaging member can automatically start taking pictures and/or recording images when voice recognition analysis of data from a sonic energy sensor indicates that a person is purchasing, ordering, preparing, and/or eating food. In an example, an imaging member can automatically start taking pictures and/or recording images when data from the transmission and/or reflection of ultrasonic energy with respect to body tissue indicates that a person is consuming food.

In an example, this invention can further comprise a wearable or implanted light energy sensor. In an example, this invention can further comprise a wearable or implanted light energy sensor, wherein an imaging member is automatically activated or triggered to start taking pictures and/or recording images when data from the light energy sensor indicates that a person is near food, purchasing food, ordering food, preparing food, and/or consuming food. In an example, a light energy sensor can be a second imaging member which is in a different location than the primary imaging member which is incorporated into eyewear. In an example, a light energy sensor can be a second camera which is worn on a person's finger, hand, wrist, arm, neck, or torso. In an example, a light energy sensor can be part of a smart watch, smart necklace, or smart shirt. In an example, a light energy sensor can be attached to an upper body garment. In an example, electronically-functional eyewear with an integrated camera in combination with a separately-located second wearable camera can comprise a system for monitoring a person's food consumption. In an example, having food images from the perspectives of two cameras at different locations can provide more accurate estimation of food types and/or quantities than images from one camera alone.

In an example, a light energy sensor can measure the amount and/or spectrum of light energy which is transmitted through body tissue. In an example, a light energy sensor can measure the amount and/or spectrum of light energy which is reflected from body tissue. In an example, a light energy sensor can be used in combination with a light energy emitter. In an example, a light energy sensor can measure the amount and/or spectrum of light energy from a light energy emitter after it has been transmitted through, or reflected from, body tissue. In an example, when data from such a light energy sensor indicates that a person is probably consuming food, then this automatically triggers a (primary) imaging member to start taking pictures and/or recording images.

In an example, a light energy sensor can be a spectroscopic sensor. In an example, a spectroscopic sensor can be worn by a person. In an example, a spectroscopic sensor can be held by a person in proximity to food. In an example, a spectroscopic sensor can be part of (or attached to) electronically-functional eyewear. In an example, a spectroscopic sensor can be part of (or attached to) a smart watch or other wrist-worn device. In an example, a spectroscopic sensor can be in wireless communication with electronically-functional eyewear. In an example, one or more light energy sensors can be selected from the group consisting of: spectrometry sensor, chromatography sensor, color sensor, analytical chromatography sensor, gas chromatography sensor, infrared spectroscopy sensor, ion mobility spectroscopic sensor, light-spectrum-analyzing sensor, mass spectrometry sensor, near infrared spectroscopy sensor, Raman spectroscopy sensor, spectral analysis sensor, spectrophotometric sensor, spectroscopy sensor, and white light spectroscopy sensor. In an example, data from a separate spectroscopic sensor can be combined with data from an imaging member to provide more accurate estimation of food types, food quantities, and food ingredients.

In an example, an imaging member can automatically start taking pictures and/or recording images when data from one or more wearable light energy sensors indicates that a person is consuming food or will probably be consuming food soon. In an example, a light energy sensor can read a code on food packaging and/or a menu which identifies a type and/or quantity of food. In an example, a light energy sensor can read a bar code on food packaging. In an example, a light energy sensor can read a food packaging and/or menu code which identifies types and/or quantities of food ingredients. In an example, a light energy sensor can read a food packaging and/or menu code or label which identifies types and/or quantities of food nutrients. In an example, a light energy sensor can be selected from the group consisting of: bar code reader, digital code reader, food package identification sensor, food logo recognition sensor, nutritional label reader, restaurant menu reader, optical text scanner, package reader, RFID sensor, menu scanner, food purchase code reader, and UPC code reader.

In an example, one or more light energy sensors whose data is analyzed to trigger activation of an imaging member can be selected from the group consisting of: separately-located camera and/or supplemental imaging device, ambient light sensor, chemiluminescence sensor, coherent light sensor, electro-optical sensor, eye gaze tracker, fluorescence sensor, holographic imaging device, infrared light sensor, light intensity sensor, near-infrared light sensor, optical glucose sensor, optoelectronic sensor, photochemical sensor, photoelectric sensor, photometer, photoplethysmographic (PPG) sensor, thermoluminescence sensor, ultraviolet light sensor, and video recorder. In an example, this invention can further comprise one or more light-sensing or light-emitting members selected from the group consisting of: birefringent material member, coherent light image projector, crystal, display screen, eye-tracking sensor, fiber optic array, fiber optic bend sensor, image display, infrared light emitter, infrared projector, laser, lens, light display matrix, light emitting diode (LED), light emitting diode (LED) array, light-conducting fiber, light-emitting member, liquid crystal display (LCD), metamaterial member, microlens array, micro-mirror array, non-coherent-light image projector, optical emitter, optical fiber, optochemical sensor, optoelectronic lens, variable-focal-length lens, and wearable image display.

In an example, this invention can further comprise one or more wearable or implanted biochemical sensors. In an example, data from one or more biochemical sensors can be analyzed to detect when a person is near food, purchasing food, ordering food, preparing food, and/or consuming food. In an example, an imaging member can be automatically triggered to begin taking pictures and/or recording images when data from one or more biochemical sensors indicates that a person is near food, purchasing food, ordering food, preparing food, and/or consuming food. In an example, data from one or more biochemical sensors can be jointly analyzed with food images recorded by an imaging member to identify food types and to estimate food quantities more accurately than either data source alone. In an example, data from one or more biochemical sensors can be jointly analyzed with food images recorded by an imaging member to identify ingredient types and quantities more accurately than either data source alone.

In an example, a biochemical sensor can be incorporated into a wearable device separate from eyewear, wherein this device is selected from the group consisting of: smart watch, wrist band, finger ring, bangle, arm band, necklace, palatal implant, dental implant, dental appliance, tongue ring, and nose ring. In an example, a separate wearable device and electronically-functional eyewear can together comprise a system for monitoring a person's food consumption. In an example, a biochemical sensor can be incorporated into an implanted device, wherein this device is in liquid or gaseous communication with a person's oral cavity, nasal passages, or (other locations along) the person's gastrointestinal tract. In an example, a biochemical sensor can extract and analyze microsamples of body fluid or body tissue. In an example, an implanted biochemical sensor and electronically-functional eyewear can together comprise a system for monitoring a person's food consumption. In an example, a biochemical sensor can be incorporated into a handheld food utensil or food probe, wherein this utensil is brought into liquid or gaseous communication with food. In an example, a handheld food utensil or probe and electronically-functional eyewear can together comprise a system for monitoring a person's food consumption.

In an example, one or more biochemical sensors whose data is used to trigger an imaging member and/or to improve the accuracy of food type and quantity estimation can be selected from the group consisting of: glucometer, glucose sensor, glucose monitor, blood glucose monitor, cellular fluid glucose monitor, spectroscopic sensor, food composition analyzer, oximeter, oximetry sensor, pulse oximeter, tissue oximetry sensor, tissue saturation oximeter, wrist oximeter, oxygen consumption monitor, oxygen level monitor, oxygen saturation monitor, ambient air sensor, gas composition sensor, blood oximeter, ear oximeter, cutaneous oxygen monitor, cerebral oximetry monitor, capnography sensor, carbon dioxide sensor, carbon monoxide sensor, artificial olfactory sensor, smell sensor, moisture sensor, humidity sensor, hydration sensor, skin moisture sensor, chemiresistor sensor, chemoreceptor sensor, electrochemical sensor, amino acid sensor, cholesterol sensor, body fat sensor, osmolality sensor, pH level sensor, sodium sensor, taste sensor, and microbial sensor.

In an example, this invention can further comprise a wearable or implanted thermal energy sensor. In an example, changes in temperature can be used to better identify when a person is near food, preparing food, and/or consuming food. In an example, changes in temperature can be used to improve measurement of food types and quantities. In an example, a thermal energy sensor can be selected from the group consisting of: ambient temperature sensor, body temperature sensor, skin temperature sensor, temperature sensor, thermistor, thermometer, and thermopile.

In an example, this invention can further comprise a physiological and/or organ function sensor. In an example, changes in physiological and/or organ function can be used to better identify when a person is near food, preparing food, anticipating consuming food, and/or consuming food. In an example, changes in physiological and/or organ function can be used to improve measurement of food types and quantities. In an example, a physiological and/or organ function sensor can be selected from the group consisting of: blood pressure sensor, breathing monitor, cardiac function monitor, cardiotachometer, cardiovascular monitor, gastric acid sensor, heart rate monitor, heart rate sensor, heart sensor, pneumography sensor, pulmonary function monitor, pulse monitor, respiration rate monitor, respiration sensor, respiratory function monitor, spirometry monitor, stomach sensor, and tidal volume sensor.

In an example, this invention can further comprise a data processing unit. With respect to FIGS. 41 through 60, a data processing unit can comprise one or more of the variations which we now discuss. In an example, a data processing unit can be part of eyewear. In an example, a data processing unit can be part of a support member, an optical member, or imaging member. In an example, a data processing unit can be in a remote location with which electronically-functional eyewear is in wireless communication. In an example, analysis of food pictures or images can occur within a data processing unit. In an example, pictures or images from an imaging member can be analyzed locally in a data processing unit which is part of electronically-functional eyewear. In an example, pictures or images from an imaging member can be analyzed remotely in a separate device with which an imaging member is in electronic communication.

In an example, this invention can further comprise one or more components selected from the group consisting of: data processing unit, power source, data communication component, human-to-computer user interface, computer-to-human interface, digital memory, one or more additional wearable sensors, one or more implanted sensors, and an external electromagnetic energy emitter. In an example, one or more of the components selected from this group can be connected to, attached to, and/or integrated into the support member. In an example, one or more of the components selected from this group can be connected to, attached to, and/or integrated into eyewear.

In an example, a data processing unit can perform one or more functions selected from the group consisting of: convert analog sensor signals to digital signals, filter sensor signals, amplify sensor signals, analyze sensor data, run software programs, and store data in memory. In an example, a data processing unit can analyze data using one or more statistical methods selected from the group consisting of: multivariate linear regression or least squares estimation; factor analysis; Fourier Transformation; mean; median; multivariate logit; principal components analysis; spline function; auto-regression; centroid analysis; correlation; covariance; decision tree analysis; Kalman filter; linear discriminant analysis; linear transform; logarithmic function; logit analysis; Markov model; multivariate parametric classifiers; non-linear programming; orthogonal transformation; pattern recognition; random forest analysis; spectroscopic analysis; variance; artificial neural network; Bayesian filter or other Bayesian statistical method; chi-squared; eigenvalue decomposition; logit model; machine learning; power spectral density; power spectrum analysis; probit model; and time-series analysis.

In an example, a power source which is part of this invention can be a battery. In an example, a power source can harvest, transduce, or generate electrical energy from kinetic energy, thermal energy, biochemical energy, ambient light energy, and/or ambient electromagnetic energy. In an example, a power source can comprise: power from a source that is internal to the device during regular operation (such as an internal battery, capacitor, energy-storing microchip, wound coil or spring); power that is obtained, harvested, or transduced from a source other than a person's body that is external to the device (such as a rechargeable battery, electromagnetic inductance from external source, solar energy, indoor lighting energy, wired connection to an external power source, ambient or localized radiofrequency energy, or ambient thermal energy); and power that is obtained, harvested, or transduced from a person's body (such as kinetic or mechanical energy from body motion, electromagnetic energy from a person's body, or thermal energy from a person's body).

In an example, a data communication component can perform one or more functions selected from the group consisting of: transmit and receive data via Bluetooth, WiFi, Zigbee, or other wireless communication modality; transmit and receive data to and from a mobile electronic device such as a cellular phone, mobile phone, smart phone, electronic tablet; transmit and receive data to and from a separate wearable device such as a smart watch or smart clothing; transmit and receive data to and from the internet; send and receive phone calls and electronic messages; and transmit and receive data to and from an implantable medical device.

In an example, a data communication component can be in wireless communication with a separate mobile device selected from the group consisting of: smart phone, mobile phone, or cellular phone; PDA; electronic tablet; electronic pad; and other electronically-functional handheld device. In an example, a data communication component can be in wireless communication with a relatively fixed-location device selected from the group consisting of: laptop computer, desktop computer, internet terminal, smart appliance, home control system, and other fixed-location electronic communication device. In an example, a data communication component can communicate with one or more other devices selected from the group consisting of: a communication tower or satellite; an appliance, home environment control system, and/or home security system; a laptop or desktop computer; a smart phone or other mobile communication device; a wearable cardiac monitor; a wearable pulmonary activity monitor; an implantable medical device; an internet server; and another type of wearable device or an array of wearable sensors.

In an example, a human-to-computer interface can further comprise one or more members selected from the group consisting of: buttons, knobs, dials, or keys; display screen; gesture-recognition interface; microphone; physical keypad or keyboard; virtual keypad or keyboard; speech or voice recognition interface; touch screen; EMG-recognition interface; and EEG-recognition interface. In an example, a computer-to-human interface can further comprise one or more members selected from the group consisting of: a display screen; a speaker or other sound-emitting member; a myostimulating member; a neurostimulating member; a speech or voice recognition interface; a synthesized voice; a vibrating or other tactile sensation creating member; MEMS actuator; an electromagnetic energy emitter; an infrared light projector; an LED or LED array; and an image projector.

In an example, this invention can further comprise methods of analyzing food pictures and/or images from the imaging member in order to estimate types and/or quantities of foods, ingredients, and/or nutrients. In an example, these analytical methods can be performed within a data processing unit. In an example, one or more methods for analyzing pictures or images from the imaging member can be selected from the group consisting of: pattern recognition or identification; human motion recognition or identification; face recognition or identification; gesture recognition or identification; food recognition or identification; word recognition or identification; logo recognition or identification; bar code recognition or identification; volumetric or 3D modeling; and spectroscopic analysis. In an example, the results of these methods can be used to provide feedback to the person in order to modify the person's consumption of food. In an example, this invention can monitor the cumulative consumption of one or more specific types of foods, ingredients, and/or nutrients during a period of time. In an example, this invention can use estimates of the types and/or quantities of foods, ingredients, and/or nutrients to modify a person's nutritional intake.

In an example, this invention can further comprise methods for analysis of food pictures and/or images which differentiate between healthy and unhealthy food. In an example, this invention can provide feedback or activate mechanisms which selectively reduce a person's consumption of unhealthy food. In an example, this invention can activate mechanisms which selectively reduce a person's absorption of nutrients from unhealthy food which the person consumes. In an example, this invention can provide feedback or activate mechanisms which selectively increase a person's consumption of healthy food. In an example, this invention can activate mechanisms which selectively increase a person's absorption of nutrients from healthy food which the person consumes.

In an example, pictures and/or images from an imaging member can be analyzed to identify the types and/or quantities of food which are located anywhere within the field of view of the imaging member. In an example, pictures and/or images from an imaging member can be analyzed to identify the types and/or quantities of food to which a person has access. In an example, pictures and/or images from an imaging member can be analyzed to identify the types and/or quantities of food which are located within the field of view of the imaging member and within a selected distance from a person. In an example, pictures and/or images from an imaging member can be analyzed to identify the types and/or quantities of food which are located within the field of view of the imaging member and within reach of a person.

In an example, pictures and/or images from an imaging member can be analyzed to identify the types and/or quantities of food which are near a person's hand, on a utensil held by the person, within a beverage container held by the person, or on a dish near the person. In an example, pictures and/or images from an imaging member can be analyzed to identify the types and/or quantities of food which are brought up to a person's mouth. In an example, pictures and/or images from an imaging member can be analyzed to identify the types and/or quantities of food which a person chews and/or swallows. In an example, pictures and/or images from an imaging member can be analyzed to identify the types and/or quantities of food which a person consumes.

In an example, pictures and/or images of food can be analyzed within a data processing unit which is part of electronically-functional eyewear. In an example, pictures and/or images of food can be analyzed within a data processing unit which is part of (or attached to) a support member. In an example, pictures and/or images of food can be analyzed in a remote device. In an example, the remote device can be in wireless communication with a data transmitter, data receiver, and/or data processing unit which is part of (or attached to) electronically-functional eyewear. In an example, there can be a chain of wireless communication between an imaging member and a remote data processing unit which analyzes food images.

In an example, this invention can comprise a method for measuring food consumption which involves taking multiple pictures of the same portion of food. In an example, this method can include taking pictures of a portion of food from at least two different angles in order to segment a meal into different types of foods, estimate the three-dimensional volume of each type of food, and/or control for lighting and shading differences. In an example, an imaging member can take pictures of food from multiple perspectives to create a virtual three-dimensional model of food in order to determine food volume. In an example, an imaging member can estimate the quantities of specific foods from pictures or images of those foods by volumetric analysis of food from multiple perspectives and/or by three-dimensional modeling of food from multiple perspectives.

In an example, an imaging member can take multiple still pictures or moving pictures of food. In an example, an imaging member can take multiple pictures of food from different angles in order to perform three-dimensional analysis or modeling of the food to better determine the volume of food. In an example, an imaging member can take multiple pictures of food from different angles in order to better control for differences in lighting and portions of food that are obscured from some perspectives. In an example, an imaging member can take multiple pictures of food from different angles in order to perform three-dimensional modeling or volumetric analysis to determine the three-dimensional volume of food in the picture. In an example, an imaging member can take multiple pictures of food at different times, such as before and after an eating event, in order to better determine how much food the person actually ate (versus the amount of food served). In an example, changes in the volume of food in sequential pictures (before and after consumption) can be compared to determine the volume of food actually consumed.

In an example, an imaging member can use an object of known size within its field of view as a fiduciary marker in order to measure the size or scale of food. In an example, an imaging member can use projected laser beams to create a virtual or optical fiduciary marker in order to measure food size or scale. In an example, images of food can be automatically analyzed in order to identify the types and quantities of food consumed. In an example, pictures of food taken by an imaging member or other picture-taking device can be automatically analyzed to estimate the types and amounts of specific foods, ingredients, or nutrients that a person is consumes. In an example, image analysis can comprise adjusting, normalizing, or standardizing image elements for better food segmentation, identification, and volume estimation. These elements can include: color, texture, shape, size, context, geographic location, adjacent food, place setting context, and temperature (infrared). In an example, specific foods can be identified from pictures or images by image segmentation, color analysis, texture analysis, and pattern recognition.

In various examples, automatic identification of food types and quantities can be based on: color and texture analysis; image segmentation; image pattern recognition; volumetric analysis based on a fiduciary marker or other object of known size; and/or three-dimensional modeling based on pictures from multiple perspectives. In an example, a device can collect food images that are used to extract a vector of food parameters (such as color, texture, shape, and size) that are automatically associated with vectors of food parameters in a database of such parameters for food identification. In an example, attributes of food in an image can be represented by a multi-dimensional food attribute vector. In an example, this food attribute vector can be statistically compared to the attribute vector of known foods in order to automate food identification. In an example, multivariate analysis can be done to identify the most likely identification category for a particular portion of food in an image. In various examples, a multi-dimensional food attribute vector can include attributes selected from the group consisting of: food color; food texture; food shape; food size or scale; geographic location of selection, purchase, or consumption; timing of day, week, or special event; common food combinations or pairings; image brightness, resolution, or lighting direction; infrared light reflection; spectroscopic analysis; and person-specific historical eating patterns.

In an example, this invention can further comprise (or be in electronic communication with) a database of different types of foods (and/or food portions) and their associated ingredients, nutrients, and/or calories. Such a database can be used to convert a type and quantity of food (and/or portion of that food) into ingredients, nutrients, and/or calories. In an example, one or more nutrients can be selected from the group consisting of: a specific sugar, a specific carbohydrate, a specific fat, a specific cholesterol, a specific sodium compound, a category of sugars, a category of carbohydrates, a category of fats, a category of cholesterols, a category of sodium compounds, sugars in general, carbohydrates in general, fats in general, cholesterols in general, and sodium compounds in general. In an example, some of the nutrients can be classified as unhealthy in general or when consumed in an excessive quantity. In an example, some of the nutrients can be classified as healthy in general or when consumed in a desired quantity.

In an example, food images from an imaging member can be automatically associated with food images in a food image database for the purposes of food identification. In an example, specific ingredients or nutrients that are associated with these selected types of food can be estimated based on a database linking foods to ingredients and nutrients. In another example, specific ingredients or nutrients can be measured directly. In various examples, a device for measuring consumption of food, ingredient, or nutrients can directly (or indirectly) measure consumption at least one selected type of food, ingredient, or nutrient.

In an example, a database of different types of foods can include one or more elements selected from the group consisting of: food color, food name, food packaging bar code or nutritional label, food packaging or logo pattern, food picture (individually or in combinations with other foods), food shape, food texture, food type, common geographic or intra-building locations for serving or consumption, common or standardized ingredients (per serving, per volume, or per weight), common or standardized nutrients (per serving, per volume, or per weight), common or standardized size (per serving), common or standardized number of calories (per serving, per volume, or per weight), common times or special events for serving or consumption, and commonly associated or jointly-served foods.

In an example, a picture of a meal as a whole can be automatically segmented into portions of different types of food for comparison with different types of food in a food database. In an example, the boundaries between different types of food in a picture of a meal can be automatically determined to segment the meal into different food types before comparison with pictures in a food database. In an example, a picture of a meal with multiple types of food can be compared as a whole with pictures of meals with multiple types of food in a food database. In an example, a picture of a food or a meal comprising multiple types of food can be compared directly with pictures of food in a food database.

In an example, selected attributes or parameters of a food image can be adjusted, standardized, or normalized before the food image is compared to images in a database of food images or otherwise analyzed for identifying the type of food. In various examples, these image attributes or parameters can be selected from the group consisting of: food color, food texture, scale, image resolution, image brightness, and light angle. In an example, analysis of food images can comprise automatically segmenting regions of a food image into different types or portions of food. In an example, boundaries can be identified between different types of food in an image that contains multiple types or portions of food. In an example, the creation of boundaries between different types of food and/or segmentation of a meal into different food types can include edge detection, shading analysis, texture analysis, and three-dimensional modeling. In an example, this process can also be informed by common patterns of jointly-served foods and common boundary characteristics of such jointly-served foods.

In an example, a food database can be used to identify the amount of calories that are associated with an identified type and amount of food. In an example, a food database can be used to identify the type and amount of at least one selected type of food that a person consumes. In an example, a food database can be used to identify the type and amount of at least one selected type of ingredient that is associated with an identified type and amount of food. In an example, a food database can be used to identify the type and amount of at least one selected type of nutrient that is associated with an identified type and amount of food. In an example, an ingredient or nutrient can be associated with a type of food on a per-portion, per-volume, or per-weight basis.

In an example, a vector of food characteristics can be extracted from a picture of food and compared with a database of such vectors for common foods. In an example, analysis of data concerning food consumption can include comparison of food consumption parameters between a specific person and a reference population. In an example, data analysis can include analysis of a person's food consumption patterns over time. In an example, such analysis can track the cumulative amount of at least one selected type of food, ingredient, or nutrient that a person consumes during a selected period of time.

In various examples, data concerning food consumption can be analyzed to identify and track consumption of selected types and amounts of foods, ingredients, or nutrient consumed using one or more methods selected from the group consisting of: linear regression and/or multivariate linear regression, logistic regression and/or probit analysis, Fourier transformation and/or fast Fourier transform (FFT), linear discriminant analysis, non-linear programming, analysis of variance, chi-squared analysis, cluster analysis, energy balance tracking, factor analysis, principal components analysis, survival analysis, time series analysis, volumetric modeling, neural network and machine learning.

In various examples, food pictures can be analyzed for automated food identification using methods selected from the group consisting of: image attribute adjustment or normalization; inter-food boundary determination and food portion segmentation; image pattern recognition and comparison with images in a food database to identify food type; comparison of a vector of food characteristics with a database of such characteristics for different types of food; scale determination based on a fiduciary marker and/or three-dimensional modeling to estimate food quantity; and association of selected types and amounts of ingredients or nutrients with selected types and amounts of food portions based on a food database that links common types and amounts of foods with common types and amounts of ingredients or nutrients.

In an example, food image information can be transmitted from a wearable or hand-held device to a remote location where automatic food identification occurs and the results can be transmitted back to the wearable or hand-held device. In an example, identification of the types and quantities of foods, ingredients, or nutrients that a person consumes from pictures of food can be a combination of, or interaction between, automated identification food methods and human-based food identification methods.

In an example, food can be identified by scanning a barcode or other machine-readable code on the food's packaging (such as a Universal Product Code or European Article Number), on a menu, on a store display sign, or otherwise in proximity to food at the point of food selection, sale, or consumption. In an example, the type of food (and/or specific ingredients or nutrients within the food) can be identified by machine-recognition of a food label, nutritional label, or logo on food packaging, menu, or display sign. However, there are many types of food and food consumption situations in which food is not accompanied by such identifying packaging. Accordingly, a robust imaged-based device and method for measuring food consumption should not rely on bar codes or other identifying material on food packaging.

In an example, selected types of foods, ingredients, and/or nutrients can be identified by the patterns of light that are reflected from, or absorbed by, the food at different wavelengths. In an example, a light-based sensor can detect food consumption or can identify consumption of a specific food, ingredient, or nutrient based on the reflection of light from food or the absorption of light by food at different wavelengths. In an example, an optical sensor can detect fluorescence. In an example, an optical sensor can detect whether food reflects light at a different wavelength than the wavelength of light shone on food. In an example, an optical sensor can be a fluorescence polarization immunoassay sensor, chemiluminescence sensor, thermoluminescence sensor, or piezoluminescence sensor.

In an example, the wavelength spectra of light reflected from, or absorbed by, food can be analyzed. In an example, an imaging member and/or light energy sensor can comprise a chromatographic sensor, a spectrographic sensor, an analytical chromatographic sensor, a liquid chromatographic sensor, a gas chromatographic sensor, an optoelectronic sensor, a photochemical sensor, and a photocell. In an example, the modulation of light wave parameters by the interaction of that light with a portion of food can be analyzed. In an example, modulation of light reflected from, or absorbed by, a receptor when the receptor is exposed to food can be analyzed. In an example, an imaging member and/or light energy sensor can emit, detect, or record patterns of white light, infrared light, or ultraviolet light.

In various examples, a selected type of food, ingredient, or nutrient can be identified based on light reflection spectra, light absorption spectra, or light emission spectra. In an example, this can be done using spectroscopy. In an example, spectral measurement can be done with a white light spectroscopy sensor, an infrared spectroscopy sensor, a near-infrared spectroscopy sensor, an ultraviolet spectroscopy sensor, an ion mobility spectroscopic sensor, a mass spectrometry sensor, a backscattering spectrometry sensor, or a spectrophotometer. In an example, light at different wavelengths can be absorbed by, or reflected off, food and the results can be analyzed via spectral analysis.

This invention can further comprise a nutritional intake modification component and/or method. With respect to FIGS. 41 through 60, an nutritional intake modification component and/or method can comprise one or more of the variations which we now discuss. In an example, this invention can comprise a nutritional intake modification component which modifies a person's nutritional intake based on the type and quantity of food consumed by the person. In an example, a nutritional intake modification component can modify a person's nutritional intake by modifying the type and/or amount of food which the person consumes. In an example, a nutritional intake modification component can modify a person's nutritional intake by modifying the absorption of nutrients from food which the person consumes.

In an example, a nutritional intake modification component can reduce a person's consumption of an unhealthy type and/or quantity of food. In an example, a nutritional intake modification component can reduce a person's absorption of nutrients from an unhealthy type and/or quantity of food which the person has consumed. In an example, a nutritional intake modification component can allow normal (or encourage additional) consumption of a healthy type and/or quantity of food. In an example, a nutritional intake modification component can allow normal absorption of nutrients from a healthy type and/or quantity of food which a person has consumed.

In an example, a type of food can be identified as being unhealthy based on analysis of images from an imaging device, analysis of data from one or more wearable sensors, analysis of data from one or more implanted sensors, or a combination thereof. In an example, unhealthy food can be identified as having a high amount or concentration of one or more nutrients selected from the group consisting of: sugars, simple sugars, simple carbohydrates, fats, saturated fats, cholesterol, and sodium. In an example, unhealthy food can be identified as having an amount of one or more nutrients selected from the group consisting of sugars, simple sugars, simple carbohydrates, fats, saturated fats, cholesterol, and sodium that is more than the recommended amount of such nutrient for the person during a given period of time.

In an example, a quantity of food or nutrient which is identified as being unhealthy can be based on one or more factors selected from the group consisting of: the type of food or nutrient; the specificity or breadth of the selected food or nutrient type; the accuracy of a sensor in detecting the selected food or nutrient; the speed or pace of food or nutrient consumption; a person's age, gender, and/or weight; changes in a person's weight; a person's diagnosed health conditions; one or more general health status indicators; the magnitude and/or certainty of the effects of past consumption of the selected nutrient on a person's health; achievement of a person's health goals; a person's exercise patterns and/or caloric expenditure; a person's physical location; the time of day; the day of the week; occurrence of a holiday or other occasion involving special meals; input from a social network and/or behavioral support group; input from a virtual health coach; the cost of food; financial payments, constraints, and/or incentives; health insurance copay and/or health insurance premium; the amount and/or duration of a person's consumption of healthy food or nutrients; a dietary plan created for a person by a health care provider; and the severity of a food allergy.

In an example, a nutritional intake modification component can be part of electronically-functional eyewear. In an example, a nutritional intake modification component can be (part of) a separate wearable device. In an example, a nutritional intake modification component can be (part of) an implanted device. In an example, a nutritional intake modification component can be (part of) a mobile and/or handheld device. In an example, a nutritional intake modification component can be a hardware component. In an example, a nutritional intake modification component can be a software component.

In an example, a nutritional intake component can provide feedback to a person and its effect on nutritional intake can depend on the person voluntarily changing their behavior in response to this feedback. In an example, a nutritional intake component can directly modify the consumption and/or absorption of nutrients in a manner which does not rely on voluntary changes in a person's behavior. In an example, a nutritional intake modification component can be in wireless communication with a data processing unit and/or data transmitting unit which is part of (or, in turn, in electronic communication with) electronically-functional eyewear.

In an example, a nutritional intake modification component can provide negative stimuli in association with unhealthy types and quantities of food and/or provide positive stimuli in association with healthy types and quantities of food. In an example, a nutritional intake modification component can allow normal absorption of nutrients from healthy types and/or quantities of food, but reduce absorption of nutrients from unhealthy types and/or quantities of food.

In an example, a nutritional intake modification component can allow normal absorption of nutrients from a healthy type of food in a person's gastrointestinal tract, but can reduce absorption of nutrients from an unhealthy type of food by releasing an absorption-affecting substance into the person's gastrointestinal tract when the person consumes an unhealthy type of food. In an example, a nutritional intake modification component can allow normal absorption of nutrients from a healthy quantity of food in a person's gastrointestinal tract, but can reduce absorption of nutrients from an unhealthy quantity of food by releasing an absorption-affecting substance into the person's gastrointestinal tract when the person consumes an unhealthy quantity of food.

In an example, a nutritional intake modification component can reduce absorption of nutrients from an unhealthy type and/or quantity of consumed food by releasing a substance which coats the food as it passes through a person's gastrointestinal tract. In an example, a nutritional intake modification component can reduce absorption of nutrients from an unhealthy type and/or quantity of consumed food by releasing a substance which coats a portion of the person's gastrointestinal tract as (or before) that food passes through the person's gastrointestinal tract. In an example, a nutritional intake modification component can reduce absorption of nutrients from an unhealthy type and/or quantity of consumed food by releasing a substance which increases the speed with which that food passes through a portion of the person's gastrointestinal tract.

In an example, a nutritional intake modification component can comprise an implanted reservoir of a food absorption affecting substance which is released in a person's gastrointestinal tract when the person consumes an unhealthy type and/or quantity of food. In an example, the amount of substance which is released degree to which absorption of food through a person's gastrointestinal tract can be remotely adjusted based on the degree to which a type and/or quantity of consumed food is identified as being unhealthy for that person. In an example, a nutritional intake modification component can reduce consumption and/or absorption of nutrients from unhealthy types and/or quantities of food by releasing an absorption-reducing substance into the person's gastrointestinal tract.

In an example, a nutritional intake modification component can allow normal consumption and absorption of healthy food, but can reduce a person's consumption and/or absorption of unhealthy food by delivering electromagnetic energy to a portion of the person's gastrointestinal tract (and/or to nerves which innervate that portion of the person's gastrointestinal tract) when the person consumes unhealthy food. In an example, a nutritional intake modification component can allow normal consumption and absorption of a healthy quantity of food, but can reduce a person's consumption and/or absorption of an unhealthy quantity of food by delivering electromagnetic energy to a portion of the person's gastrointestinal tract (and/or to nerves which innervate that portion of the person's gastrointestinal tract) when the person consumes an unhealthy quantity of food.

In an example, a nutritional intake modification component can deliver electromagnetic energy to a person's stomach and/or to a nerve which innervates the person's stomach. In an example, delivery of electromagnetic energy to a nerve can decrease transmission of natural impulses through that nerve. In an example, delivery of electromagnetic energy to a nerve can simulate natural impulse transmissions through that nerve. In an example, delivery of electromagnetic energy to a person's stomach or associated nerve can cause a feeling of satiety which, in turn, causes the person to consume less food. In an example, delivery of electromagnetic energy to a person's stomach or associated nerve can cause a feeling of nausea which, in turn, causes the person to consume less food.

In an example, delivery of electromagnetic energy to a person's stomach can interfere with the stomach's preparation to receive food, thereby causing the person to consume less food. In an example, delivery of electromagnetic energy to a person's stomach can slow the passage of food through a person's stomach, thereby causing the person to consume less food. In an example, delivery of electromagnetic energy to a person's stomach can interfere with the stomach's preparation to digest food, thereby causing less absorption of nutrients from consumed food. In an example, delivery of electromagnetic energy to a person's stomach can accelerate passage of food through a person's stomach, thereby causing less absorption of nutrients from consumed food. In an example, delivery of electromagnetic energy to a person's stomach can interfere with a person's sensory enjoyment of food and thus cause the person to consume less food.

In an example, a nutritional intake modification component can comprise a gastric electric stimulator (GES). In an example, a nutritional intake modification component can deliver electromagnetic energy to the wall of a person's stomach. In an example, a nutritional intake modification component can be a neurostimulation device. In an example, a nutritional intake modification component can be a neuroblocking device. In an example, a nutritional intake modification component can stimulate, simulate, block, or otherwise modify electromagnetic signals in a peripheral nervous system pathway. In an example, a nutritional intake modification component can deliver electromagnetic energy to the vagus nerve. In an example, the magnitude and/or pattern of electromagnetic energy which is delivered to a person's stomach (and/or to a nerve which innervates the person's stomach) can be adjusted based on the degree to which a type and/or quantity of consumed food is identified as being unhealthy for that person. Selective interference with the consumption and/or absorption of unhealthy food (versus normal consumption and absorption of healthy food) is an advantage over food-blind gastric stimulation devices and methods in the prior art. In an example, a nutritional intake modification component can reduce consumption and/or absorption of nutrients from unhealthy types and/or quantities of food by delivering electromagnetic energy to a portion of the person's gastrointestinal tract and/or to nerves which innervate that portion.

In an example, a nutritional intake modification component can allow normal sensory perception of a healthy type of food, but can modify sensory perception of unhealthy food by delivering electromagnetic energy to nerves which innervate a person's tongue and/or nasal passages when the person consumes an unhealthy type of food. In an example, a nutritional intake modification component can allow normal sensory perception of a healthy quantity of food, but can modify sensory perception of an unhealthy quantity of food by delivering electromagnetic energy to nerves which innervate a person's tongue and/or nasal passages when the person consumes an unhealthy quantity of food.

In an example, a nutritional intake modification component can cause a person to experience an unpleasant virtual taste and/or smell when the person consumes an unhealthy type or quantity of food by delivering electromagnetic energy to afferent nerves which innervate a person's tongue and/or nasal passages. In an example, a nutritional intake modification component can cause temporary dysgeusia when a person consumes an unhealthy type or quantity of food. In an example, a nutritional intake modification component can cause a person to experience reduced taste and/or smell when the person consumes an unhealthy type or quantity of food by delivering electromagnetic energy to afferent nerves which innervate a person's tongue and/or nose. In an example, a nutritional intake modification component can cause temporary ageusia when a person consumes an unhealthy type or quantity of food.

In an example, a nutritional intake modification component can stimulate, simulate, block, or otherwise modify electromagnetic signals in an afferent nerve pathway that conveys taste and/or smell information to the brain. In an example, electromagnetic energy can be delivered to synapses between taste receptors and afferent neurons. In an example, a nutritional intake modification component can deliver electromagnetic energy to a person's CN VII (Facial Nerve), CN IX (Glossopharyngeal Nerve) CN X (Vagus Nerve), and/or CN V (Trigeminal Nerve). In an example, a nutritional intake modification component can inhibit or block the afferent nerves which are associated with selected T1R receptors in order to diminish or eliminate a person's perception of sweetness. In an example, a nutritional intake modification component can stimulate or excite the afferent nerves which are associated with T2R receptors in order to create a virtual or phantom bitter taste.

In an example, a nutritional intake modification component can deliver a selected pattern of electromagnetic energy to afferent nerves in order to make unhealthy food taste and/or smell bad. In an example, a nutritional intake modification component can deliver a selected pattern of electromagnetic energy to afferent nerves in order to make healthy food taste and/or smell good. In an example, the magnitude and/or pattern of electromagnetic energy which is delivered to an afferent nerve can be adjusted based on the degree to which a type and/or quantity of consumed food is identified as being unhealthy for that person. In an example, a nutritional intake modification component can reduce consumption and/or absorption of nutrients from unhealthy types and/or quantities of food by delivering electromagnetic energy to nerves which innervate a person's tongue and/or nasal passages.

In an example, a nutritional intake modification component can allow normal sensory perception of a healthy type of food, but can modify the taste and/or smell of an unhealthy type of food by releasing a taste and/or smell modifying substance into a person's oral cavity and/or nasal passages. In an example, a nutritional intake modification component can allow normal sensory perception of a healthy quantity of food, but can modify the taste and/or smell of an unhealthy quantity of food by releasing a taste and/or smell modifying substance into a person's oral cavity and/or nasal passages. In an example, a nutritional intake modification component can release a substance with a strong flavor into a person's oral cavity when the person consumes an unhealthy type and/or quantity of food. In an example, a nutritional intake modification component can release a substance with a strong smell into a person's nasal passages when the person consumes an unhealthy type and/or quantity of food. In an example, the release of a taste-modifying or smell-modifying substance can be triggered based on analysis of the type and/or quantity of food consumed.

In an example, a taste-modifying substance can be contained in a reservoir which is attached or implanted within a person's oral cavity. In an example, a taste-modifying substance can be contained in a reservoir which is attached to a person's upper palate. In an example, a taste-modifying substance can be contained in a reservoir within a dental appliance or a dental implant. In an example, a taste-modifying substance can be contained in a reservoir which is implanted so as to be in fluid or gaseous communication with a person's oral cavity. In an example, a smell-modifying substance can be contained in a reservoir which is attached or implanted within a person's nasal passages. In an example, a smell-modifying substance can be contained in a reservoir which is implanted so as to be in gaseous or fluid communication with a person's nasal passages.

In an example, a taste-modifying substance can have a strong flavor which overpowers the natural flavor of food when the substance is released into a person's oral cavity. In an example, a taste-modifying substance can be bitter, sour, hot, or just plain noxious. In an example, a taste-modifying substance can anesthetize or otherwise reduce the taste-sensing function of taste buds on a person's tongue. In an example, a taste-modifying substance can cause temporary ageusia. In an example, a smell-modifying substance can have a strong smell which overpowers the natural smell of food when the substance is released into a person's nasal passages. In an example, a smell-modifying substance can anesthetize or otherwise reduce the smell-sensing function of olfactory receptors in a person's nasal passages. In an example, a nutritional intake modification component can reduce consumption and/or absorption of nutrients from unhealthy types and/or quantities of food by releasing a taste and/or smell modifying substance into a person's oral cavity and/or nasal passages.

In an example, a nutritional intake modification component can modify a person's food consumption by sending a communication or message to the person wearing the device and/or to another person. In an example, a nutritional intake modification component can display information on a wearable or mobile device, send a text, make a phone call, or initiate another form of electronic communication regarding food that is near a person and/or consumed food. In an example, a nutritional intake modification component can display information on a wearable or mobile device, send a text, make a phone call, or initiate another form of electronic communication when a person is near food, purchasing food, ordering food, preparing food, and/or consuming food. In an example, information concerning a person's food consumption can be stored in a remote computing device, such as via the internet, and be available for the person to view.

In an example, a nutritional intake modification component can send a communication or message to the person who is wearing the eyewear-based device. In an example, a nutritional intake modification component can send the person nutritional information concerning food that the person is near, food that the person is purchasing, food that the person is ordering, and/or food that the person is consuming. This nutritional information can include food ingredients, nutrients, and/or calories. In an example, a nutritional intake modification component can send the person information concerning the likely health effects of consuming food that the person is near, food that the person is purchasing, food that the person is ordering, and/or food that the person has already starting consuming. In an example, food information which is communicated to the person can be in text form. In an example, a communication can recommend a healthier substitute for unhealthy food which the person is considering consuming.

In an example, food information which is communicated to the person can be in graphic form. In an example, food information which is communicated to the person can be in spoken and/or voice form. In an example, a communication can be in a person's own voice. In an example, a communication can be a pre-recorded message from the person. In an example, a communication can be in the voice of a person who is significant to the person wearing the eyewear. In an example, a communication can be a pre-recorded message from that significant person. In an example, a communication can provide negative feedback in association with consumption of unhealthy food. In an example, a communication can provide positive feedback in association with consumption of healthy food and/or avoiding consumption of unhealthy food. In an example, negative information associated with unhealthy food can encourage the person to eat less unhealthy food and positive information associated with healthy foods can encourage the person to eat more healthy food.

In an example, a nutritional intake modification component can send a communication to a person other than the person who is wearing the eyewear-based device. In an example, this other person can provide encouragement and support for the person wearing the device to eat less unhealthy food and/or eat more healthy food. In an example, this other person can be a friend, support group member, family member, health care provider, nosy neighbor, or an analyst in a covert government agency who is monitoring data streams from wearable devices. In an example, the latter can be avoided by wearing an aluminum foil hat. In an example, a nutritional intake modification component can comprise connectivity with a social network website and/or an internet-based support group. In an example, a nutritional intake modification component can encourage a person to reduce consumption of unhealthy types and/or quantities of food (and increase consumption of healthy food) in order to achieve personal health goals. In an example, a nutritional intake modification component can encourage a person to reduce consumption of unhealthy types and/or quantities of food (and increase consumption of healthy food) in order to compete with friends and/or people in a peer group with respect to achievement of health goals. In an example, a nutritional intake modification component can function as a virtual dietary health coach. In an example, a nutritional intake modification component can reduce consumption and/or absorption of nutrients from unhealthy types and/or quantities of food by constricting, slowing, and/or reducing passage of food through the person's gastrointestinal tract.

In an example, a nutritional intake modification component can display images or other visual information in a person's field of view which modify the person's consumption of food. In an example, a nutritional intake modification component can display images or other visual information in proximity to food in the person's field of view in a manner which modifies the person's consumption of that food. In an example, a nutritional intake modification component can be part of an augmented reality system which displays virtual images and/or information in proximity to real world objects. In an example, a nutritional intake modification system can superimpose virtual images and/or information on food in a person's field of view.

In an example, a nutritional intake modification component can display virtual nutrition information concerning food that is in a person's field of view. In an example, a nutritional intake modification component can display information concerning the ingredients, nutrients, and/or calories in a portion of food which is within a person's field of view. In an example, this information can be based on analysis of images from the imaging device, one or more (other) wearable sensors, or both. In an example, virtual nutrition information can be displayed on a screen (or other display mode) which is separate from a person's view of their environment. In an example, virtual nutrition information can be superimposed on a person's view of their environment as part of an augmented reality system. In an augmented reality system, virtual nutrition information can be superimposed directly over the food in question. In an example, display of negative nutritional information and/or information about the potential negative effects of unhealthy nutrients can reduce a person's consumption of an unhealthy type or quantity of food. In an example, a nutritional intake modification component can display warnings about potential negative health effects and/or allergic reactions. In an example, display of positive nutritional information and/or information on the potential positive effects of healthy nutrients can increase a person's consumption of healthy food. In an example, a nutritional intake modification component can display encouraging information about potential health benefits of selected foods or nutrients.

In an example, a nutritional intake modification component can display virtual images in response to food that is in a person's field of view. In an example, virtual images can be displayed on a screen (or other display mode) which is separate from a person's view of their environment. In an example, virtual images can be superimposed on a person's view of their environment, such as part of an augmented reality system. In an augmented reality system, a virtual image can be superimposed directly over the food in question. In an example, display of unpleasant image (or one with negative connotations) can reduce a person's consumption of an unhealthy type or quantity of food. In an example, display of an appealing image (or one with positive connotations) can increase a person's consumption of healthy food. In an example, a nutritional intake modification component can display an image of a virtual person in response to food, wherein the weight, size, shape, and/or health status of this person is based on the potential effects of (repeatedly) consuming this food. In an example, this virtual person can be a modified version of the person wearing the eyewear, wherein the modification is based on the potential effects of (repeatedly) consuming the food in question. In an example, this invention can show the person how they will probably look if they (repeatedly) consume this type and/or quantity of food.

In an example, a nutritional intake modification component can be part of an augmented reality system which changes a person's visual perception of unhealthy food to make it less appealing and/or changes the person's visual perception of healthy food to make it more appealing. In an example, a change in visual perception of food can be selected from the group consisting of: a change in perceived color and/or light spectrum; a change in perceived texture or shading; and a change in perceived size or shape. In an example, a nutritional intake modification component can display an unappealing image which is unrelated to food but which, when shown in juxtaposition with unhealthy food, will decrease the appeal of that food by association. In an example, a nutritional intake modification component can display an appealing image which is unrelated to food but which, when shown in juxtaposition with healthy food, will increase the appeal of that food by association. In an example, a nutritional intake modification component can reduce consumption and/or absorption of nutrients from unhealthy types and/or quantities of food by displaying images or other visual information in a person's field of view.

In an example, a nutritional intake modification component can allow normal passage of a healthy type of food through a person's gastrointestinal tract, but can constrict, slow, and/or reduce passage of an unhealthy type of food through the person's gastrointestinal tract. In an example, a nutritional intake modification component can allow normal passage of up to a healthy cumulative quantity of food (during a meal or selected period of time) through a person's gastrointestinal tract, but can constrict, slow, and/or reduce passage of food in excess of this quantity. In an example, a type and/or quantity of food can be identified as healthy or unhealthy based on analysis of images from the imaging member. In an example, a type and/or quantity of food can be identified as unhealthy based on analysis of images from an imaging device, analysis of data from one or more wearable or implanted sensors, or both. In an example, unhealthy food can be identified as having large (relative) quantities of simple sugars, carbohydrates, saturated fats, bad cholesterol, and/or sodium compounds.

In an example, a nutritional intake modification component can selectively constrict, slow, and/or reduce passage of food through a person's gastrointestinal tract by adjustably constricting or resisting jaw movement, adjustably changing the size or shape of the person's oral cavity, adjustably changing the size or shape of the entrance to a person's stomach, adjustably changing the size, shape, or function of the pyloric sphincter, and/or adjustably changing the size or shape of the person's stomach. In an example, such adjustment can be done in a non-invasive (such as through wireless communication) and reversible manner after an operation in which a device is implanted. In an example, the degree to which passage of food through a person's gastrointestinal tract is constricted, slowed, and/or reduced can be adjusted based on the degree to which a type and/or quantity of food is identified as being unhealthy for that person.

In an example, a nutritional intake modification component can allow normal absorption of nutrients from consumed food which is identified as a healthy type of food, but can reduce absorption of nutrients from consumed food which is identified as an unhealthy type of food. In an example, a nutritional intake modification component can allow normal absorption of nutrients from consumed food up to a selected cumulative quantity (during a meal or selected period of time) which is identified as a healthy quantity of food, but can reduce absorption of nutrients from consumed food greater than this selected cumulative quantity. In an example, a type and/or quantity of food can be identified as healthy or unhealthy based on analysis of images from the imaging member. In an example, a type and/or quantity of food can be identified as unhealthy based on analysis of images from an imaging device, analysis of data from one or more wearable or implanted sensors, or both. In an example, unhealthy food can be identified as having large (relative) quantities of simple sugars, carbohydrates, saturated fats, bad cholesterol, and/or sodium compounds.

In an example, a nutritional intake modification component can selectively reduce absorption of nutrients from consumed food by changing the route through which that food passes as that food travels through the person's gastrointestinal tract. In an example, a nutritional intake modification component can comprise an adjustable valve within a person's gastrointestinal tract. In an example, an adjustable valve of an intake modification component can be located within a person's stomach. In an example, an adjustable food valve can have a first configuration which directs food through a first route through a person's gastrointestinal tract and can have a second configuration which directs food through a second configuration in a person's gastrointestinal tract. In an example, the first configuration can be shorter or bypass key nutrient-absorbing structures (such as the duodenum) in the gastrointestinal tract. In an example, a nutritional intake modification component can direct a healthy type and/or quantity of food through a longer route through a person's gastrointestinal tract and can direct an unhealthy type and/or quantity of food through a shorter route through a person's gastrointestinal tract. In an example, a nutritional intake modification component can reduce consumption and/or absorption of nutrients from unhealthy types and/or quantities of food by sending a communication to the person wearing the imaging member and/or to another person.

In an example, a nutritional intake modification component can comprise one or more actuators which exert inward pressure on the exterior surface of a person's body in response to consumption of an unhealthy type and/or quantity of food. In an example a nutritional intake modification component can comprise one or more actuators which are incorporated into an article of clothing or a clothing accessory, wherein these one or more actuators are constricted when a person consumes an unhealthy type and/or amount of food. In an example, an article of clothing can be smart shirt. In an example, a clothing accessory can be a belt. In an example, an actuator can be a piezoelectric actuator. In an example, an actuator can be a piezoelectric textile or fabric.

In an example, a nutritional intake modification component can deliver a low level of electromagnetic energy to the exterior surface of a person's body in response to consumption of an unhealthy type and/or quantity of food. In an example, this electromagnetic energy can act as an adverse stimulus which reduces a person's consumption of unhealthy food. In an example, this electromagnetic energy can interfere with the preparation of the stomach to receive and digest. In an example, a nutritional intake modification component can comprise a financial restriction function which impedes the purchase of an unhealthy type and/or quantity of food. In an example, this invention can reduce the ability of a person to purchase or order food when the food is identified as being unhealthy.

In an example, a nutritional intake modification component can be implanted so as to delivery electromagnetic energy to one or more organs or body tissues selected from the group consisting of: brain, pyloric sphincter, small intestine, large intestine, liver, pancreas, and spleen. In an example, a nutritional intake modification component can be implanted so as to delivery electromagnetic energy to the muscles which move one or more organs or body tissues selected from the group consisting of: esophagus, stomach, pyloric sphincter, small intestine, large intestine, liver, pancreas, and spleen. In an example, a nutritional intake modification component can be implanted so as to delivery electromagnetic energy to the nerves which innervate one or more organs or body tissues selected from the group consisting of: esophagus, stomach, pyloric sphincter, small intestine, large intestine, liver, pancreas, and spleen.

In an example, a nutritional intake modification component can comprise an implanted or wearable drug dispensing device which dispenses an appetite and/or digestion modifying drug in response to consumption of an unhealthy type and/or quantity of food. In an example, a nutritional intake modification component can comprise a light-based computer-to-human interface which emits light in response to consumption of an unhealthy type and/or quantity of food. In an example, this interface can comprise an LED array. In an example, a nutritional intake modification component can comprise a sound-based computer-to-human interface which emits sound in response to consumption of an unhealthy type and/or quantity of food. In an example, this sound can be a voice, tones, and/or music. In an example, a nutritional intake modification component can comprise a tactile-based computer-to-human interface which creates tactile sensations in response to consumption of an unhealthy type and/or quantity of food. In an example, this tactile sensation can be a vibration (and not a good one).

FIG. 41 shows an example of how this invention can be embodied in an eyewear-based system and device for monitoring and modifying a person's (4101) nutritional intake comprising: eyewear (further comprising support member 4103 and optical member 4104), wherein this eyewear further comprises at least one imaging member (camera 4105), wherein this imaging member automatically takes pictures or records images of food (4102) when a person is consuming food, and wherein these food pictures or images are automatically analyzed to estimate the type and quantity of food; a data processing unit (4106); and a nutritional intake modification component (4107), wherein this component modifies the person's nutritional intake based on the type and quantity of food.

FIG. 41 also shows an example of how this invention can be embodied in an eyewear-based system and device for monitoring and modifying a person's 4101 nutritional intake comprising: a support member 4103 which is configured to be worn on a person's head; at least one optical member 4104 which is configured to be held in proximity to an eye by the support member; at least one imaging member 4105, wherein the imaging member is part of or attached to the support member or optical member, wherein this imaging member automatically takes pictures or records images of food 4102 when a person is consuming food, and wherein these food pictures or images are automatically analyzed to estimate the type and quantity of food; a data processing unit 4106; and a nutritional intake modification component 4107, wherein this component modifies the person's nutritional intake based on the type and quantity of food.

In the example in FIG. 41, although not shown from this perspective, there are assumed to be two optical members (one for each eye). In this example, support member 4103 and two optical members (including 4104) together comprise eyeglasses. In this example, imaging member 4105 is a camera. In this example, camera 4105 automatically takes pictures or records images of food 4102 because it takes pictures or record images all the time. As discussed earlier, unhealthy types and/or quantities of food can be identified based on these food pictures and/or images.

In this example, nutritional intake modification component 4107 is an implanted electromagnetic energy emitter. In this example, nutritional intake modification component 4107 reduces consumption and/or absorption of nutrients from unhealthy types and/or quantities of food by delivering electromagnetic energy to a portion of the person's gastrointestinal tract and/or to nerves which innervate that portion. In this example, nutritional intake modification component 4107 delivers electromagnetic energy to the person's stomach and/or to a nerve which innervates the stomach. FIG. 41 can include other component variations which were discussed earlier.

FIG. 42 shows an example of how this invention can be embodied in an eyewear-based system and device for monitoring and modifying a person's 4101 nutritional intake comprising: a support member 4103 which is configured to be worn on a person's head; at least one optical member 4104 which is configured to be held in proximity to an eye by the support member; at least one imaging member 4105, wherein the imaging member is part of or attached to the support member or optical member, wherein this imaging member automatically takes pictures or records images of food 4102 when a person is consuming food, and wherein these food pictures or images are automatically analyzed to estimate the type and quantity of food; a data processing unit 4106; and a nutritional intake modification component 4107, wherein this component modifies the person's nutritional intake based on the type and quantity of food.

In the example in FIG. 42, there are assumed to be two optical members (one for each eye). In this example, support member 4103 and two optical members (including 4104) together comprise eyeglasses. In this example, imaging member 4105 is a camera. As discussed earlier, unhealthy types and/or quantities of food can be identified based on food pictures and/or images.

The example in FIG. 42 further comprises motion sensor 4201. In this example, imaging member 4105 is automatically activated (triggered) to take pictures or record images of food when data from one or more wearable or implanted sensors indicates that person 4101 is consuming food or will probably consume food soon. In this example, imaging member 4105 is automatically activated (triggered) to take pictures or record images of food when data from motion sensor 4201 indicates that person 4101 is consuming food or will probably consume food soon. Motion patterns indicative of food consumption were discussed earlier. In this example, motion sensor 4105 is an accelerometer. In this example, imaging member is automatically activated (triggered) to take pictures when a person eats, based on a sensor selected from the group consisting of: accelerometer, inclinometer, and motion sensor.

In this example, nutritional intake modification component 4107 is an implanted electromagnetic energy emitter. In this example, nutritional intake modification component 4107 allows normal absorption of nutrients from healthy types and/or quantities of food, but reduces absorption of nutrients from unhealthy types and/or quantities of food. In this example, nutritional intake modification component 4107 reduces consumption and/or absorption of nutrients from unhealthy types and/or quantities of food by delivering electromagnetic energy to a portion of the person's gastrointestinal tract and/or to nerves which innervate that portion. In this example, nutritional intake modification component 4107 delivers electromagnetic energy to the person's stomach and/or to a nerve which innervates the stomach. FIG. 42 can also include other component variations which were discussed earlier.

FIG. 43 shows an example of how this invention can be embodied in an eyewear-based system and device for monitoring and modifying a person's 4101 nutritional intake comprising: a support member 4103 which is configured to be worn on a person's head; at least one optical member 4104 which is configured to be held in proximity to an eye by the support member; at least one imaging member 4105, wherein the imaging member is part of or attached to the support member or optical member, wherein this imaging member automatically takes pictures or records images of food 4102 when a person is consuming food, and wherein these food pictures or images are automatically analyzed to estimate the type and quantity of food; a data processing unit 4106; and a nutritional intake modification component 4107, wherein this component modifies the person's nutritional intake based on the type and quantity of food. In this example, support member 4103 and two optical members (including 4104) together comprise eyeglasses. In this example, imaging member 4105 is a camera. As discussed earlier, unhealthy types and/or quantities of food can be identified based on food pictures and/or images.

The example in FIG. 43 further comprises electromagnetic energy sensor 4301. In this example, imaging member 4105 is automatically activated (triggered) to take pictures or record images of food when data from one or more wearable or implanted sensors indicates that person 4101 is consuming food or will probably consume food soon. In this example, imaging member 4105 is automatically activated (triggered) to take pictures or record images of food when data from electromagnetic energy sensor 4301 indicates that person 4101 is consuming food or will probably consume food soon. In this example, an electromagnetic energy sensor measures the conductivity, voltage, impedance, or resistance of electromagnetic energy transmitted through body tissue.

In this example, nutritional intake modification component 4107 is an implanted electromagnetic energy emitter. In this example, nutritional intake modification component 4107 allows normal absorption of nutrients from healthy types and/or quantities of food, but reduces absorption of nutrients from unhealthy types and/or quantities of food. In this example, nutritional intake modification component 4107 reduces consumption and/or absorption of nutrients from unhealthy types and/or quantities of food by delivering electromagnetic energy to a portion of the person's gastrointestinal tract and/or to nerves which innervate that portion. In this example, nutritional intake modification component 4107 delivers electromagnetic energy to the person's stomach and/or to a nerve which innervates the stomach. FIG. 43 can also include other component variations which were discussed earlier.

FIG. 44 shows an example of how this invention can be embodied in an eyewear-based system and device for monitoring and modifying a person's 4101 nutritional intake comprising: a support member 4103 which is configured to be worn on a person's head; at least one optical member 4104 which is configured to be held in proximity to an eye by the support member; at least one imaging member 4105, wherein the imaging member is part of or attached to the support member or optical member, wherein this imaging member automatically takes pictures or records images of food 4102 when a person is consuming food, and wherein these food pictures or images are automatically analyzed to estimate the type and quantity of food; a data processing unit 4106; and a nutritional intake modification component 4107, wherein this component modifies the person's nutritional intake based on the type and quantity of food. In this example, support member 4103 and two optical members (including 4104) together comprise eyeglasses. In this example, imaging member 4105 is a camera. As discussed earlier, unhealthy types and/or quantities of food can be identified based on food pictures and/or images.

The example in FIG. 44 further comprises intra-oral sensor 4401. In this example, imaging member 4105 is automatically activated (triggered) to take pictures or record images of food when data from one or more wearable or implanted sensors indicates that person 4101 is consuming food or will probably consume food soon. In this example, imaging member 4105 is automatically activated (triggered) to take pictures or record images of food when data from intra-oral sensor 4401 indicates that person 4101 is consuming food or will probably consume food soon. In various examples, intra-oral sensor 4401 can be selected from the group consisting of: glucometer, glucose sensor, glucose monitor, spectroscopic sensor, food composition analyzer, oximeter, oximetry sensor, gas composition sensor, artificial olfactory sensor, smell sensor, chemiresistor sensor, chemoreceptor sensor, electrochemical sensor, amino acid sensor, cholesterol sensor, osmolality sensor, pH level sensor, sodium sensor, taste sensor, and microbial sensor.

In this example, nutritional intake modification component 4107 is an implanted electromagnetic energy emitter. In this example, nutritional intake modification component 4107 allows normal absorption of nutrients from healthy types and/or quantities of food, but reduces absorption of nutrients from unhealthy types and/or quantities of food. In this example, nutritional intake modification component 4107 reduces consumption and/or absorption of nutrients from unhealthy types and/or quantities of food by delivering electromagnetic energy to a portion of the person's gastrointestinal tract and/or to nerves which innervate that portion. In this example, nutritional intake modification component 4107 delivers electromagnetic energy to the person's stomach and/or to a nerve which innervates the stomach. FIG. 44 can also include other component variations which were discussed earlier.

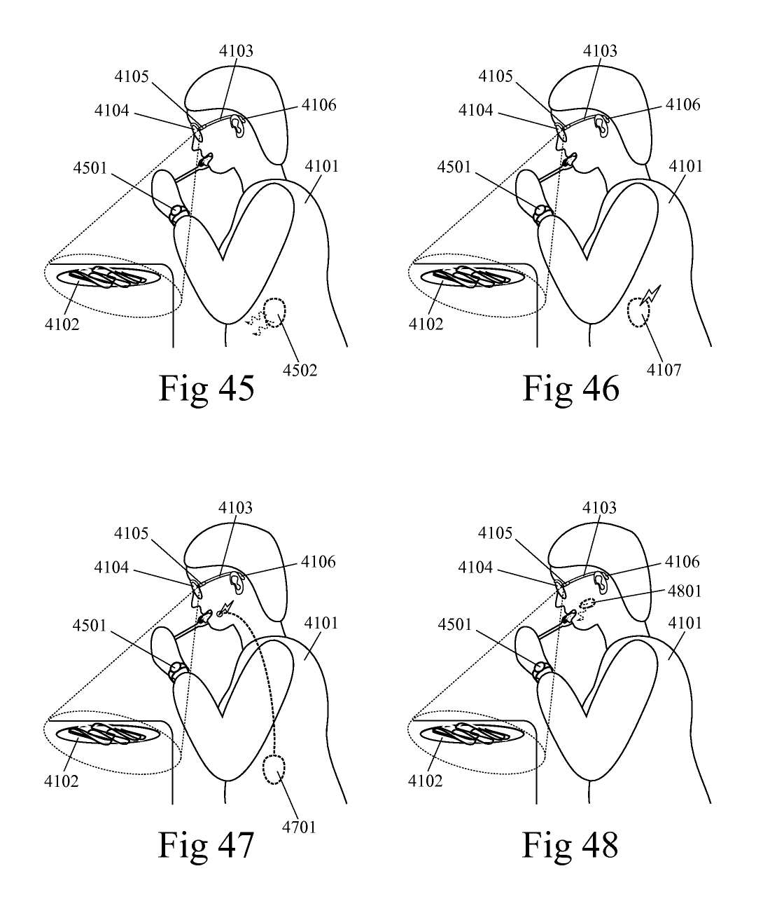

FIG. 45 shows an example of how this invention can be embodied in an eyewear-based system and device for monitoring and modifying a person's 4101 nutritional intake comprising: a support member 4103 which is configured to be worn on a person's head; at least one optical member 4104 which is configured to be held in proximity to an eye by the support member; at least one imaging member 4105, wherein the imaging member is part of or attached to the support member or optical member, wherein this imaging member automatically takes pictures or records images of food 4102 when a person is consuming food, and wherein these food pictures or images are automatically analyzed to estimate the type and quantity of food; a data processing unit 4106; and a nutritional intake modification component 4107, wherein this component modifies the person's nutritional intake based on the type and quantity of food. In this example, support member 4103 and two optical members (including 4104) together comprise eyeglasses. In this example, imaging member 4105 is a camera. As discussed earlier, unhealthy types and/or quantities of food can be identified based on food pictures and/or images.

The example in FIG. 45 further comprises wrist-worn sensor 4501. In an example, wrist-worn sensor 4501 can be selected from the group consisting of: glucometer, glucose sensor, glucose monitor, blood glucose monitor, cellular fluid glucose monitor, spectroscopic sensor, food composition analyzer, oximeter, oximetry sensor, pulse oximeter, tissue oximetry sensor, tissue saturation oximeter, wrist oximeter, oxygen consumption monitor, oxygen level monitor, oxygen saturation monitor, ambient air sensor, gas composition sensor, blood oximeter, cutaneous oxygen monitor, capnography sensor, carbon dioxide sensor, carbon monoxide sensor, artificial olfactory sensor, smell sensor, moisture sensor, humidity sensor, hydration sensor, skin moisture sensor, chemiresistor sensor, chemoreceptor sensor, electrochemical sensor, amino acid sensor, cholesterol sensor, body fat sensor, osmolality sensor, pH level sensor, sodium sensor, taste sensor, and microbial sensor.

In this example, imaging member 4105 is automatically activated (triggered) to take pictures or record images of food when data from one or more wearable or implanted sensors indicates that person 4101 is consuming food or will probably consume food soon. In this example, imaging member 4105 is automatically activated (triggered) to take pictures or record images of food when data from wrist-worn sensor 4501 indicates that person 4101 is consuming food or will probably consume food soon.

In this example, nutritional intake modification component 4502 is an implanted substance-releasing device. In this example, nutritional intake modification component 4502 allows normal absorption of nutrients from healthy types and/or quantities of food, but reduces absorption of nutrients from unhealthy types and/or quantities of food. In this example, nutritional intake modification component 4502 reduces consumption and/or absorption of nutrients from unhealthy types and/or quantities of food by releasing an absorption-reducing substance into the person's gastrointestinal tract. In this example, nutritional intake modification component 4502 releases an absorption-reducing substance into the person's stomach.

FIG. 46 shows an example of how this invention can be embodied in an eyewear-based system and device for monitoring and modifying a person's 4101 nutritional intake comprising: a support member 4103 which is configured to be worn on a person's head; at least one optical member 4104 which is configured to be held in proximity to an eye by the support member; at least one imaging member 4105, wherein the imaging member is part of or attached to the support member or optical member, wherein this imaging member automatically takes pictures or records images of food 4102 when a person is consuming food, and wherein these food pictures or images are automatically analyzed to estimate the type and quantity of food; a data processing unit 4106; and a nutritional intake modification component 4107, wherein this component modifies the person's nutritional intake based on the type and quantity of food. In this example, support member 4103 and two optical members (including 4104) together comprise eyeglasses. In this example, imaging member 4105 is a camera. As discussed earlier, unhealthy types and/or quantities of food can be identified based on food pictures and/or images.

The example in FIG. 46 further comprises wrist-worn sensor 4501. In an example, wrist-worn sensor 4501 can be selected from the group consisting of: glucometer, glucose sensor, glucose monitor, blood glucose monitor, cellular fluid glucose monitor, spectroscopic sensor, food composition analyzer, oximeter, oximetry sensor, pulse oximeter, tissue oximetry sensor, tissue saturation oximeter, wrist oximeter, oxygen consumption monitor, oxygen level monitor, oxygen saturation monitor, ambient air sensor, gas composition sensor, blood oximeter, cutaneous oxygen monitor, capnography sensor, carbon dioxide sensor, carbon monoxide sensor, artificial olfactory sensor, smell sensor, moisture sensor, humidity sensor, hydration sensor, skin moisture sensor, chemiresistor sensor, chemoreceptor sensor, electrochemical sensor, amino acid sensor, cholesterol sensor, body fat sensor, osmolality sensor, pH level sensor, sodium sensor, taste sensor, and microbial sensor.

In this example, imaging member 4105 is automatically activated (triggered) to take pictures or record images of food when data from one or more wearable or implanted sensors indicates that person 4101 is consuming food or will probably consume food soon. In this example, imaging member 4105 is automatically activated (triggered) to take pictures or record images of food when data from wrist-worn sensor 4501 indicates that person 4101 is consuming food or will probably consume food soon.

In this example, nutritional intake modification component 4107 is an implanted electromagnetic energy emitter. In this example, nutritional intake modification component 4107 allows normal absorption of nutrients from healthy types and/or quantities of food, but reduces absorption of nutrients from unhealthy types and/or quantities of food. In this example, nutritional intake modification component 4107 reduces consumption and/or absorption of nutrients from unhealthy types and/or quantities of food by delivering electromagnetic energy to a portion of the person's gastrointestinal tract and/or to nerves which innervate that portion. In this example, nutritional intake modification component 4107 delivers electromagnetic energy to the person's stomach and/or to a nerve which innervates the stomach. FIG. 46 can also include other component variations which were discussed earlier.

FIG. 47 shows an example of how this invention can be embodied in an eyewear-based system and device for monitoring and modifying a person's 4101 nutritional intake comprising: a support member 4103 which is configured to be worn on a person's head; at least one optical member 4104 which is configured to be held in proximity to an eye by the support member; at least one imaging member 4105, wherein the imaging member is part of or attached to the support member or optical member, wherein this imaging member automatically takes pictures or records images of food 4102 when a person is consuming food, and wherein these food pictures or images are automatically analyzed to estimate the type and quantity of food; a data processing unit 4106; and a nutritional intake modification component 4107, wherein this component modifies the person's nutritional intake based on the type and quantity of food. In this example, support member 4103 and two optical members (including 4104) together comprise eyeglasses. In this example, imaging member 4105 is a camera. As discussed earlier, unhealthy types and/or quantities of food can be identified based on food pictures and/or images.

The example in FIG. 47 further comprises wrist-worn sensor 4501. In an example, wrist-worn sensor 4501 can be selected from the group consisting of: glucometer, glucose sensor, glucose monitor, blood glucose monitor, cellular fluid glucose monitor, spectroscopic sensor, food composition analyzer, oximeter, oximetry sensor, pulse oximeter, tissue oximetry sensor, tissue saturation oximeter, wrist oximeter, oxygen consumption monitor, oxygen level monitor, oxygen saturation monitor, ambient air sensor, gas composition sensor, blood oximeter, cutaneous oxygen monitor, capnography sensor, carbon dioxide sensor, carbon monoxide sensor, artificial olfactory sensor, smell sensor, moisture sensor, humidity sensor, hydration sensor, skin moisture sensor, chemiresistor sensor, chemoreceptor sensor, electrochemical sensor, amino acid sensor, cholesterol sensor, body fat sensor, osmolality sensor, pH level sensor, sodium sensor, taste sensor, and microbial sensor.

In this example, imaging member 4105 is automatically activated (triggered) to take pictures or record images of food when data from one or more wearable or implanted sensors indicates that person 4101 is consuming food or will probably consume food soon. In this example, imaging member 4105 is automatically activated (triggered) to take pictures or record images of food when data from wrist-worn sensor 4501 indicates that person 4101 is consuming food or will probably consume food soon.

In this example, nutritional intake modification component 4701 is an implanted electromagnetic energy emitter. In this example, nutritional intake modification component 4701 allows normal consumption (and/or absorption) of nutrients from healthy types and/or quantities of food, but reduces consumption (and/or absorption) of nutrients from unhealthy types and/or quantities of food. In this example, nutritional intake modification component 4701 reduces consumption and/or absorption of nutrients from unhealthy types and/or quantities of food by delivering electromagnetic energy to nerves which innervate a person's tongue and/or nasal passages. In an example, this electromagnetic energy can reduce taste and/or smell sensations. In an example, this electromagnetic energy can create virtual taste and/or smell sensations. FIG. 47 can also include other component variations which were discussed earlier.

FIG. 48 shows an example of how this invention can be embodied in an eyewear-based system and device for monitoring and modifying a person's 4101 nutritional intake comprising: a support member 4103 which is configured to be worn on a person's head; at least one optical member 4104 which is configured to be held in proximity to an eye by the support member; at least one imaging member 4105, wherein the imaging member is part of or attached to the support member or optical member, wherein this imaging member automatically takes pictures or records images of food 4102 when a person is consuming food, and wherein these food pictures or images are automatically analyzed to estimate the type and quantity of food; a data processing unit 4106; and a nutritional intake modification component 4107, wherein this component modifies the person's nutritional intake based on the type and quantity of food. In this example, support member 4103 and two optical members (including 4104) together comprise eyeglasses. In this example, imaging member 4105 is a camera. As discussed earlier, unhealthy types and/or quantities of food can be identified based on food pictures and/or images.

The example in FIG. 48 further comprises wrist-worn sensor 4501. In an example, wrist-worn sensor 4501 can be selected from the group consisting of: glucometer, glucose sensor, glucose monitor, blood glucose monitor, cellular fluid glucose monitor, spectroscopic sensor, food composition analyzer, oximeter, oximetry sensor, pulse oximeter, tissue oximetry sensor, tissue saturation oximeter, wrist oximeter, oxygen consumption monitor, oxygen level monitor, oxygen saturation monitor, ambient air sensor, gas composition sensor, blood oximeter, cutaneous oxygen monitor, capnography sensor, carbon dioxide sensor, carbon monoxide sensor, artificial olfactory sensor, smell sensor, moisture sensor, humidity sensor, hydration sensor, skin moisture sensor, chemiresistor sensor, chemoreceptor sensor, electrochemical sensor, amino acid sensor, cholesterol sensor, body fat sensor, osmolality sensor, pH level sensor, sodium sensor, taste sensor, and microbial sensor.

In this example, imaging member 4105 is automatically activated (triggered) to take pictures or record images of food when data from one or more wearable or implanted sensors indicates that person 4101 is consuming food or will probably consume food soon. In this example, imaging member 4105 is automatically activated (triggered) to take pictures or record images of food when data from wrist-worn sensor 4501 indicates that person 4101 is consuming food or will probably consume food soon.

In this example, nutritional intake modification component 4801 is an implanted substance-releasing device. In this example, nutritional intake modification component 4801 allows normal consumption (and/or absorption) of nutrients from healthy types and/or quantities of food, but reduces consumption (and/or absorption) of nutrients from unhealthy types and/or quantities of food. In this example, nutritional intake modification component 4801 reduces consumption and/or absorption of nutrients from unhealthy types and/or quantities of food by releasing a taste and/or smell modifying substance into a person's oral cavity and/or nasal passages. In an example, this substance can overpower the taste and/or smell of food. In an example, this substance can be released selectively to make unhealthy food taste or smell bad. FIG. 48 can also include other component variations which were discussed earlier.

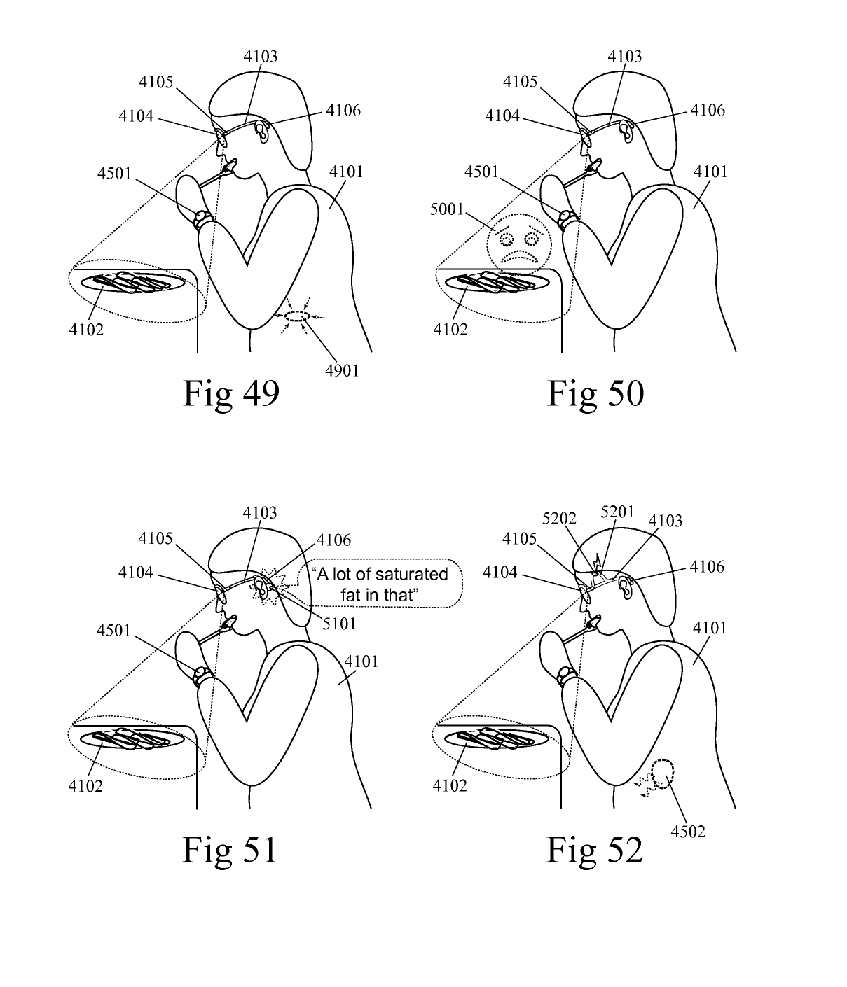

FIG. 49 shows an example of how this invention can be embodied in an eyewear-based system and device for monitoring and modifying a person's 4101 nutritional intake comprising: a support member 4103 which is configured to be worn on a person's head; at least one optical member 4104 which is configured to be held in proximity to an eye by the support member; at least one imaging member 4105, wherein the imaging member is part of or attached to the support member or optical member, wherein this imaging member automatically takes pictures or records images of food 4102 when a person is consuming food, and wherein these food pictures or images are automatically analyzed to estimate the type and quantity of food; a data processing unit 4106; and a nutritional intake modification component 4107, wherein this component modifies the person's nutritional intake based on the type and quantity of food. In this example, support member 4103 and two optical members (including 4104) together comprise eyeglasses. In this example, imaging member 4105 is a camera. As discussed earlier, unhealthy types and/or quantities of food can be identified based on food pictures and/or images.

The example in FIG. 49 further comprises wrist-worn sensor 4501. In an example, wrist-worn sensor 4501 can be selected from the group consisting of: glucometer, glucose sensor, glucose monitor, blood glucose monitor, cellular fluid glucose monitor, spectroscopic sensor, food composition analyzer, oximeter, oximetry sensor, pulse oximeter, tissue oximetry sensor, tissue saturation oximeter, wrist oximeter, oxygen consumption monitor, oxygen level monitor, oxygen saturation monitor, ambient air sensor, gas composition sensor, blood oximeter, cutaneous oxygen monitor, capnography sensor, carbon dioxide sensor, carbon monoxide sensor, artificial olfactory sensor, smell sensor, moisture sensor, humidity sensor, hydration sensor, skin moisture sensor, chemiresistor sensor, chemoreceptor sensor, electrochemical sensor, amino acid sensor, cholesterol sensor, body fat sensor, osmolality sensor, pH level sensor, sodium sensor, taste sensor, and microbial sensor.

In this example, imaging member 4105 is automatically activated (triggered) to take pictures or record images of food when data from one or more wearable or implanted sensors indicates that person 4101 is consuming food or will probably consume food soon. In this example, imaging member 4105 is automatically activated (triggered) to take pictures or record images of food when data from wrist-worn sensor 4501 indicates that person 4101 is consuming food or will probably consume food soon.

In this example, nutritional intake modification component 4901 is an implanted gastrointestinal constriction device. In this example, nutritional intake modification component 4901 allows normal consumption (and/or absorption) of nutrients from healthy types and/or quantities of food, but reduces consumption (and/or absorption) of nutrients from unhealthy types and/or quantities of food. In this example, nutritional intake modification component 4901 reduces consumption and/or absorption of nutrients from unhealthy types and/or quantities of food by constricting, slowing, and/or reducing passage of food through the person's gastrointestinal tract. In an example, this nutritional intake modification component is a remotely-adjustable gastric band. FIG. 49 can also include other component variations which were discussed earlier.

FIG. 50 shows an example of how this invention can be embodied in an eyewear-based system and device for monitoring and modifying a person's 4101 nutritional intake comprising: a support member 4103 which is configured to be worn on a person's head; at least one optical member 4104 which is configured to be held in proximity to an eye by the support member; at least one imaging member 4105, wherein the imaging member is part of or attached to the support member or optical member, wherein this imaging member automatically takes pictures or records images of food 4102 when a person is consuming food, and wherein these food pictures or images are automatically analyzed to estimate the type and quantity of food; a data processing unit 4106; and a nutritional intake modification component 5001, wherein this component modifies the person's nutritional intake based on the type and quantity of food. In this example, support member 4103 and two optical members (including 4104) together comprise eyeglasses. In this example, imaging member 4105 is a camera. As discussed earlier, unhealthy types and/or quantities of food can be identified based on food pictures and/or images.

The example in FIG. 50 further comprises wrist-worn sensor 4501. In an example, wrist-worn sensor 4501 can be selected from the group consisting of: glucometer, glucose sensor, glucose monitor, blood glucose monitor, cellular fluid glucose monitor, spectroscopic sensor, food composition analyzer, oximeter, oximetry sensor, pulse oximeter, tissue oximetry sensor, tissue saturation oximeter, wrist oximeter, oxygen consumption monitor, oxygen level monitor, oxygen saturation monitor, ambient air sensor, gas composition sensor, blood oximeter, cutaneous oxygen monitor, capnography sensor, carbon dioxide sensor, carbon monoxide sensor, artificial olfactory sensor, smell sensor, moisture sensor, humidity sensor, hydration sensor, skin moisture sensor, chemiresistor sensor, chemoreceptor sensor, electrochemical sensor, amino acid sensor, cholesterol sensor, body fat sensor, osmolality sensor, pH level sensor, sodium sensor, taste sensor, and microbial sensor.

In this example, imaging member 4105 is automatically activated (triggered) to take pictures or record images of food when data from one or more wearable or implanted sensors indicates that person 4101 is consuming food or will probably consume food soon. In this example, imaging member 4105 is automatically activated (triggered) to take pictures or record images of food when data from wrist-worn sensor 4501 indicates that person 4101 is consuming food or will probably consume food soon.

In this example, nutritional intake modification component 5001 comprises virtually-displayed information concerning food 4102. In this example, this information is frowning face 5001 which is shown in proximity to unhealthy food 4102. In an example, virtually-displayed information concerning food can be shown in a person's field of vision as part of augmented reality. In an example, virtually-displayed information concerning food can be shown on the surface of a wearable or mobile device. In this example, nutritional intake modification component 5001 allows normal consumption of nutrients from healthy types and/or quantities of food, but discourages consumption of nutrients from unhealthy types and/or quantities of food. In this example, a nutritional intake modification component discourages consumption and/or absorption of nutrients from unhealthy types and/or quantities of food by displaying images or other visual information in a person's field of view. In this example, a nutritional intake modification component provides negative stimuli in association with unhealthy types and quantities of food and/or provides positive stimuli in association with healthy types and quantities of food. This example can include other types of informational displays and other component variations which were discussed earlier.

FIG. 51 shows an example of how this invention can be embodied in an eyewear-based system and device for monitoring and modifying a person's 4101 nutritional intake comprising: a support member 4103 which is configured to be worn on a person's head; at least one optical member 4104 which is configured to be held in proximity to an eye by the support member; at least one imaging member 4105, wherein the imaging member is part of or attached to the support member or optical member, wherein this imaging member automatically takes pictures or records images of food 4102 when a person is consuming food, and wherein these food pictures or images are automatically analyzed to estimate the type and quantity of food; a data processing unit 4106; and a nutritional intake modification component 5101, wherein this component modifies the person's nutritional intake based on the type and quantity of food. In this example, support member 4103 and two optical members (including 4104) together comprise eyeglasses. In this example, imaging member 4105 is a camera. As discussed earlier, unhealthy types and/or quantities of food can be identified based on food pictures and/or images.

The example in FIG. 51 further comprises wrist-worn sensor 4501. In an example, wrist-worn sensor 4501 can be selected from the group consisting of: glucometer, glucose sensor, glucose monitor, blood glucose monitor, cellular fluid glucose monitor, spectroscopic sensor, food composition analyzer, oximeter, oximetry sensor, pulse oximeter, tissue oximetry sensor, tissue saturation oximeter, wrist oximeter, oxygen consumption monitor, oxygen level monitor, oxygen saturation monitor, ambient air sensor, gas composition sensor, blood oximeter, cutaneous oxygen monitor, capnography sensor, carbon dioxide sensor, carbon monoxide sensor, artificial olfactory sensor, smell sensor, moisture sensor, humidity sensor, hydration sensor, skin moisture sensor, chemiresistor sensor, chemoreceptor sensor, electrochemical sensor, amino acid sensor, cholesterol sensor, body fat sensor, osmolality sensor, pH level sensor, sodium sensor, taste sensor, and microbial sensor.

In this example, imaging member 4105 is automatically activated (triggered) to take pictures or record images of food when data from one or more wearable or implanted sensors indicates that person 4101 is consuming food or will probably consume food soon. In this example, imaging member 4105 is automatically activated (triggered) to take pictures or record images of food when data from wrist-worn sensor 4501 indicates that person 4101 is consuming food or will probably consume food soon.

In this example, nutritional intake modification component 5101 comprises a computer-to-human communication interface. In this example, nutritional intake modification component 5101 sends a communication to person 4101 concerning food 4102 based on evaluation of the healthy or unhealthy attributes of the food. In this example, this communication is conveyed via sonic energy. In this example, nutritional intake modification component 5101 is a speaker. In this example, this communication comprises a voice saying that food 4102 has “a lot of saturated fat”. In other example, a computer-to-human communication can be conveyed via light energy, tactile stimulus, or electromagnetic energy. In an example, a computer-to-human communication can be sent to a person other than person 4101 for dietary support from a friend, social network, and/or healthcare professional. Please see earlier discussion of variations on computer-to-human communication which can be incorporated into this example.

In this example, nutritional intake modification component 5101 allows normal consumption of nutrients from healthy types and/or quantities of food, but discourages consumption of nutrients from unhealthy types and/or quantities of food. In this example, a nutritional intake modification component discourages consumption and/or absorption of nutrients from unhealthy types and/or quantities of food by sending a communication to the person wearing the imaging member and/or to another person. In this example, a nutritional intake modification component provides negative stimuli in association with unhealthy types and quantities of food and/or provides positive stimuli in association with healthy types and quantities of food. This example can include other types of computer-to-human communication and other component variations which were discussed earlier.

FIG. 52 shows an example of how this invention can be embodied in an eyewear-based system and device for monitoring and modifying a person's 4101 nutritional intake comprising: a support member 4103 which is configured to be worn on a person's head; at least one optical member 4104 which is configured to be held in proximity to an eye by the support member; at least one imaging member 4105, wherein the imaging member is part of or attached to the support member or optical member, wherein this imaging member automatically takes pictures or records images of food 4102 when a person is consuming food, and wherein these food pictures or images are automatically analyzed to estimate the type and quantity of food; a data processing unit 4106; and a nutritional intake modification component 4107, wherein this component modifies the person's nutritional intake based on the type and quantity of food. In this example, support member 4103 and two optical members (including 4104) together comprise eyeglasses. In this example, imaging member 4105 is a camera. As discussed earlier, unhealthy types and/or quantities of food can be identified based on food pictures and/or images.

In the example shown in FIG. 52, support member 4103 further comprises at least one upward protrusion 5201 which is configured to span a portion of a person's forehead, temple, and/or a side of the person's head and wherein upward protrusion 5201 holds an electromagnetic brain activity sensor 5202. In this example, support member 4103 further comprises arcuate upward protrusion 5201 which spans a portion of the person's forehead and/or temple. This example comprises at least one electromagnetic energy sensor which measures the conductivity, voltage, impedance, or resistance of electromagnetic energy transmitted through body tissue. In this example, electromagnetic brain activity sensor 5202 is an EEG sensor which is held in place by upward protrusion 5201. In this example, imaging member 4105 is automatically activated (triggered) to take pictures when person 4101 eats, based on a sensor selected from the group consisting of EEG sensor, ECG sensor, and EMG sensor.

In this example, imaging member 4105 is automatically activated (triggered) to take pictures or record images of food when data from one or more wearable or implanted sensors indicates that person 4101 is consuming food or will probably consume food soon. In this example, imaging member 4105 is automatically activated (triggered) to take pictures or record images of food when data from electromagnetic brain activity sensor 5202 indicates that person 4101 is consuming food or will probably consume food soon.

In this example, nutritional intake modification component 4502 is an implanted substance-releasing device. In this example, nutritional intake modification component 4502 allows normal absorption of nutrients from healthy types and/or quantities of food, but reduces absorption of nutrients from unhealthy types and/or quantities of food. In this example, nutritional intake modification component 4502 reduces consumption and/or absorption of nutrients from unhealthy types and/or quantities of food by releasing an absorption-reducing substance into the person's gastrointestinal tract. In this example, nutritional intake modification component 4502 releases an absorption-reducing substance into the person's stomach. This example can include other component variations which were discussed earlier.

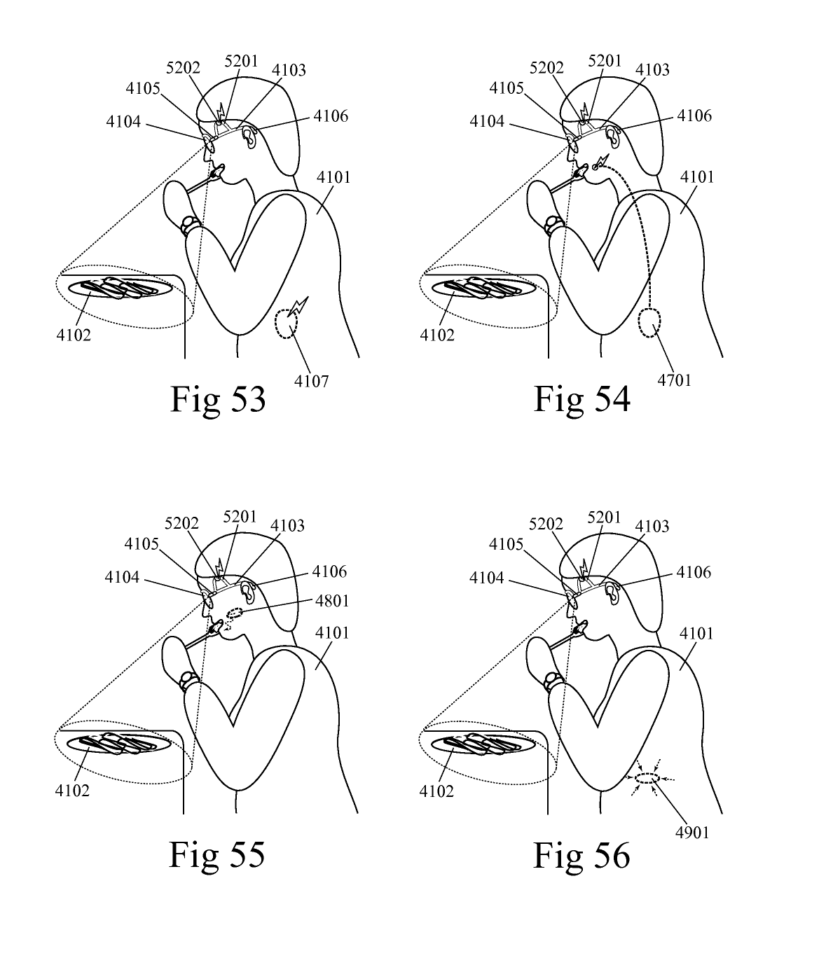

FIG. 53 shows an example of how this invention can be embodied in an eyewear-based system and device for monitoring and modifying a person's 4101 nutritional intake comprising: a support member 4103 which is configured to be worn on a person's head; at least one optical member 4104 which is configured to be held in proximity to an eye by the support member; at least one imaging member 4105, wherein the imaging member is part of or attached to the support member or optical member, wherein this imaging member automatically takes pictures or records images of food 4102 when a person is consuming food, and wherein these food pictures or images are automatically analyzed to estimate the type and quantity of food; a data processing unit 4106; and a nutritional intake modification component 4107, wherein this component modifies the person's nutritional intake based on the type and quantity of food. In this example, support member 4103 and two optical members (including 4104) together comprise eyeglasses. In this example, imaging member 4105 is a camera. As discussed earlier, unhealthy types and/or quantities of food can be identified based on food pictures and/or images.

In the example shown in FIG. 53, support member 4103 further comprises at least one upward protrusion 5201 which is configured to span a portion of a person's forehead, temple, and/or a side of the person's head and wherein upward protrusion 5201 holds an electromagnetic brain activity sensor 5202. In this example, support member 4103 further comprises arcuate upward protrusion 5201 which spans a portion of the person's forehead and/or temple. This example comprises at least one electromagnetic energy sensor which measures the conductivity, voltage, impedance, or resistance of electromagnetic energy transmitted through body tissue. In this example, electromagnetic brain activity sensor 5202 is an EEG sensor which is held in place by upward protrusion 5201. In this example, imaging member 4105 is automatically activated (triggered) to take pictures when person 4101 eats, based on a sensor selected from the group consisting of EEG sensor, ECG sensor, and EMG sensor.

In this example, imaging member 4105 is automatically activated (triggered) to take pictures or record images of food when data from one or more wearable or implanted sensors indicates that person 4101 is consuming food or will probably consume food soon. In this example, imaging member 4105 is automatically activated (triggered) to take pictures or record images of food when data from electromagnetic brain activity sensor 5202 indicates that person 4101 is consuming food or will probably consume food soon.

In this example, nutritional intake modification component 4107 is an implanted electromagnetic energy emitter. In this example, nutritional intake modification component 4107 allows normal absorption of nutrients from healthy types and/or quantities of food, but reduces absorption of nutrients from unhealthy types and/or quantities of food. In this example, nutritional intake modification component 4107 reduces consumption and/or absorption of nutrients from unhealthy types and/or quantities of food by delivering electromagnetic energy to a portion of the person's gastrointestinal tract and/or to nerves which innervate that portion. In this example, nutritional intake modification component 4107 delivers electromagnetic energy to the person's stomach and/or to a nerve which innervates the stomach. FIG. 53 can also include other component variations which were discussed earlier.

FIG. 54 shows an example of how this invention can be embodied in an eyewear-based system and device for monitoring and modifying a person's 4101 nutritional intake comprising: a support member 4103 which is configured to be worn on a person's head; at least one optical member 4104 which is configured to be held in proximity to an eye by the support member; at least one imaging member 4105, wherein the imaging member is part of or attached to the support member or optical member, wherein this imaging member automatically takes pictures or records images of food 4102 when a person is consuming food, and wherein these food pictures or images are automatically analyzed to estimate the type and quantity of food; a data processing unit 4106; and a nutritional intake modification component 4107, wherein this component modifies the person's nutritional intake based on the type and quantity of food. In this example, support member 4103 and two optical members (including 4104) together comprise eyeglasses. In this example, imaging member 4105 is a camera. As discussed earlier, unhealthy types and/or quantities of food can be identified based on food pictures and/or images.

In the example shown in FIG. 54, support member 4103 further comprises at least one upward protrusion 5201 which is configured to span a portion of a person's forehead, temple, and/or a side of the person's head and wherein upward protrusion 5201 holds an electromagnetic brain activity sensor 5202. In this example, support member 4103 further comprises arcuate upward protrusion 5201 which spans a portion of the person's forehead and/or temple. This example comprises at least one electromagnetic energy sensor which measures the conductivity, voltage, impedance, or resistance of electromagnetic energy transmitted through body tissue. In this example, electromagnetic brain activity sensor 5202 is an EEG sensor which is held in place by upward protrusion 5201. In this example, imaging member 4105 is automatically activated (triggered) to take pictures when person 4101 eats, based on a sensor selected from the group consisting of EEG sensor, ECG sensor, and EMG sensor.

In this example, imaging member 4105 is automatically activated (triggered) to take pictures or record images of food when data from one or more wearable or implanted sensors indicates that person 4101 is consuming food or will probably consume food soon. In this example, imaging member 4105 is automatically activated (triggered) to take pictures or record images of food when data from electromagnetic brain activity sensor 5202 indicates that person 4101 is consuming food or will probably consume food soon.

In this example, nutritional intake modification component 4701 is an implanted electromagnetic energy emitter. In this example, nutritional intake modification component 4701 allows normal consumption (and/or absorption) of nutrients from healthy types and/or quantities of food, but reduces consumption (and/or absorption) of nutrients from unhealthy types and/or quantities of food. In this example, nutritional intake modification component 4701 reduces consumption and/or absorption of nutrients from unhealthy types and/or quantities of food by delivering electromagnetic energy to nerves which innervate a person's tongue and/or nasal passages. In an example, this electromagnetic energy can reduce taste and/or smell sensations. In an example, this electromagnetic energy can create virtual taste and/or smell sensations. FIG. 54 can also include other component variations which were discussed earlier.

FIG. 55 shows an example of how this invention can be embodied in an eyewear-based system and device for monitoring and modifying a person's 4101 nutritional intake comprising: a support member 4103 which is configured to be worn on a person's head; at least one optical member 4104 which is configured to be held in proximity to an eye by the support member; at least one imaging member 4105, wherein the imaging member is part of or attached to the support member or optical member, wherein this imaging member automatically takes pictures or records images of food 4102 when a person is consuming food, and wherein these food pictures or images are automatically analyzed to estimate the type and quantity of food; a data processing unit 4106; and a nutritional intake modification component 4107, wherein this component modifies the person's nutritional intake based on the type and quantity of food. In this example, support member 4103 and two optical members (including 4104) together comprise eyeglasses. In this example, imaging member 4105 is a camera. As discussed earlier, unhealthy types and/or quantities of food can be identified based on food pictures and/or images.

In the example shown in FIG. 55, support member 4103 further comprises at least one upward protrusion 5201 which is configured to span a portion of a person's forehead, temple, and/or a side of the person's head and wherein upward protrusion 5201 holds an electromagnetic brain activity sensor 5202. In this example, support member 4103 further comprises arcuate upward protrusion 5201 which spans a portion of the person's forehead and/or temple. This example comprises at least one electromagnetic energy sensor which measures the conductivity, voltage, impedance, or resistance of electromagnetic energy transmitted through body tissue. In this example, electromagnetic brain activity sensor 5202 is an EEG sensor which is held in place by upward protrusion 5201. In this example, imaging member 4105 is automatically activated (triggered) to take pictures when person 4101 eats, based on a sensor selected from the group consisting of EEG sensor, ECG sensor, and EMG sensor.

In this example, imaging member 4105 is automatically activated (triggered) to take pictures or record images of food when data from one or more wearable or implanted sensors indicates that person 4101 is consuming food or will probably consume food soon. In this example, imaging member 4105 is automatically activated (triggered) to take pictures or record images of food when data from electromagnetic brain activity sensor 5202 indicates that person 4101 is consuming food or will probably consume food soon.

In this example, nutritional intake modification component 4801 is an implanted substance-releasing device. In this example, nutritional intake modification component 4801 allows normal consumption (and/or absorption) of nutrients from healthy types and/or quantities of food, but reduces consumption (and/or absorption) of nutrients from unhealthy types and/or quantities of food. In this example, nutritional intake modification component 4801 reduces consumption and/or absorption of nutrients from unhealthy types and/or quantities of food by releasing a taste and/or smell modifying substance into a person's oral cavity and/or nasal passages. In an example, this substance can overpower the taste and/or smell of food. In an example, this substance can be released selectively to make unhealthy food taste or smell bad. FIG. 55 can also include other component variations which were discussed earlier.

FIG. 56 shows an example of how this invention can be embodied in an eyewear-based system and device for monitoring and modifying a person's 4101 nutritional intake comprising: a support member 4103 which is configured to be worn on a person's head; at least one optical member 4104 which is configured to be held in proximity to an eye by the support member; at least one imaging member 4105, wherein the imaging member is part of or attached to the support member or optical member, wherein this imaging member automatically takes pictures or records images of food 4102 when a person is consuming food, and wherein these food pictures or images are automatically analyzed to estimate the type and quantity of food; a data processing unit 4106; and a nutritional intake modification component 4107, wherein this component modifies the person's nutritional intake based on the type and quantity of food. In this example, support member 4103 and two optical members (including 4104) together comprise eyeglasses. In this example, imaging member 4105 is a camera. As discussed earlier, unhealthy types and/or quantities of food can be identified based on food pictures and/or images.

In the example shown in FIG. 56, support member 4103 further comprises at least one upward protrusion 5201 which is configured to span a portion of a person's forehead, temple, and/or a side of the person's head and wherein upward protrusion 5201 holds an electromagnetic brain activity sensor 5202. In this example, support member 4103 further comprises arcuate upward protrusion 5201 which spans a portion of the person's forehead and/or temple. This example comprises at least one electromagnetic energy sensor which measures the conductivity, voltage, impedance, or resistance of electromagnetic energy transmitted through body tissue. In this example, electromagnetic brain activity sensor 5202 is an EEG sensor which is held in place by upward protrusion 5201. In this example, imaging member 4105 is automatically activated (triggered) to take pictures when person 4101 eats, based on a sensor selected from the group consisting of EEG sensor, ECG sensor, and EMG sensor.

In this example, imaging member 4105 is automatically activated (triggered) to take pictures or record images of food when data from one or more wearable or implanted sensors indicates that person 4101 is consuming food or will probably consume food soon. In this example, imaging member 4105 is automatically activated (triggered) to take pictures or record images of food when data from electromagnetic brain activity sensor 5202 indicates that person 4101 is consuming food or will probably consume food soon.

In this example, nutritional intake modification component 4901 is an implanted gastrointestinal constriction device. In this example, nutritional intake modification component 4901 allows normal consumption (and/or absorption) of nutrients from healthy types and/or quantities of food, but reduces consumption (and/or absorption) of nutrients from unhealthy types and/or quantities of food. In this example, nutritional intake modification component 4901 reduces consumption and/or absorption of nutrients from unhealthy types and/or quantities of food by constricting, slowing, and/or reducing passage of food through the person's gastrointestinal tract. In an example, this nutritional intake modification component is a remotely-adjustable gastric band. FIG. 56 can also include other component variations which were discussed earlier.

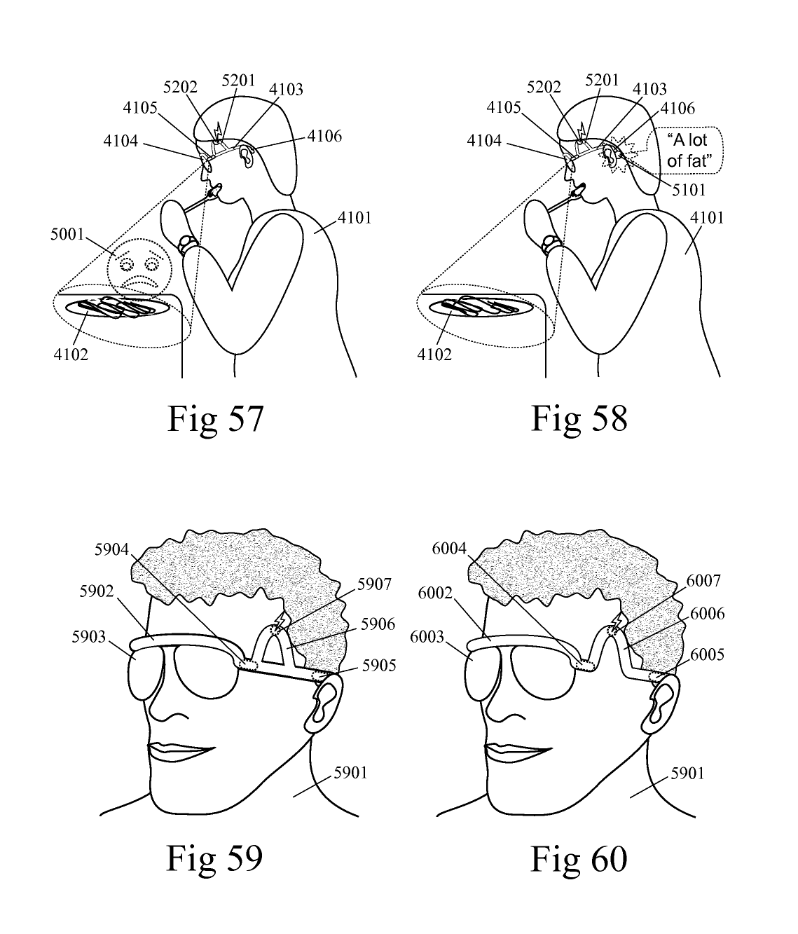

FIG. 57 shows an example of how this invention can be embodied in an eyewear-based system and device for monitoring and modifying a person's 4101 nutritional intake comprising: a support member 4103 which is configured to be worn on a person's head; at least one optical member 4104 which is configured to be held in proximity to an eye by the support member; at least one imaging member 4105, wherein the imaging member is part of or attached to the support member or optical member, wherein this imaging member automatically takes pictures or records images of food 4102 when a person is consuming food, and wherein these food pictures or images are automatically analyzed to estimate the type and quantity of food; a data processing unit 4106; and a nutritional intake modification component 5001, wherein this component modifies the person's nutritional intake based on the type and quantity of food. In this example, support member 4103 and two optical members (including 4104) together comprise eyeglasses. In this example, imaging member 4105 is a camera. As discussed earlier, unhealthy types and/or quantities of food can be identified based on food pictures and/or images.

In the example shown in FIG. 57, support member 4103 further comprises at least one upward protrusion 5201 which is configured to span a portion of a person's forehead, temple, and/or a side of the person's head and wherein upward protrusion 5201 holds an electromagnetic brain activity sensor 5202. In this example, support member 4103 further comprises arcuate upward protrusion 5201 which spans a portion of the person's forehead and/or temple. This example comprises at least one electromagnetic energy sensor which measures the conductivity, voltage, impedance, or resistance of electromagnetic energy transmitted through body tissue. In this example, electromagnetic brain activity sensor 5202 is an EEG sensor which is held in place by upward protrusion 5201. In this example, imaging member 4105 is automatically activated (triggered) to take pictures when person 4101 eats, based on a sensor selected from the group consisting of EEG sensor, ECG sensor, and EMG sensor.

In this example, imaging member 4105 is automatically activated (triggered) to take pictures or record images of food when data from one or more wearable or implanted sensors indicates that person 4101 is consuming food or will probably consume food soon. In this example, imaging member 4105 is automatically activated (triggered) to take pictures or record images of food when data from electromagnetic brain activity sensor 5202 indicates that person 4101 is consuming food or will probably consume food soon.

In this example, nutritional intake modification component 5001 comprises virtually-displayed information concerning food 4102. In this example, this information is frowning face 5001 which is shown in proximity to unhealthy food 4102. In an example, virtually-displayed information concerning food can be shown in a person's field of vision as part of augmented reality. In an example, virtually-displayed information concerning food can be shown on the surface of a wearable or mobile device. In this example, nutritional intake modification component 5001 allows normal consumption of nutrients from healthy types and/or quantities of food, but discourages consumption of nutrients from unhealthy types and/or quantities of food. In this example, a nutritional intake modification component discourages consumption and/or absorption of nutrients from unhealthy types and/or quantities of food by displaying images or other visual information in a person's field of view. In this example, a nutritional intake modification component provides negative stimuli in association with unhealthy types and quantities of food and/or provides positive stimuli in association with healthy types and quantities of food. This example can include other types of informational displays and other component variations which were discussed earlier.

FIG. 58 shows an example of how this invention can be embodied in an eyewear-based system and device for monitoring and modifying a person's 4101 nutritional intake comprising: a support member 4103 which is configured to be worn on a person's head; at least one optical member 4104 which is configured to be held in proximity to an eye by the support member; at least one imaging member 4105, wherein the imaging member is part of or attached to the support member or optical member, wherein this imaging member automatically takes pictures or records images of food 4102 when a person is consuming food, and wherein these food pictures or images are automatically analyzed to estimate the type and quantity of food; a data processing unit 4106; and a nutritional intake modification component 5101, wherein this component modifies the person's nutritional intake based on the type and quantity of food. In this example, support member 4103 and two optical members (including 4104) together comprise eyeglasses. In this example, imaging member 4105 is a camera. As discussed earlier, unhealthy types and/or quantities of food can be identified based on food pictures and/or images.

In the example shown in FIG. 58, support member 4103 further comprises at least one upward protrusion 5201 which is configured to span a portion of a person's forehead, temple, and/or a side of the person's head and wherein upward protrusion 5201 holds an electromagnetic brain activity sensor 5202. In this example, support member 4103 further comprises arcuate upward protrusion 5201 which spans a portion of the person's forehead and/or temple. This example comprises at least one electromagnetic energy sensor which measures the conductivity, voltage, impedance, or resistance of electromagnetic energy transmitted through body tissue. In this example, electromagnetic brain activity sensor 5202 is an EEG sensor which is held in place by upward protrusion 5201. In this example, imaging member 4105 is automatically activated (triggered) to take pictures when person 4101 eats, based on a sensor selected from the group consisting of EEG sensor, ECG sensor, and EMG sensor.

In this example, imaging member 4105 is automatically activated (triggered) to take pictures or record images of food when data from one or more wearable or implanted sensors indicates that person 4101 is consuming food or will probably consume food soon. In this example, imaging member 4105 is automatically activated (triggered) to take pictures or record images of food when data from electromagnetic brain activity sensor 5202 indicates that person 4101 is consuming food or will probably consume food soon.

In this example, nutritional intake modification component 5101 comprises a computer-to-human communication interface. In this example, nutritional intake modification component 5101 sends a communication to person 4101 concerning food 4102 based on evaluation of the healthy or unhealthy attributes of the food. In this example, this communication is conveyed via sonic energy. In this example, nutritional intake modification component 5101 is a speaker. In this example, this communication comprises a voice saying that food 4102 has “a lot of fat”. In other example, a computer-to-human communication can be conveyed via light energy, tactile stimulus, or electromagnetic energy. In an example, a computer-to-human communication can be sent to a person other than person 4101 for dietary support from a friend, social network, and/or healthcare professional. Please see earlier discussion of variations on computer-to-human communication which can be incorporated into this example.

In this example, nutritional intake modification component 5101 allows normal consumption of nutrients from healthy types and/or quantities of food, but discourages consumption of nutrients from unhealthy types and/or quantities of food. In this example, a nutritional intake modification component discourages consumption and/or absorption of nutrients from unhealthy types and/or quantities of food by sending a communication to the person wearing the imaging member and/or to another person. In this example, a nutritional intake modification component provides negative stimuli in association with unhealthy types and quantities of food and/or provides positive stimuli in association with healthy types and quantities of food. This example can include other types of computer-to-human communication and other component variations which were discussed earlier.

FIG. 59 shows an example of eyewear for monitoring a person's electromagnetic brain activity comprising: at least one optical member which is configured to be held in proximity to an eye; a support member with at least one upward protrusion which is configured to span a portion of a person's forehead, temple, and/or a side of the person's head; and at least one electromagnetic brain activity sensor which is held in place by the upward protrusion. The example in FIG. 59 further comprises at least one imaging member and a data processing unit.

Specifically, FIG. 59 shows an example of eyewear for monitoring a person's (5901) electromagnetic brain activity comprising: at least one optical member (5903) which is configured to be held in proximity to an eye; a support member (5902) with at least one upward protrusion (5906) which is configured to span a portion of a person's forehead, temple, and/or a side of the person's head; and at least one electromagnetic brain activity sensor (5907) which is held in place by upward protrusion (5906). The example in FIG. 59 further comprises at least one imaging member (5904) and a data processing unit (5905).

In FIG. 59, upward protrusion 5906 ascends from a side portion of support member 5902. In this example, upward protrusion 5906 has a sinusoidal section shape. In an example, an upward protrusion can have a conic section shape. In this example, upward protrusion 5906 is one of two support member pathways which span from a person's ear to the front of the person's face. In this example, the other support member pathway is relatively straight. In this example, an electromagnetic energy sensor measures the conductivity, voltage, impedance, or resistance of electromagnetic energy transmitted through body tissue. In this example, electromagnetic brain activity sensor 5907 is an EEG sensor which is held in place by upward protrusion 5906. This example can include other component variations which were discussed earlier.

FIG. 60 shows an example of eyewear for monitoring a person's electromagnetic brain activity comprising: at least one optical member which is configured to be held in proximity to an eye; a support member with at least one upward protrusion which is configured to span a portion of a person's forehead, temple, and/or a side of the person's head; and at least one electromagnetic brain activity sensor which is held in place by the upward protrusion. The example in FIG. 60 further comprises at least one imaging member and a data processing unit.

Specifically, FIG. 60 shows an example of eyewear for monitoring a person's (6001) electromagnetic brain activity comprising: at least one optical member (6003) which is configured to be held in proximity to an eye; a support member (6002) with at least one upward protrusion (6006) which is configured to span a portion of a person's forehead, temple, and/or a side of the person's head; and at least one electromagnetic brain activity sensor (6007) which is held in place by upward protrusion (6006). The example in FIG. 60 further comprises at least one imaging member (6004) and a data processing unit (6005).

In FIG. 60, upward protrusion 6006 ascends from a side portion of support member 6002. In this example, upward protrusion 6006 has a sinusoidal section shape. In an example, an upward protrusion can have a conic section shape. In this example, upward protrusion 6006 is the sole pathway which spans from a person's ear to the front of the person's face. In this example, an electromagnetic energy sensor measures the conductivity, voltage, impedance, or resistance of electromagnetic energy transmitted through body tissue. In this example, electromagnetic brain activity sensor 6007 is an EEG sensor which is held in place by upward protrusion 6006. This example can include other component variations which were discussed earlier.

I claim:

1. An eyewear-based system and device for monitoring a person's nutritional intake comprising:
eyeglasses, wherein these eyeglasses further comprise at least one camera, wherein this camera automatically takes pictures or records images of food when a person is consuming food and wherein these food pictures or images are automatically analyzed to estimate the type and quantity of food.

2. An eyewear-based system and device for monitoring and modifying a person's nutritional intake comprising:
eyewear, wherein this eyewear further comprises at least one imaging member, wherein this imaging member automatically takes pictures or records images of food when a person is consuming food, and wherein these food pictures or images are automatically analyzed to estimate the type and quantity of food;
a data processing unit; and
a nutritional intake modification component, wherein this component modifies the person's nutritional intake based on the type and quantity of food.

3. An eyewear-based system and device for monitoring and modifying a person's nutritional intake comprising:
a support member which is configured to be worn on a person's head;
at least one optical member which is configured to be held in proximity to an eye by the support member;
at least one imaging member, wherein the imaging member is part of or attached to the support member or optical member, wherein this imaging member automatically takes pictures or records images of food when a person is consuming food, and wherein these food pictures or images are automatically analyzed to estimate the type and quantity of food;
a data processing unit; and
a nutritional intake modification component, wherein this component modifies the person's nutritional intake based on the type and quantity of food.

4. The system in claim 3 wherein the support member further comprises at least one upward protrusion which is configured to span a portion of a person's forehead, temple, and/or a side of the person's head and wherein this upward protrusion holds an electromagnetic brain activity sensor.

5. The system in claim 3 wherein the imaging member is automatically activated to take pictures when a person eats based on a sensor selected from the group consisting of: accelerometer, inclinometer, and motion sensor.

6. The system in claim 3 wherein the imaging member is automatically activated to take pictures when a person eats based on a sensor selected from the group consisting of: EEG sensor, ECG sensor, and EMG sensor.

7. The system in claim 3 wherein the imaging member is automatically activated to take pictures when a person eats based on a sensor selected from the group consisting of: sound sensor, smell sensor, blood pressure sensor, heart rate sensor, electrochemical sensor, gastric activity sensor, GPS sensor, location sensor, image sensor, optical sensor, piezoelectric sensor, respiration sensor, strain gauge, electrogoniometer, chewing sensor, swallow sensor, temperature sensor, and pressure sensor.

8. The system in claim 3 wherein the imaging member is automatically activated to take pictures when data from one or more wearable or implanted sensors indicates that a person is consuming food or will probably consume food soon.

9. The system in claim 8 wherein at least one sensor is an electromagnetic energy sensor which measures the conductivity, voltage, impedance, or resistance of electromagnetic energy transmitted through body tissue.

10. The system in claim 8 wherein at least one sensor is selected from the group consisting of: glucometer, glucose sensor, glucose monitor, blood glucose monitor, cellular fluid glucose monitor, spectroscopic sensor, food composition analyzer, oximeter, oximetry sensor, pulse oximeter, tissue oximetry sensor, tissue saturation oximeter, wrist oximeter, oxygen consumption monitor, oxygen level monitor, oxygen saturation monitor, ambient air sensor, gas composition sensor, blood oximeter, ear oximeter, cutaneous oxygen monitor, cerebral oximetry monitor, capnography sensor, carbon dioxide sensor, carbon monoxide sensor, artificial olfactory sensor, smell sensor, moisture sensor, humidity sensor, hydration sensor, skin moisture sensor, chemiresistor sensor, chemoreceptor sensor, electrochemical sensor, amino acid sensor, cholesterol sensor, body fat sensor, osmolality sensor, pH level sensor, sodium sensor, taste sensor, and microbial sensor.

11. The system in claim 3 wherein unhealthy food is identified as having a high amount or concentration of one or more nutrients selected from the group consisting of: sugars, simple sugars, simple carbohydrates, fats, saturated fats, cholesterol, and sodium.

12. The system in claim 3 wherein the nutritional intake modification component provides negative stimuli in association with unhealthy types and quantities of food and/or provides positive stimuli in association with healthy types and quantities of food.

13. The system in claim 3 wherein the nutritional intake modification component allows normal absorption of nutrients from healthy types and/or quantities of food, but reduces absorption of nutrients from unhealthy types and/or quantities of food.

14. The system in claim 3 wherein the nutritional intake modification component reduces consumption and/or absorption of nutrients from unhealthy types and/or quantities of food by releasing an absorption-reducing substance into the person's gastrointestinal tract.

15. The system in claim 3 wherein the nutritional intake modification component reduces consumption and/or absorption of nutrients from unhealthy types and/or quantities of food by delivering electromagnetic energy to a portion of the person's gastrointestinal tract and/or to nerves which innervate that portion.

16. The system in claim 3 wherein the nutritional intake modification component reduces consumption and/or absorption of nutrients from unhealthy types and/or quantities of food by delivering electromagnetic energy to nerves which innervate a person's tongue and/or nasal passages.

17. The system in claim 3 wherein the nutritional intake modification component reduces consumption and/or absorption of nutrients from unhealthy types and/or quantities of food by releasing a taste and/or smell modifying substance into a person's oral cavity and/or nasal passages.

18. The system in claim 3 wherein the nutritional intake modification component reduces consumption and/or absorption of nutrients from unhealthy types and/or quantities of food by constricting, slowing, and/or reducing passage of food through the person's gastrointestinal tract.

19. The system in claim 3 wherein the nutritional intake modification component reduces consumption and/or absorption of nutrients from unhealthy types and/or quantities of food by displaying images or other visual information in a person's field of view.

20. The system in claim 3 wherein the nutritional intake modification component reduces consumption and/or absorption of nutrients from unhealthy types and/or quantities of food by sending a communication to the person wearing the imaging member and/or to another person.
